(19) United States
(12) Patent Application Publication
(10) Pub. No.: US 2006/0127894 A1
(43) Pub. Date: Jun. 15, 2006

(54) Protein associated with cell growth, differentiation, and death

(75) Inventors: Yalda Azimzai, Oakland, CA (US); Ines Barroso, Cambridge (GB); Mariah R. Baughn, Los Angeles, CA (US); Shanya d. Becha, San Francisco, CA (US); Mark L. Borowsky, Northampton, MA (US); Brendan M. Duggan, Sunnyvale, CA (US); Vicki S. Elliott, San Jose, CA (US); Brooke M. Emerling, Chicago, IL (US); Ian J. Forsythe, Edmonton, CA (US); Kimberly J. Gietzen, San Jose, CA (US); Ann E. Gorvad, Bellingham, WA (US); Richard C. Graul, San Francisco, CA (US); Jennifer A. Griffin, Fremont, CA (US); Rajagopal Gururajan, San Jose, CA (US); April JA Hafalia, Daly city, CA (US); Craig H. Ison, San Jose, CA (US); Amy E. Kable, Silver Spring, MD (US); Farrah A. Khan, Canton, MI (US); Sally Lee, San Jose, CA (US); Soo yeun Lee, Mountain View, CA (US); Joana S. Li, Millbrae, CA (US); Roopa Reddy, Fremont, CA (US); Thomas W. Richardson, Redwood City, CA (US); William W. Sprague, Sacramento, CA (US); Anita Swarnakar, San Francisco, CA (US); Y. Tom Tang, San Jose, CA (US); Bridget A. Warren, San Marcos, CA (US); Yuming Xu, Mountain View, CA (US); Monique E. Yao, Mountain View, CA (US); Henry Yue, Sunnyvale, CA (US); Huibin Yue, Cupertino, CA (US)

Correspondence Address: Incyte Corporation;Legal Department, 3160 Porter Drive, Palo Alto, CA, 94304, US

(73) Assignee: Incyte Corporation, Palo Alto, CA (US)

(21) Appl. No.: 10/486,020

(22) PCT Filed: Aug. 8, 2002
(86) PCT No.: PCT/US02/25465

§ 371 Date: Jul. 13, 2004

(30) Foreign Application Priority Data
Aug. 8, 2001 (US) 60311017
Aug. 17, 2001 (US) 60313070
Aug. 17, 2001 (US) 60313071
Aug. 24, 2001 (US) 60314678
Aug. 31, 2001 (US) 60316692
Sep. 7, 2001 (US) 60317913
Sep. 14, 2001 (US) 60322182
Dec. 7, 2001 (US) 60340747
Dec. 20, 2001 (US) 60342761
Mar. 29, 2002 (US) 60369129

(51) Int. Cl. C12Q 1/68; G01N 33/53; C07H 21/04; C12P 21/06; C07K 14/475; C07K 16/22; C40B 40/10

(52) U.S. Cl. 435/6; 435/7.1; 435/69.1; 435/320.1; 435/325; 530/388.25; 530/399; 536/23.5

(57) ABSTRACT

Various embodiments of the invention provide human proteins associated with cell growth, differentiation, and death (CGDD) and polynucleotides which identify and encode CGDD. Embodiments of the invention also provide expression vectors, host cells, antibodies, agonists, and antagonists. Other embodiments provide methods for diagnosing, treating, or preventing disorders associated with aberrant expression of CGDD.

### TECHNICAL FIELD

The invention relates to novel nucleic acids, proteins associated with cell growth, differentiation, and death encoded by these nucleic acids, and to the use of these nucleic acids and proteins in the diagnosis, treatment, and prevention of cell proliferative disorders including cancer, developmental disorders, neurological disorders, autoimmune/inflammatory disorders, reproductive disorders, and disorders of the placenta. The invention also relates to the assessment of the effects of exogenous compounds on the expression of nucleic acids and proteins associated with cell growth, differentiation, and death.

### BACKGROUND OF THE INVENTION

Human growth and development requires the spatial and temporal regulation of cell differentiation, cell proliferation, and apoptosis. These processes coordinately control reproduction, aging, embryogenesis, morphogenesis, organogenesis, and tissue repair and maintenance. At the cellular level, growth and development is governed by the cell's decision to enter into or exit from the cell division cycle and by the cell's commitment to a terminally differentiated state. These decisions are made by the cell in response to extracellular signals and other environmental cues it receives. The following discussion focuses on the molecular mechanisms of cell division, embryogenesis, cell differentiation and proliferation, and apoptosis, as well as disease states such as cancer which can result from disruption of these mechanisms.

### Cell Cycle

Cell division is the fundamental process by which all living things grow and reproduce. In unicellular organisms such as yeast and bacteria, each cell division doubles the number of organisms. In multicellular species many rounds of cell division are required to replace cells lost by wear or by programmed cell death, and for cell differentiation to produce a new tissue or organ. Progression through the cell cycle is governed by the intricate interactions of protein complexes. This regulation depends upon the appropriate expression of proteins which control cell cycle progression in response to extracellular signals, such as growth factors and other mitogens, and intracellular cues, such as DNA damage or nutrient starvation. Molecules which directly or indirectly modulate cell cycle progression fall into several categories, including cyclins, cyclin-dependent protein kinases, growth factors and their receptors, second messenger and signal transduction proteins, oncogene products, and tumor-suppressor proteins.

Details of the cell division cycle may vary, but the basic process consists of three principle events. The first event, interphase, involves preparations for cell division, replication of the DNA, and production of essential proteins. In the second event, mitosis, the nuclear material is divided and separates to opposite sides of the cell. The final event, cytokinesis, is division and fission of the cell cytoplasm. The sequence and timing of cell cycle transitions is under the control of the cell cycle regulation system which controls the process by positive or negative regulatory circuits at various check points.

Mitosis marks the end of interphase and concludes with the onset of cytokinesis. There are four stages in mitosis, occurring in the following order: prophase, metaphase, anaphase and telophase. Prophase includes the formation of bi-polar mitotic spindles, composed of microtubules and associated proteins such as dynein, which originate from polar mitotic centers. During metaphase, the nuclear material condenses and develops kinetochore fibers which aid in its physical attachment to the mitotic spindles. The ensuing movement of the nuclear material to opposite poles along the mitotic spindles occurs during anaphase. Telophase includes the disappearance of the mitotic spindles and kinetochore fibers from the nuclear material. Mitosis depends on the interaction of numerous proteins. For example, centromere-associated proteins such as CENP-A, -B, and -C, play structural roles in kinetochore formation and assembly (Saffery, R. et al. (2000) Human Mol. Gen. 9:175-185).

During the M phase of eukaryotic cell cycling, structural rearrangements occur ensuring appropriate distribution of cellular components between daughter cells. Breakdown of interphase structures into smaller subunits is common. The nuclear envelope breaks into vesicles, and nuclear lamins are disassembled. Subsequent phosphorylation of these lamins occurs and is maintained until telophase, at which time the nuclear lamina structure is reformed. cDNAs responsible for encoding M phase phosphorylation (MPPs) are components of U3 small nucleolar ribonucleoprotein (snoRNP), and relocalize to the nucleolus once mitosis is complete (Westendorf, J. M. et al. (1998) J. Biol. Chem. 9:437449). U3 snoRNPs are essential mediators of RNA processing events.

Proteins involved in the regulation of cellular processes such as mitosis include the Ser/Thr-protein phosphatases type 1 (PP-1). PP-1s act by dephosphorylation of key proteins involved in the metaphase-anaphase transition. The gene PP1R7 encodes the regulatory polypeptide sds22, having at least six splice variants (Ceulemans, H. et al. (1999) Eur. J. Biochem. 262:36-42). Sds22 modulates the activity of the catalytic subunit of PP-1s, and enhances the PP-1-dependent dephosphorylation of mitotic substrates.

Cell cycle regulatory proteins play an important role in cell proliferation and cancer. For example, failures in the proper execution and timing of cell cycle events can lead to chromosome segregation defects resulting in aneuploidy or polyploidy. This genomic instability is characteristic of transformed cells (Luca, F. C. and M. Winey (1998) Mol. Biol. Cell. 9:2946). A recently identified protein, mMOB1, is the mammalian homolog of yeast MOB1, an essential yeast gene required for completion of mitosis and maintenance of ploidy. The mammalian mMOB1 is a member of protein complexes including protein phosphatase 2A (PP2A), and its phosphorylation appears to be regulated by PP2A (Moreno, C. S. et al. (2001) J. Biol. Chem. 276:24253-24260). PP2A has been implicated in the development of human cancers, including lung and colon cancers and leukemias.

ERM proteins are responsible for the cross-linking of actin filaments to the plasma membrane. FERM domains, located at the N-terminal regions of ERM proteins, regulate interactions between the cytoplasmic domains of the integrated membrane proteins with the membrane itself. The Protein 4.1 family of molecules are responsible for linking the actin cytoskeleton to cell surface glycoproteins. For example, the neurofibromatosis 2 (NF2) tumor suppressor is a member of the Protein 4.1 family. NF2 proteins participate in suppression of cell growth, and retard other cytoskeletal-dependent functions including cell spreading, attachment and motility (Gutmann, D. H. et al. (2001) Neurobiol. Dis. 8:266-278). Recently, a novel putative tumor suppressor gene and member of the NF2/ERM4.1 superfamily has been observed to retard the growth of non-small cell lung carcinoma cells (Tran, Y. K. et al. (1999) Cancer Res. 59:35-43).

Cell cycle regulation involves numerous proteins interacting in a sequential manner. The eukaryotic cell cycle consists of several highly controlled events whose precise order ensures successful DNA replication and cell division. Cells maintain the order of these events by making later events dependent on the successful completion of earlier events. This dependency is enforced by cellular mechanisms called checkpoints. Examples of additional cell cycle regulatory proteins include the histone deacetylases (HDACs). HDACs are involved in cell cycle regulation, and modulate chromatin structure. Human HDAC1 has been found to interact in vitro with the human Hus1 gene product, whose Schizosaccharomyces pombe homolog has been implicated in G₂/M checkpoint control (Cai, R. L. et al. (2000) J. Biol. Chem 275:27909-27916).

DNA damage (G₂) and DNA replication (S-phase) checkpoints arrest eukaryotic cells at the G₂/M transition. This arrest provides time for DNA repair or DNA replication to occur before entry into mitosis. Thus, the G₂/M checkpoint ensures that mitosis only occurs upon completion of DNA replication and in the absence of chromosomal damage. The Hus1 gene of Schizosaccharomyces pombe is a cell cycle checkpoint gene, as are the rad family of genes (e.g., rad1 and rad9) (Volkmer, E. and L. M. Karnitz (1999) J. Biol. Chem. 274:567-570; Kostrub C. F. et al. (1998) EMBO J. 17:2055-2066). These genes are involved in the mitotic checkpoint, and are induced by either DNA damage or blockage of replication. Induction of DNA damage or replication block leads to loss of function of the Hus1 gene and subsequent cell death. Human homologs have been identified for most of the rad genes, including ATM and AIR, the human homologs of rad3p. Mutations in the ATM gene are correlated with the severe congenital disease ataxia-telagiectasia (Savitsky, K. et al. (1995) Science 268:1749-1753). The human Hus1 protein has been shown to act in a complex with rad1 protein which interacts with rad9, making them central components of a DNA damage-responsive protein complex of human cells (Volkner and Kamitz, supra).

The entry and exit of a cell from mitosis is regulated by the synthesis and destruction of a family of activating proteins called cyclins. Cyclins act by binding to and activating a group of cyclin-dependent protein kinases (Cdks) which then phosphorylate and activate selected proteins involved in the mitotic process. Cyclins are characterized by a large region of shared homology that is approximately 180 amino acids in length and referred to as the “cyclin box” (Chapman, D. L. and D. J. Wolgemuth (1993) Development 118:229-240). In addition, cyclins contain a conserved 9 amino acid sequence in the N-terminal region of the molecule called the “destruction box.” This sequence is believed to be a recognition code that triggers ubiquitin-mediated degradation of cyclin B (Hunt, T. (1991) Nature 349:100-101). Several types of cyclins exist (Ciechanover, A. (1994) Cell 79:13-21). Progression through G1 and S phase is driven by the G1 cyclins and their catalytic subunits, including Cdk2-cyclin A, Cdk2-cyclin E, Cdk4-cyclin D and Cdk6-cyclin D. Progression through the G2-M transition is driven by the activation of mitotic CDK-cyclin complexes such as Cdc2-cyclin A, Cdc2-cyclin B 1 and Cdc2-cyclin B2 complexes (reviewed in Yang, J. and S. Kombluth (1999) Trends Cell Biol. 9:207-210).

Cyclins are degraded through the ubiquitin conjugation system (UCS), a major pathway for the degradation of cellular proteins in eukaroytic cells and in some bacteria. The UCS mediates the elimination of abnormal proteins and regulates the half-lives of important regulatory proteins that control cellular processes such as gene transcription and cell cycle progression. The UCS is implicated in the degradation of mitotic cyclin kinases, oncoproteins, tumor suppressor genes such as p53, viral proteins, cell surface receptors associated with signal transduction, transcriptional regulators, and mutated or damaged proteins (Ciechanover, supra).

The process of ubiquitin conjugation and protein degradation occurs in five principle steps (Jentsch, S. (1992) Annu. Rev. Genet. 26:179-207). First ubiquitin (Ub), a small, heat stable protein is activated by a ubiquitin-activating enzyme (E1) in an ATP dependent reaction which binds the C-terminus of Ub to the thiol group of an internal cysteine residue in El. Second, activated Ub is transferred to one of several Ub-conjugating enzymes (E2). Different ubiquitin-dependent proteolytic pathways employ structurally similar, but distinct ubiquitin-conjugating enzymes that are associated with recognition subunits which direct them to proteins carrying a particular degradation signal. Third, E2 transfers the Ub molecule through its C-terminal glycine to a member of the ubiquitin-protein ligase family, E3. Fourth, E3 transfers the Ub molecule to the target protein. Additional Ub molecules may be added to the target protein forming a multi-Ub chain structure. Fifth, the ubiquinated protein is then recognized and degraded by the proteasome, a large, multisubunit proteolytic enzyme complex, and Ub is released for re-utilization.

Prior to activation, Ub is usually expressed as a fusion protein composed of an N-terminal ubiquitin and a C-terminal extension protein (CEP) or as a polyubiquitin protein with Ub monomers attached head to tail. CEPs have characteristics of a variety of regulatory proteins; most are highly basic, contain up to 30% lysine and arginine residues, and have nucleic acid-binding domains (Monia, B. P. et al. (1989) J. Biol. Chem. 264:4093-4103). The fusion protein is an important intermediate which appears to mediate co-regulation of the cell's translational and protein degradation activities, as well as localization of the inactive enzyme to specific cellular sites. Once delivered, C-terminal hydrolases cleave the fusion protein to release a functional Ub (Monia et al., supra).

Ub-conjugating enzymes (E2s) are important for substrate specificity in different UCS pathways. All E2s have a conserved domain of approximately 16 kDa called the UBC domain that is at least 35% identical in all E2s and contains a centrally located cysteine residue required for ubiquitin-enzyme thiolester formation (Jentsch, supra). A well conserved proline-rich element is located N-terminal to the active cysteine residue. Structural variations beyond this conserved domain are used to classify the E2 enzymes. Class I E2s consist almost exclusively of the conserved UBC domain. Class II E2s have various unrelated C-terminal extensions that contribute to substrate specificity and cellular localization. Class III E2s have unique N-terminal extensions which are believed to be involved in enzyme regulation or substrate specificity.

The E2s are important for substrate specificity in several UCS pathways. All E2s have a conserved ubiquitin conjugation (UBC) domain of approximately 16 kD, at least 35% identity with each other, and contain a centrally located cysteine residue which is necessary for ubiquitin-enzyme thiolester formation (Jentsch, supra). A highly conserved proline-rich element is located N-terminal to the active cysteine residue. Structural variations outside of this conserved domain are used to separate the E2 enzymes into classes. The E2s of class 1 (E2-1) consist of the conserved UBC domain and include yeast B2-1 and UBCs 4, 5, and 7. These E2s are thought to require E3 to carry out their activities (Jentsch, supra). UBC7 has been shown to recognize ubiquitin as a substrate and to form polyubiquitin chains in vitro (van Nocker, S. et al. (1996) J. Biol. Chem. 271:12150-58). E2s of class 2 (E2-2) have various unrelated C-terminal extensions that contribute to substrate specificity and cellular localization. The yeast E2-2 enzymes, UBC2 and UBC3, have highly acidic C-terminal extensions that promote interactions with basic substrates such as histones. Yeast UBC6 has a hydrophobic signal-anchor sequence that localizes the protein to the endoplasmic reticulum.

A mitotic cyclin-specific E2 (E2-C) is characterized by the conserved UBC domain, an N-terminal extension of 30 amino acids not found in other E2s, and a 7 amino acid unique sequence adjacent to this extension. These characteristics together with the high affinity of E2-C for cyclin identify it as a new class of E2 (Aristarkhov, A. et al. (1996) Proc. Natl. Acad. Sci. 93:4294-99).

Ubiquitin-protein ligases (E3s) catalyze the last step in the ubiquitin conjugation process, covalent attachment of ubiquitin to the substrate. E3 plays a key role in determining the specificity of the process. Only a few E3s have been identified so far. One type of E3 ligases is the HECT homologous to E6-AP C-terminus) domain protein family. One member of the family, E6-AP (E6-associated protein) is required, along with the human papillomavirus (HPV) E6 oncoprotein, for the ubiquitination and degradation of p53 (Scheffner, M. et al. (1993) Cell 75:495-505). The C-terminal domain of HECT proteins contains the highly conserved ubiquitin-binding cysteine residue. The N-terminal region of the various HECT proteins is variable and is believed to be involved in specific substrate recognition (Huibregtse, J. M. et al. (1997) Proc. Natl Acad. Sci. USA 94:3656-3661). The SCF (Skp1-Cdc53/Cullin-F box receptor) family of proteins comprise another group of ubiquitin ligases (Deshaies, R. (1999) Annu. Rev. Dev. Biol. 15:435-467). Multiple proteins are recruited into the SCF complex, including Skp1, cullin, and an F box domain containing protein. The F box protein binds the substrate for the ubiquitination reaction and may play roles in determining substrate specificity and orienting the substrate for reaction. Skp1 interacts with both the F box protein and cullin and may be involved in positioning the F box protein and cullin in the complex for transfer of ubiquitin from the E2 enzyme to the protein substrate. Substrates of SCF ligases include proteins involved in regulation of CDK activity, activation of transcription, signal transduction, assembly of kinetochores, and DNA replication.

Sgt1 was identified in a screen for genes in yeast that suppress defects in kinetochore function caused by mutations in Skp1 (Kitagawa, K. et al. (1999) Mol. Cell 4:21-33). Sgt1 interacts with Skp1 and associates with SCF ubiquitin ligase. Defects in Sgt1 cause arrest of cells at either G1 or G2 stages of the cell cycle. A yeast Sgt1 null mutant can be rescued by human Sgt1, an indication of the conservation of Sgt1 function across species. Sgt1 is required for assembly of kinetochore complexes in yeast.

Abnormal activities of the UCS are implicated in a number of diseases and disorders. These include, e.g., cachexia (Llovera, M. et al. (1995) Int. J. Cancer 61:138-141), degradation of the tumor-suppressor protein, p53 (Ciechanover, supra), and neurodegeneration such as observed in Alzheimer's disease (Gregori, L. et al. (1994) Biochem. Biophys. Res. Commun. 203:1731-1738). Since ubiquitin conjugation is a rate-limiting step in antigen presentation, the ubiquitin degradation pathway may also have a critical role in the immune response (Grant, E. P. et al. (1995) J. Immunol. 155:3750-3758).

Certain cell proliferation disorders can be identified by changes in the protein complexes that normally control progression through the cell cycle. A primary treatment strategy involves reestablishing control over cell cycle progression by manipulation of the proteins involved in cell cycle regulation (Nigg, E. A. (1995) BioEssays 17:471-480).

Regulated progression of the cell cycle depends on the integration of growth control pathways with the basic cell cycle machinery. Cell cycle regulators have been identified by selecting for human and yeast cDNAs that block or activate cell cycle arrest signals in the yeast mating pheromone pathway when they are overexpressed. Known regulators include human CPR (cell cycle progression restoration) genes, such as CPR8 and CPR2, and yeast CDC (cell division control) genes, including CDC91, that block the arrest signals. The CPR genes express a variety of proteins including cyclins, tumor suppressor binding proteins, chaperones, transcription factors, translation factors, and RNA-binding proteins (Edwards, M. C. et al.(1997) Genetics 147:1063-1076).

Several cell cycle transitions, including the entry and exit of a cell from mitosis, are dependent upon the activation and inhibition of cyclin-dependent kinases (Cdks). The Cdks are composed of a kinase subunit, Cdk, and an activating subunit, cyclin, in a complex that is subject to many levels of regulation. There appears to be a single Cdk in Saccharomyces cerevisiae and Saccharomyces pombe whereas mammals have a variety of specialized Cdks. Cyclins act by binding to and activating cyclin-dependent protein kinases which then phosphorylate and activate selected proteins involved in the mitotic process. The Cdk-cyclin complex is both positively and negatively regulated by phosphorylation, and by targeted degradation involving molecules such as CDC4 and CDC53. In addition, Cdks are further regulated by binding to inhibitors and other proteins such as Suc1 that modify their specificity or accessibility to regulators (Patra, D. and W. G. Dunphy (1996) Genes Dev. 10: 1503-1515; and Mathias, N. et al. (1996) Mol. Cell Biol. 16:6634-6643).

### Reproduction

The male and female reproductive systems are complex and involve many aspects of growth and development. The anatomy and physiology of the male and female reproductive systems are reviewed in (Guyton, A. C. (1991) Textbook of Medical Physiology, W. B. Saunders Co., Philadelphia Pa., pp. 899-928).

The male reproductive system includes the process of spermatogenesis, in which the sperm are formed, and male reproductive functions are regulated by various hormones and their effects on accessory sexual organs, cellular metabolism, growth, and other bodily functions.

Spermatogenesis begins at puberty as a result of stimulation by gonadotropic hormones released from the anterior pituitary. Immature sperm (spermatogonia) undergo several mitotic cell divisions before undergoing meiosis and full maturation. The testes secrete several male sex hormones, the most abundant being testosterone, that is essential for growth and division of the immature sperm, and for the masculine characteristics of the male body. Three other male sex hormones, gonadotropin-releasing hormone (GnRH), luteinizing hormone (LH), and follicle-stimulating hormone (FSH) control sexual function.

The uterus, ovaries, fallopian tubes, vagina, and breasts comprise the female reproductive system. The ovaries and uterus are the source of ova and the location of fetal development, respectively. The fallopian tubes and vagina are accessory organs attached to the top and bottom of the uterus, respectively. Both the uterus and ovaries have additional roles in the development and loss of reproductive capability during a female's lifetime. The primary role of the breasts is lactation. Multiple endocrine signals from the ovaries, uterus, pituitary, hypothalamus, adrenal glands, and other tissues coordinate reproduction and lactation. These signals vary during the monthly menstruation cycle and during the female's lifetime. Similarly, the sensitivity of reproductive organs to these endocrine signals varies during the female's lifetime.

A combination of positive and negative feedback to the ovaries, pituitary and hypothalamus glands controls physiologic changes during the monthly ovulation and endometrial cycles. The anterior pituitary secretes two major gonadotropin hormones, follicle-stimulating hormone (FSH) and luteinizing hormone (LH), regulated by negative feedback of steroids, most notably by ovarian estradiol. If fertilization does not occur, estrogen and progesterone levels decrease. This sudden reduction of the ovarian hormones leads to menstruation, the desquamation of the endometrium/

Hormones further govern all the steps of pregnancy, parturition, lactation, and menopause. During pregnancy large quantities of human chorionic gonadotropin (hCG), estrogens, progesterone, and human chorionic somatomammotropin (hCS) are formed by the placenta. hCG, a glycoprotein similar to luteinizing hormone, stimulates the corpus luteum to continue producing more progesterone and estrogens, rather than to involute as occurs if the ovum is not fertilized. hCS is similar to growth hormone and is crucial for fetal nutrition.

The female breast also matures during pregnancy. Large amounts of estrogen secreted by the placenta trigger growth and branching of the breast milk ductal system while lactation is initiated by the secretion of prolactin by the pituitary gland.

Parturition involves several hormonal changes that increase uterine contractility toward the end of pregnancy, as follows. The levels of estrogens increase more than those of progesterone. Oxytocin is secreted by the neurohypophysis. Concomitantly, uterine sensitivity to oxytocin increases. The fetus itself secretes oxytocin, cortisol (from adrenal glands), and prostaglandins.

Menopause occurs when most of the ovarian follicles have degenerated. The ovary then produces less estradiol, reducing the negative feedback on the pituitary and hypothalamus glands. Mean levels of circulating FSH and LH increase, even as ovulatory cycles continue. Therefore, the ovary is less responsive to gonadotropins, and there is an increase in the time between menstrual cycles. Consequently, menstrual bleeding ceases and reproductive capability ends.

### Embryogenesis

Mammalian embryogenesis is a process which encompasses the first few weeks of development following conception. During this period, embryogenesis proceeds from a single fertilized egg to the formation of the three embryonic tissues, then to an embryo which has most of its internal organs and all of its external features.

The normal course of mammalian embryogenesis depends on the correct temporal and spatial regulation of a large number of genes and tissues. These regulation processes have been intensely studied in mouse. An essential process that is still poorly understood is the activation of the embryonic genome after fertilization. As mouse oocytes grow, they accumulate transcripts that are either translated directly into proteins or stored for later activation by regulated polyadenylation. During subsequent meiotic maturation and ovulation, the maternal genome is transcriptionally inert, and most maternal transcripts are deadenylated and/or degraded prior to, or together with, the activation of the zygotic genes at the two-cell stage (Stutz, A. et al. (1998) Genes Dev. 12:2535-2548). The maternal to embryonic transition involves the degradation of oocyte, but not zygotic transcripts, the activation of the embryonic genome, and the induction of cell cycle progression to accommodate early development.

MATER (Maternal Antigen That Embryos Require) was initially identified as a target of antibodies from mice with ovarian immunity (Tong, Z-B. and L. M. Nelson (1999) Endocrinology 140:3720-3726). Expression of the gene encoding MATER is restricted to the oocyte, making it one of a limited number of known maternal-effect genes in mammals (Tong, Z-B. et al. (2000) Mamm. Genome 11:281-287). The MATER protein is required for embryonic development beyond two cells, based upon preliminary results from mice in which this gene has been inactivated. The 1111-amino acid MATER protein contains a hydrophilic repeat region in the amino terminus, and a region containing 14 leucine-rich repeats in the carboxyl terminus. These repeats resemble the sequence found in porcine ribonuclease inhibitor that is critical for protein-protein interactions.

The degradation of maternal transcripts during meiotic maturation and ovulation may involve the activation of a ribonuclease just prior to ovulation. Thus the function of MATER may be to bind to the maternal ribonuclease and prevent degradation of zygotic transcripts (Tong et al., supra). In addition to its role in oocyte development and embryogenesis, MATER may also be relevant to the pathogenesis of ovarian immunity, as it is a target of autoantibodies in mice with autoimmune oophoritis (Tong and Nelson, supra).

The maternal mRNA D7 is a moderately abundant transcript in Xenopus laevis whose expression is highest in, and perhaps restricted to, oogenesis and early embryogenesis. The D7 protein is absent from oocytes and first begins to accumulate during oocyte maturation. Its levels are highest during the first day of embryonic development and then they decrease. The loss of D7 protein affects the maturation process itself, significantly delaying the time course of germinal vesicle breakdown. Thus, D7 is a newly described protein involved in oocyte maturation (Smith, R. C. et al. (1988) Genes Dev. 2(10):1296-306.)

Many other genes are involved in subsequent stages of embryogenesis. After fertilization, the oocyte is guided by fimbria at the distal end of each fallopian tube into and through the fallopian tube and thence into the uterus. Changes in the uterine endometrium prepare the tissue to support the implantation and embryonic development of a fertilized ovum. Several stages of division have occurred before the dividing ovum, now a blastocyst with about 100 cells, enters the uterus. Upon reaching the uterus, the developing blastocyst usually remains in the uterine cavity an additional two to four days before implanting in the endometrium, the inner lining of the uterus. Implantation results from the action of trophoblast cells that develop over the surface of the blastocyst. These cells secrete proteolytic enzymes that digest and liquefy the cells of the endometrium. The invasive process is reviewed in Fisher, S. J. and C. H. Damsky (1993; Semin Cell Biol 4:183-188) and Graham, C. H. and P. K. Lala (1992; Biochem Cell Biol 70:867-874). Once implantation has taken place, the trophoblast and other sublying cells proliferate rapidly, forming the placenta and the various membranes of pregnancy. (See Guyton, A. C. (1991) Textbook of Medical Physiology, 8^{th} ed. W. B. Saunders Company, Philadelphia Pa., pp. 915-919.)

The placenta has an essential role in protecting and nourishing the developing fetus. In most species the syncytiotrophoblast layer is present on the outside of the placenta at the fetal-maternal interface. This is a continuous structure, one cell deep, formed by the fusion of the constituent trophoblast cells. The syncytiotrophoblast cells play important roles in maternal-fetal exchange, in tissue remodeling during fetal development, and in protecting the developing fetus from the maternal immune response (Stoye, J. P. and J. M. Coffin (2000) Nature 403:715-717).

A gene called syncytin is the envelope gene of a human endogenous defective provirus. Syncytin is expressed in high levels in placenta, and more weakly in testis, but is not detected in any other tissues (Mi, S. et al. (2000) Nature 403:785-789). Syncytin expression in the placenta is restricted to the syncytiotrophoblasts. Since retroviral env proteins are often involved in promoting cell fusion events, it was thought that syncytin might be involved in regulating the fusion of trophoblast cells into the syncytiotrophoblast layer. Experiments demonstrated that syncytin can mediate cell fusion in vitro, and that anti-syncytin antibodies can inhibit the fusion of placental cytotrophoblasts (Mi et al., supra). In addition, a conserved immunosuppressive domain present in retroviral envelope proteins, and found in syncytin at amino acid residues 373-397, might be involved in preventing maternal immune responses against the developing embryo.

Syncytin may also be involved in regulating trophoblast invasiveness by inducing trophoblast fusion and terminal differentiation (Mi et al., supra). Insufficient trophoblast infiltration of the uterine wall is associated with placental disorders such as preeclampsia, or pregnancy induced hypertension, while uncontrolled trophoblast invasion is observed in choriocarcinoma and other gestational trophoblastic diseases. Thus syncytin function may be involved in these diseases.

### Cell Differentiation

Multicellular organisms are comprised of diverse cell types that differ dramatically both in structure and function, despite the fact that each cell is like the others in its hereditary endowment. Cell differentiation is the process by which cells come to differ in their structure and physiological function. The cells of a multicellular organism all arise from mitotic divisions of a single-celled zygote. The zygote is totipotent, meaning that it has the ability to give rise to every type of cell in the adult body. During development the cellular descendants of the zygote lose their totipotency and become determined. Once its prospective fate is achieved, a cell is said to have differentiated. All descendants of this cell will be of the same type.

Human growth and development requires the spatial and temporal regulation of cell differentiation, along with cell proliferation and regulated cell death. These processes coordinate to control reproduction, aging, embryogenesis, morphogenesis, organogenesis, and tissue repair and maintenance. The processes involved in cell differentiation are also relevant to disease states such as cancer, in which case the factors regulating normal cell differentiation have been altered, allowing the cancerous cells to proliferate in an anaplastic, or undifferentiated, state.

The mechanisms of differentiation involve cell-specific regulation of transcription and translation, so that different genes are selectively expressed at different times in different cells. Genetic experiments using the fruit fly Drosophila melanogaster have identified regulated cascades of transcription factors which control pattern formation during development and differentiation. These include the homeotic genes, which encode transcription factors containing homeobox motifs. The products of homeotic genes determine how the insect's imaginal discs develop from masses of undifferentiated cells to specific segments containing complex organs. These proteins are critical for specifying the anterior-posterior body axis during development. Many genes found to be involved in cell differentiation and development in Drosophila have homologs in mammals. Some human genes have equivalent developmental roles to their Drosophila homologs. The human homolog of the Drosophila eyes absent gene (eya) underlies branchio-oto-renal syndrome, a developmental disorder affecting the ears and kidneys (Abdelhak, S. et al. (1997) Nat. Genet. 15:157-164). In Drosophila, Iroquois homeobox genes play roles in the control of proneural and vein-forming genes, the positional identity of sensory neurons, alula and notum formation, and the establishment of a dorsoventral pattern organizing center necessary for follicle, head and eye development. Mammalian Iroquois homeobox genes are involved in the development of the nervous system and heart (Christoffels, V. M. et al. (2000) Dev. Biol. 224:263-274).

Tesmin is a testis-specific metallothionein-like protein that is specifically expressed in spermatocytes and may play a role in spermatogenesis (Sugihara, T. et al. (1999) Genomics 57:130-136). Metallothionein proteins are low-molecular-weight cysteine-rich proteins that bind to heavy metal ions. Metallothionein proteins may bind and sequester toxic heavy metals, act as metal storage molecules, and control concentrations of essential heavy metals, particularly zinc. By regulating the availability of metal ions such as zinc required for certain enzymes and transcription factors, metallothioneins may have roles in cell growth and differentiation.

At the cellular level, growth and development are governed by the cell's decision to enter into or exit from the cell cycle and by the cell's commitment to a terminally differentiated state. Differential gene expression within cells is triggered in response to extracellular signals and other environmental cues. Such signals include growth factors and other mitogens such as retinoic acid; cell-cell and cell-matrix contacts; and environmental factors such as nutritional signals, toxic substances, and heat shock. Candidate genes that may play a role in differentiation can be identified by altered expression patterns upon induction of cell differentiation in vitro.

The final step in cell differentiation results in a specialization that is characterized by the production of particular proteins, such as contractile proteins in muscle cells, serum proteins in liver cells and globins in red blood cell precursors. The expression of these specialized proteins depends at least in part on cell-specific transcription factors. For example, the homeobox-containing transcription factor PAX-6 is essential for early eye determination, specification of ocular tissues, and normal eye development in vertebrates.

In the case of epidermal differentiation, the induction of differentiation-specific genes occurs either together with or following growth arrest and is believed to be linked to the molecular events that control irreversible growth arrest. Irreversible growth arrest is an early event which occurs when cells transit from the basal to the innermost suprabasal layer of the skin and begin expressing squamous-specific genes. These genes include those involved in the formation of the cross-linked envelope, such as transglutaminase I and III, involucrin, loricin, and small proline-rich repeat (SPRR) proteins. The SPRR proteins are 8-10 kDa in molecular mass, rich in proline, glutamine, and cysteine, and contain similar repeating sequence elements. The SPRR proteins may be structural proteins with a strong secondary structure or metal-binding proteins such as metallothioneins. (Jetten, A. M. and B. L. Harvat (1997) J. Dermatol. 24:711-725; PRINTS Entry PR00021 PRORICH Small proline-rich protein signature.)

The Wnt gene family of secreted signaling molecules is highly conserved throughout eukaryotic cells. Members of the Wnt family are involved in regulating chondrocyte differentiation within the cartilage template. Wnt-5a, Wnt-5b and Wnt-4 genes are expressed in chondrogenic regions of the chicken limb, Wnt-Sa being expressed in the perichondrium (mesenchymal cells immediately surrounding the early cartilage template). Wnt-5a misexpression delays the maturation of chondrocytes and the onset of bone collar formation in chicken limb (Hartmn, C. and C. J. Tabin (2000) Development 127:3141-3159).

Glypicans are a family of cell surface heparan sulfate proteoglycans that play an important role in cellular growth control and differentiation. Cerebroglycan, a heparan sulfate proteoglycan expressed in the nervous system, is involved with the motile behavior of developing neurons (Stipp, C. S. et al. (1994) J. Cell Biol. 124:149-160).

Notch plays an active role in the differentiation of glial cells, and influences the length and organization of neuronal processes (for a review, see Frisen, J. and U. Lendahl (2001) Bioessays 23:3-7). The Notch receptor signaling pathway is important for morphogenesis and development of many organs and tissues in multicellular species. Drosophila fringe proteins modulate the activation of the Notch signal transduction pathway at the dorsal-ventral boundary of the wing imaginal disc. Mammalian fringe-related family members participate in boundary determination during segmentation (Johnston, S. H. et al. (1997) Development 124:2245-2254).

Recently a number of proteins have been found to contain a conserved cysteine-rich domain of about 60 amino-acid residues called the LIM domain (for Lin-11 Is1-1 Mec-3) (Freyd, G. et al. (1990) Nature 344:876-879; Baltz, R. et al. (1992) Plant Cell 4:1465-1466). In the LIM domain, there are seven conserved cysteine residues and a histidine. The LIM domain binds two zinc ions (Michelsen, J. W. et al. (1993) Proc. Natl. Acad. Sci. U.S.A. 90:44044408). LIM does not bind DNA; rather, it seems to act as an interface for protein-protein interaction.

### Apoptosis

Apoptosis is the genetically controlled process by which unneeded or defective cells undergo programmed cell death. Selective elimination of cells is as important for morphogenesis and tissue remodeling as is cell proliferation and differentiation. Lack of apoptosis may result in hyperplasia and other disorders associated with increased cell proliferation. Apoptosis is also a critical component of the immune response. Immune cells such as cytotoxic T-cells and natural killer cells prevent the spread of disease by inducing apoptosis in tumor cells and virus-infected cells. In addition, immune cells that fail to distinguish self molecules from foreign molecules must be eliminated by apoptosis to avoid an autoimmune response.

Apoptotic cells undergo distinct morphological changes. Hallmarks of apoptosis include cell shrinkage, nuclear and cytoplasmic condensation, and alterations in plasma membrane topology. Biochemically, apoptotic cells are characterized by increased intracellular calcium concentration, fragmentation of chromosomal DNA, and expression of novel cell surface components.

The molecular mechanisms of apoptosis are highly conserved, and many of the key protein regulators and effectors of apoptosis have been identified. Apoptosis generally proceeds in response to a signal which is transduced intracellularly and results in altered patterns of gene expression and protein activity. Signaling molecules such as hormones and cytokines are known both to stimulate and to inhibit apoptosis through interactions with cell surface receptors. Transcription factors also play an important role in the onset of apoptosis. A number of downstream effector molecules, especially proteases, have been implicated in the degradation of cellular components and the proteolytic activation of other apoptotic effectors.

The Bcl-2 family of proteins, as well as other cytoplasmic proteins, are key regulators of apoptosis. There are at least 15 Bcl-2 family members within 3 subfamilies. These proteins have been identified in mammalian cells and in viruses, and each possesses at least one of four Bcl-2 homology domains (BH1 to BH4), which are highly conserved. Bcl-2 family proteins contain the BH1 and BH2 domains, which are found in members of the pro-survival subfamily, while those proteins which are most similar to Bcl-2 have all four conserved domains, enabling inhibition of apoptosis following encounters with a variety of cytotoxic challenges. Members of the pro-survival subfamily include Bcl-2, Bcl-X_{L}, Bcl-w, Mc1-1, and A1 in maimmals; NF-13 (chicken); CED-9 (Caenorhabditis elegans); and viral proteins BHRF1, LMW5-HL, ORF16, KS-Bc1-2, and E1B-19K. The BH3 domain is essential for the function of pro-apoptosis subfamily proteins. The two pro-apoptosis subfamilies, Bax and BH3, include Bax, Bak, and Bok (also called Mtd); and Bik, Blk, Hrk, BNIP3, Bim_{L}, Bad, Bid, and Egl-1 (C. elegans); respectively. Members of the Bax subfamily contain the BH1, BH2, and BH3 domains, and resemble Bcl-2 rather closely. In contrast, members of the BH3 subfamily have only the 9-16 residue BH3 domain, being otherwise unrelated to any known protein, and only Bik and Blk share sequence similarity. The proteins of the two pro-apoptosis subfamilies may be the antagonists of pro-survival subfamily proteins. This is illustrated in C. elegans where Egl-1, which is required for apoptosis, binds to and acts via CED-9 (for review, see Adams, J. M. and S. Cory (1998) Science 281:1322-1326).

Heterodimerization between pro-apoptosis and anti-apoptosis subfamily proteins seems to have a titrating effect on the functions of these protein subfamilies, which suggests that relative concentrations of the members of each subfamily may act to regulate apoptosis. Heterodimerization is not required for a pro-survival protein; however, it is essential in the BH3 subfamily, and less so in the Bax subfamily.

The Bcl-2 protein has 2 isoforms, alpha and beta, which are formed by alternative splicing. It forms homodimers and heterodimers with Bax and Bak proteins and the Bcl-X isoform Bcl-x_{S}. Heterodimerization with Bax requires intact BH1 and BH2 domains, and is necessary for pro-survival activity. The BH4 domain seems to be involved in pro-survival activity as well. Bcl-2 is located within the inner and outer mitochondrial membranes, as well as within the nuclear envelope and endoplasmic reticulum, and is expressed in a variety of tissues. Its involvement in follicular lymphoma (type II chronic lymphatic leukemia) is seen in a chromosomal translocation T(14;18) (q32;q21) and involves immunoglobulin gene regions.

The Bcl-x protein is a dominant regulator of apoptotic cell death. Alternative splicing results in three isoforms, Bcl-xB, a long isoform, and a short isoform The long isoform exhibits cell death repressor activity, while the short isoform promotes apoptosis. Bcl-xL forms heterodimers with Bax and Bak, although heterodimerization with Bax does not seem to be necessary for pro-survival (anti-apoptosis) activity. Bcl-xS forms heterodimers with Bcl-2. Bcl-x is found in mitochondrial membranes and the perinuclear envelope. Bcl-xS is expressed at high levels in developing lymphocytes and other cells undergoing a high rate of turnover. Bcl-xL is found in adult brain and in other tissues' long-lived post-mitotic cells. As with Bcl-2, the BH1, BH2, and BH4 domains are involved in pro-survival activity.

The Bcl-w protein is found within the cytoplasm of almost all myeloid cell lines and in numerous tissues, with the highest levels of expression in brain, colon, and salivary gland. This protein is expressed in low levels in testis, liver, heart, stomach, skeletal muscle, and placenta, and a few lymphoid cell lines. Bcl-w contains the BH1, BH2, and BH4 domains, all of which are needed for its cell survival promotion activity. Although mice in which Bcl-w gene function was disrupted by homologous recombination were viable, healthy, and normal in appearance, and adult females had normal reproductive function, the adult males were infertile. In these males, the initial, prepuberty stage of spermatogenesis was largely unaffected and the testes developed normally. However, the seminiferous tubules were disorganized, contained numerous apoptotic cells, and were incapable of producing mature sperm. This mouse model may be applicable to some cases of human male sterility and suggests that alteration of programmed cell death in the testes may be useful in modulating fertility (Print, C. G. et al. (1998) Proc. Natl. Acad. Sci. USA 95:12424-12431).

Studies in rat ischemic brain found Bcl-w to be overexpressed relative to its normal low constitutive level of expression in nonischemic brain. Furthermore, in vitro studies to examine the mechanism of action of Bcl-w revealed that isolated rat brain mitochondria were unable to respond to an addition of recombinant Bax or high concentrations of calcium when Bcl-w was also present. The normal response would be the release of cytochrome c from the mitochondria. Additionally, recombinant Bcl-w protein was found to inhibit calcium-induced loss of mitochondrial transmembrane potential, which is indicative of permeability transition. Together these findings suggest that Bcl-w may be a neuro-protectant against ischemic neuronal death and may achieve this protection via the mitochondrial death-regulatory pathway (Yan, C. et al. (2000) J. Cereb. Blood Flow Metab. 20:620-630).

The bfl-1 gene is an additional member of the Bcl-2 family, and is also a suppressor of apoptosis. The Bfl-1 protein has 175 amino acids, and contains the BH1, BH2, and BH3 conserved domains found in Bcl-2 family members. It also contains a Gln-rich NH2-terminal region and lacks an NH domain 1, unlike other Bcl-2 family members. The mouse A1 protein shares high sequence homology with Bfl-1 and has the 3 conserved domains found in Bfl-1. Apoptosis induced by the p53 tumor suppressor protein is suppressed by Bfl-1, similar to the action of Bcl-2, Bcl-xL, and EBV-BHRF1 (D'Sa-Eipper, C. et al. (1996) Cancer Res. 56:3879-3882). Bfl-1 is found intracellularly, with the highest expression in the hematopoietic compartment, i.e. blood, spleen, and bone marrow; moderate expression in lung, small intestine, and testis; and minimal expression in other tissues. It is also found in vascular smooth muscle cells and hematopoietic malignancies. A correlation has been noted between the expression level of bfl-1 and the development of stomach cancer, suggesting that the Bfl-1 protein is involved in the development of stomach cancer, either in the promotion of cancerous cell survival or in cancer (Choi, S. S. et al. (1995) Oncogene 11:1693-1698).

Cancers are characterized by continuous or uncontrolled cell proliferation. Some cancers are associated with suppression of normal apoptotic cell death. Strategies for treatment may involve either reestablishing control over cell cycle progression, or selectively stimulating apoptosis in cancerous cells (Nigg, E. A. (1995) BioEssays 17:471-480). Immunological defenses against cancer include induction of apoptosis in mutant cells by tumor suppressors, and the recognition of tumor antigens by T lymphocytes. Response to mitogenic stresses is frequently controlled at the level of transcription and is coordinated by various transcription factors. For example, the Rel/NF-kappa B family of vertebrate transcription factors plays a pivotal role in inflammatory and immune responses to radiation. The NF-kappa B family includes p50, p52, RelA, RelB, cRel, and other DNA-binding proteins. The p52 protein induces apoptosis, upregulates the transcription factor c-Jun, and activates c-Jun N-terminal kinase 1 (JNK1) (Sun, L. et al. (1998) Gene 208:157-166). Most NF-kappa B proteins form DNA-binding homodimers or heterodimers. Dimerization of many transcription factors is mediated by a conserved sequence known as the bZIP domain, characterized by a basic region followed by a leucine zipper.

The Fas/Apo-1 receptor (FAS) is a member of the tumor necrosis factor (TNF) receptor family. Upon binding its ligand (Fas ligand), the membrane-spanning FAS induces apoptosis by recruiting several cytoplasmic proteins that transmit the death signal. One such protein, termed FAS-associated protein factor 1 (FAF1), was isolated from mice, and it was demonstrated that expression of FAF1 in L cells potentiated FAS-induced apoptosis (Chu, K. et al. (1995) Proc. Natl. Acad. Sci. USA 92:11894-11898). Subsequently, FAS-associated factors have been isolated from numerous other species, including fruit fly and quail (Frohlich, T. et al. (1998) J. Cell Sci. 111:2353-2363). Another cytoplasmic protein that functions in the transmittal of the death signal from Fas is the Fas-associated death domain protein, also known as FADD. FADD transmits the death signal in both FAS-mediated and TNF receptor-mediated apoptotic pathways by activating caspase-8 (Bang, S. et al. (2000) J. Biol. Chem. 275:36217-36222).

Fragmentation of chromosomal DNA is one of the hallmarks of apoptosis. DNA fragmentation factor (DFF) is a protein composed of two subunits, a 40-kDa caspase-activated nuclease termed DFF40/CAD, and its 45-kDa inhibitor DFP45/ICAD. Two mouse homologs of DFF45/ICAD, termed CIDE-A and CIDE-B, have recently been described (Inohara, N. et al. (1998) EMBO J. 17:2526-2533). CIDE-A and CIDE-B expression in mammalian cells activated apoptosis, while expression of CIDE-A alone induced DNA fragmentation. In addition, FAS-mediated apoptosis was enhanced by CIDE-A and CIDE-B, further implicating these proteins as effectors that mediate apoptosis.

Transcription factors play an important role in the onset of apoptosis. A number of downstream effector molecules, particularly proteases such as the cysteine proteases called caspases, are involved in the initiation and execution phases of apoptosis. The activation of the caspases results from the competitive action of the pro-survival and pro-apoptosis Bcl-2-related proteins (Print, C. G. et al. (1998) Proc. Natl. Acad. Sci. USA 95:12424-12431). A pro-apoptotic signal can activate initiator caspases that trigger a proteolytic caspase cascade, leading to the hydrolysis of target proteins and the classic apoptotic death of the cell. Two active site residues, a cysteine and a histidine, have been implicated in the catalytic mechanism. Caspases are among the most specific endopeptidases, cleaving after aspartate residues.

Caspases are synthesized as inactive zymogens consisting of one large (p20) and one small (p10) subunit separated by a small spacer region, and a variable N-terminal prodomain. This prodomain interacts with cofactors that can positively or negatively affect apoptosis. An activating signal causes autoproteolytic cleavage of a specific aspartate residue (D297 in the caspase-1 numbering convention) and removal of the spacer and prodomain, leaving a p10/p20 heterodimer. Two of these heterodimers interact via their small subunits to form the catalytically active tetramer. The long prodomains of some caspase family members have been shown to promote dimerization and auto-processing of procaspases. Some caspases contain a “death effector domain” in their prodomain by which they can be recruited into self-activating complexes with other caspases and FADD protein-associated death receptors or the TNF receptor complex. In addition, two dimers from different caspase family members can associate, changing the substrate specificity of the resultant tetramer.

Tumor necrosis factor (TNF) and related cytokines induce apoptosis in lymphoid cells. (Reviewed in Nagata, S. (1997) Cell 88:355-365.) Binding of TNF to its receptor triggers a signal transduction pathway that results in the activation of a proteolytic caspase cascade. One such caspase, ICE (Interleukin-1β converting enzyme), is a cysteine protease comprised of two large and two small subunits generated by ICE auto-cleavage (Dinarello, C. A. (1994) FASEB J. 8:1314-1325). ICE is expressed primarily in monocytes. ICE processes the cytokine precursor, interleukin-1β, into its active form, which plays a central role in acute and chronic inflammation, bone resorption, myelogenous leukemia, and other pathological processes. ICE and related caspases cause apoptosis when overexpressed in transfected cell lines.

A caspase recruitment domain (CARD) is found within the prodomain of several apical caspases and is conserved in several apoptosis regulatory molecules such as Apaf-2, RAIDD, and cellular inhibitors of apoptosis proteins (IAPs) (Hofmann, K. et al. (1997) Trends Biochem. Sci. 22:155-157). The regulatory role of CARD in apoptosis may be to allow proteins such as Apaf-1 to associate with caspase-9 (Li, P. et al. (1997) Cell 91:479489). A human cDNA encoding an apoptosis repressor with a CARD (ARC) which is expressed in both skeletal and cardiac muscle has been identified and characterized. ARC functions as an inhibitor of apoptosis and interacts selectively with caspases (Koseki, T. et al. (1998) Proc. Natl. Acad. Sci. USA 95:5156-5160). All of these interactions have clear effects on the control of apoptosis (reviewed in Chan S. L. and M. P. Mattson (1999) J. Neurosci. Res. 58:167-190; Salveson, G. S. and V. M. Dixit (1999) Proc. Natl. Acad. Sci. USA 96:10964-10967).

ES18 was identified as a potential regulator of apoptosis in mouse T-cells (Park, E. J. et al. (1999) Nuc. Acid. Res. 27:1524-1530). ES18 is 428 amino acids in length, contains an N-terminal proline-rich region, an acidic glutamic acid-rich domain, and a putative LXXLL nuclear receptor binding motif. The protein is preferentially expressed in lymph nodes and thymus. The level of ES18 expression increases in T-cell thymoma S49.1 in response to treatment with dexamethasone, staurosporine, or C2-ceramide, which induce apoptosis. ES18 may play a role in stimulating apoptotic cell death in T-cells.

The rat ventral prostate (RVP) is a model system for the study of hormone-regulated apoptosis. RVP epithelial cells undergo apoptosis in response to androgen deprivation. Messenger RNA (mRNA) transcripts that are up-regulated in the apoptotic RVP have been identified (Briehl, M. M. and R. L. Miesfeld (1991) Mol. Endocrinol. 5:1381-1388). One such transcript encodes RVP.1, the precise role of which in apoptosis has not been determined. The human homolog of RVP.1, hRVP1, is 89% identical to the rat protein (Katahira, J. et al. (1997) J. Biol. Chem. 272:26652-26658). hRVP1 is 220 amino acids in length and contains four transmembrane domains. hRVP1 is highly expressed in the lung, intestine, and liver. Interestingly, hRVP1 functions as a low affinity receptor for the Clostridium perfringens enterotoxin, a causative agent of diarrhea in humans and other animals.

Cytokine-mediated apoptosis plays an important role in hematopoiesis and the immune response. Myeloid cells, which are the stem cell progenitors of macrophages, neutrophils, erythrocytes, and other blood cells, proliferate in response to specific cytokines such as granulocyte/macrophage-colony stimulating factor (GMCSF) and interleukin-3 (IL-3). When deprived of GM-CSF or IL-3, myeloid cells undergo apoptosis. The murine requiem (req) gene encodes a putative transcription factor required for this apoptotic response in the myeloid cell line FDCP-1 (Gabig, T. G. et al. (1994) J. Biol. Chem. 269:29515-29519). The Req protein is 371 amino acids in length and contains a nuclear localization signal, a single Kruppel-type zinc finger, an acidic domain, and a cluster of four unique zinc-finger motifs enriched in cysteine and histidine residues involved in metal binding. Expression of req is not myeloid- or apoptosis-specific, suggesting that additional factors regulate Req activity in myeloid cell apoptosis.

Dysregulation of apoptosis has recently been recognized as a significant factor in the pathogenesis of many human diseases. For example, excessive cell survival caused by decreased apoptosis can contribute to disorders related to cell proliferation and the immune response. Such disorders include cancer, autoimmune diseases, viral infections, and inflammation. In contrast, excessive cell death caused by increased apoptosis can lead to degenerative and immunodeficiency disorders such as AIDS, neurodegenerative diseases, and myelodysplastic syndromes. (Thompson, C. B. (1995) Science 267:1456-1462.)

Impaired regulation of apoptosis is also associated with loss of neurons in Alzheimer's disease. Alzheimer's disease is a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. B-amyloid peptide participates in signaling pathways that induce apoptosis and lead to the death of neurons (Kajkowski, C. et al. (2001) J. Biol. Chem. 276:18748-18756). Early in Alzheimer's pathology, physiological changes are visible in the cingulate cortex (Minoshima, S. et al. (1997) Annals of Neurology 42:85-94). In subjects with advanced Alzheimer's disease, accumulating plaques damage the neuronal architecture in limbic areas and eventually cripple the memory process.

### Cell Differentiation and Proliferation

Tissue growth involves complex and ordered patterns of cell proliferation, cell differentiation, and apoptosis. Cell proliferation must be regulated to maintain both the number of cells and their spatial organization. This regulation depends upon the appropriate expression of proteins which control cell cycle progression in response to extracellular signals, such as growth factors and other mitogens, and intracellular cues, such as DNA damage or nutrient starvation. Molecules which directly or indirectly modulate cell cycle progression fall into several categories, including growth factors and their receptors, second messenger and signal transduction proteins, oncogene products, tumor-suppressor proteins, and mitosis-promoting factors.

Growth factors were originally described as serum factors required to promote cell proliferation. Most growth factors are large, secreted polypeptides that act on cells in their local environment. Growth factors bind to and activate specific cell surface receptors and initiate intracellular signal transduction cascades. Many growth factor receptors are classified as receptor tyrosine kinases which undergo autophosphorylation upon ligand binding. Autophosphorylation enables the receptor to interact with signal transduction proteins characterized by the presence of SH2 or SH3 domains (Src homology regions 2 or 3). These proteins then modulate the activity state of small G-proteins, such as Ras, Rab, and Rho, along with GTPase activating proteins (GAPs), guanine nucleotide releasing proteins (GNRPs), and other guanine nucleotide exchange factors. Small G proteins act as molecular switches that activate other downstream events, such as mitogen-activated protein kinase (MAP kinase) cascades. MAP kinases ultimately activate transcription of mitosis-promoting genes.

In addition to growth factors, small signaling peptides and hormones also influence cell proliferation. These molecules bind primarily to another class of receptor, the trimeric G-protein coupled receptor (GPCR), found predominantly on the surface of immune, neuronal and neuroendocrine cells. Upon ligand binding, the GPCR activates a trimeric G protein which in turn triggers increased levels of intracellular second messengers such as phospholipase C, Ca2+, and cyclic AMP. Most GPCR-mediated signaling pathways indirectly promote cell proliferation by causing the secretion or breakdown of other signaling molecules that have direct mitogenic effects. These signaling cascades often involve activation of kinases and phosphatases. Some growth factors, such as some members of the transforming growth factor beta (TGF-β) family, act on some cells to stimulate cell proliferation and on other cells to inhibit it. Growth factors may also stimulate a cell at one concentration and inhibit the same cell at another concentration. Most growth factors also have a multitude of other actions besides the regulation of cell growth and division: they can control the proliferation, survival, differentiation, migration, or function of cells depending on the circumstance. For example, the tumor necrosis factor/nerve growth factor (TNF/NGF) family can activate or inhibit cell death, as well as regulate proliferation and differentiation. The cell response depends on the type of cell, its stage of differentiation and transformation status, which surface receptors are stimulated, and the types of stimuli acting on the cell (Smith, A. et al. (1994) Cell 76:959-962; and Nocentini, G. et al. (1997) Proc. Natl. Acad. Sci. USA 94:6216-6221).

Neighboring cells in a tissue compete for growth factors, and when provided with “unlimited” quantities in a perfused system will grow to even higher cell densities before reaching density-dependent inhibition of cell division. Cells often demonstrate an anchorage dependence of cell division as well. This anchorage dependence may be associated with the formation of focal contacts linking the cytoskeleton with the extracellular matrix (ECM). The expression of ECM components can be stimulated by growth factors. For example, TGF-β stimulates fibroblasts to produce a variety of ECM proteins, including fibronectin, collagen, and tenascin (Pearson, C. A. et al. (1988) EMBO J. 7:2677-2981). In fact, for some cell types specific ECM molecules, such as laminin or fibronectin, may act as growth factors. Tenascin-C and -R, expressed in developing and lesioned neural tissue, provide stimulatory/anti-adhesive or inhibitory properties, respectively, for axonal growth (Faissner, A. (1997) Cell Tissue Res. 290:331-341).

### Oncogenes

Cancers are associated with the activation of oncogenes which are derived from normal cellular genes. These oncogenes encode oncoproteins which convert normal cells into malignant cells. Some oncoproteins are mutant isoforms of the normal protein, and other oncoproteins are abnormally expressed with respect to location or amount of expression. The latter category of oncoprotein causes cancer by altering transcriptional control of cell proliferation. Five classes of oncoproteins are known to affect cell cycle controls. These classes include growth factors, growth factor receptors, intracellular signal transducers, nuclear transcription factors, and cell-cycle control proteins. Viral oncogenes are integrated into the human genome after infection of human cells by certain viruses. Examples of viral oncogenes include v-src, v-abl, and v-fps. Many cases related to the overexpression of proteins associated with tumors and metastasis have been reported. The Mta1 gene has been cloned in mice, in both cell lines and tissues representing metastatic tumors (Simpson, A. et al. (2001) Gene 273:29-39). Expression of the melanoma antigen-encoding gene (MAGE) family of proteins has also been detected in many tumors. GAC1, a new member of the leucine-rich repeat superfamily, is amplified and overexpressed in malignant gliomas (Almeida, A. et al. (1998) Oncogene 16:2997-3002).

Many oncogenes have been identified and characterized. These include sis, erbA, erbB, her-2, mutated Gₛ, src, abl, ras, crk, jun, fos, myc, and mutated tumor-suppressor genes such as RB, p53, mdm2, Cip1, p16, and cyclin D. Transformation of normal genes to oncogenes may also occur by chromosomal translocation. The Philadelphia chromosome, characteristic of chronic myeloid leukemia and a subset of acute lymphoblastic leukemias, results from a reciprocal translocation between chromosomes 9 and 22 that moves a truncated portion of the proto-oncogene c-abl to the breakpoint cluster region (bcr) on chromosome 22.

Tumor-suppressor genes are involved in regulating cell proliferation. Mutations which cause reduced or loss of function in tumor-suppressor genes result in uncontrolled cell proliferation. For example, the retinoblastoma gene product (RB), in a non-phosphorylated state, binds several early-response genes and suppresses their transcription, thus blocking cell division. Phosphorylation of RB causes it to dissociate from the genes, releasing the suppression, and allowing cell division to proceed.

### Angiogenesis

Angiogenesis is the process by which new capillaries are formed by sprouting from preexisting vessels. It is a vital function for the gowth of normal tissues during embryogenesis as well as for the pathological growth of tumors. Pathological proliferation fo cancer cells will not result in a proportional increse in mass without access to the blood circulation. Tumors form their own circulatory system by upregulating angiogenic stimulators and by downregulation angiogenesis inhibitors. The inability of metastases to induce an angiogenic response results in a dormant phenotype. Angiostatin is a circulating inhibitor of angiogenesis. In vitro, it inhibits endothelial cell migration, proliferation, and tube formation, and induces apoptosis in a cell type-specific manner. Angiomotin is an angiostatin-binding peptide that mediates angiostatin inhibition of migration and tube formation of endothelial cells. Angiomotin is expressed in the endothelial cells of capillaries and the larger vessels of the human placenta. Angiostatin inhibits cell migration by interfering with angiomotin activity in endothelial cells (Troyanovsky, B. et al. (2001) J. Cell Biol. 152:1247-1254).

Nucleolar protein p120 is a proliferation-associated antigen expressed by cells in early G1 phase, identified by the monoclonal antibody FB. It is very cancer specific. In fact, quantitative immunohistochemical analysis of p120 protein is an easy and reliable method for the assessment of clinical outcome and the definition of risk groups in oral carcinoma (Ventura, L. et al. (1999) Anticancer Res. 19:1423-1426). P120 contains a basic domain, an acidic domain, a hydrophobic and methionine-rich domain, and a domain rich in cysteine and proline residues (Fonagy, A. et al. (1989) Cancer Commun. 1:243-251). This protein is expressed in early G1 and has not been detected in benign tumors and most normal resting tissues. Sato et al. show that the expression level of p120 in tumor tissues can be used as an independent and powerful prognostic marker for resected lung adenocarcinoma (Sato, G. et al. (1999) J. Clin. Oncol. 17:2721-2727).

The human LGI1 gene is a leucine-rich, repeat-containing gene that was cloned from the t(10:19) breakpoint of the T98G glioblastoma cell line. The LGIL gene maps to 10q24, a region of peak LOH in malignant gliomas, and is inactivated during the transition from low to high-grade brain tumors. The mouse lgil gene is 97% homologous to the human gene at the amino acid level and 91% homologous at the nucleotide level. LGIL contains 8 exons, where each of the four leucine-rich repeat units is contained in an individual 72-bp exon. The cysteine-rich regions flanking the LRR and the single trans-membrane domain do not occupy individual exons (Somerville, R. P. et al. (2000) Mamm. Genome 11:622-627).

Viral oncogenes are integrated into the human genome after infection of human cells by certain viruses. Examples of viral oncogenes include v-src, v-abl, and v-fps. Transformation of normal genes to oncogenes may also occur by chromosomal translocation. The Philadelphia chromosome, characteristic of chronic myeloid leukemia and a subset of acute lymphoblastic leukemias, results from a reciprocal translocation between chromosomes 9 and 22 that moves a truncated portion of the proto-oncogene c-abl to the breakpoint cluster region (bcr) on chromosome 22. The hybrid c-abl-bcr gene encodes a chimeric protein that has tyrosine kinase activity. In chronic myeloid leukemia, the chimeric protein has a molecular weight of 210 kd, whereas in acute leukemias a more active 180 kd tyrosine kinase is formed (Robbins, S. L. et al. (1994) Pathologic Basis of Disease, W. B. Saunders Co., Philadelphia Pa.).

The Ras superfamily of small GTPases is involved in the regulation of a wide range of cellular signaling pathways. Ras family proteins are membrane-associated proteins acting as molecular switches that bind GTP and GDP, hydrolyzing GTP to GDP. In the active GTP-bound state Ras family proteins interact with a variety of cellular targets to activate downstream signaling pathways. For example, members of the Ras subfamily are essential in transducing signals from receptor tyrosine kinases (RTKs) to a series of serine/threonine kinases which control cell growth and differentiation. Activated Ras genes were initially found in human cancers and subsequent studies confirmed that Ras function is critical in the determination of whether cells continue to grow or become terminally differentiated (Barbacid, M. (1987) Annu. Rev. Biochem. 56:779-827; Treisman, R. (1994) Curr. Opin. Genet. Dev. 4:96-98). Mutant Ras proteins, which bind but can not hydrolyze GTP, are permanently activated, and cause continuous cell proliferation or cancer.

Activation of Ras family proteins is catalyzed by guanine nucleotide exchange factors (GEFs) which catalyze the dissociation of bound GDP and subsequent binding of GTP. A recently discovered RalGEF-like protein, RGL3, interacts with both Ras and the related protein Rit. Constitutively active Rit, like Ras, can induce oncogenic transformation, although since Rit fails to interact with most known Ras effector proteins, novel cellular targets may be involved in Rit transforming activity. RGL3 interacts with both Ras and Rit, and thus may act as a downstream effector for these proteins (Shao, H. and D. A. Andres (2000) J. Biol. Chem. 275:26914-26924).

### Tumor Antigens

Tumor antigens are cell surface molecules that are differentially expressed in tumor cells relative to non-tumor tissues. Tumor antigens make tumor cells immunologically distinct from normal cells and are potential diagnostics for human cancers. Several monoclonal antibodies have been identified which react specifically with cancerous cells such as T-cell acute lymphoblastic leukemia and neuroblastoma (Minegishi, M. et al. (1989) Leukemia Res. 13:43-51; Takagi, S. et al. (1995) Int. J. Cancer 61:706-715). In addition, the discovery of high level expression of the HER2 gene in breast tumors has led to the development of therapeutic treatments (Liu, E. et al. (1992) Oncogene 7:1027-1032; Kern, J. A. (1993) Am. J. Respir. Cell Mol. Biol. 9:448454). Tumor antigens are found on the cell surface and have been characterized either as membrane proteins or glycoproteins. For example, MAGE genes encode a family of tumor antigens recognized on melanoma cell surfaces by autologous cytolytic T lymphocytes. Among the 12 human MAGE genes isolated, half are differentially expressed in tumors of various histological types (De Plaen, E. et al. (1994) Immunogenetics 40:360-369). None of the 12 MAGE genes, however, is expressed in healthy tissues except testis and placenta.

### Tumor Suppressors

Tumor suppressor genes are generally defined as genetic elements whose loss or inactivation contributes to the deregulation of cell proliferation and the pathogenesis and progression of cancer. Tumor suppressor genes normally function to control or inhibit cell growth in response to stress and to limit the proliferative life span of the cell. Several tumor suppressor genes have been identified including the genes encoding the retinoblastoma (Rb) protein, p53, and the breast cancer 1 and 2 proteins (BRCA1 and BRCA2). Mutations in these genes are associated with acquired and inherited genetic predisposition to the development of certain cancers.

The role of p53 in the pathogenesis of cancer has been extensively studied. (Reviewed in Aggarwal, M. L. et al. (1998) J. Biol. Chem. 273:14; Levine, A. (1997) Cell 88:323-331.) About 50% of all human cancers contain mutations in the p53 gene. These mutations result in either the absence of functional p53 or, more commonly, a defective form of p53 which is overexpressed. p53 is a transcription factor that contains a central core domain required for DNA binding. Most cancer-associated mutations in p53 localize to this domain. In normal proliferating cells, p53 is expressed at low levels and is rapidly degraded. p53 expression and activity is induced in response to DNA damage, abortive mitosis, and other stressful stimuli. In these instances, p53 induces apoptosis or arrests cell growth until the stress is removed. Downstream effectors of p53 activity include apoptosis-specific proteins and cell cycle regulatory proteins, including Rb, oncogene products, cyclins, and cell cycle-dependent kinases.

A novel candidate tumor suppressor gene, LGI1 (leucine-rich gene-glioma inactivated), has been identified in glioblastoma multiforme tumors using positional cloning techniques (Chernova, O. B. et al. (1998) 17:2873-2881). Glioblastoma multiforme is characterized by abundant angiogenic activity and loss of tumor suppressor genes on chromosome 10. The human LGI1 gene is a leucine-rich, repeat-containing gene cloned from the t(10; 19) breakpoint of the T98G glioblastoma cell line (Somerville, R. P. T., et al. (2000) Mammalian Genome 11:622-627). Specifically, LGI1 has been localized to the 10q24 region, and its association with glial tumors may be the result of a t(10;19) (q24;q13) translocation event. The LGI1 gene encodes a protein with molecular mass of 60 kD and contains 3.5 leucine-rich repeats (LRR).

The metastasis-suppressor gene KAI1 (CD82) has been reported to be related to the tumor suppressor gene p53. KAI1 is involved in the progression of human prostatic cancer and possibly lung and breast cancers when expression is decreased. KAI1 encodes a member of a structurally distinct family of leukocyte surface glycoproteins. The family is known as either the tetraspan transmembrane protein family or transmembrane 4 superfamily (TM4SF) as the members of this family span the plasma membrane four times. The family is composed of integral membrane proteins having a N-terminal membrane-anchoring domain which functions as both a membrane anchor and a translocation signal during protein biosynthesis. The N-terminal membrane-anchoring domain is not cleaved during biosynthesis. TM4SF proteins have three additional transmembrane regions, seven or more conserved cysteine residues, are similar in size (218 to 284 residues), and all have a large extracellular hydrophilic domain with three potential N-glycosylation sites. The promoter region contains many putative binding motifs for various transcription factors, including five AP2 sites and nine SpI sites. Gene structure comparisons of KAIL and seven other members of the TM4SF indicate that the splicing sites relative to the different structural domains of the predicted proteins are conserved. This suggests that these genes are related evolutionarily and arose through gene duplication and divergent evolution (Levy, S. et al. (1991) J. Biol. Chem. 266:14597-14602; Dong, J. T. et al. (1995) Science 268:884-886; Dong, J. T. et al., (1997) Genomics 41:25-32).

The Leucine-rich gene-Glioma Inactivated (LGIL) protein shares homology with a number of transmembrane and extracellular proteins which function as receptors and adhesion proteins. LGI1 is encoded by an LLR (leucine-rich, repeat-containing) gene and maps to 10q24. LGI1 has four LLRs which are flanked by cysteine-rich regions and one transmembrane domain (Somerville, R. P. et al. (2000) Mamm. Genome 11:622-627). LGI1 expression is seen predominantly in neural tissues, especially brain. The loss of tumor suppressor activity is seen in the inactivation of the LGI1 protein which occurs during the transition from low to high-grade tumors in malignant gliomas. The reduction of LGI1 expression in low grade brain tumors and its significant reduction or absence of expression in malignant gliomas suggests that it could be used for diagnosis of glial tumor progression (Chernova, O. B. et al. (1998) Oncogene 17:2873-2881).

The ST13 tumor suppressor was identified in a screen for factors related to colorectal carcinomas by subtractive hybridization between cDNA of normal mucosal tissues and mRNA of colorectal carcinoma tissues (Cao, J. et al. (1997) J. Cancer Res. Clin. Oncol. 123:447-451). ST13 is down-regulated in human colorectal carcinomas.

Mutations in the von Hippel-Lindau (VHL) tumor suppressor gene are associated with retinal and central nervous system hemangioblastomas, clear cell renal carcinomas, and pheochromocytomas (Hoffman, M. et al. (2001) Hum. Mol. Genet. 10: 1019-1027; Kamada, M. (2001) Cancer Res. 61:4184-4189). Tumor progression is linked to defects or inactivation of the VHL gene. VHL regulates the expression of transforming growth factor-α, the GLUT-1 glucose transporter and vascular endothelial growth factor. The VHL protein associates with elongin B, elongin C, Cul2 and Rbx1 to form a complex that regulates the transcriptional activator hypoxia-inducible factor (HIF). HIF induces genes involved in angiogenesis such as vascular endothelial growth factor and platelet-derived growth factor B. Loss of control of HIF caused by defects in VHL results in the excessive production of angiogenic peptides. VHL may play roles in inhibition of angiogenesis, cell cycle control, fibronectin matrix assembly, cell adhesion, and proteolysis.

Mutations in tumor suppressor genes are a common feature of many cancers and often appear to affect a critical step in the pathogenesis and progression of tumors. Accordingly, Chang, F. et al. (1995; J. Clin. Oncol. 13:1009-1022) suggest that it may be possible to use either the gene or an antibody to the expressed protein 1) to screen patients at increased risk for cancer, 2) to aid in diagnosis made by traditional methods, and 3) to assess the prognosis of individual cancer patients. In addition, Hamada, K. et al. (1996; Cancer Res. 56:3047-3054) are investigating the introduction of p53 into cervical cancer cells via an adenoviral vector as an experimental therapy for cervical cancer.

The PR-domain genes were recently recognized as playing a role in human tumorigenesis. PR-domain genes normally produce two protein products: the PR-plus product, which contains the PR domain, and the PR-minus product which lacks this domain. In cancer cells, PR-plus is disrupted or overexpressed, while PR-minus is present or overexpressed. The imbalance in the amount of these two proteins appears to be an important cause of malignancy (Jiang, G. L. and S. Huang (2000) Histol. Histopathol. 15:109-117).

Many neoplastic disorders in humans can be attributed to inappropriate gene transcription. Malignant cell growth may result from either excessive expression of tumor promoting genes or insufficient expression of tumor suppressor genes (Cleary, M. L. (1992) Cancer Surv. 15:89-104). Chromosomal translocations may also produce chimeric loci which fuse the coding sequence of one gene with the regulatory regions of a second unrelated gene. An important class of transcriptional regulators are the zinc finger proteins. The zinc finger motif, which binds zinc ions, generally contains tandem repeats of about 30 amino acids consisting of periodically spaced cysteine and histidine residues. Examples of this sequence pattern include the C2H2-type, C4-type, and C3HC4-type zinc fingers, and the PHD domain (Lewin, B. (1990) Genes IV, Oxford University Press, New York, N.Y., and Cell Press, Cambridge, Mass., pp. 554-570; Aasland, R., et al. (1995) Trends Biochem. Sci. 20:56-59). One clinically relevant zinc-finger protein is WT1, a tumor-suppressor protein that is inactivated in children with Wilm's tumor. The oncogene bcl-6, which plays an important role in large-cell lymphoma, is also a zinc-finger protein (Papavassiliou, A. G. (1995) N. Engl. J. Med. 332:45-47).

### Tumor Responsive Proteins

Cancers, also called neoplasias, are characterized by continuous and uncontrolled cell proliferation. They can be divided into three categories: carcinomas, sarcomas, and leukemias. Carcinomas are malignant growths of soft epithelial cells that may infiltrate surrounding tissues and give rise to metastatic tumors. Sarcomas may be of epithelial origin or arise from connective tissue. Leukemias are progressive malignancies of blood-forming tissue characterized by proliferation of leukocytes and their precursors, and may be classified as myelogenous (granulocyte- or monocyte-derived) or lymphocytic (lymphocyte-derived). Tumorigenesis refers to the progression of a tumor's growth from its inception. Malignant cells may be quite similar to normal cells within the tissue of origin or may be undifferentiated (anaplastic). Tumor cells may possess few nuclei or one large polymorphic nucleus. Anaplastic cells may grow in a disorganized mass that is poorly vascularized and as a result contains large areas of ischemic necrosis. Differentiated neoplastic cells may secrete the same proteins as the tissue of origin. Cancers grow, infiltrate, invade, and destroy the surrounding tissue through direct seeding of body cavities or surfaces, through lymphatic spread, or through hematogenous spread. Cancer remains a major public health concern and current preventative measures and treatments do not match the needs of most patients. Understanding of the neoplastic process of tumorigenesis can be aided by the identification of molecular markers of prognostic and diagnostic importance.

Current forms of cancer treatment include the use of immunosuppressive drugs (Morisaki, T. et al. (2000) Anticancer Res. 20:3363-3373; Geoerger, B. et al. (2001) Cancer Res. 61:1527-1532). The identification of proteins involved in cell signaling, and specifically proteins that act as receptors for immunosuppressant drugs, may facilitate the development of anti-tumor agents. For example, immunophilins are a family of conserved proteins found in both prokaryotes and eukaryotes that bind to irmmunosuppressive drugs with varying degrees of specificity. One such group of immunophilic proteins is the peptidyl-prolyl cis-trans isomerase (EC 5.2.1.8) family (PPIase, rotamase). These enzymes, first isolated from porcine kidney cortex, accelerate protein folding by catalyzing the cis-trans isomerization of proline imidic peptide bonds in oligopeptides (Fischer, G. and F. X. Schmid (1990) Biochemistry 29:2205-2212). Included within the immunophilin family are the cyclophilins (e.g., peptidyl-prolyl isomerase A or PPIA) and FK-binding protein (e.g., FKBP) subfamilies. Cyclophilins are multifunctional receptor proteins which participate in signal transduction activities, including those mediated by cyclosporin (or cyclosporine). The PPIase domain of each family is highly conserved between species. Although structurally distinct, these multifunctional receptor proteins are involved in numerous signal transduction pathways, and have been implicated in folding and trafficking events.

The immunophilin protein cyclophilin binds to the immunosuppressant drug cyclosporin A. FKBP, another immunophilin, binds to FK506 (or rapamycin). Rapamycin is an immunosuppressant agent that arrests cells in the G₁ phase of growth, inducing apoptosis. Like cyclophilin, this macrolide antibiotic (produced by Streptomyces tsukubaensis) acts by binding to ubiquitous, predominantly cytosolic immunophilin receptors. These immunophiln/immunosuppressant complexes (e.g., cyclophilin A/cyclosporin A (CypA/CsA) and FKBP12/FK506) achieve their therapeutic results through inhibition of the phosphatase calcineurin, a calcium/calmodulin-dependent protein kinase that participates in T-cell activation (Hamilton, G. S. and J. P. Steiner (1998) J. Med. Chem 41: 5119-5143). The murine fkbp51 gene is abundantly expressed in immunological tissues, including the thymus and T lymphocytes (Baughman, G. et al. (1995) Molec. Cell. Biol. 15:4395-4402). FKBP12/rapamycin-directed immunosuppression occurs through binding to TOR (yeast) or FRAP (FKBP12-rapamycin-associated protein, in mammalian cells), the kinase target of rapamycin essential for maintaining normal cellular growth patterns. Dysfunctional TOR signaling has been linked to various human disorders including cancer (Metcalfe, S. M. et al. (1997) Oncogene 15:1635-1642; Emami, S. et al. (2001) FASEB J. 15:351-361), and autoimmunity (Damoiseaux, J. G. et al. (1996) Transplantation 62:994-1001).

Several cyclophilin isozymes have been identified, including cyclophilin B, cyclophilin C, mitochondrial matrix cyclophilin, bacterial cytosolic and periplasmic PPIases, and natural-killer cell cyclophilin-related protein possessing a cyclophilin-type PPIase domain, a putative tumor-recognition complex involved in the function of natural killer (NK) cells. These cells participate in the innate cellular immune response by lysing virally-infected cells or transformed cells. NK cells specifically target cells that have lost their expression of major histocompatibility complex (MHC) class I genes (common during tumorigenesis), endowing them with the potential for attenuating tumor growth. A 150-kDa molecule has been identified on the surface of human NK cells that possesses a domain which is highly homologous to cyclophilin/peptidyl-prolyl cis-trans isomerase. This cyclophilin-type protein may be a component of a putative tumor-recognition complex, a NK tumor recognition sequence (NK-TR) (Anderson, S. K. et al. (1993) Proc. Natl. Acad. Sci. USA 90:542-546). The NKTR tumor recognition sequence mediates recognition between tumor cells and large granular lymphocytes (LGLs), a subpopulation of white blood cells (comprised of activated cytotoxic T cells and natural killer cells) capable of destroying tumor targets. The protein product of the NKTR gene presents on the surface of LGLs and facilitates binding to tumor targets. More recently, a mouse Nktr gene and promoter region have been located on chromosome 9. The gene encodes a NK-cell-specific 150-kDa protein (NK-TR) that is homologous to cyclophilin and other tumor-responsive proteins (Simons-Evelyn, M. et al. (1997) Genomics 40:94-100).

Other proteins that interact with tumorigenic tissue include cytokines such as tumor necrosis factor (TNF). The TNF family of cytolines are produced by lymphocytes and macrophages, and can cause the lysis of transformed (tumor) endothelial cells. Endothelialprotein 1 (Edp1) has been identified as a human gene activated transcriptionally by TNF-alpha in endothelial cells, and a TNF-alpha inducible Edp1 gene has been identified in the mouse (Swift, S. et al. (1998) Biochim. Biophys. Acta 1442:394-398).

Crystallins are stable, long-lived structural proteins of the vertebrate eye lens. Their structure and interactions are responsible for lens transparency. Gradual accumulation of small changes to the proteins causes age-related cataracts. The lens is formed from two protein superfamilies, the alpha- and beta gamma-crystallins. The proteins of both superfamilies have a basic 2-beta-sheet domain fold, with the beta gamma-domain being made from two intercalating Greek keys. The three types of crystallin proteins: alpha, beta and gamna, are each composed of a variety of subunits. (See Slingsby, C. and Clout, N. J.(1999) Eye 13:395-402; Carver, J. A. (1999) Prog. Retin. Eye Res. 18:431-462; and Graw, J. (1997) Biol. Chem 378:1331-1348.) Some crystallins have been discovered outside the eye. AIM1, a non-lens member of the betagamma-crystallin superfamily, is associated with the control of tumorigenicity in human malignant melanoma. AIM1 is a mammalian member of the betagamma superfamily with a primarily non-lens role. AIM1 is a candidate for the putative suppressor of malignant melanoma on chromosome 6, possibly exerting its effects through interactions with the cytoskeleton (Ray, M. E. et al. (1997) Proc. Natl. Acad. Sci. U S A 94:3229-3234).

Spermatogenesis associated factor (SPAF) is an AAA-protein (ATPase associated with diverse activities) specific to early spermatogenesis and malignant conversion. SPAF is expressed in spermatogonia and early spermatocytes in the basal compartment of the seminiferous tubules (Liu, Y. et al. (2000) Oncogene 19:1579-1588).

DICE1 is a tumor suppressor locus on human chromosome 13q14, that encodes a 100 kD protein similar to the carboxy-terminal half of the mouse EGF repeat transmembrane protein DBI-1. The DICE1 protein is expressed in a wide variety of fetal and adult tissues. The DBI-1 protein interferes with the mitogenic response to insulin-like growth factor 1 (IGF-1) and is implicated in anchorage-dependent growth. Expression of the DICE1 protein is reduced or undetectable in many non-small cell lung carcinomas analysed (Wieland, I. (1999) Oncogene 18:4530-4537).

Disintegrins are proteins which inhibit fibrinogen interaction with blood platelet receptors expressed on the glycoprotein Iib-IIIa complex. They act by binding to the integrin glycoprotein IIb-IIIa receptor on the platelet surface and inhibit aggregation induced by ADP, thrombin, platelet-activating factor and collagen. Disintegrins are peptides of about 70 amino acid residues that contain many cysteines involved in disulfide bonds (Williams, J. et al. (1990) Biochim. Biophys. Acta 1039:81-89; Dennis, M. S. et al. (1990) Proc. Natl. Acad. Sci. U.S.A. 87:2471-2475).

### Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

For example, microarray analysis can be used to determine relative expression levels of polynucleotides in normal lymphocytes compared with leukemia cells. Jurkat cells are an acute T-cell leukemia cell line that have been extensively used to study gene expression in human leukemia. Treatment of Jurkat cells with PMA (a broad activator of protein kinase C-dependent pathways) and ionomycin (a calcium ionophore that permits the entry of calcium in the cell) activates two of the major signaling pathways used by mammalian cells to interact with their environment and mimics the type of secondary signaling events elicited during optimal B-cell activation. Monitoring the expression level of polynucleotides before and after the treatment of Jurkat cells with PMA and ionomycin is useful for identifying cDNAs involved in T-cell activation.

### Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. Exposure of the bronchial epithelium to tobacco smoke appears to result in changes in tissue morphology, which are thought to be precursors of cancer. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda, R. et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen, S. et al.. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang, T. et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

### Breast Cancer

There are more than 180,000 new cases of breast cancer diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

### Prostate Cancer

Prostate cancer is a common malignancy in men over the age of 50, and the incidence increases with age. In the US, there are approximately 132,000 newly diagnosed cases of prostate cancer and more than 33,000 deaths from the disorder each year.

Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most cancers, prostate cancer develops through a multistage progression ultimately resulting in an aggressive, metastatic phenotype. The initial step in tumor progression involves the hyperproliferation of normal luminal and/or basal epithelial cells that become hyperplastic and evolve into early-stage tumors. The early-stage tumors are localized in the prostate but eventually may metastasize, particularly to the bone, brain or lung. About 80% of these tumors remain responsive to androgen treatment, an important hormone controlling the growth of prostate epithelial cells. However, in its most advanced state, cancer growth becomes androgen-independent and there is currently no known treatment for this condition.

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Current areas of cancer research provide additional prospects for markers as well as potential therapeutic targets for prostate cancer. Several growth factors have been shown to play a critical role in tumor development, growth, and progression. The growth factors Epidermal Growth Factor (EGF), Fibroblast Growth Factor (FGF), and Tumor Growth Factor alpha (TGFA) are important in the growth of normal as well as hyperproliferative prostate epithelial cells, particularly at early stages of tumor development and progression, and affect signaling pathways in these cells in various ways (Lin J et al. (1999) Cancer Res. 59:2891-2897; Putz T et al. (1999) Cancer Res 59:227-233). The TGF-β family of growth factors are generally expressed. at increased levels in human cancers and the high expression levels in many cases correlates with advanced stages of malignancy and poor survival (Gold L I (1999) Crit Rev Oncog 10:303-360). Finally, there are human cell lines representing both the androgen-dependent stage of prostate cancer (LNCap) as well as the androgen-independent, hormone refractory stage of the disease (PC3 and DU-145) that have proved useful in studying gene expression patterns associated with the progression of prostate cancer, and the effects of cell treatments on these expressed genes (Chung T D (1999) Prostate 15:199-207).

### Leukemias

Leukemias can be classified into four major categories, and all involve malignant transformation of pluripotent stem cells. Acute leukemias, both lymphoblastic (ALL) and myeloid (AML) types, are characterized by the presence of immature cells in the blood. Chronic leukemias, both lymphocytic (CLL) and myelocytic (CML), are associated with mature, differentiated cells, but proportions of each cell type are abnormal. For example, CLL patients usually have clonal expansion of B cell lymphocytes. CML patients often have granulocytes of all stages of maturity present in blood, bone marrow, and other organs. Monoclonal antibodies specific for B- and T-cells are helpful diagnostic tools, in addition to histological analysis. Disease progresses as normal hematopoietic bone marrow is displaced by malignant cells. Cause has been determined to be genetic in some cases, and chemical or radiation-induced in others.

### Aging and Senescence

Studies of the aging process or senescence have shown a number of characteristic cellular and molecular changes (Fauci et al. (1998) Harrison's Principles of Internal Medicine, McGraw-Hill, New York N.Y., p.37). These characteristics include increases in chromosome structural abnormalities, DNA cross-linking, incidence of single-stranded breaks in DNA, losses in DNA methylation, and degradation of telomere regions. In addition to these DNA changes, post-translational alterations of proteins increase including, deamidation, oxidation, cross-linking, and nonenzymatic glycation. Still further molecular changes occur in the mitochondria of aging cells through deterioration of structure. These changes eventually contribute to decreased function in every organ of the body.

Alzheimer's disease is a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. Early in Alzheimer's pathology, physiological changes are visible in the cingulate cortex (Minoshima, S. et al. (1997) Annals of Neurology 42:85-94). In subjects with advanced Alzheimer's disease, accumulating plaques damage the neuronal architecture in limbic areas and eventually cripple the memory process.

### Genes Regulated in Dendritic Cell Differentiation

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for examining which genes are tissue specific, carrying out housekeeping functions, parts of a signaling cascade, or specifically related to a particular genetic predisposition, condition, disease, or disorder.

The potential application of gene expression profiling is particularly relevant to characterizing lineage differences during cellular development that will improve diagnosis, prognosis, and treatment of disease. For example, both the levels and sequences expressed in dendritic cells from subjects with autoimmunity may be compared with the levels and sequences expressed in dendritic cells from normal subjects.

Dendritic cells (DC) are antigen presenting cells (APC) that play a key role in the primary immune response because of their unique ability to present antigens to naive T cells. In addition, DC differentiate into separate subsets that sustain and regulate immune responses following initial contact with antigen. DC subsets include those that preferentially induce particular T helper 1 (Th1) or T helper 2 (Th2) responses and those that regulate B cell responses. Moreover, DC are increasingly being used to manipulate immune responses, either to downregulate an aberrant autoimmune response or to enhance vaccination or a tumor-specific response.

DC are functionally specialized in correlation with their particular differentiation state. CD34+ myeloid cells found in the bone marrow mature in response to as yet unclear signals into CD14+ CD11c+ monocytes. An innate or antigen non-specific response takes place initially when monocytes circulate to nonlymphoid tissues and respond to lipopolysaccharide (LPS), a bacterially-derived mitogen, and viruses. Such direct encounter with antigen causes secretion of pro-inflammatory cytokines that attract and regulate natural killer cells, macrophages, and eosinophils in the first line of defense against invading pathogens. Monocytes then mature into DC, which capture antigen highly efficiently through endocytosis and antigen receptor uptake. Antigen processing and presentation trigger activation and differentiation into mature DC that express MHC class II molecules on the cell surface and efficiently activate T cells, initiating antigen-specific T cell and B cell responses. In turn, T cells activate DC through CD40 ligand—CD40 interactions, which stimulate expression of the costimulatory molecules CD80 and CD86, the latter most potent in amplifying T cell responses. DC interaction via CD40 with T cells also stimulates the production of inflammatory cytokines such as TNF alpha and IL-1. Engagement of RANK, a member of the TNF receptor family by its ligand, TRANCE, which is expressed on activated T cells, enhances the survival of DC through inhibition of apoptosis, thereby enhancing T cell activation. The maturation and differentiation of monocytes into mature DC links the antigen non-specific innate immune response to the antigen-specific adaptive immune response.

The process by which monocytes differentiate into immature dendritic cells in vivo has not been fully elucidated. Incubation of monocytes with granulocyte-macrophage colony stimulating factor (GM-CSF) and interleukin (IL)-4 in vitro yields cells that exhibit functional and morphological characteristics equivalent to immature dendritic cells found in vivo. Moreover, incubation in vitro of immature dendritic cells with tumor necrosis factor alpha (TNF-α), CD40 ligand, LPS, or monocyte-conditioned medium yields mature dendritic cells that are potent activators of naive T cells.

The ability to manipulate DC in vitro and their capacity to mount an effective immune response with small numbers of DC and little antigen has led to potential immunotherapies for diseases such as cancer, AIDS, and infectious diseases; and enhancing vaccine efficacy. Spontaneous remissions of particular cancers such as renal cell carcinomas and melanomas indicate that the immune system can respond to tumor antigens and eliminate tumors. However, tumors escape immune surveillance through a number of means including secretion of IL-10, macrophage colony stimulating factor, IL-6, and vascular endothelial growth factor, all of which inhibit DC activity and promote tolerance of tumor tissue. Delivery of tumor antigen-loaded DC to tumors can induce tumor-specific rejection in animal models. Similarly, pathogens can escape immune surveillance by altering antigen processing and presentation pathways or interfering with maturation of antigen presenting cells. Rather than providing resistance, DC can complicate infection by hosting latent viruses such as Kaposi's virus and cytomegalovirus, complicating infection. HIV-1 and measles virus particles are efficiently produced in DC. Vaccines against tumors or infectious pathogens could be improved by systemic or local administration of DC loaded with tumor antigens or attenuated viral particles or components, respectively.

The expression of killer-inhibitor regulatory molecules, chemokines, chemokine receptors, and proteinases have been identified in DC through sequencing of ESTs. Continuing this search may reveal new lymphocyte-binding and antigen-processing molecules, transmembrane and secretory products, and transcription factors that may help to explain the specialized features of DC and allow manipulation of the immune system.

### Adipocyte Maturation

The primary function of adipose tissue is the ability to store and release fat during periods of feeding and fasting. White adipose tissue is the major energy reserve in periods of fasting, and its reserve is mobilized during energy deprivation. Adipose tissue is one of the primary target tissues for insulin, and adipogenesis and insulin resistance are linked in type II diabetes, non-insulin dependent diabetes mellitus (NIDDM). Cytologically the conversion of a preadipocytes into mature adipocytes is characterized by deposition of fat droplets around the nuclei. The conversion process in vivo can be induced by thiazolidinediones and other PPARγ agonists (Adams et al. (1997) J Clin Invest 100:3149-3153) which also lead to increased sensitivity to insulin and reduced plasma glucose and blood pressure.

Pickup and Crook (1998; Diabetologia 41:1241-8) have suggested that NIDDM may result from the inability of an individual with hypersensitive acute-phase immune response to carry out normal cell signaling and repair. Steps in this process are highly correlated with long-term lifestyle and environment and include: 1) high glucose stimulation of insulin and cytokine production, 2) influence of various cytokines on tissue remodeling during adipocyte differentiation and their affect on signaling pathways, and 3) occurrence of tissue damage when cytokines continue to be produced, extracellular matrix components (FCM) are not recycled, and homeostasis is not timely restored. Many cytokines and the receptors with which they interact are implicated in this process. These cytokines include tumor necrosis factor, connective tissue growth factor, transforming growth factor-beta, interleukin (IL)-13 and their receptors. Tumor necrosis factor contributes to insulin resistance by inhibiting insulin-stimulated tyrosine phosphorylation of the insulin receptor. This, in turn, prevents the insulin receptor from participating in normal signaling processes (Skolnik and Marcusohn (1996) Cytokine Growth Factor Rev 7:161-173; Hotamisligil (1999) J Intern med 245:621-625). Connective tissue growth factor mediates the buildup of mesenglial matrix (Murphy et al. (2000) J Biol Chem 274:5830-5834). Transforming growth factor-beta mediates the buildup of mesenglial matrix of the kidney and affects vascular function through its interaction with the inositol trisphosphate receptor, a key intracellular calcium channel (Sharma and McGowan (2000) Cytokine Growth Factor Rev 11:115-123).

IL-13 and IL-4 are immuno-regulatory cytokines which share many overlapping biological properties. They both promote growth of B-cells (McKenzie et al. (1993) Proc Natl Acad Sci 90:3735-3739), induce expression of germ line Cε transcripts, and direct naive B cells to switch to the synthesis of IgE and IgG4 ( Punnomen et al. (1993) Proc Natl Acad Sci 90:3730-3734). Similarly, different isoforms of the IL-13 and IL-4 receptors interact to form four types of IL-13 receptor complexes. In some instances, IL-13 utilizes a receptor complex composed of the IL-4 receptor-α chain (Rα) and the IL-13Rα1. Although the specific role of each chain in IL-13 signaling is unclear, Ba/F3 cells transfected with IL-13Rα1 display a mitogenic response to IL-13, but cells transfected with mouse IL-13Rα2 do not. In addition, a soluble IL-13Rα2/Fc fusion protein blocks the mitogenic response to IL-13 (Donaldson et al. (1998) J Immunol 161:2317-2324). This suggests that IL-13Rα2 could serve as a dominant negative inhibitor or decoy receptor for IL-13. However, in colonic carcinoma cell lines, the receptor complex displayed growth inhibition which was associated with tyrosine phosphorylation of insulin receptor substrate-1. It is evident that more research is needed to establish 1) which isoforms of the receptor complex promote cell growth and which inhibit cell growth and 2) whether this varies by cell or tissue type.

Most adipocyte research has been carried out using mouse cell lines. Recent evidence, however, indicates that culture conditions which stimulate mouse preadipocyte differentiation are different from those which induce human preadipocytes. In addition to the known genetic differences between these species, diploid human primary cells respond differently than aneuploid mouse cells.

Atherosclerosis and the associated coronary artery disease and cerebral stroke represent the most common cause of death in industrialized nations. Although certain key risk factors have been identified, a full molecular characterization that elucidates the causes and provide care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus. Vascular lesions principally involve the vascular endothelium and the surrounding smooth muscle tissue.

Development of atherosclerosis is understood to be induced by the presence of circulating lipoprotein. Lipoproteins, such as the cholesterol-rich lowdensity lipoprotein (LDL), accumulate in the extracellular space of the vascular intima, and undergo modification. Oxidation of LDL (Ox-LDL) occurs most avidly in the sub-endothelial space where circulating antioxidant defenses are less effective. Mononuclear phagocytes enter the intima, differentiate into macrophages, and ingest modified lipids including Ox-LDL. During Ox-LDL uptake, macrophages produce cytokines (e.g. tumor necrosis factor a (TNF-α) and interleukin-1 (IL-1)) and growth factors (e.g. M-CSF, VEGF, and PDGF-BB) that elicit further cellular events that modulate atherogenesis such as smooth muscle cell proliferation and production of extracellular matrix by vascular endothelium. Additionally, these macrophages may activate genes in endothelium and smooth muscle tissue involved in inflammation and tissue differentiation, including superoxide dismutatse (SOD), IL-8, and ICAM-1.

The vascular endothelium influences not only the three classically interacting components of hemostasis: the vessel, the blood platelets and the clotting and fibrinolytic systems of plasma, but also the natural sequelae: inflammation and tissue repair. Two principal modes of endothelial behavior may be differentiated, best defined as an anti- and a prothrombotic state. Under physiological conditions endothelium mediates vascular dilatation (formation of nitric oxide (NO), PGI₂, adenosine, hyperpolarising factor), prevents platelet adhesion and activation (production of adenosine, NO and PGI₂, removal of ADP), blocks thrombin formation (tissue factor pathway inhibitor, activation of protein C via thrombomodulin, activation of antithrombin III) and mitigates fibrin deposition (t- and scuplasminogen activator production). Adhesion and transmigration of inflammatory leukocytes are attenuated, e.g. by NO and IL-10, and oxygen radicals are efficiently scavenged (urate, NO, glutathione, SOD).

When the endothelium is physically disrupted or functionally perturbed by postischemic reperfusion, acute and chronic inflammation, atherosclerosis, diabetes and chronic arterial hypertension, then completely opposing actions pertain. This prothrombotic, proinflammatory state is characterised by vaso-constriction, platelet and leukocyte activation and adhesion (extemalisation, expression and upregulation of, for example, von Willebrand factor, platelet activating factor, P-selectin, ICAM-1, IL-8, MCP-1, and TNF-α), promotion of thrombin formation, coagulation and fibrin deposition at the vascular wall (expression of tissue factor, PAI-1, and phosphatidyl serine) and, in platelet-leukocyte coaggregates, additional inflammatory interactions via attachment of platelet CD40-ligand to endothelial, monocyte and B-cell CD40. Since thrombin formation and inflammatory stimulation set the stage for later tissue repair, complete abolition of such endothelial responses cannot be the goal of clinical interventions aimed at limiting procoagulatory, prothrombotic actions of a dysfunctional vascular endothelium (See, e.g., Becker et al. (2000) Z Kardiol 89:160-167.)

Tumor necrosis factor a is a pleiotropic cytoline that a mediates immune regulation and inflammatory responses. TNF-α-related cytokines generate partially overlapping cellular responses, including differentiation, proliferation, nuclear factor-κB (NF-κB) activation, and cell death, by triggering the aggregation of receptor monomers (Smith, C. A. et al. (1994) Cell 76:959-962). The cellular responses triggered by TNF-α are initiated through its interaction with distinct cell surface receptors (TNFRs). NF-κB is a transcription factor with a pivotal role in inducing genes involved in physiological processes as well as in the response to injury and infection. Activation of NF-κB involves the phosphorylation and subsequent degradation of an inhibitory protein, IKB,and many of the proximal kinases and adaptor molecules involved in this process have been elucidated. Additionally, the NP-κB activation pathway from cell membrane to nucleus for IL-1 and TNF-α is now understood (Bowie and O'Neill (2000) Biochem Pharmacol 59:13-23).

Monocyte cheinoattractant protein-1 (MCP-1) is known to play an important role in the pathogenesis of atherosclerosis by inducing monocyte migration. TNF-α treatment of human umbilical vein endothelial cells (HUVECs) increased the cellular secretions of MCP-1 119-fold compared with untreated cells. Troglitazone, an insulin-sensitizing drug, significantly inhibited this TNP-α-induced increase in MCP-1 secretions and decreased mRNA levels (Ohta et al. (2000) Diabetes Res Clin Pract 48:171-176).

Treatment of confluent cultures of vascular smooth muscle cells (SMCs) with TNF-α suppresses the incorporation of [³H]proline into both collagenase-digestible proteins (CDP) and noncollagenous proteins (NCP). Such suppression by TNF-α is not observed in confluent bovine aortic endothelial cells and human fibroblastic IMR-90 cells. TNF-α decreases the relative proportion of collagen types IV and V suggesting that TNF-α modulates collagen synthesis by SMCs depending on their cell density and therefore may modify formation of atherosclerotic lesions (Hiraga et al. (2000) Life Sci 66:235-244).

Human coronary artery smooth muscle cells (CASMC) are primary cells isolated from the tunica media (an intermediate muscular layer) of a human coronary artery. Vascular smooth muscle cells are a model of increasing significance in vascular biology. It is now well known that besides their obvious role in the regulation of vascular tone and, consequently, oxygen supply to various tissues, their behavior under inflammatory conditions is an important factor in the development of atherosclerosis and restenosis.

Human aortic endothelial cells (HAECs) are primary cells derived from the endothelium of a human aorta. HAECs have been. used as an experimental model for investigating in vitro the role of the endothelium in human vascular biology. Activation of the vascular endothelium is considered to be a central event in a wide range of both physiological and pathophysiological processes, such as vascular tone regulation, coagulation and thrombosis, atherosclerosis, and inflammation.

Thus, vascular tissue genes differentially expressed during treatment of CASMC and HAEC cell cultures with TNFA may reasonably be expected to be markers of the atherosclerotic process.

### Biopharmacological Tools for Microarray Analysis

Human umbilical vein endothelial cells (HEVECs) are a primary cell line derived from the endothelium of the human umbilical vein. HUVECs are used to study the functional biology of human endothelial cells in vitro. Activation of vascular endothelium is observed in physiological and pathophysiological processes including vascular tone regulation, coagulation and thrombosis, atherosclerosis, and inflammation.

Tumor necrosis factor-alpha (TNF-α) is a pleiotropic cytokine that plays a central role in mediation of the inflammatory response through activation of multiple signal transduction pathways. TNF-α is produced by activated lymphocytes, macrophages, and other white blood cells, and activates endothelial cells.

PMA is an agonist of protein kinase C (PKC). PKC is a calcium-activated, phospholipid-dependent serine- and threonine-specific kinase that, upon activation, phosphorylates a broad range of secondary targets. TNF-α causes translocation of PKC from the cytosol to the membrane where it phosphorylates a variety of targets.

Interleukin 1 beta (IL-lp)is a cytokine associated with acute inflammatory responses and is involved in processes such as fever induction, metabolic regulation, and bone remodeling. Both cells of the immune system (monocytes, dendritic cells, NK cells, platelets, and neutrophils) and somatic cells (osteoblasts, neurons, Schwann cells, oligodendrocytes, and adrenal cortical cells) can produce IL-1β. IL-1β can induce its own production in monocytes, the production of adhesion molecules and chemokines in endothelial cells, and interferon gamma (IFN-γ) production by NK cells in conjunction with IL-12. IL-1 is produced as a single chain pro-molecule that must be cleaved by a specialized protease—IL-1 Converting Enzyme (ICE)—to acquire its function. Interleukin 10 (L-10) is produced by CD4+ T cell clones and some CD8+ T cell clones. Human B cells, EBV-transformed lymphoblastoid cell lines, and monocytes can also produce IL-10 upon activation. IL-10 is a pleiotrophic cytokine that can exert either immunostimulatory or immunosupressive effects on a variety of cell types. It is a potent immunosuppressant of macrophage functions. In vitro, IL-10 can inhibit the accessory function and antigen-presenting capacity of monocytes by, among other effects, downregulating class II MHC expression. Thus, IL-10 can inhibit monocyte/macrophage-dependent, antigen specific proliferation of mouse Th1 clones as well as human Th0-, Th1-, and Th2-like T cells. IL-10 can also inhibit the monocyte/macrophage-dependent, antigen stimulated cytokine synthesis (especially IFN-g) by human PBMNC and NK. Additionally, IL-10 is a potent inhibitor of monocyte/macrophage activation and its resultant cytotoxic effects. It can suppress the production of numerous cytokines including TNF-a, IL-1, IL-6, and IL-10, as well as the synthesis of superoxide anion, reactive oxygen intermediates, and reactive nitrogen intermediates by activated monocytes/macrophages. As an immunostimulatory cytokine, IL-10 can act on B cells to enhance their viability, cell proliferation, Ig secretion, and class II MHC expression. Aside from B-lymphocytes, IL10 is also a growth co-stimulator for thymocytes and mast cells, as well as an enhancer of cytotoxic T cell development.

Thiazolidinediones or peroxisome proliferator-activated receptor γ (PPAR-γ) agonists are a new class of antidiabetic agents that improve insulin sensitivity and reduce plasma glucose and blood pressure in subjects with type II diabetes. These agents can bind and activate an orphan nuclear receptor and some of them induce human adipocyte differentiation.

There is a need in the art for new compositions, including nucleic acids and proteins, for the diagnosis, prevention, and treatment of cell proliferative disorders including cancer, developmental disorders, neurological disorders, autoimmune/inflammatory disorders, reproductive disorders, and disorders of the placenta.

### SUMMARY OF THE INVENTION

Various embodiments of the invention provide purified polypeptides, proteins associated with cell growth, differentiation, and death, referred to collectively as ‘CGDD’ and individually as ‘CGDD-1,’ ‘CGDD-2,’ ‘CGDD-3,’ ‘CGDD-4,’ ‘CGDD-5,’ ‘CGDD-6,’ ‘CGDD-7,’ ‘CGDD-8,’ ‘CGDD-9,’ ‘CGDD-10,’ ‘CGDD-11,’ ‘CGDD-12,’ ‘CGDD-13,’ ‘CGDD-14,’ ‘CGDD-15,’ ‘CGDD-16,’ ‘CGDD-17,’ ‘CGDD-18,’ ‘CGDD-19,’ ‘CGDD-20,’ ‘CGDD-21,’ ‘CGDD-22, ’ ‘CGDD-23,’ ‘CGDD-24,’ ‘CGDD-25,’ ‘CGDD-26,’ and ‘CGDD-27’ and methods for using these proteins and their encoding polynucleotides for the detection, diagnosis, and treatment of diseases and medical conditions. Embodiments also provide methods for utilizing the purified proteins associated with cell growth, differentiation, and death and/or their encoding polynucleotides for facilitating the drug discovery process, including determination of efficacy, dosage, toxicity, and pharmacology. Related embodiments provide methods for utilizing the purified proteins associated with cell growth, differentiation, and death and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. Another embodiment provides an isolated polypeptide comprising an amino acid sequence of SEQ ID NO:1-27.

Still another embodiment provides an isolated polynucleotide encoding a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. In another embodiment, the polynucleotide encodes a polypeptide selected from the group consisting of SEQ ID NO:1-27. In an alternative embodiment, the polynucleotide is selected from the group consisting of SEQ ID NO:28-54.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27.

Still yet another embodiment provides an isolated polynucleotide selected from the group consisting of a) a polynucleotide comprising a polynucleotide sequence selected from the group consisting of SEQ ID NO:28-54, b) a polynucleotide comprising a naturally occurring polynucleotide sequence at least 90% identical or at least about 90% identical to a polynucleotide sequence selected from the group consisting of SEQ ID NO:28-54, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:28-54, b) a polynucleotide comprising a naturally occurring polynucleotide sequence at least 90% identical or at least about 90% identical to a polynucleotide sequence selected from the group consisting of SEQ ID NO:28-54, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected.from the group consisting of SEQ ID NO:28-54, b) a polynucleotide comprising a naturally occurring polynucleotide sequence at least 90% identical or at least about 90% identical to a polynucleotide sequence selected from the group consisting of SEQ ID NO:28-54, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27, and a pharmaceutically acceptable excipient. In one embodiment, the composition can comprise an amino acid sequence selected from the group consisting of SEQ ID NO:1-27. Other embodiments provide a method of treating a disease or condition associated with decreased or abnormal expression of functional CGDD, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional CGDD, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional CGDD, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-27, b) a polypeptide comprising a naturally occurring amino acid sequence at least 90% identical or at least about 90% identical to an amino acid sequence selected from the group consisting of SEQ ID NO:1-27, c) a biologically active fragment of a polypeptide having an amino acid sequence selected from the group consisting of SEQ ID NO:1-27, and d) an immunogenic fragment of a polypeptide having an amino acid sequence selected from the group consisting of SEQ ID NO:1-27. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:28-54, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:28-54, ii) a polynucleotide comprising a naturally occurring polynucleotide sequence at least 90% identical or at least about 90% identical to a polynucleotide sequence selected from the group consisting of SEQ ID NO:28-54, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:28-54, ii) a polynucleotide comprising a naturally occurring polynucleotide sequence at least 90% identical or at least about 90% identical to a polynucleotide sequence selected from the group consisting of SEQ ID NO:28-54, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### DEFINITIONS

“CGDD” refers to the amino acid sequences of substantially purified CGDD obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of CGDD. Agonists may include proteins, nucleic acids, carbohydrates, small molecules, or any other compound or composition which modulates the activity of CGDD either by directly interacting with CGDD or by acting on components of the biological pathway in which CGDD participates.

An “allelic variant” is an alternative form of the gene encoding CGDD. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding CGDD include those sequences with deletions, insertions, or substitutions of different nucleotides, resulting in a polypeptide the same as CGDD or a polypeptide with at least one functional characteristic of CGDD. Included within this definition are polymorphisms which may or may not be readily detectable using a particular oligonucleotide probe of the polynucleotide encoding CGDD, and improper or unexpected hybridization to allelic variants, with a locus other than the normal chromosomal locus for the polynucleotide encoding CGDD. The encoded protein may also be “altered,” and may contain deletions, insertions, or substitutions of amino acid residues which produce a silent change and result in a functionally equivalent CGDD. Deliberate amino acid substitutions may be made on the basis of one or more similarities in polarity, charge, solubility, hydrophobicity, hydrophilicity, and/or the amphipathic nature of the residues, as long as the biological or immunological activity of CGDD is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule. “Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of CGDD. Antagonists may include proteins such as antibodies, anticalins, nucleic acids, carbohydrates, small molecules, or any other compound or composition which modulates the activity of CGDD either by directly interacting with CGDD or by acting on components of the biological pathway in which CGDD participates.

The term “antibody” refers to intact immunoglobulin molecules as well as to fragments thereof, such as Fab, F(ab′)₂, and Fv fragments, which are capable of binding an epitopic determinant. Antibodies that bind CGDD polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic CGDD, or of any oligopeptide thereof, to induce a specific immune response in appropriate animals or cells and to bind with specific antibodies. “Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding CGDD or fragments of CGDD may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.). “Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence. “Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide. “Differential expression” refers to increased or upregulated; or decreased, downregulated, or absent gene or protein expression, determined by comparing at least two different samples. Such comparisons may be carried out between, for example, a treated and an untreated sample, or a diseased and a normal sample. “Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of CGDD or a polynucleotide encoding CGDD which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:28-54 can comprise a region of unique polynucleotide sequence that specifically identifies SEQ ID NO:28-54, for example, as distinct from any other sequence in the genome from which the fragment was obtained. A fragment of SEQ ID NO:28-54 can be employed in one or more embodiments of methods of the invention, for example, in hybridization and amplification technologies and in analogous methods that distinguish SEQ ID NO:28-54 from related polynucleotides. The precise length of a fragment of SEQ ID NO:28-54 and the region of SEQ ID NO:28-54 to which the fragment corresponds are routinely determinable by one of ordinary skill in the art based on the intended purpose for the fragment.

A fragment of SEQ ID NO:1-27 is encoded by a fragment of SEQ ID NO:28-54. A fragment of SEQ ID NO:1-27 can comprise a region of unique amino acid sequence that specifically identifies SEQ ID NO:1-27. For example, a fragment of SEQ ID NO:1-27 can be used as an immunogenic peptide for the development of antibodies that specifically recognize SEQ ID NO:1-27. The precise length of a fragment of SEQ ID NO:1-27 and the region of SEQ ID NO:1-27 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence. “Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Aligmnent Search Tool (BLAST) (Altschul, S. P. et al. (1990) J. Mol. Biol. 215:403410), which is available from several sources, including the NCBL Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Rewardfor match: 1
Penalty for nisinatch: -2
Open Gap: 5 and Extension Gap: 2 penalties
Gap x drop-off 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap x drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured. “Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability. “Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters,. chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively. “Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of CGDD which is capable of eliciting an immune response when introduced into a living organism, for example, a mammal. The term “immunogenic fragment” also includes any polypeptide or oligopeptide fragment of CGDD which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of CGDD. For example, modulation may cause an increase or a decrease in protein activity, binding characteristics, or any other biological, functional, or immunological properties of CGDD.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. “Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell. “Post-translational modification” of an CGDD may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of CGDD. “Probe” refers to nucleic acids encoding CGDD, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemilumininescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989; Molecular Cloning: A Laboratory Manual, 2^{nd} ed., vol. 1-3, Cold Spring Harbor Press, Plainview N.Y.), Ausubel, F. M. et al. (1999; Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability. “Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing CGDD, nucleic acids encoding CGDD, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively. “Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time. “transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May, 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

Various embodiments of the invention include new human proteins associated with cell growth, differentiation, and death (CGDD), the polynucleotides encoding CGDD, and the use of these compositions for the diagnosis, treatment, or prevention of cell proliferative disorders including cancer, developmental disorders, neurological disorders, autoimmune/inflammatory disorders, reproductive disorders, and disorders of the placenta.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are proteins associated with cell growth, differentiation, and death.

For example, SEQ ID NO:2 is 51% identical, from residue G11 to residue A548, to a human leucine-rich glioma-inactivated protein precursor (GenBank ID g4091819) as determined by the Basic Local Alignment Search Tool (BLAST). (See Table 2.) The BLAST probability score is 4.3e-161, which indicates the probability of obtaining the observed polypeptide sequence alignment by chance. SEQ ID NO:2 also contains a leucine-rich repeat domain as determined by searching for statistically significant matches in the hidden Markov model (HMM)-based PFAM database of conserved protein family domains. (See Table 3.)

As another example, SEQ ID NO:3 is 90% identical, from residue M1 to residue L378, to Mus musculus Iroquois-class homeobox protein IRX2 (GenBank ID g9965418) as determined by the Basic Local Alignment Search Tool (BLAST). (See Table 2.) The BLAST probability score is 1.5e-190, which indicates the probability of obtaining the observed polypeptide sequence alignment by chance. SEQ ID NO:3 also contains a homeobox domain as determined by searching for statistically significant matches in the hidden Markov model (HMM)-based PFAM database of conserved protein family domains. (See Table 3.) Data from BLIMPS, MOTIFS, and PROFILESCAN analyses and BLAST analyses of the PRODOM and DOMO databases provide further corroborative evidence that SEQ ID NO:3 is a homeodomain containing protein.

As another example, SEQ ID NO:6 is 99% identical, from residue Q87 to residue T1723, to human non-lens beta gamma-crystallin like protein (GenBank ID g2072425) as determined by the Basic Local Alignment Search Tool (BLAST). (See Table 2.) The BLAST probability score is 0.0, which indicates the probability of obtaining the observed polypeptide sequence alignment by chance. SEQ ID NO:6 also contains a beta/gamma crystallin domain as determined by searching for statistically significant matches in the hidden Markov model (HMM)-based PFAM database of conserved protein family domains. (See Table 3.) Data from MOTIFS, and other BLAST analyses provide further corroborative evidence that SEQ ID NO:6 is a beta gamma-crystallin like protein.

As another example, SEQ ID NO:9 is 53% identical from residue M1 to residue K56, and 84% identical from residue G55 to residue G535, to Mus musculus SPAF (spermatogenesis associated factor, AAA family) (GenBank ID g4105619) as determined by the Basic Local Alignment Search Tool (BLAST). (See Table 2.) The BLAST probability score is 6.4e-218, which indicates the probability of obtaining the observed polypeptide sequence alignment by chance. SEQ ID NO:9 also contains an ATPase family associated with various cellular activities (AAA) domain as determined by searching for statistically significant matches in the hidden Markov model (HMM)-based PFAM database of conserved protein family domains. (See Table 3.) Data from BLIMPS and MOTIFS analyses provide further corroborative evidence that SEQ ID NO:9 is an AAA family protein.

As another example, SEQ ID NO:14 is 85% identical, from residue M501 to residue I1440, to rat CPG2 (candidate plasticity-related genes) protein, which is involved among other things, in postnatal cortical development (Nedivi, E. et al. (1996) Proc. Nati. Acad. Sci. U.S.A. 93 (5), 2048-2053) (GenBank ID g1177322) as determined by the Basic Local Alignment Search Tool (BLAST). (See Table 2.) The BLAST probability score is 0.0, which indicates the probability of obtaining the observed polypeptide sequence alignment by chance. SEQ ID NO:14 also contains a calponin homology (CH) domain as determined by searching for statistically significant matches in the hidden Markov model (H-based PFAM database of conserved protein family domains. (See Table 3.) Data from MOTIFS, PROFILESCAN, and fuuther BLAST analyses provide corroborative evidence that SEQ ID NO:14 is a CPG2 protein.

As another example, SEQ ID NO:17 is 95% identical, from residue M1 to residue D686, to murine metastasis associated protein 1 (GenBank ID g15077051) as determined by the Basic Local Alignment Search Tool (BLAST). (See Table 2.) The BLAST probability score is 0.0, which indicates the probability of obtaining the observed polypeptide sequence alignment by chance. SEQ ID NO:17 also contains BAH, ELM2, GATA zinc finger, and Myb-like DNA-binding domains as determined by searching for statistically significant matches in the hidden Markov model (HMM)-based PFAM database of conserved protein family domains. (See Table 3.) Data from BLAST-PRODOM, BLAST-DOMO, and MOTIFS analyses provide further corroborative evidence that SEQ ID NO:17 is a protein associated with cell growth, differentiation and death.

As another example, SEQ ID NO:21 is 97% identical, from residue M1 to residue F652, to human OS-9 precursor (GenBank ID g1322234) as determined by the Basic Local Alignment Search Tool (BLAST). (See Table 2.) The BLAST probability score is 0.0, which indicates the probability of obtaining the observed polypeptide sequence alignment by chance. Data from BLIMPS, BLAST, and MOTIFS analyses provide further corroborative evidence that SEQ ID NO:21 is a protein associated with cell growth, differentiation, and death.

As another example, SEQ ID NO:25 is 99% identical, from residue M1 to residue T533, to human OS-9 (GenBank ID g2780783) as determined by the Basic Local Alignment Search Tool (BLAST). (See Table 2.) The BLAST probability score is 1.5e-290, which indicates the probability of obtaining the observed polypeptide sequence alignment by chance. SEQ ID NO:25 also has homology to proteins that are OS-9 proteins, as determined by BLAST analysis using the PROTEOME database. Data from BLAST analyses provide further corroborative evidence that SEQ ID NO:25 is an OS-9.

SEQ ID NO:1, SEQ ID NO:4-5, SEQ ID NO:7-8, SEQ ID NO:10-13, SEQ ID NO:15-16, SEQ ID NO:18-20, SEQ ID NO:22-24, and SEQ ID NO:26-27 were analyzed and annotated in a similar manner. The algorithms and parameters for the analysis of SEQ ID NO:1-27 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:8-14 or that distinguish between SEQ ID NO:8-14 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENSr”). Alternatively, the polynucleotide fragments described in column 2 may be derived from the NCBI RefSeq Nucleotide Sequence Records Database (i.e., those sequences including the designation “NM,” or “NT”) or the NCBI RefSeq Protein Sequence Records (ie., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N₁_N₂_YYYYY_N₃_N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, Cambridge, |
| | UK). |
| GBI | Hand-edited analysis of genomic sequences. |
| FL | Stitched or stretched genomic sequences (see Example V). |
| INCY | Full length transcript and exon prediction from mapping of EST |
| | sequences to the genome. Genomic location and EST |
| | composition data are combined to predict the exons and resulting |
| | transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB 1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses CGDD variants. A preferred CGDD variant is one which has at least about 80%, or alternatively at least about 90%, or even at least about 95% amino acid sequence identity to the CGDD amino acid sequence, and which contains at least one functional or structural characteristic of CGDD.

Various embodiments also encompass polynucleotides which encode CGDD. In a particular embodiment, the invention encompasses a polynucleotide sequence comprising a sequence selected from the group consisting of SEQ ID NO:28-54, which encodes CGDD. The polynucleotide sequences of SEQ ID NO:28-54, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding CGDD. In particular, such a variant polynucleotide will have at least about 70%, or alternatively at least about 85%, or even at least about 95% polynucleotide sequence identity to a polynucleotide encoding CGDD. A particular aspect of the invention encompasses a variant of a polynucleotide comprising a sequence selected from the group consisting of SEQ ID NO:28-54 which has at least about 70%, or alternatively at least about 85%, or even at least about 95% polynucleotide sequence identity to a nucleic acid sequence selected from the group consisting of SEQ ID NO:28-54. Any one of the polynucleotide variants described above can encode a polypeptide which contains at least one functional or structural characteristic of CGDD.

In addition, or in the alternative, a polynucleotide variant of the invention is a splice variant of a polynucleotide encoding CGDD. A splice variant may have portions which have significant sequence identity to a polynucleotide encoding CGDD, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding CGDD over its entire length; however, portions of the splice variant will have at least about 70%, or alternatively at least about 85%, or alternatively at least about 95%, or alternatively 100% polynucleotide sequence identity to portions of the polynucleotide encoding CGDD. For example, a polynucleotide comprising a sequence of SEQ ID NO:51 and a polynucleotide comprising a sequence of SEQ ID NO:38 are splice variants of each other; and a polynucleotide comprising a sequence of SEQ ID NO:52, a polynucleotide comprising a sequence of SEQ ID NO:53, a polynucleotide comprising a sequence of SEQ ID NO:54, and a polynucleotide comprising a sequence of SEQ ID NO:48 are splice variants of each other. Any one of the splice variants described above can encode a polypeptide which contains at least one functional or structural characteristic of CGDD.

It will be appreciated by those skilled in the art that as a result of the degeneracy of the genetic code, a multitude of polynucleotide sequences encoding CGDD, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring CGDD, and all such variations are to be considered as being specifically disclosed.

Although polynucleotides which encode CGDD and its variants are generally capable of hybridizing to polynucleotides encoding naturally occurring CGDD under appropriately selected conditions of stringency, it may be advantageous to produce polynucleotides encoding CGDD or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding CGDD and its derivatives without altering the encoded amino acid sequences include the production of RNA transcripts having more desirable properties, such as a greater half-life, than transcripts produced from the naturally occurring sequence.

The invention also encompasses production of polynucleotides which encode CGDD and CGDD derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding CGDD or any fragment thereof.

Embodiments of the invention can also include polynucleotides that are capable of hybridizing to the claimed polynucleotides, and, in particular, to those having the sequences shown in SEQ ID NO:28-54 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (M J Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding CGDD may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode CGDD may be cloned in recombinant DNA molecules that direct expression of CGDD, or fragments or functional equivalents thereof, in appropriate host cells. Due to the inherent degeneracy of the genetic code, other polynucleotides which encode substantially the same or a functionally equivalent polypeptides may be produced and used to express CGDD.

The polynucleotides of the invention can be engineered using methods generally known in the art in order to alter CGDD-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of CGDD, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding CGDD may be synthesized, in whole or in part, using one or more chemical methods well known in the art (Caruthers, M. H. et al. (1980) Nucleic Acids Symp. Ser. 7:215-223; Hom, T. et al. (1980) Nucleic Acids Symp. Ser. 7:225-232). Alternatively, CGDD itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of CGDD, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active CGDD, the polynucleotides encoding CGDD or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding CGDD. Such elements may vary in their strength and specificity. Specific initiation signals may also be used to achieve more efficient translation of polynucleotides encoding CGDD. Such signals include the ATG initiation codon and adjacent sequences, e.g. the Kozak sequence. In cases where a polynucleotide sequence encoding CGDD and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding CGDD and appropriate transcriptional and translational control elements. These methods include in vitro recombinant DNA techniques, synthetic techniques, and in vivo genetic recombination (Sambrook, J. et al. (1989) Molecular Cloning, A Laboratory Manual, Cold Spring Harbor Press, Plainview N.Y., ch. 4, 8, and 16-17; Ausubel et al., supra, ch. 1, 3, and 15).

A variety of expression vector/host systems may be utilized to contain and express polynucleotides encoding CGDD. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding CGDD. For example, routine cloning, subcloning, and propagation of polynucleotides encoding CGDD can be achieved using a multifunctional E. coli vector such as PBLUESCRIPT (Stratagene, La Jolla Calif.) or PSPORT1 plasmid (Invitrogen). Ligation of polynucleotides encoding CGDD into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of CGDD are needed, e.g. for the production of antibodies, vectors which direct high level expression of CGDD may be used. For example, vectors containing the strong, inducible SP6 or T7 bacteriophage promoter may be used.

Yeast expression systems may be used for production of CGDD. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of CGDD. Transcription of polynucleotides encoding CGDD may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding CGDD may be ligated into an adenovirus transcription/translation complex consisting of the late promoter and tripartite leader sequence. Insertion in a non-essential E1 or E3 region of the viral genome may be used to obtain infective virus which expresses CGDD in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J.J. et al. (1997) Nat. Genet. 15:345-355).

For long term production of recombinant proteins in mammalian systems, stable expression of CGDD in cell lines is preferred. For example, polynucleotides encoding CGDD can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S.C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding CGDD is inserted within a marker gene sequence, transformed cells containing polynucleotides encoding CGDD can be identified by the absence of marker gene function. Alternatively, a marker gene can be placed in tandem with a sequence encoding CGDD under the control of a single promoter. Expression of the marker gene in response to induction or selection usually indicates expression of the tandem gene as well.

In general, host cells that contain the polynucleotide encoding CGDD and that express CGDD may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of riucleic acid or protein sequences.

Immunological methods for detecting and measuring the expression of CGDD using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on CGDD is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding CGDD include oligolabeling, nick translation, end-labeling, or PCR amplification using a labeled nucleotide. Alternatively, polynucleotides encoding CGDD, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding CGDD may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode CGDD may be designed to contain signal sequences which direct secretion of CGDD through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding CGDD may be ligated to a heterologous sequence resulting in translation of a fusion protein in any of the aforementioned host systems. For example, a chimeric CGDD protein containing a heterologous moiety that can be recognized by a commercially available antibody may facilitate the screening of peptide libraries for inhibitors of CGDD activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the CGDD encoding sequence and the heterologous protein sequence, so that CGDD may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled CGDD may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

CGDD, fragments of CGDD, or variants of CGDD may be used to screen for compounds that specifically bind to CGDD. One or more test compounds may be screened for specific binding to CGDD. In various embodiments, 1, 2, 3, 4, 5, 10, 20, 50, 100, or 200 test compounds can be screened for specific binding to CGDD. Examples of test compounds can include antibodies, anticalins, oligonucleotides, proteins (e.g., ligands or receptors), or small molecules.

In related embodiments, variants of CGDD can be used to screen for binding of test compounds, such as antibodies, to CGDD, a variant of CGDD, or a combination of CGDD and/or one or more variants CGDD. In an embodiment, a variant of CGDD can be used to screen for compounds that bind to a variant of CGDD, but not to CGDD having the exact sequence of a sequence of SEQ ID NO:1-27. CGDD variants used to perform such screening can have a range of about 50% to about 99% sequence identity to CGDD, with various embodiments having 60%, 70%, 75%, 80%, 85%, 90%, and 95% sequence identity.

In an embodiment, a compound identified in a screen for specific binding to CGDD can be closely related to the natural ligand of CGDD, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor CGDD (Howard, A. D. et al. (2001) Trends Pharmacol. Sci. 22:132-140; Wise, A. et al. (2002) Drug Discovery Today 7:235-246).

In other embodiments, a compound identified in a screen for specific binding to CGDD can be closely related to the natural receptor to which CGDD binds, at least a fragment of the receptor, or a fragment of the receptor including all or a portion of the ligand binding site or binding pocket. For example, the compound may be a receptor for CGDD which is capable of propagating a signal, or a decoy receptor for CGDD which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to CGDD, fragments of CGDD, or variants of CGDD. The binding specificity of the antibodies thus screened can thereby be selected to identify particular fragments or variants of CGDD. In one embodiment, an antibody can be selected such that its binding specificity allows for preferential identification of specific fragments or variants of CGDD. In another embodiment, an antibody can be selected such that its binding specificity allows for preferential diagnosis of a specific disease or condition having increased, decreased, or otherwise abnormal production of CGDD.

In an embodiment, anticalins can be screened for specific binding to CGDD, fragments of CGDD, or variants of CGDD. Anticalins are ligand-binding proteins that have been constructed based on a lipocalin scaffold (Weiss, G.A. and H.B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit CGDD involves producing appropriate cells which express CGDD, either as a secreted protein or on the cell membrane. Preferred cells include cells from mamals, yeast, Drosophila, or E. coli. Cells expressing CGDD or cell membrane fractions which contain CGDD are then contacted with a test compound and binding, stimulation, or inhibition of activity of either CGDD or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with CGDD, either in solution or affixed to a solid support, and detecting the binding of CGDD to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

CGDD, fragments of CGDD, or variants of CGDD may be used to screen for compounds that modulate the activity of CGDD. Such compounds may include agonists, antagonists, or partial or inverse agonists. In one embodiment, an assay is performed under conditions permissive for CGDD activity, wherein CGDD is combined with at least one test compound, and the activity of CGDD in the presence of a test compound is compared with the activity of CGDD in the absence of the test compound. A change in the activity of CGDD in the presence of the test compound is indicative of a compound that modulates the activity of CGDD. Alternatively, a test compound is combined with an in vitro or cell-free system comprising CGDD under conditions suitable for CGDD activity, and the assay is performed. In either of these assays, a test compound which modulates the activity of CGDD may do so indirectly and need not come in direct contact with the test compound. At least one and up to a plurality of test compounds may be screened.

In another embodiment, polynucleotides encoding CGDD or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-oxP system to knockout a gene of interest in a tissue- or developmental stage-specific manner (Marth, J. D. (1996) Clin. Invest. 97:1999-2002; Wagner, K.U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding CGDD may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding CGDD can also be used to create “knockin” humanized animals (pigs) or transgenic animals (mice or rats) to model human disease. With knockin technology, a region of a polynucleotide encoding CGDD is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress CGDD, e.g., by secreting CGDD in its milk may also serve as a convenient source of that protein (Janne, J. et al. (1998) Biotechnol. Annu. Rev. 4:55-74).

### THERAPEUTICS

Chemical and structural similarity, e.g., in the context of sequences and motifs, exists between regions of CGDD and proteins associated with cell growth, differentiation, and death. In addition, the expression of CGDD is closely associated with bone tumor, bronchial, brain, brain tumor, breast, endometrial, epithelial cell, lung, ovarian, ovary tumor, small intestine, spinal cord, and thyroid tissue, as well as breast fibrocystic disease, diseased cerebellum and aortic tissues, unstimulated astrocyte cells, and a transformed embryonal cell line (293-EBNA) derived from kidney epithelial tissue. In addition, examples of tissues expressing CGDD can be found in Table 6 and can also be found in Example XI. Therefore, CGDD appears to play a role in cell proliferative disorders including cancer, developmental disorders, neurological disorders, autoimmune/inflammatory disorders, reproductive disorders, and disorders of the placenta. In the treatment of disorders associated with increased CGDD expression or activity, it is desirable to decrease the expression or activity of CGDD. In the treatment of disorders associated with decreased CGDD expression or activity, it is desirable to increase the expression or activity of CGDD.

Therefore, in one embodiment, CGDD or a fragment or derivative thereof may be administered to a subject to treat or prevent a disorder associated with decreased expression or activity of CGDD. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmunelinflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumors; and a disorder of the placenta such as preeclampsia, choriocarcinoma, abruptio placentae, placenta previa, placental or maternal floor infarction, placenta accreta, increate, and percreta, extrachorial placentas, chorangioma, chorangiosis, chronic villitis, placental villous endema, widespread fibrosis of the terminal villi, intervillous thrombi, hemorraghic endovasculitis, erythroblastosis fetalis, and nonimmune fetal hydrops.

In another embodiment, a vector capable of expressing CGDD or a fragment or derivative thereof may be administered to a subject to treat or prevent a disorder associated with decreased expression or activity of CGDD including, but not limited to, those described above.

In a further embodiment, a composition comprising a substantially purified CGDD in conjunction with a suitable pharmaceutical carrier may be administered to a subject to treat or prevent a disorder associated with decreased expression or activity of CGDD including, but not limited to, those provided above.

In still another embodiment, an agonist which modulates the activity of CGDD may be administered to a subject to treat or prevent a disorder associated with decreased expression or activity of CGDD including, but not limited to, those listed above.

In a further embodiment, an antagonist of CGDD may be administered to a subject to treat or prevent a disorder associated with increased expression or activity of CGDD. Examples of such disorders include, but are not limited to, those cell proliferative disorders including cancer, developmental disorders, neurological disorders, autoimmune/inflammatory disorders, reproductive disorders, and disorders of the placenta described above. In one aspect, an antibody which specifically binds CGDD may be used directly as an antagonist or indirectly as a targeting or delivery mechanism for bringing a pharmaceutical agent to cells or tissues which express CGDD.

In an additional embodiment, a vector expressing the complement of the polynucleotide encoding CGDD may be administered to a subject to treat or prevent a disorder associated with increased expression or activity of CGDD including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of CGDD may be produced using methods which are generally known in the art. In particular, purified CGDD may be used to produce antibodies or to screen libraries of pharmaceutical agents to identify those which specifically bind CGDD. Antibodies to CGDD may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with CGDD or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to CGDD have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of CGDD amino acids may be fused with those of another protein, such as KLH, and antibodies to the chimeric molecule may be produced.

Monoclonal antibodies to CGDD may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce CGDD-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for CGDD may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CGDD and its specific antibody. A two-site, monoclonal-based immunoassay utilizing monoclonal antibodies reactive to two non-interfering CGDD epitopes is generally used, but a competitive binding assay may also be employed (Pound, supra).

Various methods such as Scatchard analysis in conjunction with radioimmunoassay techniques may be used to assess the affinity of antibodies for CGDD. Affinity is expressed as an association constant, Kₐ, which is defined as the molar concentration of CGDD-antibody complex divided by the molar concentrations of free antigen and free antibody under equilibrium conditions. The Kₐ determined for a preparation of polyclonal antibodies, which are heterogeneous in their affinities for multiple CGDD epitopes, represents the average affinity, or avidity, of the antibodies for CGDD. The Kₐ determined for a preparation of monoclonal antibodies, which are monospecific for a particular CGDD epitope, represents a true measure of affinity. High-affinity antibody preparations with Kₐ ranging from about 10⁹ to 10¹² L/mole are preferred for use in immunoassays in which the CGDD-antibody complex must withstand rigorous manipulations. Low-affinity antibody preparations with Kₐ ranging from about 10⁶ to 10⁷ L/mole are preferred for use in immunopurification and similar procedures which ultimately require dissociation of CGDD, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of CGDD-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding CGDD, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding CGDD. Such technology is well known in the art, and antisense oligonucleotides or larger fragments can be designed from various locations along the coding or control regions of sequences encoding CGDD (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding CGDD may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in CGDD expression or regulation causes disease, the expression of CGDD from an appropriate population of transduced cells may alleviate the clinical manifestations caused by the genetic deficiency.

In a further embodiment of the invention, diseases or disorders caused by deficiencies in CGDD are treated by constructing mammalian expression vectors encoding CGDD and introducing these vectors by mechanical means into CGDD-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of CGDD include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). CGDD may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding CGDD from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to CGDD expression are treated by constructing a retrovirus vector consisting of (i) the polynucleotide encoding CGDD under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M.L. (1997) J. Virol. 71:47074716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding CGDD to cells which have one or more genetic abnormalities with respect to the expression of CGDD. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P.A. et al. (1999; Annu. Rev. Nutr. 19:511-544) and Verrna, I. M. and N. Somia (1997; Nature 18:389:239-242).

In another embodiment, a herpes-based, gene therapy delivery system is used to deliver polynucleotides encoding CGDD to target cells which have one or more genetic abnormalities with respect to the expression of CGDD. The use of herpes simplex virus (HSV)-based vectors may be especially valuable for introducing CGDD to cells of the central nervous system, for which HSV has a tropism. The construction and packaging of herpes-based vectors are well known to those with ordinary skill in the art. A replication-copetent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding CGDD to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for CGDD into the alphavirus genome in place of the capsid-coding region results in the production of a large number of CGDD-coding RNAs and the synthesis of high levels of CGDD in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of CGDD into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding CGDD.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding CGDD. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding CGDD. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased CGDD expression or activity, a compound which specifically inhibits expression of the polynucleotide encoding CGDD may be therapeutically useful, and in the treatment of disorders associated with decreased CGDD expression or activity, a compound which specifically promotes expression of the polynucleotide encoding CGDD may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary. library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding CGDD is exposed to at least one test compound thus obtained. The sample may comprise, for example, an intact or permeabilized cell, or an in vitro cell-free or reconstituted biochemical system. Alterations in the expression of a polynucleotide encoding CGDD are assayed by any method commonly known in the art. Typically, the expression of a specific nucleotide is detected by hybridization with a probe having a nucleotide sequence complementary to the sequence of the polynucleotide encoding CGDD. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G.M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of CGDD, antibodies to CGDD, and mimetics, agonists, antagonists, or inhibitors of CGDD.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising CGDD or fragments thereof. For example, liposome preparations containing a cell-impermeable macromolecule may promote cell fusion and intracellular delivery of the macromolecule. Alternatively, CGDD or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example CGDD or fragments thereof, antibodies of CGDD, and agonists, antagonists or inhibitors of CGDD, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### DIAGNOSTICS

In another embodiment, antibodies which specifically bind CGDD may be used for the diagnosis of disorders characterized by expression of CGDD, or in assays to monitor patients being treated with CGDD or agonists, antagonists, or inhibitors of CGDD. Antibodies useful for diagnostic purposes may be prepared in the same manner as described above for therapeutics. Diagnostic assays for CGDD include methods which utilize the antibody and a label to detect CGDD in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring CGDD, including ELISAs, RIAs, and FACS, are known in the art and provide a basis for diagnosing altered or abnormal levels of CGDD expression. Normal or standard values for CGDD expression are established by combining body fluids or cell extracts taken from normal mammalian subjects, for example, human subjects, with antibodies to CGDD under conditions suitable for complex formation. The amount of standard complex formation may be quantitated by various methods, such as photometric means. Quantities of CGDD expressed in subject, control, and disease samples from biopsied tissues are compared with the standard values. Deviation between standard and subject values establishes the parameters for diagnosing disease.

In another embodiment of the invention, polynucleotides encoding CGDD may be used for diagnostic purposes. The polynucleotides which may be used include oligonucleotides, complementary RNA and DNA molecules, and PNAs. The polynucleotides may be used to detect and quantify gene expression in biopsied tissues in which expression of CGDD may be correlated with disease. The diagnostic assay may be used to determine absence, presence, and excess expression of CGDD, and to monitor regulation of CGDD levels during therapeutic intervention.

In one aspect, hybridization with PCR probes which are capable of detecting polynucleotides, including genomic sequences, encoding CGDD or closely related molecules may be used to identify nucleic acid sequences which encode CGDD. The specificity of the probe, whether it is made from a highly specific region, e.g., the 5′regulatory region, or from a less specific region, e.g., a conserved motif, and the stringency of the hybridization or amplification will determine whether the probe identifies only naturally occurring sequences encoding CGDD, allelic variants, or related sequences.

Probes may also be used for the detection of related sequences, and may have at least 50% sequence identity to any of the CGDD encoding sequences. The hybridization probes of the subject invention may be DNA or RNA and may be derived from the sequence of SEQ ID NO:28-54 or from genomic sequences including promoters, enhancers, and introns of the CGDD gene.

Means for producing specific hybridization probes for polynucleotides encoding CGDD include the cloning of polynucleotides encoding CGDD or CGDD derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²p or ³⁵S, or by enzymatic labels, such as alkaline phosphatase coupled to the probe via avidin/biotin coupling systems, and the like.

Polynucleotides encoding CGDD may be used for the diagnosis of disorders associated with expression of CGDD. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, iritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumors; and a disorder of the placenta such as preeclampsia, choriocarcinoma, abruptio placentae, placenta previa, placental or maternal floor infarction, placenta accreta, increate, and percreta, extrachorial placentas, chorangioma, chorangiosis, chronic villitis, placental villous endema, widespread fibrosis of the terminal villi, intervillous thrombi, hemorraghic endovasculitis, erythroblastosis fetalis, and nonimmune fetal hydrops. Polynucleotides encoding CGDD may be used in Southern or northern analysis, dot blot, or other membrane-based technologies; in PCR technologies; in dipstick, pin, and multiformat ELISA-like assays; and in microarrays utilizing fluids or tissues from patients to detect altered CGDD expression. Such qualitative or quantitative methods are well known in the art.

In a particular aspect, polynucleotides encoding CGDD may be used in assays that detect the presence of associated disorders, particularly those mentioned above. Polynucleotides complementary to sequences encoding CGDD may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding CGDD in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of CGDD, a normal or standard profile for expression is established. This may be accomplished by combining body fluids or cell extracts taken from normal subjects, either animal or human, with a sequence, or a fragment thereof, encoding CGDD, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding CGDD may involve the use of PCR. These oligomers may be chemically synthesized, generated enzymatically, or produced in vitro. Oligomers will preferably contain a fragment of a polynucleotide encoding CGDD, or a fragment of a polynucleotide complementary to the polynucleotide encoding CGDD, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding CGDD may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding CGDD are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of CGDD include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, CGDD, fragments of CGDD, or antibodies specific for CGDD may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for CGDD to quantify the levels of CGDD expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R.A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding CGDD may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding CGDD on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, CGDD, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between CGDD and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with CGDD, or fragments thereof, and washed. Bound CGDD is then detected by methods well known in the art. Purified CGDD can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding CGDD specifically compete with a test compound for binding CGDD. In this manner, antibodies can be used to detect the presence of any peptide which shares one or more antigenic determinants with CGDD.

In additional embodiments, the nucleotide sequences which encode CGDD may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent The following embodiments are, therefore, to be construed as merely illustrative, and not limitative of the remainder of the disclosure in any way whatsoever.

The disclosures of all patents, applications, and publications mentioned above and below, including U.S. Ser. No. 60/311,017, U.S. Ser. No. 60/313,070, U.S. Ser. No. 60/313,071, U.S. Ser. No. 60/314,678, U.S. Ser. No. 60/316,692, U.S. Ser. No. 60/317,913, U.S. Ser. No. 60/322,182, U.S. Ser. No. 60/342,761, U.S. Ser. No. 60/340,747, and U.S. Ser. No. 60/369,129, are hereby expressly incorporated by reference.

### EXAMPLES

### I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

### II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIA WELL 8 Plasmid, QIAW 8 Plus Plasmid, QIA WELL 8 Ultra Plasmid purification systems or the R. E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

### III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:4143); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:28-54. Fragments from about 20 to about 4000 nucleotides which are useful in hybridization and amplification technologies are described in Table 4, column 2.

### IV. Identification and Editing of Coding Sequences from Genomic DNA

Putative proteins associated with cell growth, differentiation, and death were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode proteins associated with cell growth, differentiation, and death, the encoded polypeptides were analyzed by querying against PFAM models for proteins associated with cell growth, differentiation, and death. Potential proteins associated with cell growth, differentiation, and death were also identified by homology to Incyte cDNA sequences that had been annotated as proteins associated with cell growth, differentiation, and death. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

### V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

### VI. Chromosomal Mapping of CGDD Encoding Polynucleotides

The sequences which were used to assemble SEQ ID NO:28-54 were compared with sequences from the Incyte LIFESEQ database and public domain databases using BLAST and other implementations of the Smith-Waterman algorithm. Sequences from these databases that matched SEQ ID NO:28-54 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Gen6thon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centimorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

### VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7; Ausubel et al., supra, ch. 4).

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding CGDD are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding CGDD. cDNA sequences and cDNA library/tissue information are found in the LIFESEQ GOLD database (Incyte Genomics, Palo Alto Calif.).

### VIII. Extension of CGDD Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′regulatory sequences using the above procedure along with oligonucleotides designed for such extension, and an appropriate genomic library.

### IX. Identification of Single Nucleotide Polymorphisms in CGDD Encoding Polynucleotides

Common DNA sequence variants known as single nucleotide polymorphisms (SNPs) were identified in SEQ ID NO:28-54 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in imniunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

### X. Labeling and Use of Individual Hybridization Probes

Hybridization probes derived from SEQ ID NO:28-54 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham NH). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

### XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

### Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1×first strand buffer, 0.03 units/μL RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)+ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

### Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

### Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a comer of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

### Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of CyS. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for CyS. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's enission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

### Expression

For example, SEQ ID NO:28 was differentially expressed in cancer cells versus normal cells based on microarray experimentation. SEQ ID NO:28 showed differential expression as determined by microarray analysis. Histological and molecular evaluation of breast tumors reveals that the development of breast cancer evolves through a multi-step process whereby pre-malignant mammary epithelial cells undergo a relatively defined sequence of events leading to tumor formation. A cross-comparison experimental design was used to evaluate the expression of cDNAs from two human breast tumor cell lines (MCF-7 and BT20) at various stages of tumor progression, as compared to a non-malignant mammary epithelial cell line, HMEC (Clonetics, San Diego, Calif.). All cell cultures were propagated in media according to the supplier's recommendations and grown to 70-80% confluence prior to RNA isolation.

The expression of SEQ ID NO:28 was decreased at least two-fold in MCF-7 cells, a nonmalignant breast adenocarcinoma cell line isolated from the pleural effusion of a 69-year old female. MCF-7 has retained characteristics of the mammary epithelium such as the ability to process estradiol via cytoplasmic estrogen receptors and the capacity to form domes in culture. The expression of SEQ ID NO:28 was also found to be decreased by at least two-fold in BT20 cells, a breast carcinoma cell line derived in vitro from cells emigrating out of thin slices of the tumor mass isolated from a 74-year old female. These experiments indicate that SEQ ID NO:28 was significantly underexpressed in the breast tumor cell lines tested, further establishing the utility of SEQ ID NO:1 and SEQ ID NO:28 as diagnostic markers or as therapeutic targets for breast cancer.

For example, SEQ ID NO:30 showed differential expression in lung tissue from patients with lung cancer compared to matched microscopically normal tissues from the same donors as determined by microarray analysis. The expression of CGDD-3 was decreased at least two-fold in cancerous lung tissue. In an alternative example, SEQ ID NO:30 showed differential expression in breast tissue from a patient with breast cancer compared to matched microscopically normal tissue from the same donor as determined by microarray analysis. The expression of CGDD-3 was increased at least two-fold in cancerous breast tissue. SEQ ID NO:30 also showed differential expression in brain cingulate from a patient with Alzheimer's disease compared to matched microscopically normal tissue from the same donor as determined by microarray analysis. The expression of CGDD-3 was increased at least two-fold in cingulate tissue with Alzheimer's disease. Therefore, SEQ ID NO:30 is useful in disease staging and in diagnostic assays for cell proliferative disorders, including lung cancer and breast cancer, and for neurological disorders, including Alzheimer's disease.

SEQ ID NO:33 showed differential expression as determined by microarray analysis. BT-20 cells are a breast carcinoma cell line derived in vitro from cells which emigrated out of thin slices of a tumor mass isolated from a 74-year-old female. The treatment of BT-20 cells with epidermal growth factor (EGF), a potent mitogen, revealed that the expression of SEQ ID NO:33 was down-regulated at least two-fold. Additionally, a comparison of a primary breast epithelial cell line (HMEC cells) isolated from a normal donor with BT-20, MCF7 (a nonmalignant breast adenocarcinoma cell line isolated from the pleural effusion of a 69-year old female), MDA-mb-231 (a breast tumor cell line isolated from the pleural effusion of a 51-year-old female which forms poorly differentiated adenocarcinoma in nude mice), Sk-BR-3 (a breast adenocarcinoma cell line isolated from a malignant pleural effusion of a 43-year-old female), and T47D (a breast carcinoma cell line obtained from a 54-year-old female with an infiltrating ductal carcinoma of the breast) cells also indicated that SEQ ID NO:33 was down-regulated at least two-fold in each of the cancerous breast cell lines when compared to the HMEC cell line. Furthermore, the expression of SEQ ID NO:33 was also found to be down-regulated in two out of three prostate tumor cell lines when compared to normal donor primary prostate epithelial cells. These experiments indicate that SEQ ID NO:33, encoding SEQ ID NO:6 was significantly underexpressed in the breast tumor and prostate tumor cell lines tested, further establishing the utility of SEQ ID NO:6 and SEQ ID NO:33 for disease staging, as diagnostic markers, or as therapeutic targets for both breast and prostate cancer.

In another example, SEQ ID NO:42 showed differential expression as determined by microarray analysis. Histological and molecular evaluation of breast tumors reveals that the development of breast cancer evolves through a multi-step process whereby pre-malignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Several variables that may influence the process of tumor progression and malignant transformation include genetic factors, environmental factors, growth factors, and hormones. Based on the complexity of this process, it is critical to study a population of human mammary epithelial cells undergoing the process of malignant transformation, and to associate specific stages of progression with phenotypic and molecular characteristics.

In this experiment, primary human breast epithelial cells (HMECs) were compared to breast carcinoma cell lines at various stages of tumor progression. All cells were grown in the supplier's recommended medium to 70-80% confluence prior to RNA harvest. MCF7 is a nonmalignant breast adenocarcinoma cell line isolated from the pleura effusion of a 69-year old female. MCF7 has retained characertisticg of the mammary epithelium such as the ability to process estradiol via cytoplasmic estrogen receptors and the capacity to form domes in culture. T-47D is a breast carcinoma cell line isolated from a pleural effusion obtained from a 54-year old female with infiltrating ductal carcinoma of the breast. Sk-BR-3 is a breast adenocarcinoma cell line isolated from a malignant pleural effusion of a 43-year old female. It forms poorly differentiated adenocarcinoma when injected into nude mice. The gene expression of SEQ ID NO:42 was decreased by at least two-fold in the breast cancer lines as compared to the HMEC cells.

In another experiment, the gene expression profile of SEQ ID NO:42 was evaluated in various breast cancer cell lines as compared to a nonmalignant breast epithelial cell line, MCF-10A (a breast mammary gland cell line, e.g. with luminal ductal characteristics, that was isolated from a 36-year old female with fibrocystic breast disease). The cells were grown in defined serum-free TCH medium to 70-80% confluence prior to RNA harvest. The expression of SEQ ID NO:42 was decreased by at least two-fold in the following cell lines: MCF7, and BT-20, a breast carcinoma cell line derived in vitro from the cells emigrating out of thin slices from the tumor mass isolated from a 74-year old female.

In another example, the gene expression profile of SEQ ID NO:42 was evaluated in various breast cancer lines as compared to MCF-10A cells, all of which were grown in defined serum-free H14 medium to 70-80% confluence prior to RNA harvest. Under these conditions, the expression of SEQ ID NO:42 was decreased by at least two-fold in T-47D cells as compared to MCF-10A cells.

In another example, SEQ ID NO:45 was differentially expressed in response to treatment with steroids. Steroids are a class of lipid-soluble molecules, including cholesterol, bile acids, vitamin D, and hormones, that share a common four-ring structure based on cyclopentanoperhydro-phenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing foflicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma. Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with a-fetoprotein iii) conversion of ammonia to urea and glutamine; iv) metabolize aromatic amino acids; and v) proliferate in glucose-free and insulin-free medium The C3A cell line is now well established as an in vitro model of the mature human liver (Mickelson et al. (1995) Hepatology 22:866-875; Nagendra et al. (1997) Am J Physiol 272:G408-G416).

The expression of SEQ ID NO:45 was increased by at least two-fold in drug-treated, as compared to untreated, C3A cells, under the following conditions: (1) at a dose of 10 μM medroxyprogesterone (MAH), for a duration of 1 and 3 hours of treatment; at a dose of 1 μM beclomethasone for a duration of 3 hours of treatment, and 10 μM for a duration of 1 and 3 hours of treatment; at doses of 1 and 10 μM budesonide for a duration of 1 and 3 hours of treatment, and at a dose of 100 μM for a duration of 1 and 6 hours of treatment; and lastly, at a dose of 10 μM betamethasone for a duration of 1 hour of treatment, and at a dose of 100 μM for a duration of 1, 3, and 6 hours of treatment.

These experiments demonstrate a significant differential expression profile for SEQ ID NO:42 and SEQ ID NO:45, and further establish their utility as diagnostic markers, tools for disease staging, or as therapeutic targets for diseases in which proteins associated with cell growth, differentiation, or death are involved.

In another example, SEQ ID NO:49 was differentially expressed using microarray techniques. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for approximately 70% of cases while SCLCs account for approximately 18% of cases. The molecular and cellular biology underlying the development and progression of lung caner are incompletely understood. Deletions on chromosome 3 are common in this disease and are thought to indicate the presence of a tumor suppressor gene in this region. Activating mutations in K-ras are commonly found in lung cancer and are the basis of one of the mouse models for the disease. Various experiments were performed in which normal lung was compared to lung tumor from the same donor. The expression of SEQ ID NO:49 was decreased by at least two-fold in the lung tumor samples.

The majority of ovarian cancers are derived from epithelial cells, and 70% of patients with epithelial ovarian cancers present with late-stage disease. As a result, the long-term survival rates for this disease are very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns likely vary when normal ovary is compared to ovarian tumors, we have examined gene expression in these tissues to identify possible markers for ovarian cancer. The expression of SEQ ID NO:49 was increased by at least two-fold in ovarian tumor tissue as compared to normal ovary tissue from the same donor.

These experiments indicate that a significant differential expression of SEQ ID NO:22 under various conditions further establishes the utility of SEQ ID NO:49 as a diagnostic marker, a tool for disease staging, or as a therapeutic target for cancer.

### XII. Complementary Polynucleotides

Sequences complementary to the CGDD-encoding sequences, or any parts thereof, are used to detect, decrease, or inhibit expression of naturally occurring CGDD. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of CGDD. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the CGDD-encoding transcript.

### XIII. Expression of CGDD

Expression and purification of CGDD is achieved using bacterial or virus-based expression systems. For expression of CGDD in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21 (DE3). Antibiotic resistant bacteria express CGDD upon induction with isopropyl beta-D-thiogalactopyranoside (IPTG). Expression of CGDD in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding CGDD by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum Gene Ther. 7:1937-1945).

In most expression systems, CGDD is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from CGDD at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified CGDD obtained by these methods can be used directly in the assays shown in Examples XVII, XVIII, and XIX, where applicable.

### XIV. Functional Assays

CGDD function is assessed by expressing the sequences encoding CGDD at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of CGDD on gene expression can be assessed using highly purified populations of cells transfected with sequences encoding CGDD and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding CGDD and other genes of interest can be analyzed by northern analysis or microarray techniques.

### XV. Production of CGDD Specific Antibodies

CGDD substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the CGDD amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-CGDD activity by, for example, binding the peptide or CGDD to a substrate, blocking with 1% BSA, reacting with rabbit antisera, washing, and reacting with radio-iodinated goat anti-rabbit IgG.

### XVI. Purification of Naturally Occurring CGDD Using Specific Antibodies

Naturally occurring or recombinant CGDD is substantially purified by immunoaffinity chromatography using antibodies specific for CGDD. An immunoaffinity column is constructed by covalently coupling anti-CGDD antibody to an activated chromatographic resin, such as CNBr-activated SEPHAROSE (Amersham Biosciences). After the coupling, the resin is blocked and washed according to the manufacturer's instructions.

Media containing CGDD are passed over the immunoaffinity column, and the column is washed under conditions that allow the preferential absorbance of CGDD (e.g., high ionic strength buffers in the presence of detergent). The column is eluted under conditions that disrupt antibody/CGDD binding (e.g., a buffer of pH 2 to pH 3, or a high concentration of a chaotrope, such as urea or thiocyanate ion), and CGDD is collected.

### XVII. Identification of Molecules Which Interact with CGDD

CGDD, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled CGDD, washed, and any wells with labeled CGDD complex are assayed. Data obtained using different concentrations of CGDD are used to calculate values for the number, affinity, and association of CGDD with the candidate molecules.

Alternatively, molecules interacting with CGDD are analyzed using the yeast two-hybrid system as described in Fields, S. and O. Song (1989; Nature 340:245-246), or using commercially available kits based on the two-hybrid system, such as the MATCHMAKER system (Clontech).

CGDD may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

### XVIII. Demonstration of CGDD Activity

CGDD activity is demonstrated by measuring the induction of terminal differentiation or cell cycle progression when CGDD is expressed at physiologically elevated levels in mammalian cell culture systems. cDNA is subcloned into a mammalian expression vector containing a strong promoter that drives high levels of cDNA expression. Vectors of choice include PCMV SPORT (Life Technologies, Gaithersburg, Md.) and PCR 3.1 (Invitrogen, Carlsbad, Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP) (Clontech, Palo Alto, Calif.), CD64, or a CD64-GFP fusion protein. Flow cytometry detects and quantifies the uptake of fluorescent molecules that diagnose events preceding or coincident with cell cycle progression or terminal differentiation. These events include changes in nuclear DNA content as measured by staining of DNA with propidium iodide; changes in cell size and granularity as measured by forward light scatter and 90 degree side light scatter; up or down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York, N.Y.

Alternatively, an in vitro assay for CGDD activity measures the transformation of normal human fibroblast cells overexpressing antisense CGDD RNA (Garkavtsev, I. and K. Riabowol (1997) Mol. Cell Biol. 17:2014-2019). cDNA encoding CGDD is subcloned into the pLNCX retroviral vector to enable expression of antisense CGDD RNA. The resulting construct is transfected into the ecotropic BOSC23 virus-packaging cell line. Virus contained in the BOSC23 culture supernatant is used to infect the amphotropic CAK8 virus-packaging cell line. Virus contained in the CAK8 culture supernatant is used to infect normal human fibroblast (Hs68) cells. Infected cells are assessed for the following quantifiable properties characteristic of transformed cells: growth in culture to high density associated with loss of contact inhibition, growth in suspension or in soft agar, formation of colonies or foci, lowered serum requirements, and ability to induce tumors when injected into immunodeficient mice. The activity of CGDD is proportional to the extent of transformation of Hs68 cells.

Alternatively, CGDD can be expressed in a mammalian cell line by transforming the cells with a eukaryotic expression vector encoding CGDD. Eukaryotic expression vectors are commercially available, and the techniques to introduce them into cells are well known to those skilled in the art. To assay the cellular localization of CGDD, cells are fractionated as described by Jiang, H. P. et al. (1992; Proc. Natl. Acad. Sci. 89:7856-7860). Briefly, cells pelleted by low-speed centrifugation are resusended in buffer (10 mM TRIS-HCl, pH 7.4/10 mM NaCl/3 mM MgCl₂/5 mM EDTA with 10 ug/mil aprotinin, 10 ug/ml leupeptin, 10 ug/ml pepstatin A, 0.2 mM phenylmethylsulfonyl fluoride) and homogenized. The homogenate is centrifuged at 600×g for 5 minutes. The particulate and cytosol fractions are separated by ultracentrifugation of the supernatant at 100,000×g for 60 minutes. The nuclear fraction is obtained by resuspending the 600×g pellet in sucrose solution (0.25 M sucrose/ 10 mM TRIS-HCl, pH 7.4/ 2 mM MgCl₂) and recentrifuged at 600×g. Equal amounts of protein from each fraction are applied to an SDS/10% polyacrylamide gel and blotted onto membranes. Western blot analysis is performed using CGDD anti-serum. The localization of CGDD is assessed by the intensity of the corresponding band in the nuclear fraction relative to the intensity in the other fractions. Alternatively, the presence of CGDD in cellular fractions is examined by fluorescence microscopy using a fluorescent antibody specific for CGDD.

Alternatively, CGDD activity may be demonstrated as the ability to interact with its associated Ras superfamily protein, in an in vitro binding assay. The candidate Ras superfamily proteins are expressed as fusion proteins with glutathione S-transferase (GST), and purified by affinity chromatography on glutathione-Sepharose. The Ras superfamily proteins are loaded with GDP by incubating 20 mM Tris buffer, pH 8.0, containing 100 mM NaCl, 2 mM EDTA, 5 mM MgCl2, 0.2 mM DTT, 100 μM AMP-PNP and 10 μM GDP at 30° C. for 20 minutes. CGDD is expressed as a FLAG fusion protein in a baculovirus system. Extracts of these baculovirus cells containing CGDD-FLAG fusion proteins are precleared with GST beads, then incubated with GST-Ras superfamily fusion proteins. The complexes formed are precipitated by glutathione-Sepharose and separated by SDS-polyacrylamide gel electrophoresis. The separated proteins are blotted onto nitrocellulose membranes and probed with commercially available anti-FLAG antibodies. CGDD activity is proportional to the amount of CGDD-FLAG fusion protein detected in the complex.

Alternatively, as demonstrated by Li and Cohen (Li, L. and S. N. Cohen (1995) Cell 85:319-329), the ability of CGDD to suppress tumorigenesis can be measured by designing an antisense sequence to the 5′ end of the gene and transfecting NIH 3T3 cells with a vector transcribing this sequence. The suppression of the endogenous gene will allow transformed fibroblasts to produce clumps of cells capable of forming metastatic tumors when introduced into nude mice.

Alternatively, an assay for CGDD activity measures the effect of injected CGDD on the degradation of maternal transcripts. Procedures for oocyte collection from Swiss albino mice, injection, and culture are as described in Stutz et al., (supra). A decrease in the degradation of maternal RNAs as compared to control oocytes is indicative of CGDD activity. In the alternative, CGDD activity is measured as the ability of purified CGDD to bind to RNAse as measured by the assays described in Example XVII.

Alternatively, an assay for CGDD activity measures syncytium formation in COS cells transfected with an CGDD expression plasmid, using the two-component fusion assay described in Mi (supra). This assay takes advantage of the fact that human interleukin 12 (IL-12) is a heterodimer comprising subunits with molecular weights of 35 kD (p35) and 40 kD (p40). COS cells transfected with expression plasmids carrying the gene for p35 are mixed with COS cells cotransfected with expression plasmids carrying the genes for p40 and CGDD. The level of IL-12 activity in the resulting conditioned medium corresponds to the activity of CGDD in this assay. Syncytium formation may also be measured by light microscopy (Mi et al., supra).

An alternative assay for CGDD activity measures cell proliferation as the amount of newly initiated DNA synthesis in Swiss mouse 3T3 cells. A plasmid containing polynucleotides encoding CGDD is transfected into quiescent 3T3 cultured cells using methods well known in the art. The transiently transfected cells are then incubated in the presence of [³ H]thymidine or a radioactive DNA precursor such as [α³²P]ATP. Where applicable, varying amounts of CGDD ligand are added to the transfected cells. Incorporation of [³H]thymidine into acid-precipitable DNA is measured over an appropriate time interval, and the amount incorporated is directly proportional to the amount of newly synthesized DNA and CGDD activity.

Alternatively, CGDD activity is measured by the cyclin-ubiquitin ligation assay (Townsley, F. M. et al. (1997) Proc. Natl. Acad. Sci. USA 94:2362-2367). The reaction contains in a volume of 10 μl, 40 mM Tric.HCl (pH 7.6), 5 mM Mg Cl₂, 0.5 mM ATP, 10 mM phosphocreatine, 50 μg of creatine phosphokinase/ml, 1 mg reduced carboxymethylated bovine serum albumin/ml, 50 μM ubiquitin, 1 μM ubiquitin aldehyde, 1-2 pmol ¹²⁵I-labeled cyclin B, 1 pmol E1, 1 μM okadaic acid, 10 μg of protein of M-phase fraction 1A (containing active E3-C and essentially free of E2-C), and varying amounts of CGDD. The reaction is incubated at 18° C. for 60 minutes. Samples are then separated by electrophoresis on an SDS polyacrylamide gel. The amount of ¹²⁵I- cyclin-ubiquitin formed is quantified by PHOSPHORIMAGER analysis. The amount of cyclin-ubiquitin formation is proportional to the activity of CGDD in the reaction.

Alternatively, an assay for CGDD activity uses radiolabeled nucleotides, such as [α³²P]ATP, to measure either the incorporation of radiolabel into DNA during DNA synthesis, or fragmentation of DNA that accompanies apoptosis. Mammalian cells are transfected with plasmid containing cDNA encoding CGDD by methods well known in the art. Cells are then incubated with radiolabeled nucleotide for various lengths of time. Chromosomal DNA is collected, and radioactivity is detected using a scintillation counter. Incorporation of radiolabel into chromosomal DNA is proportional to the degree of stimulation of the cell cycle. To determine if CGDD promotes apoptosis, chromosomal DNA is collected as above, and analyzed using polyacrylamide gel electrophoresis, by methods well known in the art. Fragmentation of DNA is quantified by comparison to untransfected control cells, and is proportional to the apoptotic activity of CGDD.

Alternatively, cyclophilin activity of CGDD is measured using a chymotrypsin-coupled assay to measure the rate of cis to trans interconversion (Fischer, G. et al. (1984) Biomed. Biochim. Acta 43:1101-1111). The chymotrypsin is used to estimate the trans-substrate cleavage activity at Xaa-Pro peptide bonds, wherein the rate constant for the cis to trans isomerization can be obtained by measuring the rate constant of the substrate hydrolysis at the slow phase. Samples are incubated in the presence or absence of the immunosuppressant drugs CsA or FK506, reactions initiated by addition of chymotrypsin, and the fluorescent reaction measured. The enzymatic rate constant is calculated from the equation kₐₚₚ=k_{H20}+k_{enz}, wherein first order kinetics are displayed, and where one unit of PPIase activity is defined as k_{enz} (s⁻¹).

Alternatively, cyclophilin activity of CGDD is monitored by a quantitative immunoassay that measures its affinity for stereospecific binding to the immunosuppressant drug cyclosporin (Quesniaux, V. F. et al. (1987) Eur. J. Immunol. 17:1359-1365). In this assay, the cyclophilin-cyclosporin complex is coated on a solid phase, with binding detected using anti-cyclophilin rabbit antiserum enhanced by an antiglobulin-enzyme conjugate.

Alternatively, activity of CGDD is monitored by a binding assay developed to measure the non-covalent binding between FKBPs and immunosuppressant drugs in the gas phase using electrospray ionization mass spectrometry (Trepanier, D. J. et al. (1999) Ther. Drug Monit. 21:274-280). In electrospray ionization, ions are generated by creating a fine spray of highly charged droplets in the presence of a strong electric field; as the droplet decreases in size, the charge density on the surface increases. Ions are electrostatically directed into a mass analyzer, where ions of opposite charge are generated in spatially separate sources and then swept into capillary inlets where the flows are merged and where reactions occur. By comparing the charge states of bound versus unbound CGDD/immunosuppressive drug complexes, relative binding affinities can be established and correlated with in vitro binding and immunosuppressive activity.

### XIX. CGDD Secretion Assay

A high throughput assay may be used to identify polypeptides that are secreted in eukaryotic cells. In an example of such an assay, polypeptide expression libraries are constructed by fusing 5′-biased cDNAs to the 5′-end of a leaderless β-lactamase gene. β-lactamase is a convenient genetic reporter as it provides a high signal-to-noise ratio against low endogenous background activity and retains activity upon fusion to other proteins. A dual promoter system allows the expression of β-lactamase fusion polypeptides in bacteria or eukaryotic cells, using the lac or CMV promoter, respectively.

Libraries are first transformed into bacteria, e.g., E. coli, to identify library members that encode fusion polypeptides capable of being secreted in a prokaryotic system. Mammalian signal sequences direct the translocation of β-lactamase fusion polypeptides into the periplasm of bacteria where it confers antibiotic resistance to carbenicillin. Carbenicillin-selected bacteria are isolated on solid media, individual clones are grown in liquid media, and the resulting cultures are used to isolate library member plasmid DNA.

Mammalian cells, e.g., 293 cells, are seeded into 96-well tissue culture plates at a density of about 40,000 cells/well in 100 μl phenol red-free DME supplemented with 10% fetal bovine serum (FB S) (Life Technologies, Rockville, Md.). The following day, purified plasmid DNAs isolated from carbenicillin-resistant bacteria are diluted with 15 μl OPTI-MEM I medium (Life Technologies) to a volume of 25 μl for each well of cells to be transfected. In separate plates, 1 μl LF2000 Reagent (Life Technologies) is diluted into 25 μl/well OPTI-NEM I. The 25 μl diluted LF2000 Reagent is then combined with the 25 μl diluted DNA, mixed briefly, and incubated for 20 minutes at room temperature. The resulting DNA-LF2000 reagent complexes are then added directly to each well of 293 cells. Cells are also transfected with appropriate control plasmids expressing either wild-type β-lactamase, leaderless β-lactamase, or, for example, CD4-fused leaderless β-lactamase. 24 hrs following transfection, about 90 μl of cell culture media are assayed at 37° C. with 100 μM Nitrocefin (Calbiochem, San Diego, Calif.) and 0.5 mM oleic acid (Sigma Corp. St. Louis, Mo.) in 10 mM phosphate buffer (pH 7.0). Nitrocefin is a substrate for β-lactamase that undergoes a noticeable color change from yellow to red upon hydrolysis. β-lactamase activity is monitored over 20 min in a microtiter plate reader at 486 nm. Increased color absorption at 486 nm corresponds to secretion of a β-lactamase fusion polypeptide in the transfected cell media, resulting from the presence of a eukaryotic signal sequence in the fusion polypeptide. Polynucleotide sequence analysis of the corresponding library member plasmid DNA is then used to identify the signal sequence-encoding cDNA. (Described in U.S. patent application Ser. No. 09/803,317, filed Mar. 9, 2001.)

For example, SEQ ID NO:14 was shown to be a secreted protein using this assay.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | Incyte Full Length |
| Incyte Project ID | SEQ ID NO: | Polypeptide ID | SEQ ID NO: | ID | Clones |
| 261510 | 1 | 261510CD1 | 28 | 261510CB1 | 90110645CA2 |
| 7498674 | 2 | 7498674CD1 | 29 | 7498674CB1 | 90175915CA2, |
| | | | | | 90175931CA2 |
| 1629617 | 3 | 1629617CD1 | 30 | 1629617CB1 | |
| 2369279 | 4 | 2369279CD1 | 31 | 2369279CB1 | |
| 3167506 | 5 | 3167506CD1 | 32 | 3167506CB1 | |
| 3075937 | 6 | 3075937CD1 | 33 | 3075937CB1 | |
| 5176268 | 7 | 5176268CD1 | 34 | 5176268CB1 | 90110594CA2 |
| 72830854 | 8 | 72830854CD1 | 35 | 72830854CB1 | |
| 3632052 | 9 | 3632052CD1 | 36 | 3632052CB1 | |
| 7493817 | 10 | 7493817CD1 | 37 | 7493817CB1 | |
| 6715627 | 11 | 6715627CD1 | 38 | 6715627CB1 | |
| 7727886 | 12 | 7727886CD1 | 39 | 7727886CB1 | |
| 914113 | 13 | 914113CD1 | 40 | 914113CB1 | |
| 1953711 | 14 | 1953711CD1 | 41 | 1953711CB1 | |
| 1595275 | 15 | 1595275CD1 | 42 | 1595275CB1 | |
| 72332548 | 16 | 72332548CD1 | 43 | 72332548CB1 | |
| 7322834 | 17 | 7322834CD1 | 44 | 7322834CB1 | |
| 1925714 | 18 | 1925714CD1 | 45 | 1925714CB1 | |
| 6803363 | 19 | 6803363CD1 | 46 | 6803363CB1 | 90192087CA2 |
| 7070580 | 20 | 7070580CD1 | 47 | 7070580CB1 | 90136522CA2 |
| 7500176 | 21 | 7500176CD1 | 48 | 7500176CB1 | |
| 7500506 | 22 | 7500506CD1 | 49 | 7500506CB1 | 90052311CA2 |
| 7500639 | 23 | 7500639CD1 | 50 | 7500639CB1 | 1355056CA2, |
| | | | | | 90133101CA2, |
| | | | | | 90133117CA2, |
| | | | | | 90133118CA2, |
| | | | | | 90133225CA2, |
| | | | | | 90133233CA2 |
| 7506167 | 24 | 7506167CD1 | 51 | 7506167CB1 | |
| 90081189 | 25 | 90081189CD1 | 52 | 90081189CB1 | 90081189CA2, |
| | | | | | 90081265CA2, |
| | | | | | 90129619CA2 |
| 7510095 | 26 | 7510095CD1 | 53 | 7510095CB1 | |
| 7510096 | 27 | 7510096CD1 | 54 | 7510096CB1 | |

TABLE 2

| Polypeptide | | GenBank ID NO: | | |
|---|---|---|---|---|
| SEQ | Incyte | or PROTEOME | Probability | |
| ID NO: | Polypeptide ID | ID NO: | Score | Annotation |
| 2 | 7498674CD1 | g4091819 | 4.30E−161 | [Homo sapiens] leucine-rich glioma-inactivated protein precursor |
| | | | | (Chernova, O. B. et al. (1998) Oncogene 17: 2873-2881) |
| 3 | 1629617CD1 | g9965418 | 1.50E−190 | [Mus musculus] iroquois-class homeobox protein IRX2 |
| | | | | Christoffels, V. M. et al. |
| | | | | Patterning the embryonic heart: identification of five mouse iroquois homeobox |
| | | | | genes in the developing heart |
| | | | | Dev. Biol. 224, 263-274 (2000) |
| 4 | 2369279CD1 | g4581563 | 1.90E−64 | [Homo sapiens] tesmin |
| | | | | Sugihara, T. et al. |
| | | | | A novel testis-specific metallothionein-like protein, tesmin, is an early marker of |
| | | | | male germ cell differentiation |
| | | | | Genomics 57, 130-136 (1999) |
| 5 | 3167506CD1 | g12697318 | 2.60E−177 | [Homo sapiens] PBX4 protein |
| | | | | Wagner, K. et al. |
| | | | | Pbx4, a new Pbx family member on mouse chromosome 8, is expressed during |
| | | | | spermatogenesis |
| | | | | Mech. Dev. 103, 127-131 (2001) |
| 6 | 3075937CD1 | g2072425 | 0.0 | [Homo sapiens] non-lens beta gamma-crystallin like protein |
| | | | | Ray, M. E. et al. (1997) AIM1, a novel non-lens member of the betagamma- |
| | | | | crystallin superfamily, is associated with the control of tumorigenicity in human |
| | | | | malignant melanoma. Proc. Natl. Acad. Sci. U.S.A. 94: 3229-3234 |
| 7 | 5176268CD1 | g14530665 | 1.10E−38 | [Homo sapiens] (AB052907) ALCAN-beta |
| | | g19525540 | 1.00E−148 | [fl][Homo sapiens] lymphocyte effector toxicity activation ligand |
| 8 | 72830854CD1 | g3170204 | 9.90E−162 | [Homo sapiens] antigen NY-CO-43 |
| | | | | Scanlan, M. J. et al. (1998) Characterization of human colon cancer antigens |
| | | | | recognized by autologous antibodies. Int. J. Cancer 76: 652-658 |
| 9 | 3632052CD1 | g4105619 | 6.40E−218 | [Mus musculus] SPAF (spermatogenesis associated factor, AAA family) |
| | | | | Liu, Y. et al. (2000) Oncogene 19: 1579-1588 SPAF, a new AAA-protein specific |
| | | | | to early spermatogenesis and malignant conversion. |
| 10 | 7493817CD1 | g6062874 | 5.20E−236 | [Homo sapiens] candidate tumor suppressor protein DICE1 |
| | | | | Wieland, I. et al. (1999) Oncogene 18: 4530-4537 Isolation of DICE1: a gene |
| | | | | frequently affected by LOH and downregulated in lung carcinomas |
| 11 | 6715627CD1 | g2231999 | 0.0 | [Homo sapiens] MEA6 |
| | | | | Heckel, D. et al. (1997) Hum. Mol. Genet. 6: 2031-2041 cDNA cloning and |
| | | | | chromosomal mapping of a predicted coiled-coil proline-rich protein |
| | | | | immunogenic in meningioma patients |
| 12 | 7727886CD1 | g1220353 | 2.40E−150 | [Homo sapiens] paraneoplastic antigen |
| | | | | Fathallah-Shaykh, H. et al. (1991) Proc. Natl. Acad. Sci. USA.88: 3451-3454 |
| | | | | Cloning of a leucine-zipper protein recognized by the sera of patients with |
| | | | | antibody-associated paraneoplastic cerebellar degeneration. |
| 13 | 914113CD1 | g14861177 | 8.10E−73 | [Danio rerio] (AF222996) nocA-like Zn-finger protein (Sagerstrom, C. G. et al. |
| | | | | (2001) Dev. Dyn. 220 (4), 402-408) |
| 14 | 1953711CD1 | g1177322 | 0.0 | [Rattus norvegicus] CPG2 protein (Nedivi, E. et al. (1996) Proc. Natl. Acad. Sci. |
| | | | | U.S.A. 93 (5), 2048-2053) |
| 15 | 1595275CD1 | g1297319 | 7.70E−44 | [Homo sapiens] DOC1 |
| | | | | Mok, S. C. et al. (1994) Gynecol. Oncol. 52: 247-252 |
| 16 | 72332548CD1 | g5730480 | 3.00E−91 | [Homo sapiens] candidate tumor suppressor p33 ING1 homolog |
| 17 | 7322834CD1 | g15077051 | 0.0 | [Mus musculus] (AF288137) metastasis associated protein 1 |
| | | | | Simpson, A. et al. (2001) Gene 273: 29-39 |
| 18 | 1925714CD1 | g12659140 | 8.00E−256 | [Mus musculus] mage-e1 |
| 19 | 6803363CD1 | g3253213 | 2.80E−43 | [Homo sapiens] glioma amplified on chromosome 1 protein |
| | | | | Almeida, A. et al. (1998) Oncogene 16: 2997-3002 |
| 20 | 7070580CD1 | g3041877 | 6.50E−176 | [Homo sapiens] IB3089A |
| | | | | Habuchi, T. et al. (1998)Genomics 48: 277-288 |
| 21 | 7500176CD1 | g1322234 | 0.0 | [Homo sapiens] OS-9 precurosor |
| | | | | Su, Y. A. et al. (1996) Mol. Carcinog. 15: 270-275 |
| 22 | 7500506CD1 | g13097582 | 4.60E−241 | [Homo sapiens] (BC003512) mesothelin |
| 23 | 7500639CD1 | g14039857 | 1.20E−23 | [Homo sapiens] testes development-related NYD-SP22 |
| 24 | 7506167CD1 | g1916672 | 0.0 | [Homo sapiens] meningioma-expressed antigen 11 |
| | | | | Heckel, D. et al. (1997) cDNA cloning and chromosomal mapping of a predicted |
| | | | | coiled-coil proline-rich protein immunogenic in meningioma patients. Hum. Mol. |
| | | | | Genet. 6: 2031-2041 |
| | | 343086\|MGEA6 | 1.5E−244 | [Homo sapiens][Activator; Small molecule-binding protein] Meningioma |
| | | | | expressed antigen 6, a tumor antigen with an acidic activation domain, a proline- |
| | | | | rich region and two coiled-coil domains |
| | | | | Heckel, D. et al. (1997) cDNA cloning and chromosomal mapping of a predicted |
| | | | | coiled-coil proline-rich protein immunogenic in meningioma patients. Hum. Mol. |
| | | | | Genet. 6: 2031-2041 |
| | | 6350\|USO1 | 1.2E−13 | [Saccharomyces cerevisiae][Docking protein][Golgi] Coiled-coil protein with |
| | | | | similarity to mammalian integrin, required for protein transport from ER to Golgi |
| | | | | Nakajima, H. et al. (1991) A cytoskeleton-related gene, uso1, is required for |
| | | | | intracellular protein transport in Saccharomyces cerevisiae. J. Cell Biol. 113: 245-260 |
| 25 | 90081189CD1 | g1322234 | 0.0 | [Homo sapiens] OS-9 precurosor |
| | | | | Su, Y. A. et al. Complete sequence analysis of a gene (OS-9) ubiquitously |
| | | | | expressed in human tissues and amplified in sarcomas. Mol Carcinog 15, 270-5 |
| | 90081189CD1 | 428204\|OS-9 | 1.3E−291 | [Homo sapiens] Amplified in osteosarcoma 9, a putative soluble acidic protein |
| | | | | that may positively regulate cell growth and proliferation; co-amplification of the |
| | | | | corresponding gene with cyclin-dependent kinase 4 (CDK4) is associated with |
| | | | | sarcomas. |
| | | | | Su, Y. A. et al. (1996) Complete sequence analysis of a gene (OS-9) |
| | | | | ubiquitously expressed in human tissues and amplified in sarcomas. Mol. |
| | | | | Carcinog. 15: 270-275. |
| 26 | 7510095CD1 | g1322234 | 0.0 | [Homo sapiens] OS-9 precurosor |
| | | | | Su, Y. A. et al. (supra) |
| | | 742638\|CL25084 | 6.8E−19 | [Homo sapiens] Protein of unknown function, has a region of low similarity to a |
| | | | | region of OS-9, which is an acidic soluble protein that is associated with human |
| | | | | sarcomas. |
| 27 | 7510096CD1 | g1322234 | 8.50E−21 | [Homo sapiens] OS-9 precurosor. |
| | | | | Su, Y. A. et al. (supra) |
| | | 428204\|OS-9 | 7.0E−22 | [Homo sapiens] Amplified in osteosarcoma 9, a putative soluble acidic protein |
| | | | | that may positively regulate cell growth and proliferation; coamplification of the |
| | | | | corresponding gene with cyclin-dependent kinase 4 (CDK4) is associated with |
| | | | | sarcomas. |
| | | | | Su, Y. A. et al. (supra) |
| | | | | Kimura, Y. et al. Genomic organization of the OS-9 gene amplified in human |
| | | | | sarcomas. J Biochem (Tokyo) 122, 1190-5 (1997) |
| | | | | Nakayama, T. et al. Ca2(+)-dependent interaction of N-copine, a member of the |
| | | | | two C2 domain protein family, with OS-9, the product of a gene frequently |
| | | | | amplified in osteosarcoma. FEBS Lett 453, 77-80 (1999) |

TABLE 3

| | | Ami- | | | | |
|---|---|---|---|---|---|---|
| | | no | | | | |
| SEQ | Incyte | Acid | Potential | | | |
| ID | Polypeptide | Resi- | Phosphorylation | Potential | | Analytical Methods |
| NO: | ID | dues | Sites | Glycosylation Sites | Signature Sequences, Domains and Motifs | and Databases |
| 1 | 261510CD1 | 168 | S25 S70 S90 T120 | | PROTEIN EREBP4 HOMOLOG CODED FOR BY | BLAST_PRODOM |
| | | | | | C ELEGANS CDNA YK103H12.5 PD022373: | |
| | | | | | V42-L131, L6-S17 | |
| 2 | 7498674CD1 | 548 | S29 S97 S348 S531 | N189 N311 | signal_cleavage: M1-A30 | SPSCAN |
| | | | T131 T312 T377 | | | |
| | | | T407 | | | |
| | | | | | Signal Peptide: M1-A30, R7-A30 | HMMER |
| | | | | | Leucine Rich Repeat: S137-D160, L89-S112, | HMMER_PFAM |
| | | | | | H113-K136, E65-P88 | |
| | | | | | Leucine Rich Repeat C-terminal domain: N170-P212, | HMMER_PFAM |
| | | | | | L213-I219 | |
| | | | | | Lipocalin signature: D331-A344 | MOTIFS |
| 3 | 1629617CD1 | 378 | S93 S106 S121 | N27 N91 N298 | Homeobox domain: R44-K81 | HMMER_PFAM |
| | | | S133 S143 S159 | | | |
| | | | S238 T35 T50 | | | |
| | | | T120 T196 | | | |
| | | | | | ‘Homeobox’ domain signature and profile: T33-T104 | PROFILESCAN |
| | | | | | ‘Homeobox’ domain protein BL00027: W39-K81 | BLIMPS_BLOCKS |
| | | | | | Homeobox signature PR00024: N46-L57, T61-W71, | BLIMPS_PRINTS |
| | | | | | W71-K80 | |
| | | | | | IROQUOIS CLASS HOMEODOMAIN PROTEIN | BLAST_PRODOM |
| | | | | | IRX2 HOMEOBOX DNA BINDING NUCLEAR | |
| | | | | | PD086674: S232-N304 | |
| | | | | | HOMEOBOX DM00009\|P54269\|222-288: A23-K84 | BLAST_DOMO |
| | | | | | ‘Homeobox’ domain signature: L57-K80 | MOTIFS |
| 4 | 2369279CD1 | 528 | S370 S375 T65 | N77 N228 N271 | DNA BINDING DOMAIN PROTEIN-LIKE CXC | BLAST_PRODOM |
| | | | | N305 N329 | FACTOR TSO1 CONTAINING ACTIVATING | |
| | | | | | JC8.6B PD042633: K313-N410 | |
| 5 | 3167506CD1 | 374 | S33 S67 S140 S170 | N216 N369 | Homeobox domain: R211-K270 | HMMER_PFAM |
| | | | S195 S318 T202 | | | |
| | | | T283 T286 T325 | | | |
| | | | Y282 | | | |
| | | | | | ‘Homeobox’ domain signature and profile: T222-E292 | PROFILESCAN |
| | | | | | ‘Homeobox’ domain protein BL00027: Y228-K270 | BLIMPS_BLOCKS |
| | | | | | Homeobox signature PR00024: N235-L246, | BLIMPS_PRINTS |
| | | | | | G250-W260, W260-K269 | |
| | | | | | HOMEOBOX PROTEIN DNA BINDING | BLAST_PRODOM |
| | | | | | NUCLEAR TRANSCRIPTION REGULATION | |
| | | | | | PREB CELL LEUKEMIA ACTIVATOR | |
| | | | | | ALTERNATIVE PD007515: D18-A210 | |
| | | | | | HOMEOBOX; EXTRADENTICLE; DM08777 | BLAST_DOMO |
| | | | | | \|A54863\|54-177: T30-H155 | |
| | | | | | \|P40427\|56-182: Q32-H155 | |
| | | | | | HOMEOBOX DM00009 | BLAST_DOMO |
| | | | | | \|P40425\|240-306: R206-G273 | |
| | | | | | \|P40424\|229-295: R206-G273 | |
| | | | | | ATP/GTP-binding site motif A (P-loop): A279-T286 | MOTIFS |
| 6 | 3075937CD1 | 1723 | S6 S10 S16 S61 | N52 N422 N681 | Beta/Gamma crystallin: K1219-I1312, T1503-F1583, | HMMER_PFAM |
| | | | S92 S151 S209 | N867 N907 N1462 | H1320-I1403, Q1416-I1495, H1124-L1205 | |
| | | | S274 S284 S299 | | | |
| | | | S348 S456 S558 | | | |
| | | | S626 S689 S693 | | | |
| | | | S695 S759 S785 | | | |
| | | | S827 S900 S915 | | | |
| | | | S920 S979 S989 | | | |
| | | | S990 S1002 S1028 | | | |
| | | | S1033 S1104 | | | |
| | | | S1112 S1123 | | | |
| | | | S1139 S1158 | | | |
| | | | S1190 S1201 | | BETA-CRYSTALLIN DM00094 | BLAST_DOMO |
| | | | S1265 S1308 | | \|P07318\|145-233: V1263-R1310 | |
| | | | S1325 S1334 | | \|P11842\|21-111: K1219-I1312 | |
| | | | S1386 S1431 | | \|P19141\|110-199: V1263-I1312 | |
| | | | S1437 S1444 | | | |
| | | | S1447 S1491 | | | |
| | | | S1552 S1561 | | | |
| | | | S1579 S1672 | | | |
| | | | T105 T147 T228 | | Crystallins beta and gamma ‘Greek key’ motif | MOTIFS |
| | | | T246 T339 T346 | | signature: W1064-L1079. W1166-L1181, | |
| | | | T426 T432 T450 | | V1220-L1235, W1273-L1288, | |
| | | | T530 T931 T1008 | | W1364-L1379, W1455-L1470, I1504-L1519 | |
| | | | T1129 T1253 | | | |
| | | | T1385 T1488 | | | |
| | | | T1695 | | | |
| 7 | 5176268CD1 | 255 | S5 S185 S222 T38 | N36 N82 N154 | signal_cleavage: M1-G29 | SPSCAN |
| | | | T86 T93 T142 | N212 | | |
| | | | T170 T255 | | | |
| | | | | | Signal Peptide: S8-G30, M1-M27, M1-G30, M1-S32 | HMMER |
| | | | | | Cytosolic domain: M1-R12, Q249-T255 | TMHMMER |
| | | | | | Transmembrane domain: L13-F35, R226-W248 | |
| | | | | | Non-cytosolic domain: N36-D225 | |
| | | | | | RETINOIC ACID EARLY TRANSCRIPT CELL | BLAST_PRODOM |
| | | | | | SURFACE PROTEIN MHC ALPHA GAMMA | |
| | | | | | PD023817: L18-P224 | |
| | | | | | CLASS I HISTOCOMPATIBILITY ANTIGEN | BLAST_DOMO |
| | | | | | DM00083\|P13753\|9-210: P10-L197 | |
| 8 | 72830854CD1 | 1278 | S318 S347 S352 | N1057 N1173 | Signal Peptide: M1-P25, M1-P28, M1-A26, | HMMER |
| | | | S527 S601 S637 | | M44-A69 | |
| | | | S702 S706 S724 | | | |
| | | | S786 S822 S872 | | | |
| | | | S901 S925 S937 | | | |
| | | | S953 S968 S982 | | | |
| | | | S1055 S1059 | | | |
| | | | S1072 S1123 | | | |
| | | | S1274 T348 | | | |
| | | | T388 T418 T438 | | HUMAN COLON CANCER ANTIGEN NYCO43 | BLAST_PRODOM |
| | | | T460 T481 T578 | | PD133698: M809-Q965 | |
| | | | T721 T747 T862 | | | |
| | | | T926 T1014 T1116 | | | |
| | | | T1122 T1181 | | | |
| | | | T1194 T1213 | | | |
| | | | Y1096 | | | |
| | | | | | KINASE WNK1 KIAA0344 ANTIGEN NYCO43 | BLAST_PRODOM |
| | | | | | PD041299: T966-L1154 | |
| | | | | | LUMICAN; FIBROMODULIN; T-CELL; CD2; | BLAST_DOMO |
| | | | | | DM02006\|A40678\|300-393: P113-A189 | |
| | | | | | MICROSATELLITE BAT2; DM05517 | BLAST_DOMO |
| | | | | | \|P48634\|1-1860; A17-K300 | |
| | | | | | \|S37671\|1-1870: K33-I277 | |
| | | | | | Leucine zipper pattern: L13-L34 | MOTIFS |
| | | | | | Cell attachment sequence: R762-D764 | MOTIFS |
| 9 | 3632052CD1 | 541 | S2 S3 S26 S112 | N11 N16 N296 | Signal_cleavage: M1-A30 | SPSCAN |
| | | | S171 S173 S185 | | | |
| | | | S190 S199 S237 | | | |
| | | | S258 T210 T224 | | | |
| | | | T267 T292 T298 | | | |
| | | | T361 T406 T442 | | | |
| | | | T462 T508 Y61 | | | |
| | | | Y287 | | | |
| | | | | | Non-cytosolic domain: M1-W541 | TMHMMER |
| | | | | | ATPase family associated with various cellular | HMMER_PFAM |
| | | | | | activities (chaperone): G352-L540 | |
| | | | | | AAA-protein family proteins BL00674: Y315-P335, | BLIMPS_BLOCKS |
| | | | | | P350-A371, G383-R425, T442-N488, G521-L540 | |
| | | | | | PROTEIN ATPBINDING PROTEASE SUBUNIT | BLAST_PRODOM |
| | | | | | HOMOLOG REPEAT CELL DIVISION | |
| | | | | | ATPDEPENDENT NUCLEAR PD000092: | |
| | | | | | G352-Q497 | |
| | | | | | AAA-PROTEIN FAMILY DM00024 | BLAST_DOMO |
| | | | | | \|Q07590\|207-365: K312-R473 | |
| | | | | | \|P54609\|201-360: V313-R473 | |
| | | | | | \|P23787\|198-357: K312-R473 | |
| | | | | | \|P03974\|198-357: K312-R473 | |
| | | | | | AAA-protein family signature: V456-R474 | MOTIFS |
| | | | | | ATP/GTP-binding site motif A (P-loop): G357-T364 | MOTIFS |
| 10 | 7493817CD1 | 553 | S23 S45 S94 S106 | N492 | signal_cleavage: M1-Y24 | SPSCAN |
| | | | S253 S314 S359 | | | |
| | | | S464 S471 T54 | | | |
| | | | T127 T181 T212 | | | |
| | | | T238 T467 Y367 | | | |
| | | | | | Non-cytosolic domain: M1-V553 | TMHMMER |
| | | | | | TRANSMEMBRANE CODED FOR BY C | BLAST_PRODOM |
| | | | | | ELEGANS CDNA YK25A9.5 NOTCH2-LIKE EGF | |
| | | | | | PD043679: E121-R507 | |
| | | | | | Cell attachment sequence: R46-D48 | MOTIFS |
| 11 | 6715627CD1 | 804 | S62 S65 S111 S137 | N135 N323 N664 | signal_cleavage: M1-R64 | SPSCAN |
| | | | S139 S160 S170 | | | |
| | | | S175 S182 S221 | | | |
| | | | S297 S325 S341 | | | |
| | | | S415 S577 S613 | | | |
| | | | S631 S657 | | | |
| | | | S661 S662 T193 | | Signal Peptide: M30-A54 | HMMER |
| | | | T245 T266 T268 | | | |
| | | | T286 T346 T369 | | | |
| | | | T401 T430 T489 | | | |
| | | | T539 T666 Y406 | | | |
| | | | | | Cytosolic domain: M1-Y37 | TMHMMER |
| | | | | | Transmembrane domain: G38-W60 | |
| | | | | | Non-cytosolic domain: R61-T804 | |
| | | | | | Molluscan rhodopsin C-terminal tail signature | BLIMPS_PRINTS |
| | | | | | PR00239: P755-Y762, Y754-R766, P755-R766 | |
| | | | | | MEA6 PD123689: S448-G710, P767-P773 | BLAST_PRODOM |
| | | | | | PD085638: M1-E102 PD075273: V244-K310 | |
| | | | | | PROTEIN COILED COIL CHAIN MYOSIN | BLAST_PRODOM |
| | | | | | REPEAT HEAVY ATP-BINDING FILAMENT | |
| | | | | | HEPTAD PD000002: E107-E300 | |
| | | | | | Myosin-like protein DM07884\|Q02455\|35-1728: | BLAST_DOMO |
| | | | | | R74-I446 | |
| | | | | | TRICHOHYALIN DM03839\|P37709\|632-1103: | BLAST_DOMO |
| | | | | | E87-R444 | |
| | | | | | Leucine zipper pattern: L127-L148 | MOTIFS |
| 12 | 7727886CD1 | 465 | S11 S17 S54 S167 | N337 N455 | LEUKOCYTE DNA BINDING RECEPTOR CDR2 | BLAST_PRODOM |
| | | | S179 S228 S316 | | PARANEOPLASTIC CEREBELLAR | |
| | | | S344 S397 S432 | | DEGENERATIONASSOCIATED ANTIGEN | |
| | | | S463 T37 T55 T72 | | PD024533: W18-K460 | |
| | | | T90 T154 T388 | | | |
| | | | T450 Y220 Y360 | | | |
| | | | | | Non-cytosolic: M1-K465 | TMHMMER |
| | | | | | Cell attachment sequence: R292-D294 | MOTIFS |
| | | | | | Leucine zipper pattern: L115-L136, L181-L202 | MOTIFS |
| 13 | 914113CD1 | 651 | S12 S56 S106 S135 | N43 | Type I antifreeze protein signature PR00308: | BLIMPS_PRINTS |
| | | | S178 S187 S211 | | C442-C456, T488-P497 | |
| | | | S604 T85 T227 | | | |
| | | | T536 T553 | | | |
| | | | | | LORICRIN; DM07383\|P18165\|166-480: S135-G340, | BLAST_DOMO |
| | | | | | K14-G55, C442-T477 | |
| | | | | | \|P23490\|22-315: S139-V342, H15-F60 | |
| | | | | | PHASEOLUS GLYCINE-RICH CELL WALL | BLAST_DOMO |
| | | | | | PROTEIN 1.8 DM07973 | |
| | | | | | \|P09789\|1-383: G145-A343, P5-G55 | |
| | | | | | \|P27483\|1-337: G145-G340, R11-G55, N144-G157 | |
| 14 | 1953711CD1 | 1443 | S153 S168 S206 | N110 N287 N588 | Calponin homology (CH) domain: G178-P283, | HMMER_PFAM |
| | | | S234 S350 S494 | N596 N637 N1143 | I27-Q134 | |
| | | | S535 S545 S576 | | | |
| | | | S603 S606 S613 | | Actinin-type actin-binding domain proteins | BLIMPS_BLOCKS |
| | | | S732 S777 S826 | | BL00019: Q29-S39, D56-E78, K103-L138 | |
| | | | S935 S968 S1034 | | | |
| | | | S1054 S1066 | | | |
| | | | S1141 | | | |
| | | | S1196 S1213 | | Actinin-type actin-binding domain signatures: | PROFILESCAN |
| | | | S1222 S1244 | | R9-V61, K103-S147 | |
| | | | S1286 S1319 | | | |
| | | | S1361 S1370 | | | |
| | | | S1377 S1386 | | | |
| | | | S1422 T3 T173 | | | |
| | | | T196 T229 | | | |
| | | | T244 T393 T670 | | CPG2 PROTEIN PD148409: M501-I1440 | BLAST_PRODOM |
| | | | T839 T870 T964 | | | |
| | | | T1040 T1104 | | | |
| | | | T1115 T1130 | | | |
| | | | T1175 T1282 | | | |
| | | | T1338 T1347 | | | |
| | | | T1433 Y1116 | | | |
| | | | | | PROTEIN ACTIN-BINDING REPEAT CALCIUM | BLAST_PRODOM |
| | | | | | BINDING FIMBRIN PHOSPHORYLATION | |
| | | | | | ALPHA-ACTININ MULTIGENE FAMILY | |
| | | | | | CYTOSKELETON PD000807: Q25-I130 | |
| | | | | | ALPHA-ACTININ ACTIN-BINDING DOMAIN | BLAST_DOMO |
| | | | | | DM00325 | |
| | | | | | \|P30427\|179-405: D23-L138, A180-P283 | |
| | | | | | \|A44159\|48-277: L21-T139, K175-S234 | |
| | | | | | \|P11277\|48-260: L21-V159, N179-P266 | |
| | | | | | \|P46939\|25-254: D23-S150, Q177-P283 | |
| | | | | | Actinin-type actin-binding domain | MOTIFS |
| | | | | | signature 1: Q29-N38 | |
| 15 | 1595275CD1 | 1087 | S6 S12 S24 S108 | N58 N101 N146 | Aldehyde ferredoxin oxid PF01314: D850-P886, | BLIMPS_PFAM |
| | | | S130 S140 S152 | N231 N269 N273 | T300-I343, F407-M437, E99-K131 | |
| | | | S153 S167 S181 | N413 N548 N675 | | |
| | | | S195 S233 S255 | N715 N1019 | | |
| | | | S275 S307 S338 | | | |
| | | | S366 S417 | | | |
| | | | S459 S460 S569 | | PROTEIN COILED COIL CHAIN MYOSIN | BLAST_PRODOM |
| | | | S570 S573 S574 | | REPEAT HEAVY ATP-BINDING FILAMENT | |
| | | | S608 S622 S639 | | HEPTAD PD000002: E76-K316 | |
| | | | S652 S659 S703 | | | |
| | | | S717 S720 S740 | | | |
| | | | S745 S772 | | | |
| | | | S805 S840 S926 | | CHROMOSOME PROTEIN COILED COIL | BLAST_PRODOM |
| | | | S932 S956 S961 | | HEPTAD REPEAT PATTERN ATP-BINDING I | |
| | | | S981 S989 S990 | | MAJOR PD075049: M92-E361 | |
| | | | S1023 S1056 T148 | | | |
| | | | T215 T389 T427 | | | |
| | | | T448 T491 | | | |
| | | | T507 T510 T533 | | TRICHOHYALIN DM03839\|P37709\|632-1103: | BLAST_DOMO |
| | | | T670 T776 T898 | | L16-E412 | |
| | | | T919 T953 T975 | | | |
| | | | T998 T1004 | | | |
| | | | | | INTERMEDIATE FILAMENTS | BLAST_DOMO |
| | | | | | DM00061\|P23731\|37-456: L27-L353 | |
| | | | | | MYOSIN-LIKE PROTEIN MLP1 | BLAST_DOMO |
| | | | | | DM07884\|Q02455\|35-1728: L16-S579 | |
| | | | | | TRICHOHYALIN DM03839\|P22793\|921-1475: | BLAST_DOMO |
| | | | | | R14-Q420 | |
| | | | | | Leucine zipper pattern: L16-L37, L23-L44, | MOTIFS |
| | | | | | L158-L179, L165-L186 | |
| | | | | | Mitochondrial energy transfer proteins signature: | MOTIFS |
| | | | | | P640-I649 | |
| 16 | 72332548CD1 | 240 | S118 S140 S148 | | PHD-finger: Y188-E235 | HMMER_PFAM |
| | | | S195 T52 T147 | | | |
| | | | T152 T220 | | | |
| | | | | | PHD-finger PF00628: P205-L219 | BLIMPS_PFAM |
| 17 | 7322834CD1 | 686 | S119 S122 S175 | N16 N31 N115 | signa_cleavage: M1-A63 | SPSCAN |
| | | | S215 S272 S305 | N361 N427 | | |
| | | | S443 S644 T189 | | | |
| | | | T316 T417 T458 | | | |
| | | | T496 T636 T648 | | | |
| | | | Y13 Y380 | | | |
| | | | | | BAH domain: N4-K147 | HMMER_PFAM |
| | | | | | ELM2 domain: G148-A209 | HMMER_PFAM |
| | | | | | GATA zinc finger: A372-K414 | HMMER_PFAM |
| | | | | | Myb-like DNA-binding domain: M268-K314 | HMMER_PFAM |
| | | | | | METASTASIS-ASSOCIATED PROTEIN MTA1 | BLAST_PRODOM |
| | | | | | PD040130: M415-L667 | |
| | | | | | PD023997 (T27C4.4): E65-L177 | |
| | | | | | PD011563 (SIMILAR MTA1 T27C4.4 KIAA0458 | |
| | | | | | C04A2.2 CHROMOSOME II): D231-R324 | |
| | | | | | PD024227: L325-G371 | |
| 18 | 1925714CD1 | 1595 | S60 S111 S159 | N7 N123 N383 | MAGE family: R652-Q1251, I1313-Q1505 | HMMER_PFAM |
| | | | S198 S205 S227 | N660 N775 N1115 | | |
| | | | S238 S288 S345 | N1447 | | |
| | | | S528 S564 S646 | | | |
| | | | S662 S782 S1073 | | | |
| | | | S1083 S1257 | | | |
| | | | S1297 S1370 | | DNA RNA-DIRECTED POLYMERASE | BLAST_PRODOM |
| | | | S1377 S1381 | | PUTATIVE P150 PROTEIN TRANSCRIPTASE | |
| | | | S1399 S1518 | | REVERSE L1 SEQUENCE PD003002: T257-W371 | |
| | | | S1524 T176 T181 | | | |
| | | | T215 T279 T312 | | | |
| | | | T321 T327 T354 | | | |
| | | | T456 T459 T467 | | PUTATIVE P150 DNA PROTEIN RNA-DIRECTED | BLAST_PRODOM |
| | | | T481 T504 T509 | | POLYMERASE TRANSCRIPTASE REVERSE L1 | |
| | | | T517 T533 T548 | | SEQUENCE PD002970: H393-G472 PD002839: | |
| | | | T709 T721 T733 | | M473-I552 | |
| | | | T745 T776 T781 | | | |
| | | | T793 T805 | | | |
| | | | T817 T829 T841 | | ANTIGEN MELANOMA-ASSOCIATED | BLAST_PRODOM |
| | | | T865 T877 T889 | | MULTIGENE FAMILY PROTEIN TUMOR | |
| | | | T901 T937 T949 | | RELATED POLYMORPHISM MAGE4 MAGEB1 | |
| | | | T973 T985 T1009 | | PD003141: Q1145-G1249 | |
| | | | T1021 | | | |
| | | | T1033 T1116 | | TRANSCRIPTASE; REVERSE; II; ORF2; | BLAST_DOMO |
| | | | T1122 T1301 | | DM01354\|P08547\|558-973: Q232-K458 | |
| | | | T1513 T1547 Y23 | | DM01354\|I38588\|559-974: M244-K458 | |
| | | | Y1158 | | DM01354\|JU0033\|48-463: Q232-K458 | |
| | | | | | DM01354\|S23650\|1-411: M244-K458 | |
| | | | | | Leucine zipper pattern: L212-L233 | MOTIFS |
| 19 | 6803363CD1 | 592 | S151 S166 S212 | N127 N185 N247 | Signal Peptide: M1-P20, M1-G24 | HMMER |
| | | | S242 S289 S331 | N257 N276 N324 | | |
| | | | S568 T41 T46 | N488 N512 N562 | | |
| | | | T123 T129 T145 | | Leucine Rich Repeat: S151-L174 N247-A270 | HMMER_PFAM |
| | | | T332 T346 T369 | | | |
| | | | T381 T420 T496 | | | |
| | | | T506 Y478 | | | |
| | | | | | Leucine Rich Repeat: H271-V294, | HMMER_PFAM |
| | | | | | A79-P102, R103-D126, A175-R198, | |
| | | | | | N127-H150, Q319-N342, E55-P78, | |
| | | | | | R295-R318, S199-P222 | |
| | | | | | Leucine rich repeat C-terminal domain: N352-R405 | HMMER_PFAM |
| | | | | | Leucine rich repeat N-terminal domain: G24-P53 | HMMER_PFAM |
| | | | | | Immunoglobulin domain: G422-A482 | HMMER_PFAM |
| | | | | | Cytosolic domain: W552-I592 | TMHMMER |
| | | | | | Transmembrane domain: V529-V551 | |
| | | | | | Non-cytosolic domain: M1-L528 | |
| | | | | | Leucine-rich repeat signature PR00019: L80-V93, | BLIMPS_PRINTS |
| | | | | | L101-L114 | |
| | | | | | GLYCOPROTEIN HORMONE RECEPTOR | BLAST_DOMO |
| | | | | | DM02997\|P35376\|14-202: G24-L181 | |
| | | | | | Cell attachment sequence: R386-D388 | MOTIFS |
| 20 | 7070580CD1 | 678 | S34 S130 S179 | N80 N249 N368 | IB3089A PD147692: T40-C677 | BLAST_PRODOM |
| | | | S369 S418 S435 | N474 N521 N553 | | |
| | | | S440 S451 S475 | | | |
| | | | S491 S504 S547 | | | |
| | | | S551 S657 | | | |
| | | | T47 T103 T197 | | FKBP-type peptidyl-prolyl cis-trans isomerase | MOTIFS |
| | | | T281 T330 T448 | | signature 1: L319-S335 | |
| | | | T511 T532 T673 | | | |
| 21 | 7500176CD1 | 652 | S64 S150 S179 | N177 | signal_cleavage: M1-T25 | SPSCAN |
| | | | S361 S447 S454 | | | |
| | | | S469 T158 T221 | | | |
| | | | T224 T302 T376 | | | |
| | | | T456 T518 T543 | | | |
| | | | T596 Y93 Y120 | | | |
| | | | Y268 Y632 | | | |
| | | | | | Signal Peptide: M1-P21; M1-A22; M1-T25; | HMMER |
| | | | | | M1-G27; M1-G26; M1-P21 | |
| | | | | | Cytosolic domain: M1-L6 | TMHMMER |
| | | | | | Transmembrane domain: L7-G29 | |
| | | | | | Non-cytosolic domain: S30-F652 | |
| | | | | | CheB methylesterase. PF01339: G178-E191 | BLIMPS_PFAM |
| | | | | | PROTEIN OS9 PRECURSOR SIGNAL PD141024: | BLAST_PRODOM |
| | | | | | L232-F652 | |
| | | | | | ALTERNATIVE AMPLIFIED PRECURSOR OS-9 | BLAST_PRODOM |
| | | | | | SIMILAR SPLICING SIGNAL OSTEOSARCOMA | |
| | | | | | PD090541: P21-P99 | |
| | | | | | PROTEIN F7N22.4 OS9 PRECURSOR SIGNAL | BLAST_PRODOM |
| | | | | | PD033854: E100-P231 | |
| | | | | | Cell attachment sequence: R404-D406 | MOTIFS |
| 22 | 7500506CD1 | 457 | S62 S110 S115 | N57 N223 N323 | signal_cleavage: M1-P33 | SPSCAN |
| | | | S139 S215 S280 | N350 | | |
| | | | S427 T5 T41 T79 | | | |
| | | | T169 T225 T229 | | | |
| | | | T248 T370 Y195 | | | |
| | | | Y312 | | | |
| | | | | | Signal Peptide: M1-A38 | HMMER |
| | | | | | Cytosolic domain: M1-P8 | TMHMMER |
| | | | | | Transmembrane domain: L9-V31 | |
| | | | | | Non-cytosolic domain: Q32-A457 | |
| | | | | | PRECURSOR SIGNAL POTENTIATING FACTOR | BLAST_PRODOM |
| | | | | | MESOTHELIN CAK1 ANTIGEN | |
| | | | | | PREPROMEGAKARYOCYTE | |
| | | | | | MEGAKARYOCYTE PD025470: L77-A457 | |
| | | | | | Hemopexin domain signature: F152-L166 | MOTIFS |
| 23 | 7500639CD1 | 127 | S67 S103 T44 T86 | | | |
| 24 | 7506167CD1 | 763 | S29 S62 S65 S110 | N136 N325 N623 | signal_cleavage: M1-R64 | SPSCAN |
| | | | S111 S138 S140 | | | |
| | | | S161 S176 S183 | | | |
| | | | S222 S299 S327 | | | |
| | | | S343 S417 S536 | | | |
| | | | S572 S590 | | | |
| | | | S616 S620 S621 | | Cytosolic domain: M1-Y37 | TMHMMER |
| | | | T197 T247 T287 | | Transmembrane domain: G38-W60 | |
| | | | T288 T348 T371 | | Non-cytosolic domain: R61-T763 | |
| | | | T403 T432 T491 | | | |
| | | | T625 Y37 Y408 | | | |
| | | | | | ATP synthase B/B′ CF(0). PF00430: V45-E87 | BLIMPS_PFAM |
| | | | | | Meningioma-expressed antigen 6/11 (MEA6) | BLAST_PRODOM |
| | | | | | PD123689: G515-G669, S450-P682, P726-P732 | |
| | | | | | Meningioma-expressed antigen 6/11 (MEA6) | BLAST_PRODOM |
| | | | | | PD085638: M1-E102 | |
| | | | | | Meningioma-expressed antigen 6/11 (MEA6) | BLAST_PRODOM |
| | | | | | PD075273: I246-K312 | |
| | | | | | Meningioma-expressed antigen 6/11 (MEA6) | BLAST_PRODOM |
| | | | | | PD046284: H734-T763 | |
| | | | | | MYOSIN-LIKE PROTEIN MLP1 | BLAST_DOMO |
| | | | | | DM07884\|Q02455\|35-1728: L82-E501, R74-I448 | |
| | | | | | Leucine zipper pattern: L128-L149 | MOTIFS |
| 25 | 90081189CD1 | 612 | S64 S150 S179 | N177 | signal_cleavage: M1-T25 | SPSCAN |
| | | | S361 S447 S484 | | | |
| | | | T158 T221 T224 | | | |
| | | | T302 T376 T471 | | | |
| | | | T533 T556 Y93 | | | |
| | | | Y120 Y268 Y592 | | | |
| | | | | | Signal Peptide: M1-P21, M1-A22, M1-T25, | HMMER |
| | | | | | M1-G27, M1-G26 | |
| | | | | | Cytosolic domain: M1-L6 | TMHMMER |
| | | | | | Transmembrane domain: L7-G29 | |
| | | | | | Non-cytosolic domain: S30-F612 | |
| | | | | | PROTEIN F7N22.4 OS9 PRECURSOR SIGNAL | BLAST_PRODOM |
| | | | | | PD033854: E100-P231 | |
| | | | | | PROTEIN OS9 PRECURSOR SIGNAL PD090541: | BLAST_PRODOM |
| | | | | | P21-P99 | |
| | | | | | PROTEIN OS9 PRECURSOR SIGNAL PD141024: | BLAST_PRODOM |
| | | | | | L232-T533 | |
| | | | | | 95.1 KD PROTEIN F48E8.4 IN CHROMOSOME III | BLAST_PRODOM |
| | | | | | PROTEIN PD148072: S55-K271 | |
| | | | | | Cell attachment sequence: R404-D406 | MOTIFS |
| 26 | 7510095CD1 | 638 | S64 S150 S179 | N177 | Signal cleavage: M1-T25 | SPSCAN |
| | | | S361 S447 S484 | | | |
| | | | S604 T158 T221 | | | |
| | | | T224 T302 T376 | | | |
| | | | T471 T533 T558 | | | |
| | | | Y93 Y120 Y268 | | | |
| | | | | | Signal Peptide: M1-P21, M1-A22, M1-T25, | HMMER |
| | | | | | M1-G27, M1-G26 | |
| | | | | | Cytosolic domain: M1-L6 | TMHMMER |
| | | | | | Transmembrane domain: L7-G29 | |
| | | | | | Non-cytosolic domain: S30-N638 | |
| | | | | | PROTEIN OS9 PRECURSOR SIGNAL PD141024: | BLAST_PRODOM |
| | | | | | L232-W608, E100-P231, P21-P9 | |
| | | | | | Cell attachment sequence: R404-D406 | MOTIFS |
| 27 | 7510096CD1 | 75 | | | Signal cleavage: M1-T25 | SPSCAN |
| | | | | | Signal Peptide: M1-P21, M1-A22, M1-G26, M1-G27 | HMMER |
| | | | | | Cytosolic domain: M1-L6 | TMHMMER |
| | | | | | Transmembrane domain: L7-G29 | |
| | | | | | Non-cytosolic domain: S30-W75 | |
| | | | | | PROTEIN OS9 PRECURSOR SIGNAL PD090541: | BLAST_PRODOM |
| | | | | | P21-Q54 | |

TABLE 4

| Polynucleotide | |
|---|---|
| SEQ ID NO:/ | |
| Incyte ID/Sequence | |
| Length | Sequence Fragments |
| 28/261510CB1/ | 1-535, 14-233, 36-275, 36-593, 36-726, 52-430, 56-349, 59-260, 67-264, 67-505, 67-538, 67-583, 74-324, 108-435, |
| 3343 | 122-723, 127-595, 190-565, 219-870, 240-479, 249-843, 278-524, 278-539, 278-554, 282-925, 289-545, 332-948, |
| | 347-613, 368-945, 428-808, 518-966, 520-962, 522-966, 525-792, 525-966, 526-1086, 544-964, 554-963, 556-963, |
| | 559-966, 563-964, 568-964, 577-964, 581-962, 582-873, 594-958, 603-962, 605-967, 623-962, 632-959, 644-1260, |
| | 667-896, 716-1340, 949-1601, 954-1583, 1164-1462, 1308-1592, 1429-1634, 1429-2062, 1739-1986, 1743-1976, |
| | 1776-2053, 1815-2050, 1821-2109, 1822-2046, 1822-2419, 1837-2032, 1837-2403, 1952-2309, 1991-2281, 2021-2279, |
| | 2252-2458, 2266-2514, 2302-2564, |
| | 2302-2593, 2310-2654, 2333-2578, 2333-2601, 2340-2575, 2364-2543, 2367-2622, 2387-2655, 2404-2662, 2418-2628, |
| | 2432-2715, 2433-2737, 2449-2634, 2449-2726, 2449-2740, 2453-2737, 2469-2664, 2469-3077, 2494-2692, |
| | 2507-2749, 2526-2775, 2526-2776, 2530-2760, 2531-3277, 2642-2842, 2667-2944, 2679-3002, 2717-3328, 2735-2977, |
| | 2735-3171, 2735-3314, 2737-3327, 2746-2998, 2749-2998, 2837-3321, 2838-3050, 2838-3205, 2851-3289, |
| | 2854-3075, 2854-3323, 2854-3343, 2898-3128, 2898-3313, 2898-3343, 2923-3337, 2983-3324, 3049-3277, 3183-3332 |
| 29/7498674CB1/ | 1-211, 1-477, 1-507, 1-550, 1-552, 1-556, 1-601, 1-614, 1-623, 1-634, 1-639, 1-641, 1-643, 1-657, 2-691, 10-665, 39-169, |
| 3190 | 141-799, 366-970, 712-1005, 712-1075, 712-1097, 712-1198, 845-1037, 903-1000, 925-1495, 957-1229, 957-1574, |
| | 1064-1562, 1188-1741, 1214-1746, 1238-1924, 1269-1911, 1293-1593, 1300-1601, 1318-1890, 1336-1736, |
| | 1378-1725, 1378-1736, 1410-1930, 1417-2031, 1452-1736, 1520-1771, 1577-1879, 1577-2205, 1626-1968, 1634-2149, |
| | 1688-1959, 1720-2262, 1720-2276, 1720-2297, 1720-2340, 1724-2245, 1730-2373, 1762-2042, 1774-2048, |
| | 1801-2079, 1867-2391, 1915-2196, 1917-2184, |
| | 1920-2190, 1949-2251, 2041-2312, 2076-2327, 2076-2332, 2076-2542, 2095-2372, 2095-2556, 2185-2472, 2186-2446, |
| | 2187-2332, 2248-2503, 2248-2715, 2259-2492, 2259-2785, 2265-2507, 2279-2533, 2348-2985, 2355-2620, |
| | 2356-2830, 2356-2836, 2356-2990, 2358-3000, 2370-2663, 2378-2986, 2385-2957, 2385-2989, 2428-2995, 2449-2728, |
| | 2498-2988, 2501-2988, 2503-2985, 2513-2765, 2527-2782, 2553-2969, 2592-3005, 2593-2840, 2602-2989, |
| | 2610-3190, 2611-2915, 2612-2990, 2615-2859, 2674-2893, 2822-2989 |
| 30/1629617CB1/ | 1-182, 4-2567, 103-365, 103-825, 104-450, 227-707, 253-891, 743-1064, 819-1193, 853-1162, 853-1216, 987-1401, |
| 3150 | 987-1408, 987-1418, 987-1422, 988-1197, 988-1399, 988-1400, 988-1401, 988-1414, 988-1416, 989-1114, 989-1401, |
| | 1006-1401, 1054-1401, 1063-1516, 1117-1700, 1126-1419, 1126-1684, 1266-1563, 1314-1866, 1410-1637, |
| | 1475-1983, 1509-1969, 1541-1985, 1582-1987, 1857-2127, 1916-2199, 2026-2187, 2037-2283, 2046-2568, 2084-2372, |
| | 2188-2461, 2188-2742, 2319-2580, 2333-2564, 2359-2572, 2583-2738, 2583-3150 |
| 31/2369279CB1/ | 1-405, 348-772, 351-405, 564-786, 564-1069, 804-1287, 804-1470, 817-1166, 817-1297, 873-1629, 1101-1443, |
| 2012 | 1231-1896, 1231-1907, 1231-1960, 1231-1994, 1231-2011, 1231-2012 |
| 32/3167506CB1/ | 1-1601, 147-443, 148-725, 229-605, 240-606, 297-1606, 356-605, 393-745, 571-796, 575-796 |
| 1606 | |
| 33/3075937CB1/ | 1-563, 1-566, 1-615, 1-621, 1-678, 1-818, 1-7347, 2-307, 58-761, 62-601, 96-806, 376-1254, 383-698, 383-704, 412-1254, |
| 7364 | 433-1254, 455-1180, 462-1254, 469-1254, 549-7364, 851-1263, 1705-1950, 1905-2233, 2023-2633, 2202-2700, |
| | 2208-3023, 2220-2682, 2411-2700, 2442-3074, 2631-3281, 2632-3139, 2635-2884, 2639-2924, 2778-3211, |
| | 2813-3370, 2891-3501, 2897-3348, 2918-3718, 3038-3485, 3071-3532, 3075-3416, 3331-3834, 3491-4103, 3666-4046, |
| | 3666-4047, 3687-4046, 3687-4047, 3691-4005, 3691-4046, 3691-4047, 3815-4046, 3823-4046, 4219-4470, |
| | 4219-4721, 4221-4887, 4222-4906, 4224-4897, 4235-4500, 4269-4943, 4308-4964, 4335-4976, 4383-4506, 4392-5038, |
| | 4491-5118, 4506-4651, |
| | 4539-4781, 4560-5078, 4594-4791, 4652-4781, 4652-4921, 4674-5248, 4676-5245, 4699-5340, 4700-5517, 4755-5324, |
| | 4781-5416, 4782-4921, 4782-5010, 4782-5045, 4810-5358, 4828-5357, 4842-5382, 4875-5403, 4890-5327, |
| | 4922-5045, 4922-5204, 4955-5576, 4958-5471, 4969-5630, 4977-5267, 4988-5612, 4997-5573, 4997-5630, 5002-5582, |
| | 5011-5600, 5037-5702, 5044-5403, 5045-5204, 5046-5204, 5046-5227, 5049-5612, 5070-5360, 5070-5501, |
| | 5082-5345, 5111-5576, 5149-5369, 5165-5732, 5180-5636, 5180-5748, 5187-5748, 5188-5875, 5196-5756, 5199-5558, |
| | 5217-5779, 5227-5396, 5231-5367, 5231-5459, 5237-5630, 5247-5732, 5249-5538, 5249-5912, 5291-5742, |
| | 5332-5598, 5369-5692, 5378-5847, 5381-6053, 5465-5735, 5468-6061, 5494-6093, 5519-5973, 5520-5757, 5534-6071, |
| | 5559-5786, 5568-6096, 5573-6094, 5613-6096, 5647-5886, 5660-5920, 5665-6094, 5681-6096, 5702-5952, |
| | 5730-6058, 5737-5980, 5743-6045, 5767-6019, 5767-6027, 5783-6032, 5785-6064, 5785-6282, 5795-6052, |
| 33 | 5805-6085, 5810-6042, 5814-6576, 5849-6116, 5902-6176, 5906-6165, 5934-6181, 5952-6225, 6187-6663, 6345-6852, |
| | 6377-6660, 6379-6638, 6410-6918, 6416-6627, 6416-6821, 6419-6691, 6419-6722, 6440-6724, 6452-7281, |
| | 6458-7026, 6464-6728, 6470-6920, 6470-6981, 6474-6739, 6482-6919, 6484-6735, 6487-6729, 6499-6751, 6499-6759, |
| | 6502-7062, 6524-6844, 6535-6952, 6536-6791, 6543-6800, 6549-6757, 6564-6826, 6588-6816, 6602-7278, |
| | 6629-7193, 6652-6934, 6661-7284, 6668-7060, 6672-7325, 6679-7014, 6743-7311, 6751-7013, 6751-7022, 6760-7018, |
| | 6760-7053, 6764-7333, 6775-7331, 6836-6858, 6847-7306, 6849-7364, 6868-7335, 6885-7140, 6896-7355, |
| | 6897-7347, 6927-7364, 6928-7214, 6932-7188, 6937-7204, 6974-7212, 6978-7254, 7009-7287, 7026-7364, 7052-7342, |
| | 7067-7317, 7068-7328, 7068-7332, 7068-7341, 7068-7347, 7082-7358, 7086-7364, 7100-7361 |
| 34/5176268CB1/ | 1-281, 1-599, 1-1162, 249-792, 298-580, 298-891, 417-656, 420-841, 542-1151, 876-1345 |
| 1345 | |
| 35/72830854CB1/ | 1-370, 1-453, 1-466, 1-482, 1-485, 1-510, 1-516, 1-523, 1-535, 1-560, 1-568, 1-582, 5-453, 5-598, 51-628, 54-574, |
| 4255 | 114-609, 146-478, 254-954, 278-864, 287-946, 315-953, 351-587, 351-616, 351-628, 351-633, 357-621, 359-598, |
| | 359-1046, 372-1036, 375-997, 377-648, 377-650, 427-1003, 449-572, 466-927, 486-1032, 493-598, 493-607, 493-616, |
| | 493-702, 493-992, 537-1139, 538-1139, 574-620, 574-658, 574-692, 575-656, 583-620, 596-692, 603-653, 610-662, |
| | 610-721, 610-725, 610-728, 612-728, 621-728, 622-723, 632-1237, 647-697, 647-717, 647-722, 647-727, 647-728, |
| | 648-728, 657-728, 657-1393, 668-728, |
| | 670-1481, 682-728, 684-728, 687-1254, 693-728, 695-1373, 696-1370, 702-1440, 702-1464, 712-1390, 733-1410, |
| | 746-866, 755-1561, 763-1490, 765-1363, 775-1377, 780-1413, 788-1473, 795-1281, 796-1431, 796-1636, 835-1417, |
| | 848-1449, 855-1561, 901-1458, 906-1533, 921-1368, 930-1420, 950-1492, 952-1758, 969-1617, 969-1642, 973-1652, |
| | 975-1625, 977-1655, 978-1620, 985-1626, 985-1664, 991-1749, 992-1654, 1010-1696, 1021-1559, 1025-1480, |
| | 1039-1752, 1042-1582, 1043-1640, 1049-1640, 1050-1643, 1055-1473, 1066-1784, 1068-1737, 1069-1755, |
| | 1071-1738, 1085-1679, 1099-1663, 1106-1665, 1108-1676, 1114-1760, 1132-1661, 1156-1681, 1158-1755, 1173-1760, |
| | 1179-1623, 1184-1739, 1190-1723, 1206-1712, 1213-1784, 1254-1755, 1269-1758, 1299-1840, 1301-1717, |
| | 1305-1707, 1307-1717, 1410-1840, |
| 35 | 1413-1984, 1422-1746, 1446-1840, 1507-2176, 1522-2176, 1541-1607, 1557-2166, 1697-2259, 1820-2282, 2031-2440, |
| | 2044-2219, 2044-2274, 2044-2319, 2044-2400, 2044-2426, 2044-2442, 2044-2449, 2044-2450, 2044-2519, |
| | 2044-2520, 2044-2529, 2044-2547, 2044-2553, 2044-2562, 2044-2613, 2044-2666, 2044-2694, 2044-2704, 2044-2714, |
| | 2044-2716, 2044-2721, 2044-2724, 2044-2745, 2044-2756, 2044-2769, 2044-2788, 2044-2811, 2044-2840, |
| | 2044-2881, 2047-2824, 2048-2785, 2070-2802, 2078-2695, 2102-2290, 2102-2318, 2102-2319, 2131-2855, 2216-3668, |
| | 2223-2733, 2274-2496, 2279-2475, 2314-3110, 2381-2575, 2391-2626, 2486-3135, 2612-3256, 2623-2890, |
| | 2662-3338, 2678-3483, 2691-3136, 2702-3403, 2709-3396, 2754-3413, 2756-3372, 2764-3483, 2870-3369, 2884-3460, |
| | 2892-3500, 2966-3630, 2979-3171, 2984-3255, 2984-3492, 2995-3237, 2997-3453, 2997-3588, 3019-3236, |
| | 3027-3212, 3118-3625, 3132-3668, 3158-3668, 3164-3415, 3247-3668, 3298-3668, 3375-3916, 3380-3663, 3382-3648, |
| | 3385-3682, 3390-3761, 3399-3959, 3403-3720, 3424-3890, 3424-3901, 3432-3856, 3447-3746, 3452-3787, |
| | 3490-3668, 3511-3766, 3530-3906, 3616-3725, 3620-3989, 3625-4065, 3628-3857, 3665-3853, 3665-3861, 3665-3903, |
| | 3665-3985, 3668-4101, 3668-4233, 3668-4251, 3669-3715, 3669-3754, 3669-3778, 3669-3855, 3669-3874, |
| | 3669-3875, 3669-4003, 3669-4120, 3669-4176, 3669-4193, 3669-4226, 3669-4246, 3669-4247, 3669-4251, 3669-4254, |
| | 3669-4255, 3670-3903, 3670-3914, 3670-4003, 3670-4036, 3670-4221, 3670-4250, 3687-4253, 3689-4234, |
| | 3690-4254, 3708-4251, 3738-4253, 3749-4017, 3756-4024, 3763-3907, 3790-4235, 3829-4234, 3851-4234, 3857-4234, |
| | 3892-4234, 3937-4099, 3946-4232, 3977-4153 |
| 36/3632052CB1/ | 1-352, 353-706, 408-933, 415-926, 426-696, 506-783, 507-703, 507-889, 507-894, 507-950, 507-965, 508-920, 508-960, |
| 4181 | 508-964, 509-830, 514-692, 514-696, 514-752, 514-841, 514-857, 514-860, 514-923, 514-974, 530-1046, 536-861, |
| | 539-638, 556-1310, 565-764, 581-1190, 587-1129, 587-1247, 590-1020, 590-1162, 620-1255, 657-1203, 704-1299, |
| | 730-1196, 734-1175, 782-1403, 799-1404, 840-1095, 848-1358, 849-1403, 854-1132, 871-1119, 901-1576, |
| | 946-1367, 1117-1358, 1133-1577, 1144-1447, 1144-1477, 1160-1577, 1294-1577, 1320-1570, 1323-1562, 1329-1577, |
| | 1342-1577, 1379-1577, 1385-1577, 1386-1577, 1399-1577, 1404-2546, 1412-1577, 1422-1577, 1431-1577, |
| | 1775-1823, 1775-1933, 1775-2186, 2219-2635, 2248-2635, 2316-2635, 2355-2635, 2432-2610, 2468-2849, 2525-3823, |
| | 2682-2835, 2682-3121, 2682-3269, 2682-3277, 2682-3310, 2682-3325, 2682-3326, 2682-3340, 2682-3444, |
| | 2682-3448, 2686-3488, 2818-3631, 2837-3553, 2914-3720, 2941-3687, 2992-3382, 3055-3441, 3109-3940, 3185-3860, |
| | 3207-4181 |
| 37/7493817CB1/ | 1-819, 1-2478, 290-464, 290-491, 290-776, 290-829, 290-853, 290-863, 290-891, 290-901, 290-910, 290-929, 290-931, |
| 2478 | 290-961, 290-981, 293-914, 327-872, 413-891, 424-927, 450-873, 489-751, 610-1054, 614-1069, 840-1085, |
| | 840-1141, 891-1614, 909-1612, 959-1612, 1015-1216, 1015-1545, 1367-1467, 1674-1973 |
| 38/6715627CB1/ | 1-481, 11-669, 203-809, 226-743, 242-1102, 254-959, 278-1045, 358-994, 359-383, 359-1041, 491-639, 491-674, |
| 2928 | 491-937, 523-777, 558-936, 558-1194, 558-1274, 559-930, 666-1160, 702-1089, 727-1492, 759-1386, 768-1026, |
| | 768-1236, 838-1397, 872-1124, 880-1459, 900-1146, 937-1143, 937-1358, 955-1494, 975-1674, 992-1291, 995-1594, |
| | 1136-1293, 1168-1829, 1200-1890, 1221-1889, 1224-1980, 1227-1905, 1234-1416, 1293-1567, 1294-1541, |
| | 1297-1436, 1298-1591, 1337-1777, 1358-1635, 1359-1489, 1360-1427, 1370-1589, 1370-1870, 1395-1668, 1395-2171, |
| | 1409-1662, 1410-2058, 1433-1875, 1438-2172, 1447-2184, 1454-2081, 1455-1731, 1470-1690, 1472-2219, |
| | 1473-2185, 1486-2144, 1516-2170, 1530-2150, 1535-2059, 1603-2321, 1643-2205, 1648-2085, 1650-1839, 1653-2370, |
| | 1666-2307, 1725-2354, 1806-2354, 1809-2379, 1809-2388, 1814-2243, 1814-2308, 1886-2019, 1891-2388, |
| | 1965-2057, 2049-2270, 2115-2388, 2138-2343, 2282-2786, 2310-2928, 2359-2902, 2370-2598, 2454-2485, 2568-2809, |
| | 2568-2902 |
| 39/7727886CB1/ | 1-258, 1-596, 246-974, 327-580, 580-731, 580-1787, 707-956, 713-1327, 770-1365, 866-1478, 994-1620, 1018-1326, |
| 3486 | 1102-1756, 1265-1760, 1308-1882, 1336-1616, 1394-1970, 1501-2136, 1510-2033, 1516-2145, 1585-2117, |
| | 1630-2244, 1825-2237, 1827-2274, 1831-1981, 1835-2206, 1860-2285, 1912-2161, 2040-2310, 2049-2293, 2076-2623, |
| | 2141-2414, 2142-2607, 2240-2511, 2244-2547, 2286-2531, 2317-2451, 2317-2573, 2317-2849, 2320-2663, |
| | 2321-2792, 2352-2846, 2352-2956, 2352-3013, 2354-2592, 2401-2600, 2401-2721, 2412-2992, 2448-2709, 2469-2586, |
| | 2542-3110, 2554-2793, 2573-2817, 2618-3132, 2683-3292, 2683-3298, 2711-3003, 2715-3315, 2749-3276, |
| | 2756-3007, 2760-3030, 2774-3439, |
| | 2786-3086, 2787-3485, 2808-3486, 2824-3086, 2829-3486, 2856-3092, 2856-3110, 2857-3090, 2857-3217, 2858-3120, |
| | 2862-3204, 2882-3486, 2903-3484, 2920-3486, 2927-3484, 2944-3438, 2951-3379, 2968-3486, 2976-3189, |
| | 2978-3247, 2978-3486, 2982-3109, 2986-3400, 2986-3486, 2987-3190, 2988-3486, 3028-3486, 3029-3486, 3045-3486, |
| | 3054-3486, 3059-3486, 3065-3354, 3069-3486, 3075-3340, 3076-3486, 3078-3486, 3098-3486, 3100-3315, |
| | 3102-3486, 3104-3486, 3105-3486, 3106-3486, 3108-3486, 3113-3365, 3123-3486, 3140-3438, 3159-3486, 3160-3414, |
| | 3177-3486, 3182-3486, 3183-3433, 3183-3439, 3191-3486, 3212-3486, 3230-3280, 3232-3360, 3236-3486, |
| | 3259-3486, 3281-3486, 3283-3486, 3289-3468, 3311-3484, 3311-3486, 3312-3486, 3394-3484, 3394-3486 |
| 40/914113CB1/ | 1-623, 5-570, 222-1042, 299-878, 306-865, 319-1000, 362-713, 370-1067, 389-825, 389-1054, 410-738, 410-1097, |
| 2535 | 510-630, 510-1049, 511-758, 511-789, 511-823, 511-848, 511-864, 511-871, 511-875, 511-885, 511-937, 511-976, |
| | 511-981, 511-993, 511-1045, 511-1079, 511-1142, 511-1154, 511-1165, 511-1168, 511-1186, 511-1220, 511-1241, |
| | 511-1242, 511-1286, 511-1291, 511-1319, 511-1325, 511-1366, 511-1387, 511-1434, 511-1445, 511-1453, 511-1491, |
| | 513-1093, 516-868, 522-1079, 525-602, 534-1441, 594-1286, 626-1505, 662-1505, 721-1464, 776-1505, 788-1366, |
| | 817-1430, 833-1435, 836-1469, 892-1503, 896-1416, 911-1387, 911-1505, 913-1273, 918-1505, 920-1496, |
| | 921-1503, 923-1491, 923-1500, |
| | 924-1502, 924-1503, 924-1511, 928-1505, 929-1503, 929-1505, 934-1503, 935-1503, 937-1503, 945-1505, 960-1505, |
| | 967-1503, 969-1505, 970-1500, 974-1503, 975-1503, 995-1505, 1000-1500, 1001-1503, 1001-1504, 1011-1472, |
| | 1011-1511, 1029-1507, 1036-1503, 1052-1500, 1059-1505, 1065-1503, 1073-1503, 1075-1505, 1078-1488, |
| | 1103-1503, 1105-1303, 1107-1500, 1108-1503, 1108-1511, 1115-1503, 1120-1503, 1134-1472, 1142-1482, 1156-1503, |
| | 1161-1503, 1178-1511, 1181-1788, 1183-1500, 1189-1506, 1193-1435, 1195-1511, 1215-1503, 1236-1486, |
| | 1243-1856, 1254-1480, 1274-2094, 1334-1511, 1402-1816, 1403-1816, 1513-1785, 1671-1859, 1714-1962, 1805-1962, |
| | 1812-2467, 1830-2027, 2014-2230, 2014-2231, 2014-2235, 2014-2280, 2014-2297, 2014-2300, 2014-2309, |
| | 2014-2345, 2014-2359, |
| | 2014-2385, 2014-2427, 2014-2477, 2014-2486, 2014-2487, 2014-2489, 2014-2490, 2014-2491, 2014-2492, 2014-2495, |
| | 2014-2496, 2014-2498, 2016-2100, 2016-2268, 2016-2394, 2016-2489, 2017-2508, 2017-2517, 2018-2507, |
| | 2020-2525, 2021-2273, 2021-2342, 2021-2524, 2021-2533, 2022-2509, 2022-2520, 2022-2523, 2022-2524, 2026-2441, |
| | 2026-2506, 2032-2285, 2032-2373, 2032-2389, 2032-2463, 2034-2253, 2036-2267, 2036-2524, 2041-2463, |
| | 2042-2494, 2044-2338, 2044-2495, 2049-2503, 2054-2231, 2055-2226, 2056-2486, 2062-2455, 2084-2433, 2091-2535, |
| | 2103-2308, 2104-2489, 2118-2308, 2123-2427, 2139-2307, 2166-2516, 2172-2515, 2186-2523, 2210-2489, |
| | 2231-2533, 2290-2524, 2307-2370, 2344-2511 |
| 41/1953711CB1/ | 1-403, 33-491, 261-842, 261-853, 261-878, 290-668, 594-867, 751-1247, 810-1510, 824-1507, 884-1510, 1103-1329, |
| 5514 | 1277-1845, 1381-1406, 1428-1648, 1428-1920, 1428-1954, 1492-2036, 1498-2036, 1509-2036, 1518-2036, |
| | 1540-2036, 1563-2036, 1574-2036, 1575-2028, 1580-2036, 1581-1890, 1581-1894, 1614-1890, 1616-2036, 1632-1787, |
| | 1642-2036, 1682-2036, 1693-2036, 1791-2338, 1834-2233, 1840-2362, 1854-2396, 1923-2153, 2002-2569, |
| | 2005-2174, 2040-2552, 2041-2635, 2057-2512, 2147-2419, 2163-2688, 2189-2374, 2240-2751, 2261-2759, 2404-2515, |
| | 2407-2649, 2420-3042, 2502-2761, 2509-2949, 2658-3191, 2660-3273, 2660-3287, 2698-3034, 2698-3298, |
| | 2698-3307, 2743-3333, 2805-3215, |
| | 2837-3217, 2865-3411, 2959-3468, 2979-3468, 2980-3314, 3023-3468, 3028-3461, 3030-3468, 3075-3468, 3178-3432, |
| | 3178-3468, 3180-3333, 3199-3468, 3294-3686, 3303-3452, 3373-3810, 3428-3934, 3521-4071, 3537-4140, |
| | 3660-3925, 3672-4346, 3801-4030, 3801-4087, 3801-4356, 3802-4356, 3807-4471, 3813-4394, 3865-4192, 3886-4471, |
| | 4101-4605, 4101-4620, 4139-4400, 4139-4653, 4361-4479, 4361-4600, 4361-4718, 4361-4744, 4361-4752, |
| | 4361-4773, 4361-4774, 4361-4790, 4361-4812, 4361-4834, 4361-4851, 4361-4858, 4361-4898, 4361-4974, 4361-5033, |
| | 4362-4633, 4363-4501, 4369-4734, 4369-5056, 4388-4772, 4404-5042, 4603-4788, 4619-5187, 4623-4870, |
| | 4623-4903, 4673-5311, 4693-5305, 4794-5027, 4794-5052, 4794-5055, 4818-4978, 4819-5313, 4856-5272, 5221-5359, |
| | 5221-5514, 5225-5514 |
| 42/1595275CB1/ | 1-634, 1-2199, 1-2387, 3-716, 21-768, 35-555, 35-632, 62-460, 126-660, 200-718, 201-898, 206-875, 240-788, 244-941, |
| 3715 | 250-913, 328-1014, 333-1014, 341-1077, 377-950, 437-582, 457-934, 464-881, 465-917, 487-1138, 519-939, |
| | 521-934, 577-626, 585-1077, 617-836, 617-1154, 617-1183, 633-1016, 643-1112, 651-1119, 667-1011, 670-962, |
| | 766-1160, 893-1335, 894-1344, 952-1170, 1032-1259, 1215-1323, 1232-1754, 1344-1929, 1374-1746, 1490-2195, |
| | 1638-1806, 1713-1862, 1786-2389, 1822-2389, 1852-2516, 1957-2382, 2014-2751, 2084-2393, 2106-2792, 2108-2511, |
| | 2182-2399, 2372-2928, 2372-3084, 2507-3109, 2639-3317, 2649-3160, 2649-3342, 2697-3334, 2852-3575, |
| | 2919-3715 |
| 43/72332548CB1/ | 1-230, 1-301, 1-389, 1-414, 1-428, 1-473, 1-474, 1-479, 1-523, 1-680, 2-249, 2-519, 2-523, 3-521, 3-523, 6-369, 6-431, |
| 1082 | 6-520, 6-523, 12-393, 15-262, 15-268, 15-281, 15-686, 15-692, 15-723, 15-726, 15-729, 16-262, 16-273, 16-330, |
| | 120-342, 248-500, 269-447, 299-474, 496-1082, 526-1082, 547-692, 613-1073 |
| 44/7322834CB1/ | 1-367, 24-854, 59-631, 87-569, 115-898, 127-689, 150-792, 260-1041, 275-986, 279-1079, 340-993, 340-1015, 340-1031, |
| 2621 | 359-912, 359-1018, 365-990, 365-995, 369-1131, 394-1125, 396-916, 441-1103, 479-1255, 487-1015, 513-903, |
| | 550-1255, 563-1233, 564-1226, 569-1213, 580-1253, 582-1006, 592-1259, 602-1329, 603-961, 617-1068, 696-971, |
| | 708-1381, 754-1035, 758-1018, 771-991, 776-1390, 791-1576, 804-1025, 806-1082, 809-1131, 822-1423, 828-1490, |
| | 836-1079, 845-1425, 845-1608, 846-956, 850-964, 854-1142, 855-1527, 855-1606, 860-1158, 866-1484, 876-1611, |
| | 879-1118, 882-1110, 890-1544, 908-1574, 910-1606, 912-1239, 926-1595, 930-1517, 931-1588, 931-1598, |
| | 935-1226, 953-1245, 955-1598, 961-1634, 974-1398, 980-1277, 1008-1576, 1030-1486, 1039-1622, 1053-1457, |
| | 1087-1357, 1089-1371, |
| | 1091-1698, 1100-1272, 1106-1413, 1106-1585, 1132-1713, 1146-1423, 1149-1332, 1159-1422, 1162-1499, 1162-1566, |
| | 1168-1411, 1168-1417, 1168-1423, 1177-1750, 1187-1634, 1191-1728, 1227-1619, 1242-1536, 1249-1486, |
| | 1250-1435, 1271-1542, 1366-1764, 1482-2035, 1494-2126, 1642-2134, 1647-2134, 1663-2134, 1682-2134, 1687-2108, |
| | 1690-2084, 1694-1965, 1695-2084, 1696-2084, 1704-2134, 1710-2084, 1726-2116, 1733-2134, 1737-2086, |
| | 1763-2084, 1777-1961, 1783-2106, 1784-2301, 1793-2420, 1802-2602, 1804-2130, 1811-1930, 1834-2609, 1843-2116, |
| | 1892-2069, 1896-2612, 1901-2612, 1904-2621, 1906-2084, 1917-2126, 1918-2621, 1960-2621, 2144-2621, |
| | 2197-2522, 2245-2489, 2272-2460, 2350-2514 |
| 45/1925714CB1/ | 1-419, 101-5539, 201-4986, 2060-2517, 2060-2633, 2060-5071, 2291-2707, 2291-2815, 2291-2887, 2291-3067, |
| 5539 | 2291-3083, 2292-2952, 2293-3103, 2294-3175, 2294-3283, 2296-2399, 2296-2435, 2296-3226, 2297-2734, 2297-2751, |
| | 2297-2827, 2297-2923, 2297-2935, 2297-2936, 2297-3211, 2305-2470, 2332-2471, 2332-3175, 2332-3262, |
| | 2366-2700, 2366-3355, 2402-2481, 2402-3283, 2441-2615, 2441-3355, 2507-2676, 2507-3299, 2512-2650, 2512-3355, |
| | 2513-2578, 2545-2827, 2545-3355, 2561-2611, 2579-2748, 2579-2916, 2579-3355, 2621-2784, 2621-3260, |
| | 2652-2820, 2652-2831, 2652-3312, 2693-2856, 2693-2867, 2693-3331, 2708-2734, 2729-2896, 2729-3355, 2759-2928, |
| | 2759-3355, 2801-2963, 2801-3331, 2831-3034, 2831-3355, 2873-3046, 2873-3327, 2909-3346, 2939-3355, |
| | 2989-3144, 2989-3154, |
| 45 | 3197-3262, 3269-3522, 3349-3800, 3349-3991, 3612-4036, 3669-4105, 3798-4045, 3798-4207, 3825-4325, 3885-4400, |
| | 3901-4150, 3922-4180, 3933-4197, 3962-4461, 3983-4592, 4026-4392, 4131-4645, 4167-4672, 4172-4510, |
| | 4230-4672, 4246-4936, 4247-4672, 4249-4672, 4265-4672, 4342-4602, 4369-5019, 4375-4916, 4378-4865, 4453-4746, |
| | 4453-4853, 4456-4914, 4464-5006, 4465-4716, 4510-4672, 4517-4768, 4540-5218, 4600-4824, 4709-5004, |
| | 4709-5012, 4725-5137, 4802-5075, 4843-5102, 4844-5138, 4844-5313, 4881-5497, 4891-5140, 5068-5523, 5071-5494, |
| | 5085-5348, 5087-5319, 5087-5336, 5092-5349, 5092-5380, 5093-5531, 5097-5361, 5099-5300, 5099-5355, |
| | 5108-5526, 5110-5318, 5138-5366, 5148-5352, 5160-5537, 5203-5455, 5236-5537, 5237-5535, 5239-5537, 5254-5525, |
| | 5264-5537, 5285-5523, 5322-5537, 5369-5515, 5369-5523, 5369-5526, 5383-5535 |
| 46/6803363CB1/ | 1-707, 39-2338, 51-612, 190-845, 282-589, 599-1373, 608-1234, 914-1504, 957-1504, 1036-1500, 1276-1513, 1297-1474, |
| 2338 | 1757-2089, 1763-2327 |
| 47/7070580CB1/ | 1-3190, 176-680, 448-855, 497-701, 519-815, 572-1164, 585-842, 585-984, 585-1005, 585-1043, 680-1171, 767-1401, |
| 3214 | 815-1377, 877-1108, 877-1469, 877-1471, 877-1493, 894-1129, 1045-1186, 1047-1173, 1098-1654, 1098-1748, |
| | 1144-1485, 1188-1815, 1198-1752, 1218-1485, 1262-1901, 1273-1598, 1316-1914, 1324-1936, 1325-1960, |
| | 1348-2030, 1364-1880, 1371-1685, 1371-1703, 1373-1947, 1385-1944, 1407-2057, 1418-2027, 1432-1911, 1459-1805, |
| | 1469-2027, 1479-1864, 1528-1881, 1558-2179, 1562-2126, 1570-2195, 1607-2274, 1613-2208, 1617-2147, |
| | 1620-2263, 1627-2195, 1641-2208, 1642-1984, 1646-1826, 1646-2245, 1654-2282, 1658-2127, 1669-2337, 1671-2173, |
| | 1673-2196, 1677-2264, 1686-2312, 1690-1916, 1690-2082, 1699-2189, 1712-2405, 1729-2270, 1734-2201, |
| | 1741-2354, 1749-2265, |
| | 1751-2378, 1754-2034, 1757-2392, 1764-2388, 1773-1980, 1816-2363, 1829-2492, 1839-2386, 1846-2467, 1847-2465, |
| | 1858-2239, 1874-2465, 1876-2123, 1892-2535, 1897-2415, 1900-2411, 1908-2526, 1934-2307, 1934-2393, |
| | 1934-2406, 1934-2414, 1934-2554, 1939-2545, 1950-2499, 1958-2507, 1958-2533, 1960-2625, 1971-2264, 1971-2554, |
| | 1983-2625, 1988-2526, 2004-2668, 2044-2288, 2044-2300, 2044-2572, 2055-2676, 2057-2649, 2060-2658, |
| | 2065-2667, 2066-2768, 2081-2631, 2092-2622, 2124-2623, 2124-2666, 2126-2747, 2134-2761, 2140-2782, 2153-2630, |
| | 2153-2655, 2193-2741, 2195-2556, 2205-2768, 2211-2620, 2288-2742, 2296-2535, 2298-2535, 2502-3079, |
| | 2553-3046, 2587-3062, 2591-3214, 2600-3185, 2625-3077, 2698-3051, 2701-2953, 2701-3162, 2701-3188, 2741-3189, |
| | 2744-2981, 2851-3103, 2851-3106, 2851-3168, 2889-3169, 2894-3171 |
| 48/7500176CB1/ | 1-271, 1-2791, 126-359, 154-772, 157-714, 170-474, 170-624, 170-666, 170-749, 170-759, 172-738, 172-793, 174-563, |
| 2824 | 174-730, 176-428, 177-555, 177-661, 177-715, 177-787, 181-525, 183-576, 184-421, 185-719, 185-788, 186-761, |
| | 187-293, 187-342, 187-492, 187-638, 188-639, 189-518, 194-673, 195-315, 195-434, 195-582, 198-525, 220-688, |
| | 249-570, 294-526, 335-1046, 350-996, 364-982, 395-1266, 408-867, 464-744, 511-1196, 542-1093, 545-985, |
| | 545-1034, 547-1096, 550-945, 561-1286, 590-1303, 594-1278, 599-738, 604-1135, 612-1156, 622-1294, 625-1254, |
| | 626-1231, 628-1092, 637-1149, 645-1173, 646-1086, |
| | 681-1177, 681-1324, 685-1263, 690-1106, 695-1421, 696-1252, 698-1311, 719-1482, 727-1228, 734-1283, 734-1334, |
| | 772-1330, 774-1267, 777-906, 785-1343, 790-1408, 812-1381, 813-1252, 819-1443, 834-1572, 850-1443, 856-1086, |
| | 866-1031, 866-1516, 886-1486, 892-1122, 892-1316, 897-1232, 900-1124, 910-1538, 913-1231, 914-1462, |
| | 915-1191, 918-1327, 926-1394, 938-1519, 944-1148, 951-1246, 951-1632, 955-1200, 960-1244, 961-1209, 962-1227, |
| | 964-1316, 969-1249, 970-1241, 974-1259, 976-1682, 978-1208, 978-1250, 986-1277, 988-1687, 989-1276, |
| | 1014-1595, 1020-1275, 1020-1293, 1030-1280, |
| | 1041-1166, 1050-1317, 1060-1292, 1063-1351, 1074-1409, 1078-1556, 1083-1356, 1091-1379, 1099-1367, 1118-1343, |
| | 1118-1385, 1125-1801, 1127-1350, 1134-1317, 1150-1387, 1152-1426, 1155-1421, 1160-1496, 1176-1712, |
| | 1178-1379, 1196-1471, 1197-1531, 1215-1922, 1225-1844, 1226-1511, 1226-1530, 1237-1512, 1308-1501, 1312-1561, |
| | 1332-1665, 1337-1823, 1344-1823, 1350-1589, 1523-1766, 1523-2047, 1527-1696, 1556-2032, 1559-2047, |
| | 1563-1813, 1575-1744, 1575-1762, 1582-2131, 1584-2120, 1586-1809, 1607-1874, 1611-2253, 1622-1926, 1622-1974, |
| | 1628-1885, 1628-1903, 1634-1921, 1640-1862, 1652-1922, 1653-2119, 1655-1905, 1655-1928, 1662-1906, |
| | 1663-1905, 1663-1925, 1663-1926, 1664-1922, 1664-1924, 1665-1952, 1670-1969, 1673-1796, 1673-1901, 1673-1947, |
| | 1678-1931, 1678-1964, 1679-1911, 1681-2159, 1683-1958, 1684-1956, 1687-1888, 1689-1896, 1689-1980, |
| 48 | 1701-1969, 1704-1944, 1711-1903, 1716-1861, 1735-2021, 1754-2019, 1757-1998, 1761-1995, 1768-2029, 1797-2050, |
| | 1801-2546, 1820-2037, 1827-2632, 1831-2530, 1860-2730, 1872-2130, 1888-2574, 1892-2137, 1929-2724, |
| | 1933-2728, 1936-2416, 1939-2741, 1945-2519, 1949-2384, 1949-2751, 1963-2726, 1966-2052, 1966-2076, 1971-2724, |
| | 1991-2725, 2003-2757, 2005-2724, 2014-2459, 2024-2496, 2024-2728, 2033-2660, 2034-2572, 2037-2537, |
| | 2038-2727, 2042-2729, 2044-2687, 2045-2456, 2046-2725, 2047-2505, 2064-2758, 2066-2639, 2075-2383, 2079-2438, |
| | 2079-2745, 2081-2771, 2082-2435, 2084-2597, 2091-2565, 2091-2724, 2092-2726, 2095-2754, 2097-2697, |
| | 2099-2726, 2099-2752, 2100-2756, 2108-2667, 2108-2715, 2108-2726, 2108-2745, 2112-2717, 2114-2623, 2114-2752, |
| | 2115-2718, 2115-2760, 2120-2416, 2121-2765, 2126-2445, 2130-2576, 2133-2426, 2134-2381, 2134-2384, |
| | 2135-2372, 2138-2388, 2139-2375, 2139-2458, 2141-2424, 2141-2427, 2142-2391, 2142-2443, 2143-2419, |
| | 2147-2787, 2149-2701, 2152-2686, 2155-2341, 2155-2393, 2155-2428, 2157-2438, 2158-2414, 2160-2421, 2160-2774, |
| | 2163-2429, 2163-2622, 2164-2404, 2164-2429, 2165-2428, 2167-2756, 2170-2728, 2172-2443, 2176-2413, |
| | 2176-2418, 2177-2327, 2177-2399, 2177-2455, 2178-2678, 2182-2624, 2183-2784, 2184-2422, 2185-2785, 2186-2485, |
| | 2188-2451, 2188-2477, 2188-2728, 2191-2481, 2196-2528, 2196-2706, 2199-2490, 2201-2398, 2203-2478, |
| | 2203-2580, 2205-2432, 2208-2667, 2208-2747, 2210-2401, 2221-2443, 2223-2521, 2227-2752, 2228-2472, 2230-2479, |
| | 2233-2471, 2237-2446, 2240-2746, 2243-2726, 2244-2667, 2246-2743, 2248-2449, 2249-2666, 2249-2779, |
| | 2251-2530, 2251-2545, 2254-2505, 2258-2685, 2259-2689, 2261-2470, 2262-2531, 2264-2546, 2264-2690, 2266-2718, |
| | 2266-2725, 2269-2534, 2269-2547, 2270-2749, 2275-2515, 2277-2582, 2278-2542, 2280-2547, 2287-2583, |
| | 2287-2703, 2298-2559, 2298-2788, 2302-2545, 2304-2493, 2304-2527, 2304-2543, 2307-2490, 2307-2549, |
| | 2307-2824, 2308-2769, 2314-2612, 2315-2509, 2315-2564, 2318-2790, 2325-2587, 2325-2614, 2325-2725, 2325-2743, |
| | 2325-2752, 2325-2753, 2326-2696, 2327-2726, 2339-2689, 2346-2743, 2346-2757, 2347-2632, 2347-2642, |
| | 2351-2619, 2351-2650, 2360-2522, 2364-2636, 2364-2638, 2368-2619, 2372-2712, 2379-2609, 2379-2744, 2380-2757, |
| | 2381-2608, 2381-2627, 2381-2631, 2382-2618, 2392-2645, 2392-2682, 2394-2659, 2400-2598, 2400-2682, |
| | 2400-2694, 2403-2656, 2403-2669, 2403-2797, 2410-2757, 2411-2636, 2414-2797, 2418-2635, 2422-2799, 2425-2751, |
| | 2434-2797, 2437-2707, 2438-2723, 2440-2692, 2446-2651, 2460-2706, 2461-2719, 2472-2750, 2474-2596, |
| | 2482-2706, 2484-2713, 2484-2724, 2497-2760, 2499-2761, 2504-2721, 2510-2797, 2512-2791, 2513-2749, 2518-2757, |
| | 2540-2737, 2544-2797, 2545-2757, 2590-2783 |
| 49/7500506CB1/ | 1-225, 46-698, 46-774, 46-982, 48-190, 63-250, 351-917, 352-1063, 389-911, 392-768, 409-1070, 419-968, 432-911, |
| 1570 | 444-1111, 448-1307, 458-702, 470-854, 493-985, 495-1161, 523-800, 525-993, 527-793, 527-891, 556-658, |
| | 573-857, 574-967, 583-1158, 641-945, 653-883, 661-929, 682-979, 684-870, 688-1204, 688-1212, 692-1547, 695-955, |
| | 704-1382, 712-975, 747-1015, 747-1269, 788-1547, 803-1199, 838-1313, 860-1132, 865-1076, 872-1143, 880-1182, |
| | 883-1250, 919-1547, 945-1248, 985-1236, 991-1562, 1006-1245, 1089-1557, 1097-1570, 1117-1570, 1128-1570, |
| | 1135-1570, 1137-1556, 1156-1553, 1298-1557, 1470-1570, 1472-1570 |
| 50/7500639CB1/ | 1-209, 1-605, 4-272, 8-273, 8-278, 8-283, 8-534, 11-273, 18-274, 33-303, 38-328, 43-310, 49-285, 57-454, 115-566, |
| 725 | 117-370, 117-382, 120-686, 133-400, 169-725, 172-432, 362-669, 376-690, 566-723 |
| 51/7506167CB1/ | 1-803, 61-729, 72-3098, 124-853, 124-855, 124-874, 124-958, 124-1008, 136-576, 297-565, 385-1103, 664-1172, |
| 3108 | 668-1410, 895-1193, 923-1151, 951-1232, 1009-1118, 1058-1302, 1107-1331, 1129-1410, 1268-1425, 1353-1971, |
| | 1356-1880, 1425-1699, 1426-1673, 1469-1909, 1490-1767, 1502-1721, 1502-1971, 1527-1800, 1602-1822, 1657-1971, |
| | 1662-1945, 1706-2004, 1764-2116, 1782-2398, 1787-2482, 1799-2004, 1803-2482, 1804-2047, 1862-2251, |
| | 1893-2531, 1898-2143, 1968-2400, 1971-2237, 1972-2620, 1979-2508, 1981-2167, 1993-2275, 2011-2525, 2034-2599, |
| | 2041-2293, 2052-2273, 2066-2540, 2073-2498, 2074-2524, 2085-2716, 2139-2839, 2141-2346, 2141-2349, |
| | 2154-2609, 2158-2354, 2161-2364, 2161-2411, 2167-2682, 2168-2407, 2171-2682, 2184-2400, 2190-2796, 2191-2757, |
| | 2198-2678, 2201-2431, 2201-2453, 2211-2490, 2211-2738, 2215-2760, 2217-2448, 2230-2846, 2243-2873, |
| | 2245-2861, 2248-2506, 2252-2515, 2252-2558, 2260-2934, 2262-2702, 2279-2503, 2284-2549, 2285-2789, |
| | 2286-2851, 2287-2914, 2309-2437, 2313-2933, 2345-2593, 2357-2962, 2396-2854, 2405-2871, 2412-2873, 2414-3063, |
| | 2420-2871, 2422-2873, 2426-3072, 2430-2843, 2447-2873, 2449-2860, 2452-2873, 2459-2872, 2461-2870, |
| | 2466-2872, 2466-2873, 2467-2873, 2488-3090, 2493-2868, 2493-2871, 2495-2873, 2496-3108, 2501-2871, 2502-2863, |
| | 2508-2873, 2548-2864, 2565-2871, 2578-2849, 2581-2801, 2581-2872, 2601-2851, 2614-3095, 2620-2838, |
| | 2621-2868, 2623-2871, 2632-2863, 2638-2802, 2641-3024, 2649-3093, 2666-3095, 2668-3007, 2678-3098, 2681-3088, |
| | 2691-2944, 2692-3095, 2703-2955, 2731-3005, 2751-2998, 2753-3108, 2762-3091, 2779-2871, 2796-3041, |
| | 2807-3099, 2807-3108, 2808-3095, 2813-3089, 2816-3095, 2943-3043 |
| 52/90081189CB1/ | 1-652, 1-744, 1-1962, 340-1262, 596-1488, 1039-1962, 1071-1962 |
| 1962 | |
| 53/7510095CB1/ | 1-271, 1-3005, 126-359, 137-788, 137-848, 137-869, 137-870, 137-880, 142-805, 170-460, 170-474, 170-531, 170-586, |
| 3005 | 170-666, 170-749, 170-759, 170-772, 170-812, 176-404, 176-428, 176-435, 177-445, 177-555, 177-661, 177-666, |
| | 177-776, 180-475, 181-386, 181-406, 181-419, 181-427, 181-441, 181-454, 181-462, 181-468, 181-493, 181-525, |
| | 181-721, 182-374, 182-459, 182-475, 182-509, 182-787, 182-866, 182-870, 183-423, 183-442, 183-576, 183-870, |
| | 184-421, 184-438, 184-457, 184-849, 184-870, 185-475, 185-516, 185-643, 185-719, 185-730, 185-746, 185-750, |
| | 185-788, 185-805, 185-814, 185-824, 185-849, 185-854, 185-870, 186-438, 186-454, 186-463, 186-621, 186-761, |
| | 186-869, 187-266, 187-293, 187-341, 187-371, 187-429, 187-434, 187-435, 187-443, 187-449, 187-450, 187-451, |
| | 187-455, 187-460, 187-463, 187-466, 187-475, 187-476, 187-478, 187-479, 187-487, 187-492, 187-511, 187-538, |
| | 187-544, 187-556, 187-566, 187-638, 187-643, 187-682, 187-726, 187-728, 187-730, 187-753, 187-787, 187-788, |
| | 187-789, 187-824, |
| | 187-849, 187-869, 187-870, 187-872, 188-273, 188-306, 188-425, 188-430, 188-432, 188-436, 188-439, 188-447, |
| | 188-450, 188-451, 188-456, 188-457, 188-463, 188-485, 188-495, 188-499, 188-553, 188-639, 188-726, 188-793, |
| | 188-806, 188-863, 188-870, 189-413, 189-518, 189-661, 189-693, 189-788, 190-417, 190-426, 190-438, 190-447, |
| | 190-711, 190-729, 190-774, 190-867, 190-870, 191-469, 191-693, 191-729, 192-419, 192-451, 192-870, 193-385, |
| | 193-425, 193-438, 193-450, 193-455, 193-738, 193-749, 193-788, 193-797, 194-321, 194-502, 194-673, 195-315, |
| | 195-399, 195-416, 195-427, 195-428, 195-431, 195-434, 195-441, 195-452, 195-453, 195-458, 195-474, 195-478, |
| | 195-483, 195-488, 195-498, 195-582, 195-715, 195-718, 195-813, 195-869, 195-870, 195-875, 196-314, 196-434, |
| | 196-443, 196-447, 196-452, 196-663, 196-745, 197-446, 197-466, 197-704, 197-740, 197-772, 197-787, 198-435, |
| | 198-436, 198-525, 198-630, |
| 53 | 198-728, 198-770, 198-782, 199-560, 199-742, 199-861, 200-402, 200-410, 200-468, 200-472, 200-488, 200-686, |
| | 200-711, 201-432, 201-490, 201-722, 202-647, 203-446, 203-761, 204-432, 204-491, 204-553, 205-501, 206-872, |
| | 207-657, 208-516, 209-824, 211-767, 215-797, 216-655, 218-446, 220-688, 224-511, 226-468, 226-469, 234-516, |
| | 234-860, 240-537, 245-798, 249-570, 249-870, 253-507, 255-661, 255-751, 256-842, 260-718, 266-879, 269-794, |
| | 269-913, 288-607, 294-526, 297-866, 298-1104, 299-847, 302-539, 308-870, 316-864, 316-869, 322-876, 335-1046, |
| | 350-996, 364-982, 370-647, 379-616, 408-867, 410-633, 410-672, 410-978, 410-1003, 438-678, 449-851, 454-654, |
| | 455-868, 456-709, 456-720, 457-715, 461-871, 464-742, 464-744, 481-877, 488-1109, 511-1012, 511-1196, 525-1035, |
| | 531-789, 538-788, 542-1093, 545-822, 545-887, 545-979, 547-1096, 550-945, 559-909, 577-1169, 577-1328, |
| | 589-860, 590-1166, 591-852, 595-874, 599-738, 599-822, 600-886, 604-879, 604-1135, 606-890, 612-1089, 612-1156, |
| | 616-930, |
| | 622-1294, 625-864, 625-1166, 626-1231, 627-1172, 628-1092, 634-871, 635-868, 635-915, 635-923, 635-1095, 637-1149, |
| | 640-1149, 646-1086, 653-852, 653-907, 658-908, 658-924, 681-1177, 681-1324, 685-1263, 690-1106, 695-1321, |
| | 696-1252, 698-1286, 698-1311, 703-937, 707-924, 708-950, 710-904, 722-1140, 723-1168, 727-1228, 734-1283, |
| | 745-1156, 748-979, 754-975, 754-994, 754-1021, 754-1304, 759-982, 762-1029, 772-1330, 774-1267, 777-906, |
| | 784-1278, 785-1343, 790-1408, 810-940, 812-1381, 813-1252, 819-1443, 850-1443, 856-1086, 866-1031, 885-1516, |
| | 886-1486, 892-1122, 892-1316, 897-1232, 898-1163, 900-1124, 900-1741, 910-1170, 910-1432, 913-1231, |
| | 914-1462, 915-1191, 918-1327, 921-1334, 926-1394, 938-1195, 938-1519, 941-1568, 944-1173, 951-1246, 955-1200, |
| | 960-1244, 961-1209, 962-1227, 964-1316, 969-1249, 970-1241, 979-1538, 979-1557, 980-1455, 981-1265, |
| | 982-1211, 983-1208, |
| | 983-1250, 983-1259, 986-1277, 989-1276, 1002-1574, 1004-1586, 1011-1315, 1014-1557, 1020-1275, 1020-1293, |
| | 1030-1280, 1036-1610, 1041-1166, 1042-1733, 1050-1317, 1050-1319, 1060-1292, 1060-1317, 1063-1351, 1071-1359, |
| | 1074-1409, 1078-1556, 1083-1356, 1091-1379, 1099-1367, 1118-1343, 1118-1385, 1125-1846, 1127-1350, |
| | 1127-1351, 1127-1584, 1134-1317, 1150-1387, 1152-1426, 1155-1421, 1163-1533, 1176-1757, 1178-1379, 1178-1450, |
| | 1196-1479, 1196-1499, 1209-1488, 1226-1511, 1229-1453, 1252-1561, 1257-1511, 1258-1569, 1263-1481, |
| | 1267-1517, 1279-1539, 1279-1602, 1299-1549, 1308-1552, 1312-1541, 1523-1811, 1809-2083, 1825-2135, 1896-2361, |
| | 1930-2221, 1930-2511, 2097-2388, 2099-2338, 2099-2355, 2099-2390, 2099-2437, 2100-2350, 2110-2351, |
| | 2117-2369, 2128-2357, 2128-2382, 2131-2319, 2131-2355, 2131-2373, 2133-2446, 2136-2571, 2138-2390, 2162-2369, |
| | 2179-2461, 2222-2452, 2224-2724, 2437-2666, 2437-2669 |
| 54/7510096CB1/ | 1-271, 1-2906, 186-912, 255-724, 555-1059, 569-1082, 621-1278, 650-934, 679-912, 758-834, 798-1351, 806-1076, |
| 2906 | 942-1210, 954-1217, 988-1195, 1085-1518, 1301-1927, 1365-1778, 1390-2310, 1391-1933, 1397-1915, 1410-2015, |
| | 1414-1941, 1416-1645, 1416-1856, 1425-1661, 1438-1748, 1438-2040, 1466-1922, 1475-1748, 1478-1737, 1478-1769, |
| | 1479-1831, 1487-2046, 1495-2105, 1502-1816, 1511-1822, 1521-1793, 1522-1727, 1525-1842, 1528-1781, |
| | 1528-1784, 1528-1785, 1528-2107, 1533-2177, 1537-2310, 1542-1842, 1549-1886, 1552-1778, 1553-1785, 1557-1810, |
| | 1560-1830, 1567-1901, 1567-1902, 1567-1904, 1567-1996, 1567-1998, 1567-2045, 1567-2053, 1567-2057, |
| | 1567-2064, 1567-2078, 1567-2087, |
| | 1567-2089, 1569-2073, 1580-2037, 1581-1819, 1582-2309, 1586-2179, 1590-1829, 1593-1842, 1593-1998, 1594-1741, |
| | 1613-2087, 1623-1823, 1648-2124, 1655-1905, 1667-1836, 1667-1854, 1674-2223, 1676-2135, 1678-1901, |
| | 1694-1795, 1699-1966, 1703-2232, 1714-1842, 1714-2066, 1720-1977, 1720-1995, 1726-2013, 1744-2014, 1745-2211, |
| | 1747-1997, 1747-2020, 1754-1998, 1755-1997, 1755-2017, 1755-2018, 1756-2014, 1756-2016, 1757-2044, |
| | 1759-2245, 1762-2061, 1765-1888, 1765-1993, 1765-2039, 1770-2023, 1770-2056, 1771-2003, 1771-2369, 1773-2181, |
| | 1775-2050, 1776-2048, 1779-1980, 1781-1988, 1781-2072, 1783-2248, 1793-2061, 1793-2443, 1796-2036, |
| | 1803-1995, 1813-2381, |
| | 1821-2539, 1827-2113, 1835-2139, 1837-2268, 1840-2641, 1841-2132, 1846-2111, 1849-2090, 1853-2087, 1860-2121, |
| | 1866-2157, 1866-2161, 1882-2152, 1883-2299, 1889-2099, 1889-2142, 1889-2158, 1893-2638, 1896-2140, |
| | 1902-2138, 1902-2167, 1912-2129, 1923-2353, 1923-2470, 1930-2111, 1931-2172, 1931-2191, 1936-2246, 1943-2393, |
| | 1951-2196, 1963-2358, 1964-2222, 1967-2232, 1967-2261, 1969-2191, 1974-2417, 1978-2579, 1979-2190, |
| | 1980-2666, 1984-2229, 1985-2455, 1992-2245, 1999-2447, 2003-2112, 2004-2243, 2004-2260, 2004-2295, 2004-2342, |
| | 2005-2255, 2005-2571, 2012-2257, 2012-2271, 2015-2366, 2018-2304, 2018-2430, 2021-2816, 2028-2508, |
| | 2034-2299, 2036-2224, 2036-2260, 2036-2278, 2037-2611, 2038-2307, 2038-2351, 2041-2476, 2043-2279, 2043-2296, |
| | 2043-2306, 2043-2354, 2044-2303, 2044-2318, 2049-2273, 2051-2181, 2051-2329, 2055-2833, 2056-2457, |
| | 2058-2353, 2061-2310, 2065-2356, 2067-2274, 2071-2568, 2072-2362, 2076-2290, 2083-2387, 2083-2602, |
| 54 | 2084-2340, 2084-2346, 2084-2347, 2084-2366, 2086-2336, 2086-2337, 2086-2399, 2089-2335, 2092-2632, 2095-2353, |
| | 2095-2849, 2096-2328, 2097-2816, 2102-2368, 2105-2323, 2105-2407, 2106-2551, 2114-2354, 2114-2360, |
| | 2116-2588, 2116-2820, 2117-2307, 2122-2382, 2123-2343, 2123-2347, 2125-2752, 2126-2664, 2127-2357, 2129-2629, |
| | 2130-2415, 2136-2779, 2137-2403, 2137-2548, 2139-2597, 2140-2425, 2143-2416, 2144-2311, 2144-2369, |
| | 2144-2436, 2146-2271, 2146-2392, 2158-2731, 2167-2475, 2171-2418, 2171-2530, 2173-2863, 2174-2527, 2176-2689, |
| | 2177-2421, 2183-2657, 2187-2846, 2189-2789, 2191-2844, 2192-2848, 2197-2437, 2197-2462, 2199-2424, |
| | 2200-2807, 2204-2809, 2205-2454, 2206-2715, 2207-2824, 2207-2852, 2211-2509, 2212-2508, 2213-2482, 2217-2486, |
| | 2217-2819, 2218-2537, 2222-2668, 2225-2518, 2226-2473, 2226-2476, 2227-2462, 2227-2464, 2227-2491, |
| | 2227-2531, 2230-2480, 2230-2489, 2231-2467, 2231-2550, 2233-2516, 2233-2519, 2234-2483, 2234-2535, |
| | 2235-2511, 2241-2793, 2244-2474, 2244-2479, 2244-2778, 2245-2470, 2247-2433, 2247-2437, 2247-2485, 2247-2520, |
| | 2249-2530, 2250-2509, 2252-2485, 2252-2513, 2252-2866, 2255-2481, 2255-2521, 2255-2714, 2256-2476, |
| | 2256-2496, 2256-2521, 2257-2520, 2258-2814, 2259-2848, 2262-2821, 2264-2535, 2268-2505, 2268-2510, 2269-2419, |
| | 2269-2491, 2269-2547, 2270-2601, 2273-2455, 2274-2716, 2276-2514, 2277-2421, 2278-2577, 2280-2543, |
| | 2280-2555, 2280-2569, 2280-2820, 2281-2485, 2283-2573, 2288-2620, 2288-2798, 2291-2582, 2293-2490, 2295-2514, |
| | 2295-2570, 2295-2601, 2297-2524, 2300-2759, 2302-2493, 2313-2535, 2315-2613, 2319-2844, 2320-2564, |
| | 2322-2571, 2323-2851, 2325-2563, 2328-2817, 2329-2538, 2336-2759, 2338-2849, 2340-2541, 2341-2758, 2342-2571, |
| | 2342-2574, 2343-2622, 2343-2637, 2346-2597, 2350-2777, 2351-2781, 2353-2562, 2354-2623, 2356-2638, |
| | 2356-2782, 2357-2853, 2358-2810, 2361-2626, 2361-2639, 2362-2841, 2365-2648, 2367-2607, 2369-2674, |
| | 2370-2634, 2372-2639, 2372-2849, 2379-2675, 2379-2795, 2390-2651, 2394-2637, 2396-2585, 2396-2619, 2396-2635, |
| | 2397-2854, 2399-2582, 2399-2641, 2399-2906, 2400-2861, 2406-2704, 2407-2601, 2407-2656, 2410-2871, |
| | 2417-2679, 2417-2706, 2417-2817, 2417-2818, 2417-2844, 2417-2845, 2418-2788, 2419-2858, 2438-2849, 2439-2724, |
| | 2439-2734, 2443-2711, 2443-2742, 2450-2853, 2452-2614, 2456-2728, 2456-2730, 2460-2711, 2471-2701, |
| | 2471-2836, 2472-2849, 2473-2700, 2473-2719, 2473-2723, 2477-2660, 2477-2710, 2484-2737, 2484-2774, 2485-2840, |
| | 2486-2751, 2489-2748, 2492-2690, 2492-2774, 2492-2786, 2495-2748, 2495-2761, 2502-2849, 2503-2728, |
| | 2510-2727, 2517-2843, 2529-2799, 2530-2815, 2532-2784, 2538-2743, 2540-2764, 2550-2781, 2552-2798, 2553-2811, |
| | 2564-2842, 2566-2688, 2574-2798, 2576-2805, 2576-2816, 2589-2852, 2591-2853, 2596-2813, 2604-2878, |
| | 2605-2841, 2610-2849, 2682-2844 |

TABLE 5

| Polynucleotide | Incyte | Representative |
|---|---|---|
| SEQ ID NO: | Project ID: | Library |
| 28 | 261510CB1 | THYRNOT03 |
| 29 | 7498674CB1 | BRAUNOR01 |
| 30 | 1629617CB1 | BRSTNOT07 |
| 31 | 2369279CB1 | BRAXTDR15 |
| 32 | 3167506CB1 | OVARNOE02 |
| 33 | 3075937CB1 | BONEUNR01 |
| 34 | 5176268CB1 | EPIMNON05 |
| 35 | 72830854CB1 | BRAIFEF02 |
| 36 | 3632052CB1 | LIVRNON08 |
| 37 | 7493817CB1 | OVARDIJ01 |
| 38 | 6715627CB1 | BRAINOY02 |
| 39 | 7727886CB1 | BLADTUT02 |
| 40 | 914113CB1 | HEAONOR01 |
| 41 | 1953711CB1 | BRABDIR01 |
| 42 | 1595275CB1 | SINTTMR02 |
| 43 | 72332548CB1 | SCORNON02 |
| 44 | 7322834CB1 | KIDETXF04 |
| 45 | 1925714CB1 | BRAVUNT02 |
| 46 | 6803363CB1 | BRAUNOR01 |
| 47 | 7070580CB1 | BRAITUT08 |
| 48 | 7500176CB1 | UTRSDIC01 |
| 49 | 7500506CB1 | OVARTUE01 |
| 50 | 7500639CB1 | BRONNOT02 |
| 51 | 7506167CB1 | ISLTNOT01 |
| 53 | 7510095CB1 | BRAIFEC01 |
| 54 | 7510096CB1 | BRAYDIN03 |

TABLE 6

| Library | Vector | Library Description |
|---|---|---|
| BLADTUT02 | pINCY | Library was constructed using RNA isolated from bladder tumor tissue removed from an 80-year-old Caucasian female |
| | | during a radical cystectomy and lymph node excision. Pathology indicated grade 3 invasive transitional cell carcinoma. |
| | | Family history included acute renal failure, osteoarthritis, and atherosclerosis. |
| BONEUNR01 | PCDNA2.1 | This random primed library was constructed using pooled cDNA from two different donors. cDNA was generated using |
| | | mRNA isolated from an untreated MG-63 cell line derived from an osteosarcoma tumor removed from a 14-year-old |
| | | Caucasian male (donor A) and using mRNA isolated from sacral bone tumor tissue removed from an 18-year-old |
| | | Caucasian female (donor B) during an exploratory laparotomy and soft tissue excision. Pathology indicated giant cell |
| | | tumor of the sacrum in donor B. Donor B's history included pelvic joint pain, constipation, urinary incontinence, |
| | | unspecified abdominal/pelvic symptoms, and a pelvic soft tissue malignant neoplasm. Family history included prostate |
| | | cancer in donor B. |
| BRABDIR01 | pINCY | Library was constructed using RNA isolated from diseased cerebellum tissue removed from the brain of a 57-year-old |
| | | Caucasian male, who died from a cerebrovascular accident. Patient history included Huntington's disease, emphysema, |
| | | and tobacco abuse. |
| BRAIFEC01 | pINCY | This large size-fractionated library was constructed using RNA isolated from brain tissue removed from a Caucasian male |
| | | fetus who was stillborn with a hypoplastic left heart at 23 weeks' gestation. |
| BRAIFEF02 | PCMV-ICIS | This full-length enriched library was constructed using RNA isolated from brain tissue removed from a Caucasian male |
| | | fetus who was stillborn with a hypoplastic left heart at 23 weeks' gestation. Serologies were negative. |
| BRAINOY02 | pINCY | This large size-fractionated and normalized library was constructed using pooled cDNA generated using mRNA isolated |
| | | from midbrain, inferior temporal cortex, medulla, and posterior parietal cortex tissues removed from a 35-year-old |
| | | Caucasian male who died from cardiac failure. Pathology indicated moderate leptomeningeal fibrosis and multiple |
| | | microinfarctions of the cerebral neocortex. Microscopically, the cerebral hemisphere revealed moderate fibrosis of the |
| | | leptomeninges with focal calcifications. There was evidence of shrunken and slightly eosinophilic pyramidal neurons |
| | | throughout the cerebral hemispheres. Scattered throughout the cerebral cortex, there were multiple small microscopic |
| | | areas of cavitation with surrounding gliosis. Patient history included dilated cardiomyopathy, congestive heart failure, |
| | | cardiomegaly and an enlarged spleen and liver. 0.28 million independent clones from this size-selected library were |
| | | normalized in two rounds using conditions adapted from Soares et al., PNAS (1994) 91: 9228-9232 and Bonaldo et al., |
| | | Genome Research 6 (1996): 791, except that a significantly longer (48 hours/round) reannealing hybridization was used. |
| BRAITUT08 | pINCY | Library was constructed using RNA isolated from brain tumor tissue removed from the left frontal lobe of a 47-year-old |
| | | Caucasian male during excision of cerebral meningeal tissue. Pathology indicated grade 4 fibrillary astrocytoma with |
| | | focal tumoral radionecrosis. Patient history included cerebrovascular disease, deficiency anemia, hyperlipidemia, |
| | | epilepsy, and tobacco use. Family history included cerebrovascular disease and a malignant prostate neoplasm. |
| BRAUNOR01 | pINCY | This random primed library was constructed using RNA isolated from striatum, globus pallidus and posterior putamen |
| | | tissue removed from an 81-year-old Caucasian female who died from a hemorrhage and ruptured thoracic aorta due to |
| | | atherosclerosis. Pathology indicated moderate atherosclerosis involving the internal carotids, bilaterally; microscopic |
| | | infarcts of the frontal cortex and hippocampus; and scattered diffuse amyloid plaques and neurofibrillary tangles, |
| | | consistent with age. Grossly, the leptomeninges showed only mild thickening and hyalinization along the superior |
| | | sagittal sinus. The remainder of the leptomeninges was thin and contained some congested blood vessels. Mild |
| | | atrophy was found mostly in the frontal poles and lobes, and temporal lobes, bilaterally. Microscopically, |
| | | there were pairs of Alzheimer type II astrocytes within the deep layers of the neocortex. There was |
| | | increased satellitosis around neurons in the deep gray matter |
| | | in the middle frontal cortex. The amygdala contained rare diffuse plaques and neurofibrillary tangles. |
| | | The posterior hippocampus contained a microscopic area of cystic cavitation with hemosiderin-laden macrophages |
| | | surrounded by reactive gliosis. Patient history included sepsis, cholangitis, post-operative atelectasis, pneumonia CAD, |
| | | cardiomegaly due to left ventricular hypertrophy, splenomegaly, arteriolonephrosclerosis, nodular colloidal goiter, |
| | | emphysema, CHF, hypothyroidism, and peripheral vascular disease. |
| BRAVUNT02 | PSPORT1 | Library was constructed using pooled RNA isolated from separate populations of unstimulated astrocytes. |
| BRAXTDR15 | PCDNA2.1 | This random primed library was constructed using RNA isolated from superior parietal neocortex tissue removed |
| | | from a 55-year-old Caucasian female who died from cholangiocarcinoma. Pathology indicated mild meningeal |
| | | fibrosis predominately over the convexities, scattered axonal spheroids in the white matter of the cingulate cortex and the |
| | | thalamus, and a few scattered neurofibrillary tangles in the entorhinal cortex and the periaqueductal gray region. |
| | | Pathology for the associated tumor tissue indicated well-differentiated cholangiocarcinoma of the liver with residual |
| | | or relapsed tumor. Patient history included cholangiocarcinoma, post-operative Budd-Chiari syndrome, biliary ascites, |
| | | hydrothorax, dehydration, malnutrition, oliguria and acute renal failure. Previous surgeries included cholecystectomy |
| | | and resection of 85% of the liver. |
| BRAYDIN03 | pINCY | This normalized library was constructed from 6.7 million independent clones from a brain tissue library. Starting RNA |
| | | was made from RNA isolated from diseased hypothalamus tissue removed from a 57-year-old Caucasian male who died |
| | | from a cerebrovascular accident. Patient history included Huntington's disease and emphysema. The library was |
| | | normalized in 2 rounds using conditions adapted from Soares et al., PNAS (1994) 91: 9228 and Bonaldo et al., |
| | | Genome Research (1996) 6: 791, except that a significantly longer (48-hours/round) reannealing hybridization was used. |
| | | The library was linearized and recircularized to select for insert containing clones. |
| BRONNOT02 | pINCY | Library was constructed using RNA isolated from right lower lobe bronchial tissue removed from a pool of |
| | | 9 nonasthmatic Caucasian male and female donors, 18- to 55-years-old during bronchial pinch biopsies. Patient history |
| | | included atopy as determined by positive skin tests to common aero-allergens with no bronchial hyperresponsiveness |
| | | to histamine. The donors were not current smokers and had no history of alcohol or drug abuse. |
| BRSTNOT07 | pINCY | Library was constructed using RNA isolated from diseased breast tissue removed from a 43-year-old Caucasian female |
| | | during a unilateral extended simple mastectomy. Pathology indicated mildly proliferative fibrocystic changes with |
| | | epithelial hyperplasia, papillomatosis, and duct ectasia. Pathology for the associated tumor tissue indicated invasive grade |
| | | 4, nuclear grade 3 mammary adenocarcinoma with extensive comedo necrosis. Family history included epilepsy, |
| | | cardiovascular disease, and type II diabetes. |
| EPIMNON05 | pINCY | This normalized mammary epithelial cell tissue library was constructed from 3.28 million independent clones from an |
| | | epithelial cell tissue library. Starting RNA was made from untreated mammary epithelial cell tissue removed from a 21- |
| | | year-old female. The library was normalized in two rounds using conditions adapted from Soares et al., PNAS (1994) |
| | | 91: 9228 and Bonaldo et al., Genome Research 6 (1996): 791, except that a significantly longer (48 -hours/round) |
| | | reannealing hybridization was used. |
| HEAONOR01 | pINCY | This random primed library was constructed using pooled RNA isolated from aorta tissue removed from a 10-year-old |
| | | Caucasian male (donor A) who died from anoxia and a 27-year-old Caucasian female (donor B) who died from an |
| | | intracranial bleed. Patient history included asthma and suicidal tendency in donor A, and cerebral agenesis in donor B. |
| ISLTNOT01 | pINCY | Library was constructed using RNA isolated from a pooled collection of pancreatic islet cells. |
| KIDETXF04 | PCMV-ICIS | Library was constructed using RNA isolated from a treated, transformed embryonal cell line (293-EBNA) derived from |
| | | kidney epithelial tissue. The cells were treated with 5-aza-2′-deoxycytidine (5AZA) for 72 hours and Trichostatin |
| | | A for 24 hours and transformed with adenovirus 5 DNA. |
| LIVRNON08 | pINCY | This normalized library was constructed from 5.7 million independent clones from a pooled liver tissue library. Starting |
| | | RNA was made from pooled liver tissue removed from a 4-year-old Hispanic male who died from anoxia and a 16 week |
| | | female fetus who died after 16-weeks gestation from anencephaly. Serologies were positive for cytolomegalovirus in |
| | | the 4-year-old. Patient history included asthma in the 4-year-old. Family history included taking daily prenatal |
| | | vitamins and mitral valve prolapse in the mother of the fetus. The library was normalized in 2 rounds using |
| | | conditions adapted from Soares et al., PNAS (1994) 91: 9228 and Bonaldo et al., Genome Research 6 (1996): 791, except |
| | | that a significantly longer (48 hours/round) reannealing hybridization was used. |
| OVARDIJ01 | pIGEN | This random primed 5′ cap isolated library was constructed using RNA isolated from diseased right ovary tissue removed |
| | | from a 47-year-old Caucasian female during total abdominal hysterectomy, dilation and curettage, bilateral salpingo- |
| | | oophorectomy, repair of ureter, and incidental appendectomy. Pathology indicated endometriosis. Pathology for the |
| | | associated tumor tissue indicated multiple leiomyomata. The left ovary contained a corpus luteum. There was |
| | | endometriosis involving the posterior serosa. The patient presented with metrorrhagia and a benign neoplasm of the |
| | | ovary. Patient history included normal delivery, joint pain in multiple joints, and unilateral congenital hip |
| | | dislocation. Previous surgeries included total hip replacement. Patient medications included calcium. Family history |
| | | included kidney cancer in the mother; atherosclerotic coronary artery disease and aortocoronary bypass of 3 coronary |
| | | arteries in the father; benign hypertension and Hodgkin's disease in the sibling(s); and benign hypertension and |
| | | cerebrovascular accident in the grandparent(s). |
| OVARNOE02 | PCDNA2.1 | This 5′ biased random primed library was constructed using RNA isolated from right ovary tissue removed from a |
| | | 47-year-old Caucasian female during total abdominal hysterectomy, bilateral salpingo-oophorectomy, incisional hernia |
| | | repair, and panniculectomy. The patient presented with premenopausal menorrhagia. Patient history included |
| | | osteoarthritis, tubal pregnancy, and polio osteopathy of the left leg. Previous surgeries included gastroenterostomy, |
| | | plastic repair of the palate, adenotonsillectomy, dilation and curettage, cholecystectomy, and bladder reconstruction. |
| | | Patient medications included vitamins, iron, and zinc. Family history included benign hypertension and type II diabetes |
| | | in the father; and type II diabetes in the sibling(s). |
| OVARTUE01 | PCDNA2.1 | This 5′ biased random primed library was constructed using RNA isolated from left ovary tumor tissue removed from a |
| | | 44-year-old female. Pathology indicated grade 4 (of 4) serous carcinoma replacing both the right and left ovaries |
| | | forming solid mass cystic masses. Neoplastic deposits were identified in para-ovarian soft tissue. |
| SCORNON02 | PSPORT1 | This normalized spinal cord library was constructed from 3.24M independent clones from the a spinal cord tissue library. |
| | | RNA was isolated from the spinal cord tissue removed from a 71-year-old Caucasian male who died from respiratory |
| | | arrest. Patient history included myocardial infarction, gangrene, and end stage renal disease. The normalization and |
| | | hybridization conditions were adapted from Soares et al.(PNAS (1994) 91: 9228). |
| SINTTMR02 | PCDNA2.1 | This random primed library was constructed using RNA isolated from small intestine tissue removed from a 59-year-old |
| | | male. Pathology for the matched tumor tissue indicated multiple (9) carcinoid tumors, grade 1, in the small bowel. The |
| | | largest tumor was associated with a large mesenteric mass. Multiple convoluted segments of bowel were adhered to the |
| | | tumor. A single (1 of 13) regional lymph node was positive for malignancy. The peritoneal biopsy indicated focal fat |
| | | necrosis. |
| THYRNOT03 | pINCY | Library was constructed using RNA isolated from thyroid tissue removed from the left thyroid of a 28-year-old Caucasian |
| | | female during a complete thyroidectomy. Pathology indicated a small nodule of adenomatous hyperplasia present in |
| | | the left thyroid. Pathology for the associated tumor tissue indicated dominant follicular adenoma, forming a |
| | | well-encapsulated mass in the left thyroid. |
| UTRSDIC01 | PSPORT1 | This large size fractionated library was constructed using pooled cDNA from eight donors. cDNA was generated using |
| | | mRNA isolated from endometrial tissue removed from a 32-year-old female (donor A); endometrial tissue removed |
| | | from a 32-year-old Caucasian female (donor B) during abdominal hysterectomy, bilateral salpingo-oophorectomy, |
| | | and cystocele repair; from diseased endometrium and myometrium tissue removed from a 38-year-old Caucasian |
| | | female (donor C) during abdominal hysterectomy, bilateral salpingo-oophorectomy, and exploratory laparotomy; |
| | | from endometrial tissue removed from a 41-year-old Caucasian female (donor D) during abdominal hysterectomy with |
| | | removal of a solitary ovary; from endometrial tissue removed from a 43-year-old Caucasian female (donor E) during |
| | | vaginal hysterectomy, dilation and curettage, cystocele repair, rectocele repair and cystostomy; and from endometrial |
| | | tissue removed from a 48-year-old Caucasian female (donor F) during a vaginal hysterectomy, rectocele repair, and |
| | | bilateral salpingo-oophorectomy. Pathology (A) indicated the endometrium was in secretory phase. Pathology (B) |
| | | indicated the endometrium was in the proliferative phase. Pathology (C) indicated extensive adenomatous hyperplasia |
| | | with squamous metaplasia and focal atypia, forming a polypoid mass within the endometrial cavity. The cervix showed |
| | | chronic cervicitis and squamous metaplasia. Pathology (D, E) indicated the endometrium was secretory phase. Pathology |
| | | (F) indicated the endometrium was weakly proliferative. |

TABLE 7

| Program | Description | Reference | Parameter Threshold |
|---|---|---|---|
| ABI | A program that removes vector sequences and masks | Applied Biosystems, Foster City, CA. | |
| FACTURA | ambiguous bases in nucleic acid sequences. | | |
| ABI/ | A Fast Data Finder useful in comparing and | Applied Biosystems, Foster City, CA; | Mismatch <50% |
| PARACEL | annotating amino acid or nucleic acid sequences. | Paracel Inc., Pasadena, CA. | |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability value = |
| | sequence similarity search for amino acid and | 215: 403-410; Altschul, S. F. et al. (1997) | 1.0E−8 or less |
| | nucleic acid sequences. BLAST includes five | Nucleic Acids Res. 25: 3389-3402. | Full Length sequences: |
| | functions: blastp, blastn, blastx, tblastn, and tblastx. | | Probability value = 1.0E−10 |
| | | | or less |
| FASTA | A Pearson and Lipman algorithm that searches for | Pearson, W. R. and D. J. Lipman (1988) Proc. | ESTs: fasta E value = |
| | similarity between a query sequence and a group of | Natl. Acad Sci. USA 85: 2444-2448; Pearson, | 1.06E−6 Assembled ESTs: |
| | sequences of the same type. FASTA comprises as | W. R. (1990) Methods Enzymol. 183: 63-98; | fasta Identity = 95% or |
| | least five functions: fasta, tfasta, fastx, tfastx, and | and Smith, T. F. and M. S. Waterman (1981) | greater and Match length = |
| | ssearch. | Adv. Appl. Math. 2: 482-489. | 200 bases or greater; fastx |
| | | | E value = 1.0E−8 or less |
| | | | Full Length sequences: |
| | | | fastx score = 100 or greater |
| BLIMPS | A BLocks IMProved Searcher that matches a | Henikoff, S. and J. G. Henikoff (1991) | Probability value = 1.0E−3 |
| | sequence against those in BLOCKS, PRINTS, DOMO, | Nucleic Acids Res. 19: 6565-6572; Henikoff, | or less |
| | PRODOM, and PFAM databases to search for gene | J. G. & S. Henikoff (1996) Methods Enzymol. | |
| | families, sequence homology, and structural | 266: 88-105; and Attwood, T. K. et al. (1997) | |
| | fingerprint regions. | J. Chem. Inf. Comput. Sci. 37: 417-424. | |
| HMMER | An algorithm for searching a query sequence against | Krogh, A. et al. (1994) J. Mol. Biol. | PFAM, INCY, SMART, or |
| | hidden Markov model (HMM)-based databases of | 235: 1501-1531; Sonnhammer, E. L. L. et al. | TIGRFAM hits: Probability |
| | protein family consensus sequences, such as PFAM, | (1988) Nucleic Acids Res. 26: 320-322; | value = 1.0E−3 or less |
| | INCY, SMART, and TIGRFAM. | Durbin, R. et al. (1998) Our World View, | Signal peptide hits: Score = |
| | | in a Nutshell, Cambridge Univ. Press, p. 1-350 | 0 or greater |
| ProfileScan | An algorithm that searches for structural and sequence | Gribskov, M. et al. (1988) CABIOS 4: 61-66; | Normalized quality score ≧ |
| | motifs in protein sequences that match sequence | Gribskov, M. et al. (1989) Methods | GCG-specified “HIGH” value |
| | patterns defined in Prosite. | Enzymol. 183: 146-159; Bairoch, A. et al. | for that particular Prosite |
| | | (1997) Nucleic Acids Res. 25: 217-221. | motif. Generally, |
| | | | score = 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT | Smith, T. F. and M. S. Waterman (1981) Adv. | Score = 120 or greater; |
| | and CrossMatch, programs based on efficient | Appl. Math. 2: 482-489; Smith, T. F. and | Match length = 56 or greater |
| | implementation of the Smith-Waterman algorithm, | M. S. Waterman (1981) J. Mol. Biol. | |
| | useful in searching sequence homology and assembling | 147: 195-197; and Green, P., University of | |
| | DNA sequences. | Washington, Seattle, WA. | |
| Consed | A graphical tool for viewing and editing Phrap | Gordon, D. et al. (1998) Genome Res. 8: | |
| | assemblies. | 195-202. | |
| SPScan | A weight matrix analysis program that scans protein | Nielson, H. et al. (1997) Protein | Score = 3.5 or greater |
| | sequences for the presence of secretory signal peptides. | Engineering 10: 1-6; Claverie, J. M. and | |
| | | S. Audic (1997) CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) | Sonnhammer, E. L. et al. (1998) Proc. Sixth | |
| | to delineate transmembrane segments on protein | Intl. Conf. on Intelligent Systems for Mol. | |
| | sequences and determine orientation. | Biol., Glasgow et al., eds., The Am. Assoc. for | |
| | | Artificial Intelligence Press, Menlo Park, CA, | |
| | | pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| | | | | | | | | | | | | Asian | |
|---|---|---|---|---|---|---|---|---|---|---|---|---|---|
| SEQ | | | | | | | | | | Caucasian | African | Allele 1 | Hispanic |
| ID | | | | EST | CB1 | EST | | | Amino | Allele 1 | Allele 1 | fre- | Allele 1 |
| NO: | PID | EST ID | SNP ID | SNP | SNP | Allele | Allele 1 | Allele 2 | Acid | frequency | frequency | quency | frequency |
| 53 | 7510095 | 1217456H1 | SNP00105562 | 83 | 2816 | A | A | G | noncoding | n/d | n/d | n/d | n/d |
| 53 | 7510095 | 1218806H1 | SNP00004978 | 169 | 1587 | A | G | A | E465 | n/a | n/a | n/a | n/a |
| 53 | 7510095 | 1286173H1 | SNP00096962 | 43 | 2180 | A | A | G | noncoding | n/d | n/d | n/d | 0.97 |
| 53 | 7510095 | 1351490H1 | SNP00139100 | 226 | 1770 | C | C | T | P526 | n/a | n/a | n/a | n/a |
| 53 | 7510095 | 1386861H1 | SNP00025844 | 183 | 936 | T | T | G | P248 | n/d | n/a | n/a | n/a |
| 53 | 7510095 | 1674463H1 | SNP00139100 | 183 | 1771 | C | C | T | P527 | n/a | n/a | n/a | n/a |
| 53 | 7510095 | 1686583H1 | SNP00025845 | 105 | 1231 | C | C | T | P347 | n/a | n/a | n/a | n/a |
| 53 | 7510095 | 1798253H1 | SNP00004978 | 194 | 1584 | A | G | A | L464 | n/a | n/a | n/a | n/a |
| 53 | 7510095 | 1814731H1 | SNP00025845 | 253 | 1232 | C | C | T | P347 | n/a | n/a | n/a | n/a |
| 53 | 7510095 | 1860842H1 | SNP00004978 | 141 | 1581 | A | G | A | E463 | n/a | n/a | n/a | n/a |
| 53 | 7510095 | 1908048H1 | SNP00105562 | 132 | 2817 | A | A | G | noncoding | n/d | n/d | n/d | n/d |
| 53 | 7510095 | 2061203H1 | SNP00105562 | 34 | 2810 | A | A | G | noncoding | n/d | n/d | n/d | n/d |
| 53 | 7510095 | 2109864H1 | SNP00096962 | 84 | 2179 | A | A | G | noncoding | n/d | n/d | n/d | 0.97 |
| 53 | 7510095 | 2500612H1 | SNP00025844 | 178 | 937 | T | T | G | S249 | n/d | n/a | n/a | n/a |
| 53 | 7510095 | 2600068H1 | SNP00004978 | 80 | 1583 | A | G | A | Q464 | n/a | n/a | n/a | n/a |
| 53 | 7510095 | 2600068H1 | SNP00139100 | 264 | 1767 | C | C | T | N525 | n/a | n/a | n/a | n/a |
| 53 | 7510095 | 2745235H1 | SNP00025845 | 170 | 1229 | C | C | T | A346 | n/a | n/a | n/a | n/a |
| 53 | 7510095 | 2972896H2 | SNP00139100 | 67 | 1768 | C | C | T | P526 | n/a | n/a | n/a | n/a |
| 53 | 7510095 | 3050947H1 | SNP00096962 | 27 | 2178 | A | A | G | noncoding | n/d | n/d | n/d | 0.97 |
| 53 | 7510095 | 3074463H2 | SNP00025845 | 280 | 1230 | C | C | T | A346 | n/a | n/a | n/a | n/a |
| 53 | 7510095 | 3246779H1 | SNP00025844 | 186 | 930 | T | T | G | C246 | n/d | n/a | n/a | n/a |
| 53 | 7510095 | 3336225H1 | SNP00004978 | 36 | 1585 | A | G | A | K465 | n/a | n/a | n/a | n/a |
| 53 | 7510095 | 3336225H1 | SNP00139100 | 220 | 1769 | C | C | T | P526 | n/a | n/a | n/a | n/a |
| 53 | 7510095 | 3352432H1 | SNP00105562 | 116 | 2814 | A | A | G | noncoding | n/d | n/d | n/d | n/d |
| 53 | 7510095 | 3700090H1 | SNP00025845 | 66 | 1220 | C | C | T | A343 | n/a | n/a | n/a | n/a |
| 53 | 7510095 | 3831266H1 | SNP00025844 | 69 | 934 | T | T | G | S248 | n/d | n/a | n/a | n/a |
| 53 | 7510095 | 3859108H1 | SNP00004978 | 64 | 1586 | A | G | A | E465 | n/a | n/a | n/a | n/a |
| 53 | 7510095 | 3901943H1 | SNP00004978 | 160 | 1579 | G | G | A | E463 | n/a | n/a | n/a | n/a |
| 53 | 7510095 | 4199364H1 | SNP00139100 | 50 | 1765 | C | C | T | Q525 | n/a | n/a | n/a | n/a |
| 53 | 7510095 | 4433120H1 | SNP00025844 | 37 | 935 | G | T | G | R248 | n/d | n/a | n/a | n/a |
| 53 | 7510095 | 4708619H1 | SNP00025845 | 158 | 1228 | C | C | T | P346 | n/a | n/a | n/a | n/a |
| 53 | 7510095 | 4858491H1 | SNP00004978 | 78 | 1582 | G | G | A | V464 | n/a | n/a | n/a | n/a |
| 53 | 7510095 | 4921175H1 | SNP00025845 | 189 | 1218 | C | C | T | S342 | n/a | n/a | n/a | n/a |
| 53 | 7510095 | 6044386J1 | SNP00004979 | 65 | 2917 | T | C | T | noncoding | n/a | n/a | n/a | n/a |
| 53 | 7510095 | 6190082H1 | SNP00025844 | 37 | 933 | T | T | G | H247 | n/d | n/a | n/a | n/a |
| 53 | 7510095 | 6453815H1 | SNP00025844 | 390 | 931 | T | T | G | Y247 | n/d | n/a | n/a | n/a |
| 53 | 7510095 | 7972266H1 | SNP00130128 | 232 | 977 | C | T | C | P262 | n/a | n/a | n/a | n/a |
| 53 | 7510095 | 865177H1 | SNP00105562 | 113 | 2813 | A | A | G | noncoding | n/d | n/d | n/d | n/d |
| 53 | 7510095 | 916007H1 | SNP00025844 | 15 | 929 | T | T | G | F246 | n/d | n/a | n/a | n/a |
| 54 | 7510096 | 1217456H1 | SNP00105562 | 83 | 2721 | A | A | G | noncoding | n/d | n/d | n/d | n/d |
| 54 | 7510096 | 1218806H1 | SNP00004978 | 169 | 1634 | A | G | A | noncoding | n/a | n/a | n/a | n/a |
| 54 | 7510096 | 1286173H1 | SNP00096962 | 43 | 2085 | A | A | G | noncoding | n/d | n/d | n/d | 0.97 |
| 54 | 7510096 | 1351490H1 | SNP00139100 | 226 | 1817 | C | C | T | noncoding | n/a | n/a | n/a | n/a |
| 54 | 7510096 | 1386861H1 | SNP00025844 | 183 | 980 | T | T | G | noncoding | n/d | n/a | n/a | n/a |
| 54 | 7510096 | 1674463H1 | SNP00139100 | 183 | 1818 | C | C | T | noncoding | n/a | n/a | n/a | n/a |
| 54 | 7510096 | 1686583H1 | SNP00025845 | 105 | 1278 | C | C | T | noncoding | n/a | n/a | n/a | n/a |
| 54 | 7510096 | 1798253H1 | SNP00004978 | 194 | 1631 | A | G | A | noncoding | n/a | n/a | n/a | n/a |
| 54 | 7510096 | 1814731H1 | SNP00025845 | 253 | 1279 | C | C | T | noncoding | n/a | n/a | n/a | n/a |
| 54 | 7510096 | 1860842H1 | SNP00004978 | 141 | 1628 | A | G | A | noncoding | n/a | n/a | n/a | n/a |
| 54 | 7510096 | 1908048H1 | SNP00105562 | 132 | 2722 | A | A | G | noncoding | n/d | n/d | n/d | n/d |
| 54 | 7510096 | 191849H1 | SNP00096962 | 157 | 2086 | A | A | G | noncoding | n/d | n/d | n/d | 0.97 |
| 54 | 7510096 | 2061203H1 | SNP00105562 | 34 | 2715 | A | A | G | noncoding | n/d | n/d | n/d | n/d |
| 54 | 7510096 | 2109864H1 | SNP00096962 | 84 | 2084 | A | A | G | noncoding | n/d | n/d | n/d | 0.97 |
| 54 | 7510096 | 2250586H1 | SNP00105562 | 43 | 2720 | A | A | G | noncoding | n/d | n/d | n/d | n/d |
| 54 | 7510096 | 2490326H1 | SNP00096962 | 153 | 2083 | A | A | G | noncoding | n/d | n/d | n/d | 0.97 |
| 54 | 7510096 | 2500612H1 | SNP00025844 | 178 | 981 | T | T | G | noncoding | n/d | n/a | n/a | n/a |
| 54 | 7510096 | 2600068H1 | SNP00004978 | 80 | 1630 | A | G | A | noncoding | n/a | n/a | n/a | n/a |
| 54 | 7510096 | 2600068H1 | SNP00139100 | 264 | 1814 | C | C | T | noncoding | n/a | n/a | n/a | n/a |
| 54 | 7510096 | 2745235H1 | SNP00025845 | 170 | 1276 | C | C | T | noncoding | n/a | n/a | n/a | n/a |
| 54 | 7510096 | 2972896H2 | SNP00139100 | 67 | 1815 | C | C | T | noncoding | n/a | n/a | n/a | n/a |
| 54 | 7510096 | 3074463H2 | SNP00025845 | 280 | 1277 | C | C | T | noncoding | n/a | n/a | n/a | n/a |
| 54 | 7510096 | 3246779H1 | SNP00025844 | 186 | 974 | T | T | G | noncoding | n/d | n/a | n/a | n/a |
| 54 | 7510096 | 3336225H1 | SNP00004978 | 36 | 1632 | A | G | A | noncoding | n/a | n/a | n/a | n/a |
| 54 | 7510096 | 3336225H1 | SNP00139100 | 220 | 1816 | C | C | T | noncoding | n/a | n/a | n/a | n/a |
| 54 | 7510096 | 3352432H1 | SNP00105562 | 116 | 2719 | A | A | G | noncoding | n/d | n/d | n/d | n/d |
| 54 | 7510096 | 3700090H1 | SNP00025845 | 66 | 1267 | C | C | T | noncoding | n/a | n/a | n/a | n/a |
| 54 | 7510096 | 3831266H1 | SNP00025844 | 69 | 978 | T | T | G | noncoding | n/d | n/a | n/a | n/a |
| 54 | 7510096 | 3859108H1 | SNP00004978 | 64 | 1633 | A | G | A | noncoding | n/a | n/a | n/a | n/a |
| 54 | 7510096 | 3901943H1 | SNP00004978 | 160 | 1626 | G | G | A | noncoding | n/a | n/a | n/a | n/a |
| 54 | 7510096 | 4199364H1 | SNP00139100 | 50 | 1812 | C | C | T | noncoding | n/a | n/a | n/a | n/a |
| 54 | 7510096 | 4433120H1 | SNP00025844 | 37 | 979 | G | T | G | noncoding | n/d | n/a | n/a | n/a |
| 54 | 7510096 | 4708619H1 | SNP00025845 | 158 | 1275 | C | C | T | noncoding | n/a | n/a | n/a | n/a |
| 54 | 7510096 | 4858491H1 | SNP00004978 | 78 | 1629 | G | G | A | noncoding | n/a | n/a | n/a | n/a |
| 54 | 7510096 | 4921175H1 | SNP00025845 | 189 | 1265 | C | C | T | noncoding | n/a | n/a | n/a | n/a |
| 54 | 7510096 | 5903972H1 | SNP00096962 | 116 | 2082 | A | A | G | noncoding | n/d | n/d | n/d | 0.97 |
| 54 | 7510096 | 6044386J1 | SNP00004979 | 65 | 2822 | T | C | T | noncoding | n/a | n/a | n/a | n/a |
| 54 | 7510096 | 6190082H1 | SNP00025844 | 37 | 977 | T | T | G | noncoding | n/d | n/a | n/a | n/a |
| 54 | 7510096 | 6453815H1 | SNP00025844 | 390 | 975 | T | T | G | noncoding | n/d | n/a | n/a | n/a |
| 54 | 7510096 | 7972266H1 | SNP00130128 | 232 | 1024 | C | T | C | noncoding | n/a | n/a | n/a | n/a |
| 54 | 7510096 | 865177H1 | SNP00105562 | 113 | 2718 | A | A | G | noncoding | n/d | n/d | n/d | n/d |
| 54 | 7510096 | 916007H1 | SNP00025844 | 15 | 973 | T | T | G | noncoding | n/d | n/a | n/a | n/a |

1. An isolated polypeptide selected from the group consisting of:
a) a polypeptide comprising an amino acid sequence selected from the group consisting of SEQ ID NO:1-27,
b) a polypeptide comprising a naturally occurring amino acid sequence at least 90% identical to an amino acid sequence selected from the group consisting of SEQ ID NO:2-5, SEQ ID NO:7, SEQ ID NO:9-10, SEQ ID NO:12, SEQ ID NO:14-15, SEQ ID NO:18-19, SEQ ID NO:22-23, and SEQ ID NO:27,
c) a polypeptide comprising a naturally occurring amino acid sequence at least 98% identical to an amino acid sequence selected from the group consisting of SEQ ID NO:1 and SEQ ID NO:17,
d) a polypeptide comprising a naturally occurring amino acid sequence at least 96% identical to an amino acid sequence selected from the group consisting of SEQ ID NO:6 and SEQ ID NO:8,
e) a polypeptide comprising a naturally occurring amino acid sequence at least 99% identical to the amino acid sequence of SEQ ID NO:11,
f) a polypeptide comprising a naturally occurring amino acid sequence at least 95% identical to an amino acid sequence selected from the group consisting of SEQ ID NO:13 and SEQ ID NO:16,
g) A polypeptide comprising a naturally occurring amino acid sequenc at least 94% identical to an amino acid squence selected from the group consisting of SEQ ID NO:20, SEQ ID NO:24, and SEQ ID NO:26,
h) a biologically active fragment of a polypeptide having an amino acid sequence selected from the group consisting of SEQ ID NO:1-27, and
i) an immunogenic fragment of a polypeptide having an amino acid sequence selected from the group consisting of SEQ ID NO:1-27.

2. An isolated polypeptide of claim 1 comprising an amino acid sequence selected from the group consisting of SEQ ID NO:1-27.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:28-54.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-27.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:28-54,
b) a polynucleotide comprising a naturally occurring polynucleotide sequence at least 90% identical to a polynucleotide sequence selected from the group consisting of SEQ ID NO:28-37, SEQ ID NO:39-51, and SEQ ID NO:54,
c) a polynucleotide comprising a naturally occurring polynucleotide sequence at least 99% identical to a polynucleotide sequence of SEQ ID NO:52,
d) a polynucleotide comprising a naturally occurring polynucleotide sequence at least 94% identical to a polynucleotide sequence of SEQ ID NO:54,
e) a polynucleotide complementary to a polynucleotide of a),
f) a polynucleotide complementary to a polynucleotide of b),
g) a polynucleotide complementary to a polynucleotide of c),
h) a polynucleotide complementary to a polynucleotide of d), and
i) an RNA equivalent of a)-h).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-27.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample. wherein a difference in the amount of hybridization complex in the treated biological sample is indicative of toxicity of the test compound.

30-109. (canceled)


SEQUENCE LISTING (54 sequences)

SEQ ID NO: 1 - protein, 168 aa, Homo sapiens
  misc_feature: Incyte ID No 261510CD1
MEPPNLYPVKLYVYDLSKGLARRLSPIMLGKQLEGIWHTSIVVHKDEFFFGSGGISSCPPGGTLLGPPDSVVDVGSTEVTEEIFLEYLSSLGESLFRGEAYNLFEHNCNTFSNEVAQFLTGRKIPSYITDLPSEVLSTPFGQALRPLLDSIQIQPPGGSSVGRPNGQS

SEQ ID NO: 2 - protein, 548 aa, Homo sapiens
  misc_feature: Incyte ID No 7498674CD1
MAGLRARGGPGPGLLALSALGFCLMLQVSAKRPPKTPPCPPSCSCTRDTAFCVDSKAVPRNLPSEVISLTLVNAAFSEIQDGAFSHLPLLQFLLLNSNKFTLIGDNAFTGLSHLQYLFIENNDIWALSKFTFRGLKSLTHLSLANNNLQTLPRDIFRPLDILNDLDLRGNSLNCDCKVKWLVEWLAHTNTTVAPIYCASPPRFQEHKVQDLPLREFDCITTDFVLYQTLAFPAVSAEPFLYSSDLYLALAQPGVSACTILKWDYVERQLRDYDRIPAPSAVHCKPMVVDSQLYVVVAQLFGGSYIYHWDPNTTRFTRLQDIDPQRVRKPNDLEAFRIDGDWYFAVADSSKAGATSLYRWHQNGFYSHQALHPWHRDTDLEFVDGEGKPRLIVSSSSQAPVIYQWSRTQKQFVAQGEVTQVPDAQAVKHFRAGRDSYLCLSRYIGDSKILRWEGTRFSEVQALPSRGSLALQPFLVGGRRYLALGSDFSFTQIYQWDEGRQKFVRFQELAVQAPRAFCYMPAGDAQLLLAPSFKGQTLVYRHIVVDLSA

SEQ ID NO: 3 - protein, 378 aa, Homo sapiens
  misc_feature: Incyte ID No 1629617CD1
MTGAISYHPYGSAAYPYQLNDPAYRKNATRDATATLKAWLNEHRKNPYPTKGEKIMLAIITKMTLTQVSTWFANARRRLKKENKMTWAPRNKSEDEDEDEGDATRSKDESPDKAQEGTETSAEDEGISLHVDSLTDHSCSAESDGEKLPCRAGDPLCESGSECKDKYDDLEDDEDDDEEGERGLAPPKPVTSSPLTGLEAPLLSPPPEAAPRGGRKTPQGSRTSPGAPPPASKPKLWSLAEIATSDLKQPSLGPGCGPPGLPAAAAPASTGAPPGGSPYPASPLLGRPLYYTSPFYGNYTNYGNLNAALQGQGLLRYNSAAAAPGEALHTAPKAASDAGKAGAHPLESHYRSPGGGYEPKKDASEGCTVVGGGVQPYL

SEQ ID NO: 4 - protein, 528 aa, Homo sapiens
  misc_feature: Incyte ID No 2369279CD1
MEVVPAEIAKKPRTPTSGPVITKLIFAKPINSKAVTGQTTQVSPPVIAGRVLSQSTPGTPSKTITISESGVIGSTLNSTTQTPNKIAISPLKSPNKAVKSTVQTITVGGVSTSQFKTIIPLATAPNVQQIQVPGSKFHYVRLVTATSASSSTQPVSQNPSTNTQPLQQAKPVVVNTTPVRMSVPIVSAQAVKQVVPKPINPTSQIVTTSQPQQRLIMPATPLPQIQPNLTNLPPGTVLAPAPGTGNVGYAVLPAQYVTQLQQSSYVSIASNSTFTGTSGIQTQARLPFNGIIPSESASRPRKPCNCTKSLCLKLYCDCFANGEFCNNCNCTNCYNNLEHENERQKAIKACLDRNPEAFKPKIGKGKEGESDRRHSKGCNCKRSGCLKNYCECYEAKIMCSSICKCIGCKNFEESPERKTLMHLADAAEVRVQQQTAAKTKLSSQISDLLTRPTPALNSGGGKLPFTFVTKEVAEATCNCLLAQAEQADKKGKSKAAAERMILEEFGRCLMSVINSAGKAKSDPCAMNC

SEQ ID NO: 5 - protein, 374 aa, Homo sapiens
  misc_feature: Incyte ID No 3167506CD1
MAAPPRPAPSPPAPRRLDTSDVLQQIMAITDQSLDEAQARKHALNCHRMKPALFSVLCEIKEKTVVSIRGIQDEDPPDAQLLRLDNMLLAEGVCRPEKRGRGGAVARAGTATPGGCPNDNSIEHSDYRAKLSQIRQIYHSELEKYEQACREFTTHVTNLLQEQSRMRPVSPKEIERMVGAIHGKFSAIQMQLKQSTCEAVMTLRSRLLDARRKRRNFSKQATEVLNEYFYSHLNNPYPSEEAKEELARKGGLTISQVSNWFGNKRIRYKKNMGKFQEEATIYTGKTAVDTTEVGVPGNHASCLSTPSSGSSGPFPLPSAGDAFLTLRTLASLQPPPGGGCLQSQAQGSWQGATPQPATASPAGDPGSINSSTSN

SEQ ID NO: 6 - protein, 1723 aa, Homo sapiens
  misc_feature: Incyte ID No 3075937CD1
MEKRSSGRRSGRRRGSQKSTDSPGADAELPESAARDDAVFDDEVAPNAASDNASAEKKVKSPRAALDGGVASAASPESKPSPGTKGQLRGESDRSKQPPPASSPTKRKGRSRALEAVPAPPASGPRAPAKESPPKRVPDPSPVTKGTAAESGEEAARAISRELPVKSSSLLPEIKPEHKRGPLPNHFNGRAEGGRSRELGRAAGAPGASDADGLKPRNHFGVGRSTVTTKVTLPAKPKHVELNLKTPKNLDSLGNEHNPFSQPVHKGNTATKISLFENKRTNSSPRHTDIRGPRNTPASSKTFVGRAKLNLAKKAKEMEQPEKKVMPNSPQNGVLVKETAIETKVTVSEEEILPATRGMNGDSSENQALGPQPNQDDKADVQTDAGCLSEPVASALIPVKDHKLLEKEDSEAADSKSLVLENVTDTAQDIPTTVDTKDLPPTAMPKPQHTFSDSQSPAESSPGPSLSLSAPAPGDVPKDTCVQSPISSFPCTDLKVSENHKGCVLPVSRQNNEKMPLLELGGETTPPLSTERSPEAVGSECPSRVLVQVRSFVLPVESTQDVSSQVIPESSEVREVQLPTCHSNEPEVVSVASCAPPQEEVLGNEHSHCTAELAAKSGPQVIPPASEKTLPIQAQSQGSRTPLMAESSPTNSPSSGNHLATPQRPDQTVTNGQDSPASLLNISAGSDDSVFDSSSDMEKFTEIIKQMDSAVCMPMKRKKARMPNSPAPHFAMPPIHEDHLEKVFDPKVFTFGLGKKKESQPEMSPALHLMQNLDTKSKLRPKRASAEQSVLFKSLHTNTNGNSEPLVMPEINDKENRDVTNGGIKRSRLEKSALFSSLLSSLPQDKIFSPSVTSVNTMTTAFSTSQNGSLSQSSVSQPTTEGAPPCGLNKEQSNLLPDNSLKVFNFNSSSTSHSSLKSPSHMEKYPQKEKTKEDLDSRSNLHLPETKFSELSKLKNDDMEKANHIESVIKSNLPNCANSDTDFMGLFKSSRYDPSISFSGMSLSDTMTLRGSVQNKLNPRPGKVVIYSEPDVSEKCIEVFSDIQDCSSWSLSPVILIKVVRGCWILYEQPNFEGHSIPLEEGELELSGLWGIEDILERHEEAESDKPVVIGSIRHVVQDYRVSHIDLFTEPEGLGILSSYFDDTEEMQGFGVMQKTCSMKVHWGTWLIYEEPGFQGVPFILEPGEYPDLSFWDTEAAYIGSMRPLKMGGRKVEFPTDPKVVVYEKPFFEGKCVELETGMCSFVMEGGETEEATGDDHLPFTSVGSMKVLRGIWVAYEKPGFTGHQYLLEEGEYRDWKAWGGYNGELQSLRPILGDFSNAHMIMYSEKNFGSKGSSIDVLGIVANLKETGYGVKTQSINVLSGVWVAYENPDFTGEQYILDKGFYTSFEDWGGKNCKISSVQPICLDSFTGPRRRNQIHLFSEPQFQGHSQSFEETTSQIDDSFSTKSCRVSGGSWVVYDGENFTGNQYVLEEGHYPCLSAMGCPPGATFKSLRFIDVEFSEPTIILFEREDFKGKKIELNAETVNLRSLGFNTQIRSVQVIGGIWVTYEYGSYRGRQFLLSPAEVPNWYEFSGCRQIGSLRPFVQKRIYFRLRNKATGLFMSTNGNLEDLKLLRIQVMEDVGADDQIWIYQEGCIKCRIAEDCCLTIVGSLVTSGSKLGLALDQNADSQFWSLKSDGRIYSKLKPNLVLDIKGGTQYDQNHIILNTVSKEKFTQVWEAMVLYT

SEQ ID NO: 7 - protein, 255 aa, Homo sapiens [sequence omitted]

SEQ ID NO: 8 - protein, 1278 aa, Homo sapiens
  misc_feature: Incyte ID No 72830854CD1
MPPPQYFSPAVILPSLAAPLPPASPALPLQAVKLPHPPGAPLAMPCRTIVPNAPATIPLLAVAPPGVAALSIHSAVAQLPGQPVYPAAFPQMAPTDVPPSPHHTVQNMRATPPQPALPPQPTLPPQPVLPPQPTLPPQPVLPPQPTRPPQPVLPPQPMLPPQPVLPPQPALPVRPEPLQPHLPEQAAPAATPGSQILLGHPAPYAVDVAAQVPTVPVPPAAVLSPPLPEVLLPAAPELLPQFPSSLATVSASVQSVPTQTATLLPPANPPLPGGPGIASPCPTVQLTVEPVQEEQASQDKPPGLPQSCESYGGSDVTSGKELSDSCEGAFGGGRLEGRAARKHHRRSTRARSRQERASRPRLTILNVCNTGDKMVECQLETHNHKMVTFKFDLDGDAPDEIATYMVEHDFILQAERETFIEQMKDVMDKAEDMLSEDTDADRGSDPGTSPPHLSTCGLGTGEESRQSQANAPVYQQNVLHTGKRWFIICPVAEHPAPEAPESSPPLPLSSLPPEASQDSAPYKDQLSSKEQPSFLASQQLLSQAGPSNPPGAPPAPLAPSSPPVTALPQDGAAPATSTMPEPASGTASQAGGPGTPQGLTSELETSQPLAETHEAPLAVQPLVVGLAPCTPAPEAASTRDASAPREPLPPPAPEPSPHSGTPQPALGQPAPLLPAAVGAVSLATSQLPSPPLGPTVPPQPPSALESDGEGPPPRVGFVDSTIKSLDEKLRTLLYQEHVPTSSASAGTPVEVGDRDFTLEPLRGDQPRSEVCGGDLALPPVPKEAVSGRVQLPQPLVEKSELAPTRGAVMEQGTSSSMTAESSPRSMLGYDRDGRQVASDSHVVPSVPQDVPAFVRPARVEPTDRDGGEAGESSAEPPPSDMGTVGGQASHPQTLGARALGSPRKRPEQQDVSSPAKTVGRFSVVSTQDEWTLASPHSLRYSAPPDVYLDEAPSSPDVKLAVRRAQTASSIEVGVGEPVSSDSGDEGPRARPPVQKQASLPVSGSVAGDFVKKATAFLQRPSRAGSLGPETPSRVGMKVPTISVTSFHSQSSYISSDNDSELEDADIKKELQSLREKHLKEISELQSQQKQEIEALYRRLGKPLPPNVGFFHTAPPTGRRRKTSKSKLKAGKLLNPLVRQLKVVASSTGSSTSSLAPGPEPGPQPALHVQAQVNNSNNKKGTFTDDLHKLVDEWTSKTVGAAQLKPTLNQLKQTQKLQDMEAQAGWAAPGEARAMTAPRAGVGMPRLPPAPGPLSTTVIPGAAPTLSVPTPDPESEKPD

SEQ ID NO: 9 - protein, 541 aa, Homo sapiens
  misc_feature: Incyte ID No 3632052CD1
MSSKKNRKRLNQSAENGSSLPSAASSCAEARAPSAGSDFAATSGTLTVTNLLEKGKEFRVYTAWPMAGFPGGKVGLSEMAQKNVGVRPGDAIQVQPLVGAVLQAEEMDVALSDKDMEINEEELTGCILRKLDGKIVLPGNFLYCTFYGRPYKLQVLRVKGADGMILGGPQSDSDTDAQRMAFEQSSMETSSLELSLQLSQLDLEDTQIPTSRSTPYKPIDDRITNKASDVLLDVTQSPGDGSGLMLEEVTGLKCNFESAREGNEQLTEEERLLKFSIGAKCNTDTFYFISSTTRVNFTEIDKNSKEQDNQFKVTYDMIGGLSSQLKAIREIIELPLKQPELFKSYGIPAPRGLLLYGPPCTGKTMIARAVANEVGAYVSVINGPEIISKFYGETEAKLRQIFAEATLRHPSIIFIDELDALCPKREGAQNEVEKRVVASLLTLMDGIGSEVSEGQVLVLGATNRPHALDAALRRPGRFDKEIEIGVPNAQDRLDILQKLLRRVPHLLTEAELLQLANSAHGYVGADLKVLCNEAGECGLLW

SEQ ID NO: 10 - protein, 553 aa, Homo sapiens
  misc_feature: Incyte ID No 7493817CD1
MPILLFLIDTSASMNQRTDLGTSYLDIAKGAVELFLKLRARDPASRGDRYMLVTYDEPPYCIKAGWKENHATFMSELKNLQASGLTTLGQALRSSFDLLNLNRLISGIDNYGQGRNPFFLEPSILITITDGNKLTSTAGVQEELHPPLNSPLPGRELTKEPFRWDQRLFALVLRLPGVASTEPEQLGSVPTDESAITQMCEVTGGRSYCVRTQRMLNQCLESLVQKVQSGVVINFEKTGPDPLPIGEDGLMDSSRPSNSFAAQPWHSCHKLIYVRPNSKTGVPVGHWPIPESFWPDQNLPSLPPRTSHPVVRFSCVDCEPMVIDKLPFDKYELEPSPLTQYILERKSPHTCWQVFVTSSGKYNELGYPFGYLKASTTLTCVNLFVMPYNYPVLLPLLDDLFKVHKLKPNLKWRQAFDSYLKTLPPYYLLPLKKALRMMGAPNLISDNLDCGLSYSVISYLKKLSQQTKLESERILASVGKKPPQEIGIKVKNHSGGGMSLTHNKNFRKLLKEITGETALRLTELNTKEFAGFQIGLLNKVNCDINSLYLFELV

SEQ ID NO: 11 - protein, 804 aa, Homo sapiens
  misc_feature: Incyte ID No 6715627CD1
MEEPGATPQPYLGLLLEELRRVVAALPEGMRPDSNLYGFPWELVICAAVVGFFAVLFFLWRSFRSVRSRLYVGREKKLALMLSGLIEEKSKLLEKFSLVQKEYEGYEVESSLKDASFEKEATEAQSLEATCEKLNRSNSELEDEILCLEKELKEEKSKHSEQDELMADISKRIQSLEDESKSLKSQVAEAKMTFKIFQMNEERLKIAIKDALNENSQLQESQKQPLQEAEVWKEQVSELNKQKVTFEDSKVHAEQVLNDKESHIKTLTERLLKMKDWAAMLGEDITDDDNLELEMNSESENGAYLDNPPKGALKKLIHAAKLNASLKTLEGERNQIYIQLSEVDKTKEELTEHIKNLQTEQASLQSENTHFENENQKLQQKLKVMTELYQENEMKLHRKLTVEENYRLEKEEKLSKVDEKISHATEELETYRKRAKDLEEELERTIHSYQGQIISHEKKAHDNWLAARNAERNLNDLRKENAHNRQKLTETELKFELLEKDPYALDVPNTAFGREHSPYGPSPLGWPSSETRAFLSPPTLLEGPLRLSPLLPGGGGRGSRGPGNPLDHQITNERGESSCDRLTDPHRAPSDTGSLSPPWDQDRRMMFPPPGQSYPDSALPPQRQDRFCSNSGRLSGPAELRSFNMPSLDKMDGSMPSEMESSRNDTKDDLGNLNVPDSSLPAENEATGPGLFLHLLLPIRGPLFPVDARGPFLRRGPPFPPPPPGAMFGASRDYFPPGDFPGPPPAPFAMRNVYPPRGFPPYLPPRPGFFPPPPHSEGRSEFPSGLIPPSNEPATEHPEPQQET

SEQ ID NO: 12 - protein, 465 aa, Homo sapiens
  misc_feature: Incyte ID No 7727886CD1
MRRAAGMEDFSAEEEESWYDQQDLEQDLHLAAELGKTLLERNKELEGSLQQMYSTNEEQVQEIEYLTKQLDTLRHVNEQHAKVYEQLDLTARDLELTNHRLVLESKAAQQKIHGLTETIERLQAQVEELQAQVEQLRGLEQLRVLREKRERRRTIHTFPCLKELCTSPRCKDAFRLHSSSLELGPRPLEQENERLQTLVGALRSQVSQERQRKERAEREYTAVLQEYSELERQLCEMEACRLRVQELEAELLELQQMKQAKTYLLGPDDHLAEALLAPLTQAPEADDPQPGRGDDLGAQDGVSSPAASPGHVVRKSCSDTALNAIVAKDPASRHAGNLTLHANSVRKRGMSILREVDEQYHALLEKYEELLSKCRQHGAGVRHAGVQTSRPISRDSSWRDLRGGEEGQGEVKAGEKSLSQHVEAVDKRLEQSQPEYKALFKEIFSRIQKTKADINATKVKTHSSK

SEQ ID NO: 13 - protein, 651 aa, Homo sapiens
  misc_feature: Incyte ID No 914113CD1
MSTAPSLSALRSSKHSGGGGGGGGGGGGGGGADPAWTSALSGNSSGPGPGSSPAGSTKPFVHAVPPSDPLRQANRLPIKVLKMLTARTGHILHPEYLQPLPSTPVSPIELDAKKSPLALLAQTCSQIGKPDPSPSSKLSSVASNGGGAGGAGGGAAGDKDTKSGPLKLSDIGVEDKSSFKPYSKPGSDKKEPGGGGGGGGGGGGGGGGVSSEKSGFRVPSATCQPFTPRTGSPELQRLGLLAGRYAVLGRGCPGGQGRQERHRRGRPVARAPGGASAEGGPTGLAHGRISCGGGINVDVNQHPDGGPGGKALGSDCGGSSGSSSGSGPSAPTSSSVLGSGLVAPVSPYKPGQTVFPLPPAGMTYPGSLAGAYAGYPPQFLPHGVALDPTKPGSLVGAQLAAAAAGSLGCSKPAGSSPLAGASPPSVMTASLCRDPYCLSYHCASHLAGAAAASASCAHDPAAAAAALKSGYPLVYPTHPLHGVHSSLTAAAAAGATPPSLAGHPLYPYGFMLPNDPLPHICNWVSANGPCDKRFATSEELLSHLRTHTAFPGTDKLLSGYPSSSSLASAAAAAMACHMHIPTSGAPGSPGTLALRSPHHALGLSSRYHPYSKSPLPTPGAPVPVPAATGPYYSPYALYGQRLTTASALGYQ

SEQ ID NO: 14 - protein, 1443 aa, Homo sapiens
  misc_feature: Incyte ID No 1953711CD1
MATSRGASRCPRDIANVMQRLQDEQEIVQKRTFTKWINSHLAKRKPPMVVDDLFEDMKDGVKLLALLEVLSGQKLPCEQGRRMKRIHAVANIGTALKFLEGRKIKLVNINSTDIADGRPSIVLGLMWTIILYFQIEELTSNLPQLQSLSSSASSVDSIVSSETPSPPSKRKVTTKIQGNAKKALLKWVQYTAGKQTGIEVKDFGKSWRSGVAFHSVIHAIRPELVDLETVKGRSNRENLEDAFTIAETELGIPRLLDPEDVDVDKPDEKSIMTYVAQFLKHYPDIHNASTDGQEDDEILPGFPSFANSVQNFKREDRVIFKEMKVWIEQFERDLTRAQMVESNLQDKYQSFKHFRVQYEMKRKQIEHLIQPLHRDGKLSLDQALVKQSWDRVTSRLFDWHIQLDKSLPAPLGTIGAWLYRAEVALREEITVQQVHEETANTIQRKLEQHKDLLQNTDAHKRAFHEIYRTRSVNGIPVPPDQLEDMAERFHFVSSTSELHLMKMEFLELKYRLLSLLVLAESKLKSWIIKYGRRESVEQLLQNYVSFIENSKFFEQYEVTYQILKQTAEMYVKADGSVEEAENVIKFMNETTAQWRNLSVEVRSVRSMLEEVISNWDRYGNTVASLQAWLEDAEKMLNQSENAKKDFFRNLPHWIQQHTAMNDAGNFLIETCDEMVSRDLKQQLLLLNGRWRELFMGVKQYAQADEMDRMKKEYTDCVVTLSAFATEAHKKLSEPLEVSFMNVKLLIQDLEDIEQRVPVMDAQYKIITKTAHLITKESPQEEGKEMFATMSKLKEQLTKVRECYSPLLYESQQLLIPLEELEKQMTSFYDSLGKINEIITVLEREAQSSALFKQKHQELLACQENCKKTLTLIEKGSQSVQKFVTLSNVLKHFDQTRLQRQIADIHVAFQSMVKKTGDWKKHVETNSRLMKKFEESRAELEKVLRIAQEGLEEKGDPEELLRRHTEFFSQLDQRVLNAFLKACDELTDILPEQEQQGLQEAVRKLHKQWKDLQGEAPYHLLHLKIDVEKNRFLASVEECRTELDRETKLMPQEGSEKIIKEHRVFFSDKGPHHLCEKRLQLIEELCVKLPVRDPVRDTPGTCHVTLKELRAAIDSTYRKLMEDPDKWKDYTSRFSEFSSWISTNETQLKGIKGEAIDTANHGEVKRAVEEIRNGVTKRGETLSWLKSRLKVLTEVSSENEAQKQGDELAKLSSSFKALVTLLSEVEKMLSNFGDCVQYKEIVKNSLEELISGSKEVQEQAEKILDTENLFEAQQLLLHHQQKTKRISAKKRDVQQQIAQAQQGEGGLPDRGHEELRKLESTLDGLERSRERQERRIQVTLRKWERFETNKETVVRYLFQTGSSHERFLSFSSLESLSSELEQTKEFSKRTESIAVQAENLVKEASEIPLGPQNKQLLQQQAKSIKEQVKKLEDTLEEEYVIDKS

SEQ ID NO: 15 - protein, 1087 aa, Homo sapiens
  misc_feature: Incyte ID No 1595275CD1
MAEFTSYKETASSRHLRFKLQSLSRRLDELEEATKNLQKAEDELLDLQDKVIQAEGSNSSMLAEIEVLRQRVLRIEGKDEEIKRAEDLCRLMKEKLEEEENLTRELKSEIERLQKRMAELEKLEEAFSRSKNDCTQLCLSLNEERNLTKKISSELEMLRVKVKELESSEDRLDKTEQSLASELEKLKSLTLSFVSERKYLNEKEKENEKLIKELTQKLEQNKKMNRDYTRNASNLERNDLRIEDGISSTLPSKESRRKGGLDYLKQVENETRNKSENEKNRNQEDNKVKDLNQEIEKLKTQIKHFESLEEELKKMKSKNNDLQDNYLSEQNKNKLLASQLEEIKLQIKKQKELENGEVEGEDAFLSSKGRHERTKFRGHGSEASVSKHTARELSPQHKRERLRNREFALNNENYSLSNRQVSSPSFTNRRAAKASHMGVSTDSGTQETKKTEDRFVPSSSKSEGKKSREQPSVLSRYPPAAQEHSKAWKGTSKPGTESGLKGKVEKTTRTFSDTTHGSVPSDPLGRADKASDTSSETVFGKRGHVLGNGSQVTQAANSGCSKAIGALASSRRSSSEGLSKGKKAANGLEADNSCPNSKAPVLSKYPYSCRSQENILQGFSTSHKEGVNQPAAVVMEDSSPHEALRCRVIKSSGREKPDSDDDLDIASLVTAKLVNTTITPEPEPKPQPNSREKAKTRGAPRTSLFENDKDAGMENESVKSVRASTNTMELPDTNGAGVKSQRPFSPREALRSRAIIKPVIVDKDVKKIMGGSGTETTLEKQKPVSKPGPNKVTSSITIYPSDSSSPRAAPGEALRERHTSTSNIQVGLAELTSVSNHVSSPFELSIHKHDITLQLAEAERMADGPLKNRPETVVSRSSIIIKPSDPVERNSHAPPAETIRWKSHSAPSEVGFSDARHVTVRNAWKSRRDLKSLEDPPTRIGKNVESTNSNAYTQRSSTDFSELEQPRSCLFEQGTRRVGPSSGDAPEPSSRRTQSSLTVSEVLTRRNRVGDTITVAAWNHSASMEEEGEDCTLSVYRQLHNPWIRLNCLGSRGCQSLGEYGRGNDYGPNRPCAEETLSPHGCLLSAL

SEQ ID NO: 16 - protein, 240 aa, Homo sapiens
  misc_feature: Incyte ID No 72332548CD1
MATAMYLEHYLDSIENLPCELQRNFQLMRELDQRTEDKKAEIDILAAEYISTVKTLSPDQRVERLQKIQNAYSKCKEYSDDKVQLAMQTYEMVDKHIRRLDADLARFEADLKDKMEGSDFESSGGRGLKKGRGQKEKRGSRGRGRRTSEEDTPKKKKHKGGSEFTDTILPVHPSDVLDMPVDPNEPTYCLCHQVSYGEMIGCDNPDCPIEWFHFACVDLTTKPKGKWFCPRCVQEKRKKK

SEQ ID NO: 17 - protein, 686 aa, Homo sapiens
  misc_feature: Incyte ID No 7322834CD1
MAANMYRVGDYVYFENSSSNPYLIRRIEELNKTANGNVEAKVVCFYRRRDISSTLIALADKHAREIEEEMENPEMVDLPEKLKHQLRHRELFLSRQLESLPATHIRGKCSVTLLNETESLKSYLEREDFFFYSLVYDPQQKTLLADKGEIRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAEAESKLKQVYIPNYNKPNPNQISVNNVKAGVVNGTGAPGQSPGAGRACESCYTTQSYQWYSWGPPNMQCRLCASCWTYWKKYGGLKMPTRLDGERPGPNRSNMSPHGLPARSSGSPKFAMKTRQAFYLHTTKLTRIARRLCREILRPWHAARHPYLPINSAAIKAECTARLPEASQSPLVLKQAVRKPLEAVLRYLETHPRPPKPDPVKSVSSVLSSLTPAKVAPVINNGSPTILGKRSYEQHNGVDGLANHGQTRHMGPSRNLLLNGKSYPTKVRLIRGGSLPPVKRRRMNWIDAPDDVFYMATEETRKIRKLLSSSETKRAARRPYKPIALRQSQALPPRPPPPAPVNDEPIVIED

SEQ ID NO: 18 - protein, 1595 aa, Homo sapiens [sequence omitted]

SEQ ID NO: 19 - protein, 592 aa, Homo sapiens
  misc_feature: Incyte ID No 6803363CD1
MTCWLCVLSLPLLLLPAAPPPAGGCPARCECTVQTRAVACTRRRLTAVPDGIPAETRLLELSRNRIRCLNPGDLAALPALEELDLSENAIAHVEPGAFANLPRLRVLRLRGNQLKLIPPGVFTRLDNLTLLDLSENKLVILLDYTFQDLHSLRRLEVGDNDLVFVSRRAFAGLLALEELTLERCNLTALSGESLGHLRSLGALRLRHLAIASLEDQNFRRLPGLLHLEIDNWPLLEEVAAGSLRGLNLTSLSVTHTNITAVPAAALRHQAHLTCLNLSHNPISTVPRGSFRDLVRLRELHLAGALLAVVEPQAFLGLRQIRLLNLSNNLLSTLEESTFHSVNTLETLRVDGNPLACDCRLLWIVQRRKTLNFDGRLPACATPAEVRGDALRNLPDSVLFEYFVCRKPKIRERRLQRVTATAGEDVRFLCRAEGEPAPTVAWVTPQHRPVTATSAGRARVLPGGTLEIQDARPQDSGTYTCVASNAGGNDTYFATLTVRPEPAANRTPGEAHNETLAALRAPLDLTTILVSTAMGCITFLGVVLFCFVLLFVWSRGRGQHKNNFSGEYSFRKVDGPAAAAGQGGARKFNMKMI

SEQ ID NO: 20 - protein, 678 aa, Homo sapiens
  misc_feature: Incyte ID No 7070580CD1
MVRAGGRAWQQGLHSLSAVSILAKGFMFLLVGDSRAESVTNTLVLFTIGEESLTIFVDKQKLGRKTETTGGASIIGGSGNSTAVSLETLHQLAASYFIDRESTLRRLHHIQIATGAIKVTETRTGPLGCSNYDNLDSVSSVLVQSPENKVQLLGLQVLLPEYLRERFVAAALSYITCSSEGELVCKENDCWCKCSPTFPECNCPDADIQAMEDSLLQIQDSWATHNRQFEESEEFQALLKRLPDDRFLNSTAISQFWAMDTSLQHRYQQLGAGLKVLFKKTHRILRRLFNLCKRCHRQPRFRLPKERSLSYWWNRIQSLLYCGESTFPGTFLEQSHSCTCPYDQSSCQGPIPCALGEGPACAHCAPDNSTRCGSCNPGYVLAQGLCRPEVAESLENFLGLETDLQDLELKYLLQKQDSRIEVHSIFISNDMRLGSWFDPSWRKRMLLTLKSNKYKPGLVHVMLALSLQICLTKNSTLEPVMAIYVNPFGGSHSESWFMPVNEGSFPDWERTNVDAAAQCQNWTITLGNRWKTFFETVHVYLRSRIKSLDDSSNETIYYEPLEMTDPSKNLGYMKINTLQVFGYSLPFDPDAIRDLILQFDYPYTQGSQDSALLQLIELRDRVNQLSPPGKVRLDLFSCLLRHRLKLANNEVGRIQSSLRAFNSKLPNPVEYETGKLCS

SEQ ID NO: 21 - protein, 652 aa, Homo sapiens
  misc_feature: Incyte ID No 7500176CD1
MAAETLLSSLLGLLLLGLLLPASLTGGVGSLNLEELSEMRYGIEILPLPVMGGQSQSSDVVIVSSKYKQRYECRLPAGAIHFQREREEETPAYQGPGIPELLSPMRDAPCLLKTKDWWTYEFCYGRHIQQYHMEDSEIKGEVLYLGYYQSAFDWDDETAKASKQHRLKRYHSQTYGNGSKCDLNGRPREAEVRFLCDEGAGISGDYIDRVDEPLSCSYVLTIRTPRLCPHPLLRPPPSAAPQAILCHPSLQPEEYMAYVQRQADSKQYGDKIIEELQDLGPQVWSETKSGVAPQKMAGASPTKDDSKDSDFWKMLNEPEDQAPGGEEVPAEEQDPSPEAADSASGAPNDFQNNVQVKVIRSPADLIRFIEELKGGTKKGKPNIGQEQPVDDAAEVPQREPEKERGDPERQREMEEEEDEDEDEDEDEDERQLLGEFEKELEGILLPSDRDRLRSETEKELDPDGLKKESERDRAMLALTSTLNKLIKRLEEKQSPELVKKHKKKRVVPKKPPPSPQPTEEDPEHRVRVRVTKLRLGGPNQDLTVLEMKRENPQLKQIEGLVKELLEREGLTAAGKIEIKIVRPWAEGTEEGARWLTDEDTRNLKEIFFNILVPGAEEAQKERQRQKELESNYRRVWGSPGGEGTGDLDEFDF

SEQ ID NO: 22 - protein, 457 aa, Homo sapiens
  misc_feature: Incyte ID No 7500506CD1
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSVQEALSGTPCLLGPGPVLTVLALLLASTLA

SEQ ID NO: 23 - protein, 127 aa, Homo sapiens
  misc_feature: Incyte ID No 7500639CD1
METAVRGMPLECPPRPERLNAYEREVMVNMLNSLSRNQQLPRITPRCGCVDPLPGRLPFHGYESACSGRHYCLRGMDYYASGAPCTDRRLRPWCREQPTMCTSLRAPARNAVCCYNSPAVILPISEP

SEQ ID NO: 24 - protein, 763 aa, Homo sapiens
  misc_feature: Incyte ID No 7506167CD1
MEEPGATPQPYLGLVLEELRRVVAALPESMRPDENPYGFPSELVVCAAVIGFFVVLLFLWRSFRSVRSRLYVGREQKLGATLSGLIEEKCKLLEKFSLIQKEYEGYEVESSLEDASFEKAAAEEARSLEATCEKLNRSNSELEDEILCLEKDLKQEKSKHSQQDELMADISKSIQSLEDESKSLKSQIAEAKIICKTFKMSEERRAIAIKDALNENSQLQTSHKQLFQQEAEVWKGEVSELNKQKITFEDSKVHAEQVLNDKENHIKTLTGHLPMMKDQAAVLEEDTTDDDNLELEVNSQWENGAYLDNPPKGALKKLIHAAKLNASLKTLEGERNQIYIQLSEVDKTKEELTEHIKNLQTQQASLQSENTHFENENQKLQQKLKVMTELYQENEMKLHRKLTVEENYRLEKEEKLSKVDEKISHATEELETYRKRAKDLEEELERTIHSYQGQIISHEKKAHDNWLAARNAERNLNDLRKENAHNRQKLTETELKFELLEKDPYALDVPNTAFGRGSRGPGNPLDHQITNERGESSCDRLTDPHRAPSDTGSLSPPWDQDRRMMFPPPGQSYPDSALPPQRQDRFCSNSGRLSGPAELRSFNMPSLDKMDGSMPSEMESSRNDTKDDLGNLNVPDSSLPAENEATGPGFVPPPLAPIRGPLFPVDARGPFLRRGPPFPPPPPGAMFGASRDYFPPRDFPGPPPAPFAMRNVYPPRGFPPYLPPRPGFFPPPPHSEGRSEFPSGLIPPSNEPATEHPEPQQET

SEQ ID NO: 25 - protein, 612 aa, Homo sapiens
  misc_feature: Incyte ID No 90081189CD1
MAAETLLSSLLGLLLLGLLLPASLTGGVGSLNLEELSEMRYGIEILPLPVMGGQSQSSDVVIVSSKYKQRYECRLPAGAIHFQREREEETPAYQGPGIPELLSPMRDAPCLLKTKDWWTYEFCYGRHIQQYHMEDSEIKGEVLYLGYYQSAFDWDDETAKASKQHRLKRYHSQTYGNGSKCDLNGRPREAEVRFLCDEGAGISGDYIDRVDEPLSCSYVLTIRTPRLCPHPLLRPPPSAAPQAILCHPSLQPEEYMAYVQRQADSKQYGDKIIEELQDLGPQVWSETKSGVAPQKMAGASPTKDDSKDPDFWKMLNEPEDQAPGGEEVPAEEQDPSPEAADSASGAPNDFQNNVQVKVIRSPADLIRFIEELKGGTKKGKPNIGQEQPVDDAAEVPQREPEKERGDPERQREMEEEEDEDEDEDEDEDERQLLGEFEKELEGILLPSDRDRLRSEVKAGMERELENIIQETEKELDPDGLKKESERDRAMLALTSTLNKLIKRLEEKQSPELVKKHKKKRVVPKKPPPSPQPTGKIEIKIVRPWAEGTEEGARWLTDEDTRNLKEIFFNILVPGAEEAQKERQRQKELESNYRRVWGSPGGEGTGDLDEFDF

SEQ ID NO: 26 - protein, 638 aa, Homo sapiens
  misc_feature: Incyte ID No 7510095CD1
MAAETLLSSLLGLLLLGLLLPASLTGGVGSLNLEELSEMRYGIEILPLPVMGGQSQSSDVVIVSSKYKQRYECRLPAGAIHFQREREEETPAYQGPGIPELLSPMRDAPCLLKTKDWWTYEFCYGRHIQQYHMEDSEIKGEVLYLGYYQSAFDWDDETAKASKQHRLKRYHSQTYGNGSKCDLNGRPREAEVRFLCDEGAGISGDYIDRVDEPLSCSYVLTIRTPRLCPHPLLRPPPSAAPQAILCHPSLQPEEYMAYVQRQADSKQYGDKIIEELQDLGPQVWSETKSGVAPQKMAGASPTKDDSKDSDFWKMLNEPEDQAPGGEEVPAEEQDPSPEAADSASGAPNDFQNNVQVKVIRSPADLIRFIEELKGGTKKGKPNIGQEQPVDDAAEVPQREPEKERGDPERQREMEEEEDEDEDEDEDEDERQLLGEFEKELEGILLPSDRDRLRSEVKAGMERELENIIQETEKELDPDGLKKESERDRAMLALTSTLNKLIKRLEEKQSPELVKKHKKKRVVPKKPPPSPQPTEEDPEHRVRVRVTKLRLGGPNQDLTVLEMKRENPQLKQIEGLVKELLEREGLTAAGGPWRAAGPSAVGRASCRKWRGWDQWGRGPALRLPTILSSHCILLLSREN

SEQ ID NO: 27 - protein, 75 aa, Homo sapiens
  misc_feature: Incyte ID No 7510096CD1
MAAETLLSSLLGLLLLGLLLPASLTGGVGSLNLEELSEMRYGIEILPLPVMGGQPFFASLPPDPPFFPRANLRTW

SEQ ID NO: 28 - DNA, 3343 bp, Homo sapiens
  misc_feature: Incyte ID No 261510CB1
ccctcttccgtgtgggccctccctgtcccctctctgtcacctcacagtagtcgttgccggtagcgggtgtgggggcgcgagcagcgcagggcggcctctcccccacccctcagcccgggcccgggctcggtccccccctctccctccgcttcgcactcccgtcgggtacgggaaggtccaagccgctgccgggtgcccgagggccgtcgtggccgccgtcgccacgggtcccaatggagccgccgaatctctatccggtgaagctctacgtgtacgacctgtccaaaggcctggcccggcggctcagccccatcatgctggggaaacaactggaaggcatctggcacacatccatagttgtgcacaaggatgagttcttcttcggcagtggtggtatctccagctgccccccgggagggacattgcttgggcctccagactctgtggttgatgtggggagtacagaagtcacagaagaaatctttctggagtacctctcctccctgggggagtccctgttccgaggtgaggcctacaacctctttgaacacaattgtaacaccttcagcaacgaagtggcacagttcctgactgggcggaagattccttcttacatcacagacctgccctctgaagttctctccacgccctttggacaggcacttcggcccctcctggactccattcagatccagcctccaggagggagctccgtgggcagacccaacggccagagctaacaggactgcctgggaccgccctgcctcaccagggcttttcctttttaaacaaaacaaaccctaccagatttctattttataattttacatcagagctaacaaccaggggacggctttttaaatttcccagggaaggagaccgtcaggccgcatgtagacaatgctgctaagaaacagaacaaaatgccaccccttctaatagtattatactaatttattaaggctgtcttgtctgtgcgttcacttcggatgctgtttcctaagcgtcctccacactggagaaagggaggggctttgtttaaacagaaagcgagtcacagtttgcgggagccccaacctagaactctttccagagaacactcacgctctacccaagttgccaacacctgtgggacccaggtgagttttagaacatccgtgacccagaaagttactctagtgttaaatgaaccaaaggatttggtgtacatgggaccggtttcaggatgactaacctaacctaagtgaactctttagccacaaccctcaccttccctcccttaccccaagcaaggccagtgctggagcagccattgcctccggatcaagggagtaacagcaagtatcaggccggagaaaacaggataaagccattcctccttccacctagatggattgccaatcttctgccatcaattttgggaaacctgcctttgtgcccccgaggctgcctttcctcagcatagctgacttgtcatagtggatgaggtctgggctggagcagtagaagccaaggccttttttctgcaagtaatgatgtaggttatttgcagtctctgtaaccaccatccaggccagtacgcagtatttagctggggaccaagggatgggattccctactctaagaaaatacctttattttaattattattattttttgagacggagtctcgctctaccacccaggctggtgtgcagtggcatgatcccgggaggcagagattgcgatgagccgagatcgtaccagtgcactccagcctgggcgacagagtgagactccatctcaaaaaaataaaataatgaagtggaatcaaggtggcaagaaaatttagaatcgtttctgatagggactttcccttatcctaaatgtgttttgaaggcacacgttatttccattgtcattctgactttagaaaggcaggttggggcatgagtgggcattttcctaatgacttgctgctaggaggttataggcagggaacagtctgggagtctctaatccagagagactttcagattccctctcttcacatgaacctccattagttagattttgggaaagtgagcatgaagaagggactcagagctgccaggtacccgagtctcgggtctgacatccttgggccccttctgccacaacttcttgccacttcacggacttgaattatgttgcttcttcctggaagtctggcctgcagcacaggcctttcctctccaaaaagctgctgctcatttctggccccatctgctgctatctttgccatcagccatcacagtttggcatcatcagagtctcagctagaggagctgtcattctgccttagtgtttgcctgcgacctgaccaggggcatgaccagctgggatgtcaagtgacatttgggaacttgtagaggccaaatgattgaaagtttaattcttctcaagacccactgatttgccagtgtgcatggaaataatagattagagcagaaactagcagggactgttgtataatcgtgatctactagcagaattgggcccaaagagaaagaatttctgaggctgggtttcttctgggagggtagcaggttggtcttggccagtgctctgtttcccaccagtgttgttcccttttccagcttgcctggcccggctctgtcttccagggtcctgctccagacccctctgcctggggtctcccctccatccagggggttctgggcatcacagctccttttttttcctttctcctcttccagatccagtttcagctggtttgcttcttccagcaggcacccttaggcccaggagcagctcccctgagagaatttgagtcactcatttccttgaacagcacccccctttatactagcagccatttgtgccattgcctgtgccctagggtttgtggggagagagcgagggatcactgagcagttttcccagagctccatgggaaggcaagctctccctcccaatgggagccccactgtcactaactgtaaactcaggctcaggcttcaactgcctacccccatcctcatatttctgtctgtcccagcacctcaggagcattctcattgtggccggctaactccgcctggatgtgaacaggcaagcacagtgggaaatgagtcacgtacttgtattgcacagtggacacctctagaggtccattggtttaaagggatagggaaggaggagggatgagaccatcaccccctcccagaagtaaatctagtatctgagttttctttatgcccttgagtcaaactaataactgtctagtacggaggtgtttgctggttttcttcggtgttttttctaatgcaataaactcatttctgcctgctcgaaaaaaaaa

SEQ ID NO: 29 - DNA, 3190 bp, Homo sapiens
  misc_feature: Incyte ID No 7498674CB1
cggccccagggagagatcgcgggcagcatggcggggctgcgggccagggggggcccggggccggggctgctggcgctctccgcgctcggcttctgcctcatgctgcaagtcagcgctaagaggccccccaagacgcccccctgcccgcccagctgctcttgcaccagggacaccgccttctgcgtggactcaaaggcggtgcccaggaacctgccctcggaggtcatctccctgaccctggtgaatgccgccttctcagagatccaggatggagcgttctcccacctcccgctgctgcagttcttgttactcaactccaacaagtttacactgattggagacaacgccttcacaggactgtcgcacctgcagtatctcttcattgagaacaatgacatctgggcactatccaagttcaccttccgaggactcaagtccttgactcacctctcgctggccaacaataacctgcagacactgcccagagacatcttccggcccctggacatcctgaatgacttggacctgcggggcaactcactcaactgtgactgcaaggtgaagtggttggtggagtggctggcacacaccaacaccacggtggcacccatctactgcgccagcccgccccgcttccaggagcacaaggtgcaggacctgccgctgcgggagttcgattgcatcaccacagattttgtgttgtaccagaccctggccttcccagcagtgtcggctgagcccttcctctactccagtgacctctatttggctctggcccagccaggagtcagtgcctgcaccatcctgaagtgggactatgttgagcggcagcttcgagactatgatagaatcccagccccctctgcagtgcactgcaagccgatggtggtggacagccagctgtacgtggtcgtggcccagctgtttggcggctcttacatttaccactgggatcccaacaccacgcgcttcaccaggctgcaagacattgacccgcagcgcgtgcgcaagcctaacgacctagaagccttccgcatcgacggtgactggtactttgccgtggctgacagctccaaggcaggcgccaccagcctctaccgctggcaccagaatggcttctactcccaccaggcactgcacccctggcaccgtgacaccgacctggagtttgtggacggcgagggcaagccacggctgattgtgtccagcagctcccaggcacccgtcatctatcagtggagtcgcacccagaagcagtttgtggcccagggtgaggtgacccaggtgcctgatgcccaagctgtgaaacactttcgtgccggccgcgacagctacctgtgcctcagccgctacattggcgactccaagatcctgcgctgggagggtacccgcttctcggaggtgcaggccctgccctcccggggctcgctggccctgcagcccttccttgtgggtggccgccgctacctggcactgggcagtgatttctccttcacccagatctaccagtgggatgagggacgacagaagtttgtacggttccaggagctggctgtgcaggctcctcgggccttctgctacatgcctgctggggacgcccagctactcctggcccccagcttcaagggacagacgctggtgtatagacacattgtggtggatctcagtgcctagggggtgccgaggcctctggtctcctcagggtggccactggaggatgggtgggggcctctgtgagcaccacgtatgcctgtgtgaagacaagcgaccaacctgcattcatgcccttgtatccgcacacgcccagtgtgtacaggcgcaccccgtggactggcccaggaggacgtgcatcatcactgcaatcagcatgaacaccaggcacccctgggacacctaacgcccagttcccgtgaccccaactgcaggccccccctactctgcagcctttcccatgctctgccccctgtctcatgcacgagtggtgcggggtgctgggagcgaggggggccgcatcctcctgtccttgtgcttctccttctgcccctctggtctttcctgttggtgctccaggcattgtttacctgctcttgctccccgctgtagcccacagccatatctgtcatgcttccccggggccactcacccctgccccaccccgtctgcatgctcacatggacgcggacggacacactcattacacactcacacgccccgactgcacccggtgtccatctataaacatgtcagggcacgtgtgcatacaagcagattcagcactgcctaccaggctctcctgttaccttgcctctctgcttggagggggccccctctgctcacccccagcgtgccccccccggaactgataaggatgagaatggtggtgccagcttctgagggcctgcttggcctcctgggggcaaagcccctctgcccgaagcaataacaacagcagcagaagcaatcccggggagctgccaggggtgtctgtctctgttctccttggcgtttccttcatggccgtgaatcccagctcacccatggacagaagacctcccctcatctgaaagcctttctgccttccttcctgtagctgtcgctttctcaggcttggactgtcccctggttcaccctgtctttgggatgtggcccttgagctgggcgggctccttgggtggattagagagctggccgagctgcaagcacctgagttacctgcgagggtgcccagagtggagcttggcctgagggcgtgttgcagccctggccccaccagtccacagaggcccccaggagcactgcctttcagtggaccgggtgggggctggtgggaggggccccactagctgagcttctgctgccctctgctgtctgctgagatgccaggctagtggagagggcacaccatgtgcccaaatggtgtgatgctccttgtagcaggataccagggtactggggggcaatgctatgattaacttgcttcaaataaaaagttcccgccccataaaaaaaaaaaaataacgcatccacagcaaccacgcaccaccatccacagccacttccccaccaggattctctcaccaaccgctgtccccggcctatcacacacccagtcataccgtcgtgtatacctctgtcccactcagctacagacatcggcgccccccctcccctccaccaccctcttccatct

SEQ ID NO: 30 - DNA, 3150 bp, Homo sapiens
  misc_feature: Incyte ID No 1629617CB1
ggcgcaccctacgacgcgcacaccaccggcatgaccggcgccatcagctaccacccgtacggcagcgcggcctacccgtaccagctcaacgaccccgcgtaccgcaagaacgccacgcgggacgccacggccactctcaaggcctggctcaacgagcaccgcaagaacccctaccccaccaagggcgagaagatcatgctagccatcatcaccaagatgaccctcacccaggtctccacctggttcgccaacgcgcgccggcgcctcaagaaggagaacaagatgacctgggccccgagaaacaaaagcgaagatgaggacgaggacgagggcgacgctaccagaagcaaggacgagagtcccgacaaggcgcaggagggcacggagacctcggcagaggacgaagggatcagcctgcacgtggactcgctcacggatcactcgtgctcggccgagtcggacggggagaagcttccgtgccgcgccggggaccccctgtgcgaatcgggctcggagtgcaaggacaagtatgacgacctggaggacgacgaggacgacgacgaggagggcgagcggggcctggcgccgcccaagcccgtgacctcgtcgccgcttaccggcttggaggcgccgctgctgagccccccgcccgaggccgcgccccgcggtggccgcaagacgccccagggcagccggacgtctccgggcgcgccgccccccgccagcaagcccaagctgtggtcgctggccgagatcgccacgtcggacctcaagcagccgagcctgggcccgggctgcgggccaccggggctgcccgcggccgccgcgccggcctcaaccggggcaccgccaggaggctcgccctaccctgcctcgccgctgctgggccgccccctctactacacgtcgcccttctacggcaactacacaaactacgggaacttgaacgcggcgctgcagggccagggtctcctgcggtacaactctgcggccgcggcccccggcgaggccctgcacaccgcgccaaaggcggccagcgacgcgggcaaggcgggcgcgcacccgctcgagtcccactaccggtccccgggcggcggctacgagcccaagaaagatgccagcgagggctgcaccgtggttggcgggggcgtccagccctacctatagaagggccgagcacagcaatgcaagtaagtgggcaccctggtgcttcccagaccctgtggagcctcccagcaccaacttgcttaatgtgtttttcttgcttctggtcatggggttagaagacagagcatttcaatcttgtccagcttttctttccttaagggggaaactatattttctgttttatagattccctctttttggcacaagttctatcactcctttacctagggaaggaaaaaaaaatatatatatatatgtgtgtgtgtgtgtgtgtaatatatatatgtatatatatagtacatgtatatatatagtacatgtgtatatatatattacacactactgttgttttcttttgaaacaatagattattaaaatctgggcatttatatgcaatttagagccatttttactatgctagacacataatagacacatgctttgatgagcttgggctaccaaaacttggagtagcaaaaacctttatagatgatgtttgcaaaataagactgaagagacacgaggcgtactagtcttttatcagccctgtccaacctcccctccacccatgtccaagtggacaggactctgacattctcccggggctcacaacagtatctcaaaaaatatgttgctttgcaacgagtcagaattactaatcatagatcttgaagataagaaagatttggaaccacttcagatcaactcgtttttataatggtgttgttgtggaactgaaagattggggatgatttggttttttccttggtccacaggtaggtgtcacaattgctttgaaaaaaaaaaaaaaaaaaaaaagtcagcaggccattctctcttcccaagctcataaatttacagatacaacaaacagatgtctaaatccagaccacatcttgtgccactgtaaaagagaaggatccacacagcactgtcagcccacagactctaacaattggaacagactattgttggacatatgtgaatttgttggcatagtatagtttccccgatgtatgtgtgacttttgaaaacaatctgtttttctcaggatatcttgaattgatcacttcattgccacggtatatattctataggtgtgataggatttactgtaaagtttatttgtaaaagatgtacacagtagaaataaaacgtgatggaagaacttgagtacttcagaagtggaaacaaatttgatatttatttttataatgatataaagcttctagtaatttatgcaagttgtattgcaatggaatctaaactttttgtaaataaattctgcctggtttttatttgaacattgctggttatacaatctttgttggttgtttaacacgaattctttactaatttatatcttaaattgtaaataaaaacagactagtctacaataaaaaaaaaaaaaaagggcggccgccgactagtgagctcgtcgacccgggaattaattccggaccggtacctgcaggcgtaccagctttccctatagtgagtcgtattagagcttggcgtaatcatggtcatagctgtttcctgtgtgaaattgttatccgctcacaattccacacaacatacggaagcatggtttggactgcgccaagcgagaaccaaggaatggccaccgcggaaccgcggggcgggtcgagggcccaaggcacacacgagaaggcccgcaggcggagcactaacacagtaccgcatactaaagcgcgtgaacacgacttacgctagacatacagggcggatgcagtgagacaatatatgacgcacagcagtcggaagcacgaggcagataccgtcacggaggcaagtgcacaatacaacgacactaacaccgcaacgacgaggcgccagaagcagcagtgactatcaccgcacaggccacacgtaaaatgcacaaccacccgccacgacccacacaagaatccaaacgcgtaccgccgcgaccacagacacagaacaccacgcacaagccaa

SEQ ID NO: 31 - DNA, 2012 bp, Homo sapiens
  misc_feature: Incyte ID No 2369279CB1
aagggggaggttactggtggtggcggcggcggcggcggcccgggctccggctgtcggggaagctgctgagctgtgatggtgatgacgaggtgaaaatggcggatctttcgaaatacaatcccggcccctgacataccagaggcggcggcgacggcgacgtcgccaccccggctcccttctcggtccccgggtaccctggagcgctccagtttggacaaactgggaaaggaagctgctggccagagctatcgtgcacgggcaaaacaagccccgccggggctcgggattgcccaggaccttctggagcccctacctcggagcccgagggaggagaaagcggcagccgctggagtgcctgatcaactttctagcgggagaacgatcatggaggtggtgccagctgagattgctaagaagcctcgaacgccaacctctggtccagtaatcacgaagctgatctttgcaaaaccaattaatagtaaagcagttacaggacagacaactcaagtttcaccaccagtcattgcaggtagggttctttcacagtctactcccggaactccatcaaagaccataacaatatctgaaagtggtgttattggatcaactttaaattctacaacacagacaccaaataaaatagccatctcacctttgaaatcgccaaataaggcagtgaaatcaactgtgcagaccatcactgttggaggagtgagcacatcacagtttaagacaattattcctctggcaactgctcccaatgtccagcagattcaagtgcctggaagcaagtttcattatgtccgacttgttactgccacatcagccagtagctcaacccagccagttagtcagaatcccagtacaaacactcagcctcttcagcaagcaaagccagtggttgttaatacaaccccagtgcggatgtcagttccaattgtctcagctcaggctgtcaaacaagttgttccaaaaccaatcaatccaacttcacaaatagtaactactagccagccacagcaacggcttatcatgcctgccacaccactgccacagatccagcccaacctcactaacctgccaccaggcactgtcctggcaccagctccgggaacagggaatgtgggttatgcagtgcttccagctcagtatgttactcagctacagcagtcttcatatgtatcaatagcaagcaactctacctttactggaacatctggtatccagacccaggcacggcttccattcaatggcataatcccatcagagtcggccagtcggccccgaaagccctgtaattgtacaaaatcactgtgtttgaaattgtattgtgattgctttgcaaatggtgaattttgcaacaactgcaattgtactaattgttacaacaatttggaacatgaaaatgaaaggcaaaaagcaataaaggcatgccttgacagaaatccagaagcctttaagcctaagatagggaaaggaaaggagggagaatctgatcgacgtcatagcaaagggtgtaattgcaaacgatcaggatgtcttaaaaactactgtgaatgctatgaggcaaaaataatgtgttcctcaatatgcaaatgtattggctgtaagaattttgaagaaagcccggaaaggaagacattgatgcatttggcagatgcagctgaagtaagggtacagcaacaaacagcagccaagacgaagttatcctctcaaatttcagacttgcttactaggccaacaccagctttaaatagtggaggcggaaaattgccatttacatttgtaactaaggaagtagctgaagccacatgtaattgcctccttgcccaggcagagcaggcagacaagaagggaaaatcaaaggcagcagcggaacggatgatacttgaggaattcggacgatgtttgatgagtgtcatcaactctgcaggaaaggcaaaaagtgacccttgtgccatgaattgctaactcttgcacaaaagactgataaaatggaactgtacagaaaa

SEQ ID NO: 32 - DNA, 1606 bp, Homo sapiens
  misc_feature: Incyte ID No 3167506CB1
aggcggtgcaatacacgggttggctggcgatagcggcggtgggcgtggcttaggcgcgagcgagagcgggcgcggcgctcaggcagcggcggttggctgcggcgccagtggtagtgctccaggctcgacggcaccctcacagcgcccgcccggccctgccgctcatggccgccccgccgcgccccgcgccatcgccccccgccccgcggcgcctcgacacgagcgacgtcctgcagcagatcatggccatcaccgaccagagcctggacgaggcacaggccagaaagcatgctctgaattgccatcggatgaagcctgctctgttcagcgtgctctgtgagatcaaggaaaagacagtggtaagcatccgtggcattcaagacgaagatccccctgacgcccagctcctgaggctggataacatgctgctggctgagggcgtgtgcaggcccgagaagagaggaagaggaggagcggtggccagggccggcacagcaacaccaggtggctgtccaaatgacaatagcattgagcactctgactacagggccaagctgtcccagatccgacagatttaccactctgagctagagaaatatgaacaggcctgtcgtgagttcaccacgcacgtcaccaacctcctccaggagcagagcaggatgaggcctgtctcccctaaggagattgagcgcatggtcggcgccattcacggcaagttcagcgccatccagatgcagttgaagcagagcacctgtgaggcagtgatgaccctgcgttcgcggctgctcgatgccaggcgcaagcggcggaatttcagcaagcaggcgacggaagtgctgaatgagtatttttactcccatctgaacaacccttaccccagcgaagaagccaaagaagagctggccaggaagggcggcctcaccatctcccaggtctctaactggtttggcaacaaaagaatccggtataaaaagaacatggggaagtttcaagaagaggctaccatttacacgggtaaaacggctgtggataccacggaagttggggtcccagggaaccacgccagctgcctgtcaacacctagctccggctcctctggacccttcccgctgcccagcgctggggacgccttcctcaccctgcggactctggcctctctccagcctcctcctgggggaggctgcctgcagtcccaggcccagggtagctggcagggggccaccccccaacctgcaactgcctcacctgctggagaccctggcagcatcaactccagtacatctaattaagtttgggggataagcaggaaagagcgctgcgtgagctgccatgtatcgccagccgttgctttgttactgaacgtgccgccgacgacctcagaaaacccagatgggtggtggtgcccatgagcccctgctcctcagccaggcccgtggcgccggctcatgtgtctgctgcgactcgagatggcctgaaacgccactcattctcccacttcagttcgtttttttgacagtaattttatggtaacgctatgaattgaattgtctgttctaggactgggcacagattttcccattaaaatttttgacttattttaaaaaaa

SEQ ID NO: 33 - DNA, 7364 bp, Homo sapiens
  misc_feature: Incyte ID No 3075937CB1
cccctaacggagcccccagtgtgtgtgccgaagagggctccctggggccccgcaacgcccgcagccagccccgcaagggcgcgtctgatttgccaggtgagcctccggccgagggcgcagcgcacacggccagctccgcgcaggcagactgcacagcccgccccaagggtcacgcccaccctgctaaggtgctaactttggacatctacttgagtaagactgagggggcacaagtggacgagccggtcgtgattactcccagagcggaagattgcggtgactgggacgacatggagaagaggtccagcggccgtaggtcggggaggcggagggggtcgcagaaatccaccgactcccccggcgcggacgccgagctccctgagagcgctgccagggacgacgcggtgttcgacgacgaggtggcgccaaacgcggccagcgataacgcctcggcggaaaagaaagtgaaatctccgcgggcagccctcgacgggggcgttgcctccgctgcgagcccagagtccaagcccagccccggtaccaaagggcagctccgaggggagtcggaccggagcaaacagccacccccggcttcgtcccccacgaagaggaagggcaggagccgtgccctcgaggccgtgcccgccccgcccgccagcggcccccgggctcccgccaaggagtccccacccaagagggtgcccgatcccagcccagtcaccaagggcactgcggccgagagcggggaggaggcggcgcgggccatctcccgcgagctcccggtcaagagcagctcgctgctgccggagatcaagcccgagcacaagaggggcccgctccccaaccacttcaacggccgggcagagggaggtcgaagcagagagctgggcagagcggccggagcgcctggagcttctgacgccgacggcttgaagcccaggaaccatttcggcgtgggcaggtcgacagtgaccactaaagtgaccctccctgccaagcccaaacatgtggaactaaatcttaaaacccctaagaatcttgacagtttgggaaatgagcacaatccatttagccagccagttcacaaaggcaacactgccaccaaaatctccttatttgaaaacaaacggacaaacagtagcccaagacacactgacattcgaggcccaaggaatactcctgcctctagtaaaacgtttgttgggagggcaaagctgaatttagccaaaaaagccaaagaaatggagcaacctgaaaagaaagtaatgccaaacagtccccagaatggtgtgctggttaaggaaactgctatagaaaccaaagttaccgtctcggaagaagagattctgccagcaaccagaggaatgaatggagactcttctgagaatcaagctcttggtcctcagcctaaccaagatgataaagcagatgtacaaacagatgctggctgcctttcagaaccagtggcttctgctctgattcctgtcaaggatcataagctcttagagaaggaggactcagaggctgcagacagcaaaagccttgtacttgaaaatgtaaccgatacagcacaagacatccccaccactgtggataccaaagatttacctccaacggccatgccaaagccacagcatacattttctgactcacagtcccctgctgagtcatctcctgggccttctctttcactgtctgcacccgctcctggggatgttcccaaagacacatgtgttcaatcacccataagcagtttcccatgcactgatctaaaagtgtcagaaaaccataaaggatgtgttttgcctgtgtctcgtcagaacaatgagaaaatgccacttttagaacttggaggagaaacaacccctcctttgtccacagagcgtagtccagaagctgtgggaagtgagtgtccatccagagtcctcgtccaggtcaggtccttcgtgctccccgtggagagcacccaggatgtgagctcccaggtcatcccagagagctctgaagttagagaagtgcagttgccaacttgtcacagtaatgaacctgaagtggtttccgttgcaagttgtgctcccccacaagaggaagtactgggcaatgaacactctcattgcacagcagagctcgcggcaaaatctggcccacaagtcataccgccagcatcagagaaaactctgcctattcaggctcaaagtcagggcagcagaacacccctgatggctgaatccagtcccaccaactctcccagcagcggaaatcacttagccactcctcaaaggccagatcagactgttacaaatggccaggatagccctgccagccttttgaacatttctgctggtagtgatgatagtgtatttgattcttcttctgatatggaaaaattcactgaaattataaaacagatggatagcgcagtttgtatgcccatgaaaagaaagaaggccaggatgccaaactctcctgctcctcactttgccatgcctcctattcacgaagaccatttagaaaaggtgtttgatcccaaagtgtttacctttggtttggggaagaagaaggaaagtcagccagaaatgtcaccggctttacatttgatgcagaaccttgacacaaaatccaaactgagacccaaacgtgcatctgctgaacagagcgtcctcttcaagtccctgcacaccaacactaatgggaacagtgagcctctggtgatgccggaaatcaatgacaaagagaacagggacgtcacaaatggtggcattaagagatcgagactagaaaaaagtgcacttttctcaagcttgttatcttctttaccacaagacaaaatcttttctccttctgtgacatcagtcaacactatgaccacggctttcagtacttctcagaacggttccctatctcagtcttcagtgtcacagcccacgactgagggtgccccgccctgtggtttgaacaaagaacagtcaaatcttctgcccgacaactccttaaaggtcttcaatttcaactcgtcaagtacatcacactccagtttgaaaagtccaagccacatggaaaaatacccgcaaaaagagaaaaccaaagaagatctggattcacgaagcaacctacacttgccagaaactaaattttctgaattgtcaaaactgaagaatgatgatatggaaaaggctaatcatattgaaagtgttattaaatcaaacttgccaaactgtgcaaacagtgacaccgacttcatgggtcttttcaaatcaagccggtatgacccaagcatttctttttctggaatgtcattatcagacacaatgacacttagaggaagtgtccaaaataaactcaatccccgacctggaaaggtagtgatatatagtgaacccgacgtctctgagaagtgcattgaagttttcagtgacattcaggattgcagttcttggagcctctctccagtgatactcataaaagttgttagaggatgttggattttgtatgagcaaccaaattttgaagggcactccatccccttagaagaaggagaattggaactctctggtctctggggtatagaagacattttggaaaggcacgaagaagcagagtctgataagccagtggtgattggttccatcagacatgtggttcaggattacagagttagtcacattgacttatttactgaaccagaagggttaggaatcctaagttcctactttgatgatactgaagaaatgcagggatttggtgtaatgcagaagacttgttccatgaaagtacattggggcacgtggctgatttatgaagaacctggatttcagggtgttcctttcatcctggaacctggtgaataccctgacttgtccttctgggatacagaagcagcgtacattggatccatgcggcctctgaaaatgggtggccgtaaagttgaattccctacagatccaaaggtagttgtttatgaaaagcctttctttgaaggaaaatgtgtggaactagaaacaggaatgtgtagttttgtcatggagggaggtgaaacagaagaggcgactggagacgatcatttgccgtttacgtcagtggggtctatgaaagttctaagaggcatttgggttgcatatgagaagcctggatttaccggtcatcagtatttgctagaagaaggagaatacagggactggaaagcctggggaggttacaatggagagcttcagtctttacgacctatattaggtgatttttcaaatgctcacatgataatgtacagtgaaaaaaactttggatccaaaggttccagtattgatgtattgggaattgttgctaatttaaaggagactggatatggagtgaagacacagtctattaatgtactgagtggagtatgggtagcctatgaaaatcctgacttcacaggagaacagtatatactggataaaggattttataccagttttgaggactggggaggcaaaaattgtaagatctcttctgttcaacctatatgtttggattctttcactggcccaaggagacgaaatcagattcacttgttttcagaaccacagtttcaaggtcacagtcaaagttttgaagaaacaacaagtcaaattgatgattcattttctaccaagtcttgcagagtttcaggaggcagctgggttgtatatgatggagaaaatttcactggtaatcaatacgtgttggaagaaggccattatccttgtctgtctgcaatgggatgcccgcctggagcaactttcaagtctcttcgttttatagatgttgaattttctgaaccaacaattattctctttgaaagagaagacttcaaaggaaaaaagattgaacttaatgcagaaactgtcaatctccgatccctgggattcaacacacaaatacgctctgttcaggttattggtggcatatgggttacttatgaatatggcagttacagagggcgacagttcctattgtcacctgcagaagtacctaattggtatgaattcagtggctgtcgccaaataggttctctacgaccttttgttcagaagcgaatttatttcagacttcgaaacaaagcaacagggttattcatgtcaaccaatggaaacttagaggatctgaagcttctgaggatacaggtcatggaggatgtcggggccgatgatcagatttggatctatcaagaaggatgtatcaaatgcaggatagcagaagactgctgcctgacgattgtgggcagcctggtaacatctggctccaagctaggcctggccctggaccagaatgctgacagccagttctggagcttgaagtccgatggcaggatttacagcaagttgaagccaaatttagttttagacattaaagggggcacacagtatgatcaaaatcacattatcctcaacactgtcagcaaagagaagtttacacaagtgtgggaagccatggtcctatatacctgaacaaagaaggaagaagaatcttctggaggtccttccagccaccttatttcttaaaaaggacaatgctgatggaagaccagactggaaagtggatcgactcctccttcattgattctaaattcaaccttaaatcatgctgccatgactcagagaacttactcatcgtttcaaaagactatcatagctttaaaccaataatttgtcctcctttcatttcttgcctttcatttttggtagctgcttaaacaggttgcctaattagcagcttttgggtgattttgtaaaatgttatatcaagatttcaagactgtgtacattttaaattatttccaaagatagtgacaggagagaactggaacaaatttaccaactttgtggacctacaaagcccttacactttaaagggtaagacaaaggcttaagtttgaaaggtagagaactgtttagcatctgagaagaaatactttattaggcctgtaattttggttcttggccttaaacactttctggaacctttaaatatgctgcatagcacaatgggaaagccttaggtattcacacatttaaggaactctaaacaaaatactattttcctttagttcatattaaaaattaatacattttaaaaatttaatgtcaaagtctggtaacatttgttagtaggatttgagttattattttttgagacaggatctcaggctggagtgcagtggcacaatcacggctcactgcagcctctacctccccaggctcaggtgatcctcccacctcagcctcccaagtagctgggactataggcacacatcaccaagcccagccaaattttgttttttttttgtagagatggggtttcatcacgttgcccaggctgatctcgaacctctgggctcaagcaattcactcgcctcggcctcccaaaatgctgggattacaggcctgagccactgcgcccagccaggatttgaattattttaactcatccatgggctgccctagaatgtcacaaatgagggttgtttaatgcctttcttatagctgctactggaacactattatgacctaatttatgagccatccttactcatctacaagtgctgaagcaatgttacatacttttttgctaaactcagattttttagcctaatttcttgtcctcctatccacctgcatccacacatggcctgcatggggctgccttccctgcagtgttctgcagccatgcttcagggtatagctgttggtggacagcctcaggtcttgggggcactatagccactaaacgaggtgtgaaaggctcaagaggatgaccagcaattaattatccccagaaagtgaaggaaaagagacctttagggatgttgctggtcaagtcttgatttgaccggagtcaaatcaatcttcaagcaatcttggaatcctcaactgcagtaagcatttcaaaatgcaaacaaactgcttaacaactgacaagacaccagcccatacgctgctcttccaacagtgggttctagctttgaacaaaagtgctaaacatttccttgaatatattcttcctctttttgtcctcatcactcaatactggtgctcttgtcacaggtagaacagcttgtttcttttccatctattcaagtgtgtttctaattctaaaatgctgatcttctctggagtctatggtaggcaattatggtcactggaatagtttgtcttgttttaaaatattattggtgcatgtacaacagcatccaacatatctgtcttgttcctagatatatagctctgattttaggccttttgtgcataccattacaatatggtggggtaagacattctacagtagcctgtgctgaactgatctcttaaataaacttgcttctggttaactatgaaaaaaaaaaaaaaaaa

SEQ ID NO: 34 - DNA, 1345 bp, Homo sapiens
  misc_feature: Incyte ID No 5176268CB1
gtgggattaagaagtgataggagagcttgctgtctgtctctgctctccactgtgtgaggatacaacaggaagacagccatctggtgaggaagagagggccctcgccagataccggacctgctgacaccttgatcttggacttcccatcttccaggaaggcctgacctcagttgttccagggtaaagaatttgggcagtgcccacacccacgctgttggataacatttcttcaccataccagtgagggtgaatgtgtacacgcccagcttcctgcctgttactctccacagtatgcgaagaatatccctgacttctagccctgtgcgccttcttttgtttctgctgttgctactaatagccttggagatcatggttggtggtcactctctttgcttcaacttcactataaaatcattgtccagacctggacagccctggtgtgaagcgcaggtcttcttgaataaaaatcttttccttcagtacaacagtgacaacaacatggtcaaacctctgggcctcctggggaagaaggtaaatgccaccagcacttggggagaattgacccaaacgctgggagaagtggggcgagacctcaggatgctcctttgtgacatcaaaccccagataaagaccagtgatccttccactctgcaagtcgagatgttttgtcaacgtgaagcagaacggtgcactggtgcatcctggcagttcgccaccaatggagagaaatccctcctctttgacgcaatgaacatgacctggacagtaattaatcatgaagccagtaagatcaaggagacatggaagaaagacagagggctggaaaagtatttcaggaagctctcaaagggagactgcgatcactggctcagggaattcttagggcactgggaggcaatgccagaaccgacagtgtcaccagtaaatgcttcagatatccactggtcttcttctagtctaccagatagatggatcatcctgggggcattcatcctgttagttttaatgggaattgttctcatctgtgtctggtggcaaaatggcagaagatccacctagaggtgataccacggcggcgcagagttgttcacctgtggtcctcgatcgctgacagccttggctcccactgctgtgtgttccctgagtcaagtggaggcggagcctttgctcgtgagattgtggacctatgatcctaccaccagcccacctggttctaacaccccctcctctatgtgtgagagggagagaagaaaagtgagggagaaaagagagataagcaagaccagagaggaaaaatctgtccagcacctgcgccgtaactagtgatccagctcgtccccacagc

SEQ ID NO: 35 - DNA, 4255 bp, Homo sapiens
  misc_feature: Incyte ID No 72830854CB1
agccggttcccccccacctgccaccgtacctggctccagcctcccaggtgggggcccccgctcagctgaagcccctccagatgccacaggcgcccctgcagccgcttgctcaagtccctccgcagatgcccccgattcctgttgtgccccccatcacgcccctggcggggaatcgacggcctccctccggccctcccagacctgccgaccgcgactgtgcctcccatgccaccacctcagtatttctctccagccgtgatcttgccgagcctcgctgccccactcccccctgcgtccccagccttgcctctgcaggctgtgaagctgccccacccccctggggcgcccctggccatgccctgccggaccattgtgccaaatgcaccggccactatccccctgctggccgtagccccaccgggcgtggctgccctgtccattcattctgccgtggcccagctcccaggccaacctgtgtacccagcggccttcccacagatggcgcctactgacgtccctccttccccccatcacacggtgcagaatatgagggccacccctccacagccggcactgcctccacaacccacactgcccccacaacccgtgctgcccccgcaacccacgctgccccctcaacctgtgttgcccccgcaacccacacggccccctcaacctgtgctgcccccgcaacccatgctgcccccacaacctgtgctgcccccgcagccggcactgcctgtgcgccctgagcccctccagccccaccttcctgaacaagctgctccagctgctacaccagggagccagattctgcttggccacccagctccctatgctgtggacgtcgccgctcaggtccccaccgtgcctgtgccaccggctgcggtcctctcgccgcctctgccggaagtgctgctgcctgccgcccctgagctcctgcctcagttccccagctccctggccacggtgtctgcctctgtgcagagtgtgcccacccagactgccacacttctgccaccagcaaacccaccgctgcctggcgggcccgggatcgccagcccttgcccaactgtccagctgacggtggaaccagtccaagaggagcaggcctcacaggacaagccgcccggcctcccgcagagctgtgagagctatggaggttctgatgtcacttctggaaaagagctgagtgacagctgtgaaggcgcctttggagggggcaggctggagggcagggcagcccgaaaacaccaccgcaggtccacgcgtgcgcgctcccggcaggagagggccagccggccccggcttaccatcttgaacgtgtgcaacactggggacaagatggtggagtgccagctggagacgcacaaccacaagatggtgaccttcaagttcgacttggacggggacgcacccgatgaaattgccacgtatatggtggagcatgactttatcctgcaggccgagcgggaaacgttcatcgagcagatgaaggatgtcatggacaaggcagaggacatgctcagcgaggacacagacgccgaccgtggctccgacccagggaccagcccgccacacctcagcacctgcggcctgggcaccggggaggagagccgacaatcccaagccaacgcccccgtgtatcagcagaacgtcctgcacaccgggaagaggtggttcatcatctgtccggtggctgagcaccccgcccccgaggcccctgaatcttcgcccccacttcctctaagctccctgccgccagaagccagccaagattcagcgccctataaagaccagctgtcctcgaaggaacaacccagctttctagccagtcagcagctcctgagccaggcgggccccagcaaccctcctggggcacccccagcccctttggccccctcctcccctcctgtgactgctctgccccaagatggagcagctccagccaccagcaccatgccagagccagcgtcaggaactgccagccaggcagggggtccagggacacctcaggggctgaccagtgagctcgagacgtctcagccactagcggagactcacgaggccccgcttgctgtgcagcccctcgtggtgggcctagcaccttgcactccagctccagaggctgcctcaaccagggacgccagtgccccaagggagcccctgccacctcctgcacctgagcccagcccccacagcgggaccccacagcccgccttgggtcagcctgctcccctgcttcctgccgcagtgggggccgtcagcctggccacctcccagctcccaagcccacccctggggcccaccgtccccccacagccaccctcggccctggagtcggatggggaagggccgccccccagggtgggctttgtggacagcaccatcaagagcctggacgagaagctgcggactctgctctaccaggagcacgtgcccacctcctcagcctcagctgggacccctgtggaggtgggcgacagagacttcaccctggagcccctgagaggggaccagccccgctcagaggtctgcgggggggacctggccctgcccccagtgcctaaggaggcggtctcagggcgtgtccagctgccccagcccttggtggagaagtcagaactggcccccactcgaggggccgtgatggagcagggcacgtcctcgtcaatgacagcagagtcgtctcccaggagtatgctaggctatgacagagatggaaggcaggtggcctcagactcccatgtggtccccagcgtcccccaggatgtacctgcttttgtgagacctgcacgtgtggagcccacagacagggatggtggagaagctggagaaagctcggcagagcccccgccgagtgacatgggcacagtggggggccaggctagccacccccagacactcggcgctcgagctttggggtcccctcggaaacgtccagagcagcaggatgtcagctcaccagccaagactgtgggccgtttctcggtggtcagcactcaggacgagtggaccctggcctccccccacagcctgagatactctgccccacccgacgtctacctggacgaggccccctccagccccgacgtgaagctggcagtgcggcgggcgcagacggcctcctccatcgaggtcggcgtgggcgagcccgtgtccagcgactctggggacgagggccctcgggcgagacccccggtgcagaagcaggcgtccctgcccgtgagtggcagcgtggctggcgacttcgtgaagaaggccaccgccttcctgcagaggccttctcgggccggctcgctgggccccgagacacccagcagggtgggcatgaaggtccccacgatcagcgtgacctccttccattcccagtcgtcctacatcagcagcgacaatgattcggagctcgaggatgctgacataaagaaggagctgcagagtctgcgggagaagcacctgaaggagatctcggagctgcagagccagcagaagcaggagatcgaagctctgtaccgccgcctgggcaagccactgccccccaacgtgggcttcttccacacggcaccccccactggccgccggagaaaaaccagcaagagcaagctgaaggcaggcaagctgctaaatcccctggtgcggcagctcaaggtcgtggcctccagcacagggtcctccaccagcagcctggccccaggccctgagccaggcccccagcccgccctgcacgtccaggcgcaggtgaacaacagcaacaacaagaagggtaccttcacggacgacctgcacaagctggtggacgagtggacgagcaagacggtgggggccgcgcagctgaagcccacgctcaaccagctgaagcagacccagaagctgcaagacatggaggcccaggcaggctgggctgcccctggcgaggcgcgggctatgaccgcacctcgagcaggagtggggatgccacgtctgcccccagcgcccggccctctgtccaccacggtcattcccggagccgccccgaccctgtccgtgcccacaccagatcctgagagtgagaagcctgactgaccccgcctagacgccaggcccacttcacgccgtctaagtggagaagtgacggaccctcagggccagctgctcctcctgtccagttcacgctgttttgtaaccactttctaagcattttttattcacaattggaaacacaaatgtaatgcaagaataaaaaatattttggggccaaaaaaaaaaaaaaaagggc

SEQ ID NO: 36 - DNA, 4181 bp, Homo sapiens
  misc_feature: Incyte ID No 3632052CB1
ttgttatttcatatgtgctttattctccctctcctatcgtcttatgttattttatcagcgtccctttgaatttattcttctttcttctcttctgatttctgttatttttgtgtggttgtgtgtgattttgttatgcgtgtctcgtcttcttattgtctcttcattttcttcttgtttcattatgtcgttttttgtctctttttctattctctccttcttttctattttttctgttccgtcttttgtcttgtgtctcctgccttcgcaaagggggatgtgctgccaggcgattaagttgggtaacgccagggttttcccagtcacgacgttgtaaaacgacggccagtgaattctcagtttggataattggtcaaacaattttttatccagtagtaaaatatgtaaccattgtcccagtaaagaaaaataacaaaagttgtaaaatgtatattctcccttttatattgcatctgctgttacccagtgaagcttacctagagcaatgatctttttcacgcatttgctttattcgaaaagaggcttttaaaatgtgcatgtttagaaacaaaatttcttcatggaaatcatatacattagaaaatcacagtcagatgtttaatcaatccaaaatgtccactatttcttatgtcattcgttagtctacatgtttctaaacatataaatgtgaatttaatcaattcctttcatagttttataattctctggcagttccttatgatagagtttataaaacagtcctgtgtaaactgctggaagttcttccacagtcaggtcaattttgtcaaacccttctctgtacccatacagcagcagcctagcaactctgctggtgatgggagttgtattttcagtcttcgccaggtcattgagatccatccactcacatcttaagcattcttcctggcaaaaatttatggtgaatgaatatggctttaggcggcagatgatatacatatctgacttcccaaaagctccaggatttgtgtgctgttgccgaatactcaggacggacctgaattctgattttataccagtctcttcaaaaacttctcgaaccgctgtgtctccaatatcttcttcaggctctgacaggcctcctggaaacttccacatatttttcaatttatttcgatcttgtacaaccagtatttttctagtactttcatcaaatacagctcctgcaactcctacttgatgtgaagcatatcctggtaatctgctgggcccttctctcagccacagagtcaacgttgatgaatccgattctgcgtggtgaaagcggaagcccagggaagcagcaggggcaataaatcggctttggaggatgggaatgtgcagccatacagctgttctaccttctgatcgccattgctgtactgcagcctgcaagcccttctggaaggcggcagcgtccaggcggtccagcgcatcgagccgcgccaggcgcaccgagatgcccccgaatctgtccagctcgccctgcagatcgcacgctccaactggcggattccgcacgtaaccctgtgcgcccgaggcccagcggtaacccgccgaagcgggggcggtaccttgtgaccatgtcttccaagaagaatagaaagcggttgaaccaaagcgcggaaaatggttcgtccttgccctctgctgcttcctcttgtgcggaggcacgggctccttctgctggatcagacttcgcggcaacctccgggactctgacggtgaccaacttattagaaaagggtaaagaattccgggtgtatacagcctggcctatggcaggatttcctggaggcaaggtcggcctgagtgaaatggcacagaaaaatgtgggtgtgaggcctggtgatgccatccaggtccagcctcttgtgggtgctgtgctacaggctgaggaaatggatgtggcactgagtgacaaagatatggaaattaatgaagaagaactgactggttgtatcctgagaaaactagatggcaagattgttttaccaggcaactttctgtattgtacattctatggacgaccgtacaagctgcaagtattgcgagtgaaaggggcagatggcatgatattgggagggcctcagagtgactctgacactgatgcccaaagaatggcctttgaacagtccagcatggaaaccagtagcctggagttatccttacagctaagccagttagatctggaggatacccagatcccaacatcaagaagtactccttataaaccaattgatgacagaattacaaataaagccagtgatgttttgctggatgttacacagagccctggagatggcagtggacttatgctagaggaagtcacaggtcttaaatgtaattttgaatctgccagagaaggaaatgagcaacttactgaagaagagagactgctaaagttcagcataggagcaaagtgcaatactgatactttttattttatttcttcaacaacaagagtcaattttacagagattgataaaaattcaaaagagcaagacaaccaattcaaagtaacttatgacatgataggaggattaagtagccagctgaaagcaattagagaaataattgaattgcccctcaaacagcctgagcttttcaagagttatggaattcctgcccctagaggattgttactttatggtcctccatgtactggaaaaacaatgatcgccagggctgttgctaatgaagttggagcctatgtttctgtaattaatggtcctgaaattataagcaaattctatggtgagactgaagcaaagttacgtcagatatttgctgaagccactctacgacacccatcaattatttttattgatgagctggatgcactttgtccgaaaagagagggggcccagaatgaagtggaaaaaagagttgtggcttcactcttaacactgatggatggcattggttcagaagtaagtgaaggacaagtgttggttcttggggccacaaatcgccctcatgccttggatgctgctctccgaagacctgggcgatttgataaagagattgagattggagttcccaatgctcaggaccggctagatattctccagaaactgcttcgaagggtaccccatttgctcactgaggctgagctgctgcagctggcaaatagtgctcatggatacgttggagcagacttgaaagtcttgtgtaatgaagcaggtgagtgtggtttgctatggtgagtctctattgatgcacttatctccagtttacttacatacaaataatttatattttacagatttcttaatggaagtagctttgtttctaattataaaatgtgtaatttttatttgaaaaaatttttattggaaaaacctagatgattcagaagactataaaaaaaaataaaaatcactcaatcctacctctaaaggataaccaccattatagtgtatacagtcagccctccatatccttgggctcagcatctgaggagtcaaccaactgtggtctgaaaatattcaggaaaaaaaagttaaaacagtaaaaaataataaaaacttaaaaaatatagtatcacaactacagttgacccttgaactatgagggcttgaactgcatgggtccacttatatatgaatatttttcaataaatatattggaaaattttttggagatttgcaacaatttgaaaaagcttgctttttctttagcttactttattgtaagaatacagcatataatgcatataaaaaatatggttaatcagctgtttatattattggcaaggcttcaggtcaacaataggctattagtagttagtttttgggggagtcagaagttatgcatagttttttgactgcacagggattggtgtccctgacccccaagttgttgaggggtcaactgtatttacatagcatttacatgggatcaggtaaaagtaatctagagaattattttaacttatacgggaggacgtgtgttagccaattttgcacagtataatgcccttaagggtttagacgccttaaatttgggatcttaaggggccgggacaaataatcccagaaataggggaaaattcgctaaaatttccaagattatacacaaaagaacacccgaagggttgttttttttccccacatcttctcttaaaattaataccggtccaacctcggaagaaaaaattattg

SEQ ID NO: 37 - DNA, 2478 bp, Homo sapiens
  misc_feature: Incyte ID No 7493817CB1
gggctgcagaaagaggaggccaggagcggtcccatccgtcccgtcccgtcccgtctccccctcttcctcttgctccttgccccccggctctgcgagagttgagggttcaggtggccgtacgcggcagtgagggcaagagggccgggagagtggggagcggaggcaggagtgcgggggaagatgcccatcctgctgttcctcatagacacgtccgcctctatgaaccagcgcactgacctgggcacctcttatttggacattgccaaaggcgctgtggagttattcttgaagctgcgcgcccgggacccggccagccgtggagacaggtacatgctggtcacctacgacgaacccccgtactgcatcaaggctggttggaaggaaaatcatgcaacattcatgagcgaactaaaaaatcttcaggcttctggactgactactctcggtcaggctctaagatcctcatttgatttgttaaatctcaatagattaatatctggaatagacaattatggacaggggagaaatccattttttttagaaccatctattttaattaccatcacagatggaaacaagttaacaagtactgctggtgttcaagaagagctccatcctcctttgaattcccctctgcctggaagggaactaaccaaagaaccttttcgttgggatcaaaggttatttgccctggtgttgcgtttgcctggagtggcttctaccgaaccagagcaactagggagcgtaccaactgatgaatctgccatcacacagatgtgtgaagtcacaggaggtcgctcctactgtgtgagaacacaaagaatgttgaatcaatgtttagaatctctagttcaaaaagttcagagtggtgtagttattaattttgaaaaaacaggaccagatccacttcctattggagaagatggacttatggattcatccaggccaagcaattcatttgctgctcagccatggcatagttgtcataaactcatttatgtacgacctaactctaaaactggtgttcctgttggacattggccaattccagaatctttttggccagatcagaatttaccttcactacctccacgaacatctcatcctgttgtgaggttctcctgtgtagattgtgagccaatggtaatagacaaacttccttttgacaaatatgaacttgaaccttcgcccttaactcagtatatcttggaacgaaagtctccccatacctgctggcaggtatttgttactagcagtggaaagtacaatgaacttggatatccatttggctatttaaaagccagtacaactttaacttgtgtaaacctctttgtgatgccttacaactacccagttttacttcctcttttagatgacttgtttaaagttcacaagcttaagccaaatctgaagtggcgacaggcttttgacagctacttaaaaactctgcctccatactacctattaccattaaagaaagcactaaggatgatgggagctccaaatctgatatcagataatttagattgtggacttagttacagtgttatctcttaccttaaaaaactcagccaacagaccaaactagagtcagaacgaatactagcatcagtggggaagaaacctccccaggaaattggaattaaagtgaaaaatcattctggaggtggcatgtccttgactcacaataaaaattttagaaaactattgaaagaaatcacaggggaaactgcacttagactgacagaattgaacaccaaagaatttgctggcttccaaattgggctcttaaacaaggtaaattgtgacataaatagtttgtatttgtttgaacttgtctgatacatttatgtctttcatattcctttagttttgaaataatggacaatcctattttggagttagtagttaataatggaccagtttaggaattgtcagcagatatcaatatggaattaaatggacaagatatgttttgtgggtaacgcccatgaaagataaaggggagatgaagctggagtaggcaggcagagcttccaagctacaaagctggcccatcacctgtgagaggaaggaggaatgaaggagaattgggtgtgaagaagctcagacactggtgtagcttggagaaagtcttggccagcacagtggggacctctagcaccaagatttcctacagaggagggccacactgggcagagctggccaggccctaacaccctgccattgtctgttactggctatgggcagcctgggaagggcatggcctcagctcagtgctgcagcggatccctatggcaccacagcagcgggaggctctctgctaactgcactccttgcaggatcagccattgctttcctgaagggagatcccagcagctcacctcccctccatggctgccagaggaatacatctagagtacgagcagtggtcctgcaaggcggccacaggtatcaatctcgggatttggtgtttta

SEQ ID NO: 38 - DNA, 2928 bp, Homo sapiens
  misc_feature: Incyte ID No 6715627CB1
ttcggctcgaggcgaggacagggtacgtcgcaggcttgtgcgggtcgggctcggacctgcgctgcctcgggatgtaaagtataacaagagggtcgggatggtcagcgtaggcctgtgtaggcctgtcgggtgcccctgtcccccatgctccccccagcagccggctccgcagtggtccactccggttgccgggtgcggattcgggttccggaccgaaggctgtgtgttctccgccgtttattgtggccccgacaggccggggttactgtggcgaccacgagagcagctttggcgctatggaggagcccggggctacccctcaaccgtatttggggctgctcctggaggagctacgcagggttgtggcagcactgcctgaaggtatgagaccagattctaatctttatggttttccatgggaattggtgatatgtgcagctgttgttggattttttgctgttctcttttttttgtggagaagttttagatcggttaggagtcggctttatgtgggacgagagaaaaagcttgctctaatgctttctggactaattgaagaaaaaagtaaactacttgaaaaatttagccttgttcaaaaagagtatgaaggctatgaagtagagtcatctttaaaggatgccagctttgagaaggaggcaacagaagcacaaagtttggaggcaacctgtgaaaagctgaacaggtccaattctgaacttgaggatgaaatactctgtctagaaaaagagttaaaagaagagaaatccaaacattctgaacaagatgaattgatggcggatatttcaaaaaggatacagtctctagaagatgagtcaaaatccctcaaatcacaagtagctgaagccaaaatgaccttcaagatatttcaaatgaatgaagaacgactgaagatagcaataaaagatgctttgaatgaaaattctcaacttcaggaaagccagaaacagcctttgcaagaagctgaagtatggaaagaacaagtgagtgaacttaataaacagaaagtaacatttgaagactccaaagtacatgcagaacaagttctaaatgataaagaaagtcacatcaagactctgactgaacgcttgttaaagatgaaagattgggctgctatgcttggagaagacataacggatgatgataacttggaattagaaatgaacagtgaatcggaaaatggtgcttacttagataatcctccaaaaggagctttgaagaaactgattcatgctgctaagttaaatgcttctttaaaaaccttagaaggagaaagaaaccaaatttatattcagttgtctgaagttgataaaacaaaggaagagcttacagagcatattaaaaatcttcagactgaacaagcatctttgcagtcagaaaacacacattttgaaaatgagaatcagaagcttcaacagaaacttaaagtaatgactgaattatatcaagaaaatgaaatgaaactccacaggaaattaacagtagaggaaaattatcggttagagaaagaagagaaactttctaaagtagatgaaaagatcagccatgccactgaagagctggagacctatagaaagcgagccaaagatcttgaagaagaattggagagaactattcattcttatcaagggcagattatttcccatgagaaaaaagcacatgataattggttggcagctcggaatgctgaaagaaacctcaatgatttaaggaaagaaaatgctcacaacagacaaaaattaactgaaacagagcttaaatttgaacttttagaaaaagatccttatgcactcgatgttccaaatacagcatttggcagagagcattccccatatggtccctcaccattgggttggccttcatctgaaacaagagcttttctctctcctccaactttgttggagggtccactcagactctcacctttgcttccagggggaggaggaagaggctcacgaggcccagggaatcctctggaccatcagattaccaatgaaagaggagaatcaagctgtgataggttaaccgatcctcatagggctccctctgacactgggtctctgtcacctccatgggaccaggaccgtaggatgatgtttcctccgccaggacaatcatatcctgattcagcccttcctccacaaaggcaagacagattttgttctaattctggtagactgtctggaccagcagaactcagaagttttaatatgccttctttggataaaatggatgggtcaatgccttcagaaatggaatccagtagaaatgataccaaagatgatcttggtaatttaaatgtgcctgattcatctctccctgctgaaaatgaagccactggccctggcttgttcctccacctcttgctcccaatcagaggtccattgtttccagtggatgcaagaggcccattcttgagaagaggacctcctttccccccacctcctccaggagccatgtttggagcttctcgagattattttccaccaggggatttcccaggtccaccacctgctccatttgcaatgagaaatgtctatccaccgaggggttttcctccttaccttcccccaagacctggatttttccccccacccccacattctgaaggtagaagtgagttcccctcaggtttgattccaccttcaaatgagcctgctactgaacatccagaaccacagcaagaaacctgacaatatttttgctctcttcaaaagtaattttgactgatctcattttcagtttaagtaactgctgttacttaagtgattacactttggctcaaattgaagcttaatgggaattataattctcaggatagtatttgtaaatcaagatgatttacatatgaatcttatgagtaaattattccaattttattttagacggtatactatttcacttgattaa

SEQ ID NO: 39 - DNA, 3486 bp, Homo sapiens
  misc_feature: Incyte ID No 7727886CB1
gtcgccgggcgggccaggagcagcgcggacccgagccgggcagggggcgcccgccacggcacccgcgcgctcctagcgccccagacccgcctgcgggcccggatcctccttgccactgtcccacccgccgtccctgcccctccaccctttgtgtcgccgcagcccggtgcccccggctctgcgggaccccggccgggccggaccctggcaaagcgccaggccccgcgtgggctcccggcgagcggttgatggcgagggggcgcggcgcgggctctgtagcccgagttcccgacgctggaggcccggcccgcctcagccgcattgtcccgggccgcgcgcaccggccctgagctgcgccgccgcagcacccgcccgccgcccgcggggccatgcggagagccgccgggatggaggacttctccgcggaggaagaggagtcctggtacgaccagcaggacctggagcaggacttgcacctagctgcggagctggggaagactctgctggagaggaacaaggagctggaggggtccctgcagcagatgtactccaccaatgaggaacaggtgcaggagatcgagtacctaaccaagcagctggacacgctgcggcacgtgaacgagcagcacgccaaagtctatgagcagctggacctgacagcccgggacctggagctgaccaaccacaggctggtgctggagagtaaggctgcccagcagaagatccatgggctgacggagaccattgagcgcctccaggctcaggtggaggagctgcaggcccaggtggagcaactgagaggcctggaacagctgcgagtgctccgggagaagcgggaacgcaggcgtaccatccacaccttcccctgcctcaaggagctgtgcaccagcccccggtgcaaggatgctttccgcctacacagttcctccctggagctgggcccgcggcccctggagcaggagaacgagcggctgcagaccctggtgggggcgctgcgctcccaggtgagccaggagcggcagcgcaaggagcgggcggagcgcgagtacaccgcggtgctgcaggagtactcggagctggagcgccagctgtgcgagatggaggcctgtcgcctgcgtgtgcaggagctggaggccgagctgctggagctgcagcagatgaagcaggccaagacctacctactgggtccggacgaccacctggccgaggccctgctcgcacccctcacgcaggcccctgaggccgacgatccccagcccggccgcggggacgacttgggcgcccaggacggggtctcctcaccggcagcctctccaggccacgtggtgcgcaagagctgcagcgacactgcgctcaacgccatcgtggccaaagacccagccagccggcacgcgggcaacctcacactgcacgccaacagcgtgcgcaagcggggcatgtccatcctgcgggaggtggacgagcagtaccacgcgctgctggagaagtacgaggagctgctgagcaagtgccggcagcacggggccggagtgcggcacgccggcgtgcagacctcgcgccccatctcccgggacagctcgtggagggacctgcgcgggggtgaggagggccagggtgaggtcaaggcaggagagaagagcctgagccagcacgtggaggccgtggacaagcggctggaacagagccagcccgagtacaaggcgctcttcaaagagatcttctccaggatccagaagaccaaggctgacatcaacgccaccaaagtcaagacgcacagcagcaagtgacccttctccggcctgcagcctcccccagggtggaagccgtggggtccctcaggcctgggcggtgcagcttccagagagcgagcgccctttagcggcctgccaccacagcacgcggcctcctgatccggaagcacgcagcatgttccctgctgagcggaggcagcccacctgtcctgcctcccaggagcccttggccacctcgcgccagcccaaaggcgcagctctgagttcaaagccaaatgtccccactaccccagggatcccccagctcccccagcccctggcttcctgaccctgcgcctcaccctcagactggtgaccaggcttctgaaagccattctggatcagttggctttttttttttttttggttaagtttgttttttctaagagatttgcaatgcaaggtctccttgaccccttgccacaactggaaacacttgaaaggggaccccagggccagctgtttcaggggtttcctggaccacccactgcttctccccaaccctgatgcgctgacattcccttagcaccagctgtcccacctccagggtcctgaccaggtcagagatgtcccctgccatgcagagcaggaagcctcagctgggcctggagtgtccctgctccagccctgccagggacaggtttctccctggatactcttggcccaccgcagatctgtagccagtcagaggaggaggagaaggagcccctcagcagagtggtgcagtttcgctcagagcttgtctccttggcttccaaccccagaaatgcctgctgggccttaagctttccaggggccggggcagtggggagcccccatcccttcacaccgccaccaactaaccaagcttggcctctgactcccgtctctgtgcttgcccccatctcagggaccatgatgtctcagtcactccacgctccccacaggccaaccctggcacaggtcatgtctgcagcccccagaatgttctggacatgcaccaccagccggtggtcccaatgtccacccctgcctccccttcactggggactggggttttcgccccatgctgcatcgtgttgtattgggatggggctgaggaacatgctccctccctccctcccataaaatgcctgctcttcacctcccacctttgtggggggcttttgaggacccagctgcgtcaggagttttgcttcaagatgtcagaaagtcaagttcagctaagagacacccaggtccccagcttgccctgagcagcccttcagggcttctggttccttctgccgcccttcctgagaccttagaaaccagaagagccatacagtcagtggaaggcgggggggccctggcctctgcaccgggatcccagtgggaaccttcatgccttatttatttctaatgggtaaaggggttttcttaccaagcatccctgacctcctggagacaccacctgctttccgggcggcactgtgatgggagctggtggcgactgagtccttctgtacgtgcaactgggaaacttttgtcctttgaggctaggcagctccctgccctccgtgtgtgtctgttatctgggggagaggagtgtggaagggttgggggaagagctccagcctgtctgctccccagctctgtagtggcagaccagcgtcacctttgaagtatacgtgagagaaatatatttacaaatgctttattctcttctttaataaaa

SEQ ID NO: 40 - DNA, 2535 bp, Homo sapiens
  misc_feature: Incyte ID No 914113CB1
agcgccggcgcggggccgcgagaacgcagcgcaggggagcagcccgaggcggacaccgcgagccgcccggcactcccgcagtccagccggctcctctagcccggccacggctccgctgcgggccacccaggattactcgcgtctggctccaggcgccgagaaggcgcgctgggcgcccgtggccgccgcgccagctcctcctcctcccgctgctcctgctcccggggcgagcgcgcagccccgagcccgccccgcgcctcccggagccctcccccccgctgctcccatgcgcgcgggtgggtcatgagcacagcgccctcgctttctgccctaagaagcagtaagcacagcggcggcggcggcggcggaggcggaggcggaggcggaggcggcggtgcagaccctgcctggaccagcgcgctctctggaaatagctccggccccggcccaggctcgtccccggccggcagcaccaagccttttgtgcacgccgtgcccccctctgaccccctgcgccaggccaaccgcctgccaatcaaggtgctgaagatgctgacggcacgaactggccacattttgcaccccgagtacctgcagcccctgccttccacgccggtcagccccatcgagctcgatgccaagaagagcccgctggcgctgttggcgcaaacatgttcgcagatcgggaagcccgacccctcgccctcctccaaactctcctcggttgcctccaacgggggcggcgcgggcggtgccggcggcggtgctgcgggcgacaaggacaccaaatcgggccccctgaagctgagcgacatcggcgtggaggacaagtcgagtttcaagccgtactccaaacccggctcggataagaaggagccgggaggcggcggtggaggcggtggcggtggcgggggcggcggcgggggtgtttcgtcggagaagtcgggattccgggtaccgagcgccacctgccagccattcacgcccaggacaggcagccccgagctccagcgcctcggcctgctcgccgggaggtatgctgtcctcggccgggggtgccccggagggcaaggacgacaagaaagacaccgacgtgggcggccggtggcaagggcaccggggggcgcctcggccgaagggggacccacggggctggcacacggccggattagctgcggcggcgggattaatgtggatgtgaaccagcatccggatgggggcccgggaggcaaggctctgggctcggactgcggcggttcatcgggctccagctccggctccggccccagcgcgcccacctcctcctcagtgttgggctctgggctggtggctcccgtgtcaccctacaagccgggccagacagtgttccctctgcctcccgcgggtatgacctacccaggcagcctggccggggcctacgccggctacccgccccagttcctgccacacggcgtggcacttgaccccaccaagccgggcagcctggtgggggcgcagctggcggcggccgcggccgggtctctgggctgcagtaagccggccggctccagccctttggccggagcgtctccgccgtccgtgatgacagccagtttgtgccgggacccttactgcctcagctaccactgcgctagccacctggcaggggcggcggccgccagcgcttcttgcgcacatgatccggctgctgcggctgcggcgctgaagtccggatacccgctggtgtaccccacgcacccgctgcacggtgtgcactcctcgctaacggccgccgcggctgctggcgccacaccgccctccctggccggccaccccctctacccctacggctttatgctccctaacgacccactcccccacatctgcaactgggtgtcggccaacgggccgtgcgacaagcgcttcgccacgtccgaagagctgctgagccacttgcggacccatacggcatttcccgggacagacaaactgctgtcgggctaccccagctcgtcgtctctggccagcgctgccgcggccgccatggcttgccacatgcacatccccacctcgggcgcaccgggcagccctgggacgctggcgctgcgcagcccccaccacgcgctgggactcagcagccgctaccacccctactccaagagcccgcttcccacgcctggcgcccccgtgccggtgcccgccgccaccggaccgtactactccccctacgccctctacggacagagactgaccaccgcctcggcgctggggtatcagtgagggcggccgggagggcgagcgagggagaggagggagagggggaggggaggagtccagggagaggcgggatcacggcccaggctgctgacacccgcgcgtggggaggactcgggccacgaaaggaaagaaatgtataccgtatctatctacccgacagcagcgaccgagacccggtgggacactccccttctccccactttcacctccccacccaaactttataaaagttgaaaaaatatcatttgactttttatagaaaaaaaaaaaaaaaaaaaa

SEQ ID NO: 41 - DNA, 5514 bp, Homo sapiens
  misc_feature: Incyte ID No 1953711CB1
cggccgccggccgcagcgggctgagattgttgtcctctgtcaccagggcggctgggctcccgcagtcctgcagaccgcgcccgatcccggcgacagggcgggcggacagccgcgcatccccggggtcccgccgagcctgggcgcagagagccgggaggaagcgttcgctcgcttcgccttgctgctgggaaactgaacgaagggcccngagagagaaggttcttgagttcatgtaagaggacagtcttaaaacggaagaagaaagagaagcagttcagtctttgggagagctgcctccttgttgagtgctgcaaaggcctggaattcatttatgacagaatagatctagaaaagtccaagcatgttttctagagtggtgtagccctgtgctgcctccagtgaagagtctcttggtgttggcttcgtgcttccggagggaccatggcaacctccagaggggcctcccggtgtcctcgggatatcgccaatgtgatgcagaggctgcaagatgagcaagagatagtacaaaaacgaactttcacaaaatggatcaactctcatctggccaagcggaaacctccaatggtggtggacgatctttttgaagacatgaaagatggtgttaaactgcttgcccttctggaggtcctgtctgggcagaaactgccttgtgaacaaggacgccggatgaagcgaatccatgctgtggctaacattggcacggcactcaagttcctcgaaggaagaaagattaaattagtcaacattaactccaccgatatagctgatggccgaccctcaatagttcttggattgatgtggaccattattctatatttccagattgaagagttgaccagcaacctgccccagctccagtctttgtccagcagcgcatcctccgtggacagcatagttagctctgagactcccagcccaccaagtaaacggaaggtgaccaccaagatccaaggaaatgctaagaaggctttattaaagtgggttcagtacacagctggcaagcagactggaatagaagtaaaagattttgggaagagttggagaagcggggttgcctttcattcagttattcatgccattcgaccggaattggtggacttggagacagtgaaaggcagatccaaccgagaaaatttggaggatgctttcactatcgccgaaacagaactggggatcccaagactgctagatcctgaagacgttgatgtggataaaccagatgagaaatctattatgacctatgtagcccagtttctgaaacattatcctgacatccacaatgcaagcactgatgggcaagaggatgatgaaatacttccaggtttcccatcttttgcaaattctgtacaaaattttaagagagaagacagagtaatttttaaggaaatgaaagtttggatagaacaatttgagagagatttgacaagagcacagatggtggaatcaaatttacaggataaatatcagtcatttaagcacttcagagttcaatatgaaatgaagaggaaacagattgaacatttaatacaaccattacacagagacggtaaattgtcacttgaccaagcattggtaaaacaatcttgggatagagtgacctccaggctctttgactggcatatacagcttgataaatctcttcctgcacctctgggcaccataggtgcctggctgtacagagcggaggtggccctgagagaggaaataaccgttcaacaggtccacgaggaaacagcaaacacgatacaacggaaacttgagcaacataaggatctgcttcaaaacacggatgcccacaaaagagcattccatgaaatctaccggaccaggtctgttaacgggattccagtgccacctgatcaattagaggacatggccgagaggtttcattttgtttcctccacatcagagctacacctaatgaaaatggaatttttagaattaaagtaccgtctgctctcactgctggttcttgcagagtcaaagctgaagtcttggatcattaagtacgggaggagagagtcagtggagcagcttctacaaaactacgtgtcttttatagaaaatagcaagttctttgaacaatatgaggtgacataccagatcttgaaacagacagctgagatgtatgtcaaagcagatggttcagtggaagaagctgagaatgtgataaaattcatgaatgaaaccaccgctcagtggaggaatctctcagtagaagtgaggagtgtgaggagcatgctggaagaagtgatctctaactgggatcgctatggcaatacagtggctagtctgcaagcctggctagaggatgctgaaaaaatgctcaatcaatcagaaaacgccaaaaaggatttttttcgaaatttacctcattggattcagcagcatactgccatgaacgatgctggcaattttctaattgaaacctgtgatgagatggtttcccgtgacctgaagcagcaattactgttgctaaatgggcggtggagggagttgtttatgggagtcaagcaatatgctcaagctgatgagatggacagaatgaagaaggaatacacagactgtgttgttaccctgtctgcttttgcaacggaagcccataagaaactttctgaacccttagaagtctcttttatgaatgtcaagctattaattcaagacttggaggatattgagcagagggtgcctgtgatggatgcccaatacaagataattacaaagacagcacacctcattaccaaagaaagcccccaagaagaaggaaaagaaatgtttgcgaccatgtcaaagctcaaagagcagctaaccaaggtcagagaatgttactccccactcctttatgagtctcagcagctgttgattccgttggaggaattagaaaagcagatgacgtccttttatgactcacttgggaaaatcaatgaaattatcacagttcttgagcgtgaggcacaatcgagtgccctttttaaacaaaaacatcaggaactgttagcttgtcaagaaaactgtaagaaaaccttgacacttattgagaaaggcagtcaaagtgttcaaaagtttgtgaccttgagcaacgtgttaaagcattttgatcagacgaggctacaaagacagattgcagatattcatgttgcttttcagagtatggtaaagaaaactggagattggaagaagcatgtggaaaccaacagtcgcttgatgaagaagtttgaggagtctcgagcagagttggagaaggtactgcggattgctcaggagggcctggaggaaaagggggatccagaggagctcctgcggagacacactgagtttttcagtcagctggatcagagggtgctcaatgctttcctgaaagcttgtgatgaactcaccgacatccttccagagcaggagcagcaggggctgcaggaagctgttcgaaagctccacaaacaatggaaggatcttcaaggagaagccccttatcatttgcttcatctgaagattgatgtggagaagaataggttcttagcctctgtagaagaatgcagaactgagctggatcgagagaccaagctgatgccccaggaaggcagtgaaaagataattaaagagcacagggttttcttcagtgacaaaggtcctcatcatctctgtgagaaaaggttacagctcatcgaggaactctgtgtgaaactcccagtgcgggacccagtaagggacacacctggaacctgtcacgtgactctcaaagagctcagagctgccattgacagcacctacaggaagctcatggaagacccagacaagtggaaggactacactagcagattctctgagttctcatcttggatatctacaaatgagacacaattaaaggggatcaagggtgaggccatcgatactgccaaccacggagaggttaaacgtgccgttgaagagatcagaaatggtgttaccaaaaggggtgagaccctcagctggctgaaatccaggctgaaagttttgacagaagtttcttctgagaatgaagcccaaaagcagggagatgagctggcaaaattatccagctctttcaaggctcttgtgacgctgctgtcagaggttgaaaagatgctaagcaattttggggactgtgtccagtacaaagaaatagtcaaaaattctctcgaagaattaatttctggctctaaagaagtccaggaacaagctgagaagatcttggatactgaaaatctgtttgaagcacagcagttacttcttcatcaccagcaaaagacaaagcggatctcagcaaagaagagagatgtgcagcagcagatcgcgcaggcgcagcagggagaaggggggctgcctgaccgaggccacgaggagctgcggaagctggagagcacactggatggcctggagcgcagccgggagaggcaggaacgccgcatccaggtcacattaagaaaatgggagcgatttgaaacaaacaaagaaacagtagtaagatacctttttcaaacaggttccagtcatgaacgcttcttgagttttagcagtttggaaagtttatcttcagaactggaacaaacaaaggagttttctaaacggacagaaagtattgcagtccaggctgagaaccttgtaaaggaagcttcagagataccgcttgggccccaaaataagcagctgcttcaacagcaggccaagtcaatcaaagaacaagtcaaaaaattagaagacacgcttgaagaagagtatgtgattgacaagtcctaaactttcttctctgagataaagtttcatacaatctttcctgtaccttgtattcaaaacactcttttaaaatctcaaagtgtctgtgtatttcagcatgttttgaggaaacaactcacagttcaaaagaaagtatcgctaatacagaaaccaatatctataacagagcccaaaaaatataaaggatgtgggttttgcatcttaaactgatcatgttcatgagaaagccatatctattctattctgtggcctttgtacattgtagagggaatcttgaaaaagaactaatatttaaaataatttttttactatattattctgctgtcaccatttagagcgaaaaggagatattttgttagtgtagattccaggcctaaatacacatcacatagaccatatatctccaacctgaagaagctcctggagcttgtttacagtgcctcggtattcaagttatcctgactaatatgctctttccagaaattaactttaaaatattttatttttaaacttttaatgtttgtttatctgaaaaaaaaaaaactgcttggcctattgatatcctgttatatacccaaaatatacaaaaaagttgggaggggaaaaacatatgaggatacagccaatattattcaaaacaatatggctaaacatttctatttttaacataatttagtgggcaaatcaatgatacataatagcatattattattaagggacaggccatcatttctaaggtggcgt

SEQ ID NO: 42 - DNA, 3715 bp, Homo sapiens
  misc_feature: Incyte ID No 1595275CB1
ggtcgctggcagctgagcggcggaacccttcgctggcgggagcgcggttcggggccgcagaggggacattgaataaacctgagtattgtgtgcctgtattttgactgcgacctatcagatgagatcaggtcaagagacaactgcctttgaaagagagtgagtgactgtttccaaaacagcaagcatagggagagccgccggaagtattgacggaaagcttgaattacctacatgattgatgatggtctccagctgtcaaagaagattgtggttgccaagagaaaggtaaccatttctcatccccttgagtggaattggatgcctctaggagcccacattggctggcagtagacatggccgaatttacaagctacaaggagacggcctccagccgccacttgcggtttaagctacagagtctaagccgccgccttgatgagttggaggaagccacaaaaaacctccagaaagcagaggatgaactcctggatctccaggacaaggtgattcaggcagaaggtagcaactccagcatgttggcggagattgaagtgctgcgccagcgggtgctgagaattgaaggcaaagacgaggaaattaagagagcagaggatctgtgtcgtctgatgaaggagaaacttgaagaggaagaaaacctcacccgggagctgaaatctgagattgagcggcttcagaaacgaatggctgaattagagaagctagaagaggccttcagcaggagtaagaatgactgtacccagctgtgtctgagcctgaatgaggagagaaatctgaccaagaaaatctcctctgagctggaaatgctcagagtcaaagtgaaagaactagaatcttctgaggaccgcctggataaaactgagcagagtttagcgtcagagttagaaaagctgaaatcattaactctgagctttgtaagtgagagaaaatacttgaatgaaaaggagaaagaaaatgagaaattgataaaagaactcactcaaaaactagagcagaacaaaaaaatgaaccgagattatacaaggaatgcttctaatctggaaagaaatgacctacggattgaggatggcatctcttccacactgccgtccaaagaatcaagaaggaagggtggtctggactacctaaagcaggtagagaatgaaacaagaaacaaatcagaaaatgaaaagaaccgcaatcaggaagacaacaaagtcaaagaccttaaccaagagattgagaaacttaagacgcaaatcaaacactttgaatcgttggaagaagagcttaagaaaatgaagtccaaaaataacgaccttcaggataattacctaagtgaacaaaataaaaacaaattattagccagccaactggaggagataaagctacaaatcaagaaacagaaagagttagaaaatggagaggtagaaggggaagatgctttcctgtccagcaaaggcagacatgagaggactaagtttagaggccacggaagtgaagcttctgtgtccaagcacacagcgcgggaactgtctcctcagcataagcgggaacgactccggaacagggagtttgctctcaacaatgaaaactattctctgagcaacaggcaggtttcttctcccagtttcaccaacaggagggcagcaaaagcttctcacatgggggtgagtacagacagtgggactcaggagacaaagaaaactgaagaccggtttgtacccagctcctccaagagcgaagggaagaagtctagggagcagccctcagtgctgagtcgctaccccccggctgctcaggagcacagtaaagcgtggaaggggacttccaagccaggcaccgagagtggactgaagggaaaagtggagaagacaacacgaacgtttagtgacaccacccatggatctgttcccagtgacccattgggtagagctgacaaggcttctgacacctcctctgagactgtctttggcaagaggggacacgtgcttggcaacggaagtcaagtaactcaggctgcaaactctggctgttctaaggccattggagccctggcctcatctcgaagatcctcctcagaagggctctctaaaggcaaaaaggctgccaatggccttgaggctgataacagttgcccgaattccaaggctcctgttctatcgaagtatccttatagctgtagaagccaagagaacatccttcagggattttcaacctcacataaagaaggggttaatcaacctgcagcagttgtgatggaagacagcagtccgcatgaagccttgaggtgtcgagtcatcaaatccagtggcagagagaagccagactcagatgatgacttggacatagcatctcttgttactgccaagttggtaaatacaaccatcactccagagccagagcccaaaccacagcctaactctagagaaaaggctaaaacccgaggggcacctagaacctccctatttgagaatgataaagatgctggaatggagaatgaatctgtgaaatctgtcagagcctccaccaataccatggagctcccagataccaatggtgctggggtaaaaagccaaaggccctttagccccagagaggcgttgcggtctagagccatcatcaaacctgttattgttgataaggatgtgaaaaaaatcatgggaggatctggaacagagactacgttggagaaacagaaacccgtctccaaaccagggccaaacaaagtgacaagtagcattactatatatccatctgacagcagcagccccagagctgctccgggtgaggccctgagggagaggcacacatccactagcaatatccaggtggggctggcagagctcacatcagttagcaaccatgtcagctccccttttgagctctccattcacaaacatgacatcaccctgcagcttgcagaagcggagagaatggcagatgggcccctgaagaacaggccagaaacagtggtctctcggagcagcattataatcaagccatcggatcctgtggagaggaatagccatgcacctccagcggagacaatcaggtggaaaagccatagtgccccttcagaagtgggcttctcagatgccagacatgttactgtgcggaatgcctggaagagtaggcgagacttgaaatctttagaagaccccccaactcgaataggtaaaaacgtggaatctaccaatagcaatgcctacacccagaggtcttccacagacttctcagaactcgaacagcccaggtcctgcctttttgagcagggcactcgaagggtaggaccaagttcaggggatgcccctgagccctcctccagaaggacccaaagtagcctcactgtgtcagaggtgcttacccgtcggaatcgggtaggagacaccatcactgtcgcagcctggaaccactcagcaagcatggaggaagaaggggaagactgtacactcagtgtctacaggcaactgcacaatccctggatccgtctgaactgcctgggaagcaggggctgccagagtctgggcgagtacgggcgagggaacgattacgggccaaacaggccctgtgctgaggagacactgagcccgcatgggtgtctgttgtctgccctctgactcctgcttctcaggatcttccaccgtactgccccacgaaggatcaaggccagttaaccaggcaagacacaagcctttgctgggaaaactgtggtaga

SEQ ID NO: 43 - DNA, 1082 bp, Homo sapiens
  misc_feature: Incyte ID No 72332548CB1
ccgcggacgaagatggcgaccgccatgtacttggagcactatctggacagtatcgagaaccttccctgcgaacttcagaggaacttccagctgatgcgagagctggaccagaggacggaagataagaaagcagagattgacatcctggctgcagagtacatctccacggtgaagacgctgtctccagaccagcgcgtggagcgcctgcagaagatccagaacgcctacagcaagtgcaaggaatacagtgacgacaaagtgcagctggccatgcagacctacgagatggtggataaacacattcgaaggcttgatgcagacctggcgcgctttgaagcagatctgaaggacaagatggagggcagtgattttgaaagctccggagggcgagggttaaaaaaaggccggggtcagaaagaaaaaagagggtcccggggccgaggcaggaggacatcagaggaagacacaccaaagaaaaagaagcacaaaggagggtctgagttcactgacaccatcctgcccgtgcacccctctgatgtgctggacatgcccgtggacccaaacgaacccacgtactgcctgtgccaccaggtctcctatggggagatgattggctgtgacaatccagactgtccaattgagtggtttcactttgcctgcgtggaccttaccacgaaacccaaaggaaaatggttctgtccacggtgtgtccaggaaaagaggaagaagaagtaggaggagcatgtgtgcccaggatccgaggagcaagttaatctgtcccttcattcgtgtcgcaatatttcccttccttttaaaactaccttgttcggttgatacttagtaactccgtggccagttgaagcgctggatgcttcctagaacaagaaccaccaaagcctgttcgcacagaagggcgaccttgcagggactcgccgccgcgacctcagtgtggcttttacaggacatccaccccgagcatcagcagggaccccggcggacgtgggcagggcgcgcgtgagctcgggctgcccggccgggcgtgcgggcggggacatggtaacctggtccacggagggcggcca

SEQ ID NO: 44 - DNA, 2621 bp, Homo sapiens
  misc_feature: Incyte ID No 7322834CB1
gcgccgagcgccgcgccgccccgggcccctccgccgccgccggcccggacatggccgccaacatgtacagggtcggagactacgtctactttgagaactcctccagcaacccatacctgatccggagaatcgaggagctcaacaagacggccaatgggaacgtggaggccaaagtggtgtgcttctaccggaggcgggacatctccagcaccctcatcgccctggccgacaagcacgcaagggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggttggagacccaggtgtgggaggcgcacaacccactcacagacaagcagatcgaccagttcctggtggtggcccgctctgtgggcaccttcgcacgggccctggactgcagcagctccgtccgacagcccagcctgcacatgagcgccgcagctgcctcccgagacatcaccctgttccacgccatggatactctccacaagaacatctacgacatctccaaggccatctcggcgctggtgccgcagggcgggcccgtgctctgcagggacgagatggaggagtggtctgcatcagaggccaaccttttcgaggaagccctggaaaaatatgggaaggatttcacggacattcagcaagattttctcccgtggaagtcgctgaccagcatcattgagtactactacatgtggaagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacaccacacagtcttaccagtggtattcttggggtccccctaacatgcagtgtcgtctctgcgcatcttgttggacatattggaagaaatatggtggcttgaaaatgccaacccggttagatggagagaggccaggaccaaaccgcagtaacatgagtccccacggcctcccagcccggagcagcgggagccccaagtttgccatgaagaccaggcaggctttctatctgcacacgacgaagctgacgcggatcgcccggcgcctgtgccgtgagatcctgcgcccgtggcacgctgcgcggcacccctacctgcccatcaacagcgcggccatcaaggccgagtgcacggcgcggctgcccgaagcctcccagagcccgctggtgctgaagcaggcggtacgcaagccgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggtctggcaaaccacggacagaccaggcacatgggaccaagccggaacctcctgctcaacgggaagtcctaccccaccaaagtgcgcctgatccgggggggctccctgcccccagtcaagcggcggcggatgaactggatcgacgccccggatgacgtgttctacatggccacagaggagaccaggaagatccgcaagctgctctcatcctcggaaaccaagcgtgctgcccgccggccctacaagcccatcgccctgcgccagagccaggccctgccgccgcggccaccgccacctgcgcccgtcaacgacgagcccatcgtcatcgaggactaggggccgcccccacctgcggccgccccccgcccctcgcccgcccacacggccccttcccagccagcccgccgcccgcccctcagtttggtagtgccccacctcccgccctcacctgcagagaaacgcgctccttggcggacactgggggaggagaggaagaagcgcggctaacttattccgagaatgccgaggagttgtcgtttttagctttgtgtttactttttggctggagcggagatgaggggccaccccgtgcccctgtgctgcggggccttttgcccggaggccgggccctaaggttttgttgtgttctgttgaaggtgccattttaaattttatttttattactttttttgtagatgaacttgagctctgtaacttacacctggaatgttaggatcgtgcggccgcggccggccgagctgcctggcggggttggcccttgtcttttcaagtaattttcatattaaacaaaacaagaaaaaaaatcttataaaaaggaaaaaaacc

SEQ ID NO: 45 - DNA, 5539 bp, Homo sapiens
  misc_feature: Incyte ID No 1925714CB1
aaaccaaaacctgaatccagcagaagaaagaaaagaaccaagattagagcagaactaaatgaaattgaaacaaaaaaaatagaaaaaataaataaaacaaaaagctgttttttgaaataatacataaaattgatagaccattagaaaaattaaccgagaaaagaagagagaggattaaaataagctcaattagaaacaaaatgggagatattacaaccaacaccacagaaatacaaaagatcactaaaggctactatgaacacctttatgtgcataaactagaatacctagaggagatgaataaattcctgaaaaaatacaatccttctagcttaaatcagaaagaattagaaatcctgaacagaccaataacaagcagccagattgaactggtaattaagaaattaccaacaggctggactccacctctgggggcagggcatagccaaacaaaaggcagcagaaacctctgcagacttaaatgtccctgtctgacagcggtgaagagaatagttgttctcccaggacgcagctggagatctgagaacggacagactgcctcctcaaatgggtccctgaccccaattccagaggaacgatcaggcagcaacatttgctgttcaccaatatcagctgttctgcagcctctgctgctgatacccaggcaaacagggtctggagtggacctccggcaaactccaacacgtctgcagctgagggttctgactgttagaaggaaaactaacaaacagaaaggacatccacaaccataccccatctgtacatcaccatcatcaaagaacaaagagaagtccttaaaggacctgatggagctgaaaaccacggcacgagaactacatgatgaatgcacaagcctcagtagacgatttgatcaactggaagaaagggtatcagtgatggaagatcaaatgagtgaaatgaaaataaaatacctaggaatccaacttacaagggatgtgaaggaactcttcaaggagaactacaaaccactgctcaatgaaataaaagaagatacaaacaaatggaagaatattccatgctcatgtgtagaaagagtcaatatcgtgaaaatggccatactgcccaaggtaatttatagattcaatgccatcaccatcaagctacaaattactttcttcacagaattggaaaaaactactttaaagctcatatggaaccaaaaaagagctcacattgccaagtcaatcctaagccaaaagaacaaagctggaggcatcacgctacctgacttcaagctatactacaagactacagtaaccaaaacagcatggtactggtaccaaaacagaggtacagaccaatggaacagaacagagccctcagaaataatgctgcatatctacaactatctgatctttgacaaacctgacaaaaacaagaaatgtggaaaggattccctatttaataaatggtgctgggaaaactggctagccatatggagaaagctgaaactggatcccttccttacaccttacacaaaaattaattctagatggattaaagacttaaatattagacctacaaccataaaaaccttagaagaaaacctaggcaataccattcaggacataggcatgggtaagaacttcatgtctaaaacaccaaaagcagtagcaacaaaagccaaaatttacaaatgggatctaaataaactaaagagcttctgcactgcaaaagaaattaccatcagagtgaacaggcaacctacaaaatgggagaaaatttttgcaatctactcatctgacaaagggctaacatccagaatctgcaatgaactcaaacacatttacaagaaaaaaacaaacaaccccatcaaaaagtgggtgaaggatatgaacagacacttctcaaaagaagacatttatgcagtcaaaagacatgaaaaaatgttcatcatcactggccatcagagaaatgcaaatcaaaaccacaatgagaagacatggacacaggaaggggaacatgacacaccggggcctgttgtggggtggggggagggtggagggatagcatttggagatcgcgggcctgtcggtgtctgctcctacacgccaacgccggtgggcaggaccatgtctctggtaagccagaattcgcgccgccgccgccgccgcgttgcaaaggctactgcgcacaacagcagctggggcgaaatgcaggcccctaatgcccccggtctccccgctgatgtgccaggctcagacgtcccccagggtcccagcgattcccagatcctccagggcctctgcgcctctgagggcccaagcacctccgttctgcccacctccgctgagggcccaagcacctttgtgccgcccaccatctctgaggcctcaagcgcctccgggcagcccaccatctctgagggacctggcacctccgtgctgcccacccccagtgagggcctaagcacctccgggcctcccaccatctctaaggggctgtgcacctctgtgacgcttgccgcctctgagggccggaacacctccaggccgcccacttcctctgaggaacctagcacctccgtgccgcccaccgcctctgaggtaccgagcacctccctgccgcccacccctggtgagggaacgagcacctccgtgccgcccacagcctatgagggaccaagcacctccgtggtgcccacccctgatgagggaccaagcacctccgtgctgcctacacctggtgagggaccaggcacctccgtgccgctcgccgccactgagggcctgagcacctccgtgcaggccactcctgatgagggaccgagcacctccgtgccgcccaccgccactgagggcctaagcacccccgtgccacccacccgtgatgagggaccgagcacctccgtgccggccactcctggtgagggaccgagcacctccgtgctgcccgccgcctctgacggacaaagcatctccttggtgcccacccgcggtaagggatcaagcacctccgtgccccccaccgccaccgagggcctgagcacctccgtgcagcccactgctggtgagggatcgagcacctccgtgccgcccacccctggtgggggactgagcacctccgtgccgcccaccgccactgaggagttgagcacctccgtgccgcccactcccggtgagggaccaagcacttccgtactgccaatccccggtgagggactgagcacctctgtgccgcccaccgcctctgatggatcggacacctccgtgccgcccactcctggtgagggcgcaagcaccttagtgcagcccaccgcccctgacggaccgggaagctccgtgctgcctaaccctggtgagggcccgagcacattgtttagctctagtgcttctgtggaccggaacccctccaagtgttcccttgttttgccaagccctagggtaaccaaggcctccgtggactcagattctgagggtcctaagggtgcagaaggccctatagaattcgaggtcctgagagactgtgagagccccaactccattagtattatgggcctcaatacttcccgggttgcaattaccctgaagccccaagaccctatggaacagaacgtagctgagctgttgcagttcctgctggtgaaggatcagagcaagtaccctatccgggagtctgaaatgcgggaatatattgttaaagaatatcgcaaccagtttcctgagatactcaggcgagcagcagcccacctggagtgcatttttaggtttgaattgagagaacttgaccctgaggcacacacctacattctgttaaacaaactgggacctgtgccctttgaagggttagaagagagcccaaatgggccaaagatgggcctcctgatgatgattctaggccaaatattcctgaatggcaaccaagccaaggaggctgagatttgggaaatgctctggaggatgggggtgcagcgggaaaggaggctttccatttttgggaacccaaagagacttctgtctgtggagtttgtatggcagcgttacttagactacaggccagtaactgactgtaaaccagtggagtatgagtttttctggggcccaagatcccacctagaaaccaccaagatgaaaattctgaagttcatggcgaaaatatataacaaagatcctatggattggccagagaaatacaacgaagctctggaagaagatgctgccagagcctttgctgagggttggcaggctctccctcactttaggaggcccttttttgaggaagctgctgcagaggtaccatcccctgattcagaggtttccagctattcctcaaaatatgccccacattcatggcctgagtcaagattggagagcaaggcaaggaagctggtgcagttatttctgcttatggattcaactaagctgcctataccaaagaaaggaattctgtactacattggccgagagtgcagcaaagtgttccctgacctcctgaatcgtgctgcccgcaccctgaaccatgtctatgggacagaactagtggtacttgatcccaggaatcactcctatactctgtacaaccgaagggagatggaagaaactgaggagatcgtagacagtccaaacaggcctggcaacaactttttgatgcaggtcctaagcttcatctttattatgggcaaccatgccagggagtctgcagtctgggcctttctgcggggcttaggggttcaagctgggagaaagcatgtgattacctgcagatacttgagtcagcgctatatagacagtttacgggttcctgacagtgatccagtgcaatatgagtttgtatggggtcctagagcccgtttggaaacctctaagatgaaagccttgcgatatgtggccagaatccacagaaaggaaccacaggactggccacagcagtacagggaggcaatggaagatgaggccaatagagctgatgttgggcacaggcaaatctttgttcacaacttcaggtagaggaatgcatggcagtcagaggggccttgcaaggaggggcctttgagcctcagttctcatgtattggggggtgggggtgggtacatattgtatttggtatttgtgttccagttatatttatgtcttttcatatttagttctggtgtggtgtctggaaatgtttcaactgttttaatatattgtctgattgggtaaatgtgattgaccacttgctgtctctgttgtattttggtatgagttttgatagctctataaaatgttttggaaatctttccatcttgttgcattatctagaaaagaatatagcatatagctatagatataggcttttccttgaaagcttgaaaaaaattcgccagtaaaataatctagcttaaagtgataaactaacaaacaaatataacaacaactgaaaaggcactctaaattgtggctggcttccctttctgtctgctattgtatgaagaatactgatgtttacctgtatttgctttgctgtactaaaatgtaatgaaaaataaaagattaataaatgaaacataatgctaata

SEQ ID NO: 46 - DNA, 2338 bp, Homo sapiens
  misc_feature: Incyte ID No 6803363CB1
cggaggggtgctaacggtgctaacgctgcccgctccccgcccgcccgcaggtgcgcaggaggatggtggcgcggccctaggcccacgctccgcaccatgacctgctggctgtgcgtcctgagcctgcccctgctcctgctgcccgcggcgccgcccccggctggaggctgcccggcccgctgcgagtgcaccgtgcagacccgcgcggtggcctgcacgcgccgccgcctgaccgccgtgcccgacggcatcccggccgagacccgcctgctggagctcagccgcaaccgcatccgctgcctgaacccgggcgacctggccgcgctgcccgcgctggaggagctggacctgagcgagaacgccatcgcgcacgtggagcccggcgccttcgccaacctgccgcgcctgcgcgtcctgcgtctccgtggcaaccagctgaagctcatcccgcccggggtcttcacgcgcctggacaacctcacgctgctggacctgagcgagaacaagctggtaatcctgctggactacactttccaggacctgcacagcctgcgccggctggaagtgggcgacaacgacctggtattcgtctcgcgccgcgccttcgcggggctgctggccctggaggagctgaccctggagcgctgcaacctcacggctctgtccggggagtcgctgggccatctgcgcagcctgggcgccctgcggctgcgccacctggccatcgcctccctggaggaccagaacttccgcaggctgcccgggctgctgcacctggagattgacaactggccgctgctggaggaggtggcggcgggcagcctgcggggcctgaacctgacctcgctgtcggtcacccacaccaacatcaccgccgtgccggccgccgcgctgcggcaccaggcgcacctcacctgcctcaatctgtcgcacaaccccatcagcacggtgccgcgggggtcgttccgggacctggtccgcctgcgcgagctgcacctggccggggccctgctggctgtggtggagccgcaggccttcctgggcctgcgccagatccgcctgctcaacctctccaacaacctgctctccacgttggaggagagcaccttccactcggtgaacacgctagagacgctgcgcgtggacgggaacccgctggcctgcgactgtcgcctgctgtggatcgtgcagcgtcgcaagaccctcaacttcgacgggcggctgccggcctgcgccaccccggccgaggtgcgcggcgacgcgctgcgaaacctgccggactccgtgctgttcgagtacttcgtgtgccgcaaacccaagatccgggagcggcggctgcagcgcgtcacggccaccgcgggcgaagacgtccgcttcctctgccgcgccgagggcgagccggcgcccaccgtggcctgggtgaccccccagcaccggccggtgacggccaccagcgcgggccgggcgcgcgtgctccccggggggacgctggagatccaggacgcgcggccgcaggacagcggcacctacacgtgcgtggccagcaacgcgggcggcaacgacacctacttcgccacgctgaccgtgcgccccgagccggccgccaaccggaccccgggcgaggcccacaacgagacgctggcggccctgcgcgcgccgctcgacctcaccaccatcctggtgtccaccgccatgggctgcatcaccttcctgggcgtggtcctcttctgcttcgtgctgctgttcgtgtggagccgcggccgcgggcagcacaaaaacaacttctcgggggagtactccttccgcaaggtggatgggccggccgccgcggcgggccagggaggcgcgcgcaagttcaacatgaagatgatctgaggggtccccagggcggaccctcccctcccctcccccgcgggccggccgctcgcgtgtccacctatgcatttcccggaggggaaggggacggctgcacggcgctccccaggcagaacttcccctttttttgtagacgcccaaccgcaggactgtttttcatcagcatgcgtcttttttgcagtttctcaagcgttttctaaagatttcaacccgtcttcctgtccctgctatggtggctgagctggggggtgggggtgcgggctgcaaggatggggggatgggctcccgtctaccaacccggggtggctgggggcgctcagagaccccaggatcctcaggggccgcaattcacacccaggaggcacaagactgctgcccccaagccctgggcttccgctcccccagccctttgtacacagatcgcatcggggagacggtggacacccgggtcccacaaagggcc

SEQ ID NO: 47 - DNA, 3214 bp, Homo sapiens
  misc_feature: Incyte ID No 7070580CB1
ggagatgccccttcaaggcttttgcccttcctgagggagcagaaaaagccacgaagactcctcgtaaacaacttgagagagagaattgtgtctcccctatgaagatggattgcttgagacagagttcaatagaagatgttaaacagataagctgagggcagggggactgagctttggaagagggcctttcaggcagcgaggccgctggcctgtggctccctctgatgagcttcttctaaagatggtaagggctgggggaagagcttggcagcaggggctgcactctctctcagctgtgtctatcttggccaagggttttatgttcctattggtaggggattcgagagcagagagcgtcaccaatacactcgtgttgttcaccataggagaagagtccctgaccatttttgtggacaagcagaaactgggaagaaagacagagacaacaggaggtgcctctataatcgggggcagtgggaacagcacagctgtgtccctggagaccctgcaccagctggccgcctcctacttcatcgacagagagagcacgctgcgacggctgcaccatatccagatagccacgggggccatcaaggtcaccgagaccaggaccggtcctctgggctgcagcaactatgacaatctggactcagtcagttctgtcttggtacagagtccagagaacaaagtacagttacttggccttcaggtgctgctgcctgagtatctgcgtgagcgctttgtagctgcagcactcagctacatcacatgcagctctgagggtgagctcgtctgcaaggagaatgactgctggtgcaagtgcagccccaccttccctgaatgcaactgccctgatgctgacatccaggccatggaggacagcctgctgcagatccaggactcctgggccactcacaaccggcagtttgaagagtcagaagagttccaggccctgctgaaaaggctgcccgatgaccggttcctgaactccacagctatctcccagttctgggccatggacaccagccttcagcaccgctaccagcagctgggagctggcttgaaagtgctgttcaaaaagacccatcggatcctacgccggctcttcaacctctgcaagcgctgccatcgccagcctcgcttccgcctgcccaaggagaggtccttgtcctactggtggaaccgaatccagtccctcctctactgtggggaaagcacctttcctggcactttcctggaacagagccacagctgcacctgcccctatgaccaatcttcctgccagggccccatcccatgtgccttgggcgaagggcccgcgtgtgcccactgtgctccagacaatagcacacgctgtgggagctgcaacccgggctatgtgctggcccaggggctgtgccggccagaggtggccgagtccctggaaaactttcttgggctggagacagacttgcaggacctggagctaaagtacctgctgcagaagcaggatagccgcattgaggtacactccatcttcatcagcaatgacatgcggctgggcagctggtttgacccttcctggaggaagcgcatgctgctcaccctgaagagcaacaagtacaagcctgggctggtgcacgtgatgttggccttgtccttgcagatctgtctcaccaagaacagcaccctggagcctgtcatggccatctacgtcaacccctttgggggcagccactctgagagctggttcatgcctgtgaatgagggcagctttcctgactgggaaaggactaacgtggatgcagctgcccagtgccaaaactggactatcaccttggggaataggtggaagactttctttgagacagttcatgtttacctacggagccgaatcaagtccctggatgacagctccaatgagacaatctactatgagcccctggagatgactgatccctctaagaatttgggttacatgaaaattaacaccttgcaggtctttggctacagcctgccctttgacccagatgctatccgggacttaattctccagttcgactacccatatactcaaggttcccaggactctgcactcttgcagctcattgagctcagggaccgggtgaaccagctttctccacctggcaaagtccgacttgaccttttctcctgcttgctccggcatcggcttaagctggccaacaatgaggtgggcaggatccagtcctccctgagggctttcaattctaagctgccaaaccctgtggaatatgagaccggcaaactctgtagctaatgggcggcccacttcagcactgggcaaggaggggatccatgaatctggggtacaaagataatctaagccctcaccttagtgccaacagggtgtgctcccacgagactttcagcatccagtagatgggacctcgaggctcgagctgaagcaggcgagagagaaacagctactgcgtgcgtgcgcgcacgcatacacacacacacacacacactggcacagggaggctacaactaagcagcctcagatctgtaaagttgattggtgctttctaaaatgaatgcaattgaaagaaaggagccaaggaagagattaagaaaaagaaccagctgaaccatgaaacaaacaaaaatcctaaaagtgattcttgtcattcaagaaagcaagaggggacaacaatgggaggggtcggagctcttccttcccctctgtggagtcacttttgtattctttttaaccagatttcttaaaatgttgttgttttgtgaatcctgacattggttcttacttttgtatgctgcctcctctgtgccctcccagacgctgactgggaaacacaagaagtacaaccaacaggaaccagcgccaagggcaggcagcggcctccttgctcccctcccttactcctccctctgctgcctcctccccccaccaagtttcagggccctggattgttcccagttcccattgtggtcccttcagagctcctttccaacagcatctctctgtcgaagaaagaagctctgtcaagttagagagagacaatgtgtaggaaatgttcttttttaaaaaaaaataacaaaaacaaaacaaaactatttattttgtgattgttttccttgttaatctgctccaaccacctgaacatctaagtaaacatttatctggtttaatataaaaaaaaaaaaaaaaaaaattctgggcccaagaattagctgt

SEQ ID NO: 48 - DNA, 2824 bp, Homo sapiens
  misc_feature: Incyte ID No 7500176CB1
cagcgattgatatttctaatcgtcgtgatccacctccccaggaccttggagccacgtttacaaataggaatagggtacgtgggagggatagaacgtacagccaataaaatcatgtggcgccgatgggcgtgttgaggccgctgcctggcttagggcggaaacagattctctgcataagaaggggaacgaaagatggcggcggaaacgctgctgtccagtttgttaggactgctgcttctgggactcctgttacccgcaagtctgaccggcggtgtcgggagcctgaacctggaggagctgagtgagatgcgttatgggatcgagatcctgccgttgcctgtcatgggagggcagagccaatcttcggacgtggtgattgtctcctctaagtacaaacagcgctatgagtgtcgcctgccagctggagctattcacttccagcgtgaaagggaggaggaaacacctgcttaccaagggcctgggatccctgagttgttgagcccaatgagagatgctccctgcttgctgaagacaaaggactggtggacatatgaattctgttatggacgccacatccagcaataccacatggaagattcagagatcaaaggtgaagtcctctatctcggctactaccaatcagccttcgactgggatgatgaaacagccaaggcctccaagcagcatcgtcttaaacgctaccacagccagacctatggcaatgggtccaagtgcgaccttaatgggaggccccgggaggccgaggttcggttcctctgtgacgagggtgcaggtatctctggggactacatcgatcgcgtggacgagcccttgtcctgctcttatgtgctgaccattcgcactcctcggctctgcccccaccctctcctccggcccccacccagtgctgcaccgcaggccatcctctgtcacccttccctacagcctgaggagtacatggcctacgttcagaggcaagccgactcaaagcagtatggagataaaatcatagaggagctgcaagatctaggcccccaagtgtggagtgagaccaagtctggggtggcaccccaaaagatggcaggtgcgagcccgaccaaggatgacagtaaggactcagatttctggaagatgcttaatgagccagaggaccaggccccaggaggggaggaggtgccggctgaggagcaggacccaagccctgaggcagcagattcagcttctggtgctcccaatgattttcagaacaacgtgcaggtcaaagtcattcgaagccctgcggatttgattcgattcatagaggagctgaaaggtggaacaaaaaaggggaagccaaatataggccaagagcagcctgtggatgatgctgcagaagtccctcagagggaaccagagaaggaaaggggtgatccagaacggcagagagagatggaagaagaggaggatgaggatgaggatgaggatgaagatgaggatgaacggcagttactgggagaatttgagaaggaactggaagggatcctgcttccgtcagaccgagaccggctccgttcggagacagagaaagagctggacccagatgggctgaagaaggagtcagagcgggatcgggcaatgctggctctcacatccactctcaacaaactcatcaaaagactggaggaaaaacagagtccagagctggtgaagaagcacaagaaaaagagggttgtccccaaaaagcctcccccatcaccccaacctacagaggaggatcctgagcacagagtccgggtccgggtcaccaagctccgtctcggaggccctaatcaggatctgactgtcctcgagatgaaacgggaaaacccacagctgaaacaaatcgaggggctggtgaaggagctgctggagagggagggactcacagctgcagggaaaattgagatcaaaattgtccgcccatgggctgaagggactgaagagggtgcacgttggctgactgatgaggacacgagaaacctcaaggagatcttcttcaatatcttggtgccgggagctgaagaggcccagaaggaacgccagcggcagaaagagctggagagcaattaccgccgggtgtggggctctccaggtggggagggcacaggggacctggacgaatttgacttctgagaccaacactacacttgacccttcacggaatccagactcttcctggactggcttgcctcctccccacctccccaccctggaacccctgagggccaaacagcagagtggagctgagctgtggacctctcgggcaactctgtgggtgtgggggccctgggtgaatgctgctgcccctgctggcagccaccttgagacctcaccgggcctgtgatatttgctctcctgaactctcactcaatcctcttcctctcctctgtggcttttcctgttattgtcccctaatgataggatattccctgctgcctacctggagattcagtaggatcttttgagtggaggtgggtagagagagcaaggagggcaggacacttagcaggcactgagcaagcaggcccccacctgcccttagtgatgtttggagtcgttttaccctcttctattgaattgccttgggatttccttctccctttccctgcccaccctgtcccctacaatttgtgcttctgagttgaggagccttcacctctgttgctgaggaaatggtagaatgctgcctatcacctccagcacaatcccagtgaaaaaggtgtgaagcacccaccatgttcttgaacaatcaggtttctaaataaacaactggaccatcaaagaaaagaaaaaaaaaacgcggcgg

SEQ ID NO: 49 - DNA, 1570 bp, Homo sapiens
  misc_feature: Incyte ID No 7500506CB1
ctcagtggctggagggcagggccagggtgcggacacaagctgcagacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtgatccccgttccaccccaagagaactcgcgctcagtaaacgggaacatgccccctgcagacaaaaaaaaaaaaaa

SEQ ID NO: 50 - DNA, 725 bp, Homo sapiens
  misc_feature: Incyte ID No 7500639CB1
ctggagaggtggggagctgctgaagggcgggagaagttgcggcgtggcgataggtctgggtgttctgggaggacccctaaaagggagcagctctgccaggtcagagcttgaggtagcatgtgtgccgcttccaggaggccaggatggagacagcagttcgaggaatgcccttggaatgccctcctaggccggagcggctcaatgcctacgagcgcgaagtgatggtgaacatgctgaactcactgtcgcggaaccagcagctgccgcggatcacgccccgatgcgggtgcgtggacccgctgcccggccgcctgcccttccatggttacgaaagtgcttgctcgggccgccactactgtctgcgcgggatggactactacgccagcggggcgccctgcaccgaccgccgcctgcggccttggtgccgggagcaaccgactatgtgtacctccctacgagcaccggcccggaatgcagtgtgctgttacaactcccccgccgtcatactacccatatccgaaccttagatgggacacaagtcacttcaagaagtctggtggtccccagagaaacaactatgttatccatcctgagtttgtgtctgagacctatcccgactatcgttgctggtagagcctgggcttgctgtgccaagggggcagagcaataaataaactcttcaaccccaaaaaaaaaaaaaaaaaaaaaaaaaaaaaaaaaaaatg

SEQ ID NO: 51 - DNA, 3108 bp, Homo sapiens
  misc_feature: Incyte ID No 7506167CB1
gcaagagtgtctgtgtgcatatcagataaagcaaagaaaactaccaaaaccagagattatataatgatcaaaggtttaatccatgaagaagacagcaattttaaaagtgtattcaccaaaaaataaagcttcaaaatatgtgatgtgaaaactgccagaactaaggcgggccgggctcagaccagcgctgcctcaggatgtgaagtgtaacaagagggccaggggaggtggtgggggacaacatgggcctgtgaggcctgtgggtgcccgcgttccccagctccccccgcagcccgctccacagtggtccgctccggttggttgtcacgtgcgcattcgggttccagacccaaggctgcgtgttctccaccgcttgttgtggccagtgttactgcggtgaccgccagagcagcctcgacgctatggaggagcctggtgctacccctcagccctacctggggctggtcctggaggagctacgcagagttgtggcagcactacctgagagtatgagaccagatgagaatccttatggttttccatcggaactggtggtatgtgcagctgttattggattttttgttgttctcctttttttgtggagaagttttagatcggttaggagtcggctttacgtgggaagagagcaaaaacttggtgcaacgctttctggactaattgaagaaaaatgtaaactacttgaaaagtttagccttattcaaaaagagtatgaaggctatgaagtagagtcatctttagaggatgccagctttgagaaggcggcagcagaagaagcacgaagtttggaggcaacctgtgaaaagctgaacaggtccaattctgaacttgaggatgaaatcctctgtctagaaaaagacttaaaacaagagaaatctaaacattctcaacaagatgaattgatggcggatatttcaaaaagtatacagtctctagaagatgagtcaaaatccctcaaatcacaaatagctgaagccaaaatcatctgcaagacatttaaaatgagtgaagaacgacgggctatagcaataaaagatgctttgaatgaaaattctcaacttcagacaagccataaacagctttttcagcaagaagctgaagtatggaaaggagaagtgagtgaacttaataaacagaaaataacatttgaagactccaaagtacacgcagaacaagttctgaatgataaagaaaatcacatcaagaccctgactggacacttgccaatgatgaaagatcaggctgctgtgcttgaagaagacacaacggatgatgataacctggaattagaagtgaacagtcaatgggaaaatggtgcttacttagataatcctccaaaaggagctttgaagaaactgattcatgctgctaagttaaatgcttctttaaaaaccttagaaggagaaagaaaccaaatttatattcagttgtctgaagttgataaaacaaaggaagagcttacagagcatattaaaaatcttcagactcaacaagcatctttgcagtcagaaaacacacattttgaaaatgagaatcagaagcttcaacagaaacttaaagtaatgactgaattatatcaagaaaatgaaatgaaactccacaggaaattaacagtagaggaaaattatcggttagagaaagaagagaaactttctaaagtagatgaaaagatcagccatgccactgaagagctggagacctatagaaagcgagccaaagatcttgaagaagaattggagagaactattcattcttatcaagggcagattatttcccatgagaaaaaagcacatgataattggttggcagctcggaatgctgaaagaaacctcaatgatttaaggaaagaaaatgctcacaacagacaaaaattaactgaaacagagcttaaatttgaacttttagaaaaagatccttatgcactcgatgttccaaatacagcatttggcagaggctcacgaggcccagggaatcctctggaccatcagattaccaatgaaagaggagaatcaagctgtgataggttaaccgatcctcatagggctccctctgacactgggtctctgtcacctccatgggaccaggaccgtaggatgatgtttcctccgccaggacaatcatatcctgattcagcccttcctccacaaaggcaagacagattttgttctaattctggtagactgtctggaccagcagaactcagaagttttaatatgccttctttggataaaatggatgggtcaatgccttcagaaatggaatccagtagaaatgataccaaagatgatcttggtaatttaaatgtgcctgattcatctctccctgctgaaaatgaagccactggccctggctttgttcctccacctcttgctccaatcagaggtccattgtttccagtggatgcaagaggcccattcttgagaagaggacctcctttccccccacctcctccaggagccatgtttggagcttctcgagattattttccaccaagggatttcccaggtccaccacctgctccatttgcaatgagaaatgtctatccaccgaggggttttcctccttaccttcccccaagacctggatttttccccccacccccacattctgaaggtagaagtgagttcccctcaggtttgattccaccttcaaatgagcctgctactgaacatccagaaccacagcaagaaacctgacaatatttttgctctcttcaaaagtaattttgactgatctcattttcagtttaagtaactgctgttacttaagtgattacacttttgctcaaattgaagcttaatggaattataattctcaggatagtattttgtaaataaagatgatttaaatatgaatcttatgagtaaattatttcaattttattttagacggtataactatttcaatttgattaatccactattatataaacaatagtgggagttttatatatgtaatctttcaggtggggaggctttaaattctgaagtctgtgtctttatgccaagaactgtatttactgtggttgtggacaaatgtgaaagtaactttatgcttaaataaattatagttgatttaaagatttgtttg

SEQ ID NO: 52 - DNA, 1962 bp, Homo sapiens
  misc_feature: Incyte ID No 90081189CB1
gccgctgcctggcttagggcggaaacagattctctgcataagaaggggaacgaaagatggcggcggaaacgctgctgtccagtttgttaggactgctgcttctgggactcctgttacccgcaagtctgaccggcggtgtcgggagcctgaacctggaggagctgagtgagatgcgttatgggatcgagatcctgccgttgcctgtcatgggagggcagagccaatcttcggacgtggtgattgtctcctctaagtacaaacagcgctatgagtgtcgcctgccagctggagctattcacttccagcgtgaaagggaggaggaaacacctgcttaccaagggcctgggatccctgagttgttgagcccaatgagagatgctccctgcttgctgaagacaaaggactggtggacatatgaattctgttatggacgccacatccagcaataccacatggaagattcagagatcaaaggtgaagtcctctatctcggctactaccaatcagccttcgactgggatgatgaaacagccaaggcctccaagcagcatcgtcttaaacgctaccacagccagacctatggcaatgggtccaagtgcgaccttaatgggaggccccgggaggccgaggttcggttcctctgtgacgagggtgcaggtatctctggggactacatcgatcgcgtggacgagcccttgtcctgctcttatgtgctgaccattcgcactcctcggctctgcccccaccctctcctccggcccccacccagtgctgcaccgcaggccatcctctgtcacccttccctacagcctgaggagtacatggcctacgttcagaggcaagccgactcaaagcagtatggagataaaatcatagaggagctgcaagatctaggcccccaagtgtggagtgagaccaagtctggggtggcaccccaaaagatggcaggtgcgagcccgaccaaggatgacagtaaggacccagatttctggaagatgcttaatgagccagaggaccaggccccaggaggggaggaggtgccggctgaggagcaggacccaagccctgaggcagcagattcagcttctggtgctcccaatgattttcagaacaacgtgcaggtcaaagtcattcgaagccctgcggatttgattcgattcatagaggagctgaaaggtggaacaaaaaaggggaagccaaatataggccaagagcagcctgtggatgatgctgcagaagtccctcagagggaaccagagaaggaaaggggtgatccagaacggcagagagagatggaagaagaggaggatgaggatgaggatgaggatgaagatgaggatgaacggcagttactgggagaatttgagaaggaactggaagggatcctgcttccgtcagaccgagaccggctccgttcggaggtgaaggctggcatggagcgggaactggaaaacatcatccaggagacagagaaagagctggacccagatgggctgaagaaggagtcagagcgggatcgggcaatgctggctctcacatccactctcaacaaactcatcaaaagactggaggaaaaacagagtccagagctggtgaagaagcacaagaaaaagagggttgtccccaaaaagcctcccccatcaccccaacctacagggaaaattgagatcaaaattgtccgcccatgggctgaagggactgaagagggtgcacgttggctgactgatgaggacacgagaaacctcaaggagatcttcttcaatatcttggtgccgggagctgaagaggcccagaaggaacgccagcggcagaaagagctggagagcaattaccgccgggtgtggggctctccaggtggggagggcacaggggacctggacgaatttgacttctgagaccaacactacacttgacccttcacggaatccagactcttcctggactggcttgcctcctccccac

SEQ ID NO: 53 - DNA, 3005 bp, Homo sapiens
  misc_feature: Incyte ID No 7510095CB1
cagcgattgatatttctaatcgtcgtgatccacctccccaggaccttggagccacgtttacaaataggaatagggtacgtgggagggatagaacgtacagccaataaaatcatgtggcgccgatgggcgtgttgaggccgctgcctggcttagggcggaaacagattctctgcataagaaggggaacgaaagatggcggcggaaacgctgctgtccagtttgttaggactgctgcttctgggactcctgttacccgcaagtctgaccggcggtgtcgggagcctgaacctggaggagctgagtgagatgcgttatgggatcgagatcctgccgttgcctgtcatgggagggcagagccaatcttcggacgtggtgattgtctcctctaagtacaaacagcgctatgagtgtcgcctgccagctggagctattcacttccagcgtgaaagggaggaggaaacacctgcttaccaagggcctgggatccctgagttgttgagcccaatgagagatgctccctgcttgctgaagacaaaggactggtggacatatgaattctgttatggacgccacatccagcaataccacatggaagattcagagatcaaaggtgaagtcctctatctcggctactaccaatcagccttcgactgggatgatgaaacagccaaggcctccaagcagcatcgtcttaaacgctaccacagccagacctatggcaatgggtccaagtgcgaccttaatgggaggccccgggaggccgaggttcggttcctctgtgacgagggtgcaggtatctctggggactacatcgatcgcgtggacgagcccttgtcctgctcttatgtgctgaccattcgcactcctcggctctgcccccaccctctcctccggcccccacccagtgctgcaccgcaggccatcctctgtcacccttccctacagcctgaggagtacatggcctacgttcagaggcaagccgactcaaagcagtatggagataaaatcatagaggagctgcaagatctaggcccccaagtgtggagtgagaccaagtctggggtggcaccccaaaagatggcaggtgcgagcccgaccaaggatgacagtaaggactcagatttctggaagatgcttaatgagccagaggaccaggccccaggaggggaggaggtgccggctgaggagcaggacccaagccctgaggcagcagattcagcttctggtgctcccaatgattttcagaacaacgtgcaggtcaaagtcattcgaagccctgcggatttgattcgattcatagaggagctgaaaggtggaacaaaaaaggggaagccaaatataggccaagagcagcctgtggatgatgctgcagaagtccctcagagggaaccagagaaggaaaggggtgatccagaacggcagagagagatggaagaagaggaggatgaggatgaggatgaggatgaagatgaggatgaacggcagttactgggagaatttgagaaggaactggaagggatcctgcttccgtcagaccgagaccggctccgttcggaggtgaaggctggcatggagcgggaactggaaaacatcatccaggagacagagaaagagctggacccagatgggctgaagaaggagtcagagcgggatcgggcaatgctggctctcacatccactctcaacaaactcatcaaaagactggaggaaaaacagagtccagagctggtgaagaagcacaagaaaaagagggttgtccccaaaaagcctcccccatcaccccaacctacagaggaggatcctgagcacagagtccgggtccgggtcaccaagctccgtctcggaggccctaatcaggatctgactgtcctcgagatgaaacgggaaaacccacagctgaaacaaatcgaggggctggtgaaggagctgctggagagggagggactcacagctgcaggtgggccctggagggcggctggacccagtgctgtcggaagggcaagctgccggaagtggaggggctgggaccagtggggcagggggcctgccctgaggctcccaaccatcctctcctctcactgcatactgctcctttccagggaaaattgagatcaaaattgtccgcccatgggctgaagggactgaagagggtgcacgttggctgactgatgaggacacgagaaacctcaaggagatcttcttcaatatcttggtgccgggagctgaagaggcccagaaggaacgccagcggcagaaagagctggagagcaattaccgccgggtgtggggctctccaggtggggagggcacaggggacctggacgaatttgacttctgagaccaacactacacttgacccttcacggaatccagactcttcctggactggcttgcctcctccccacctccccaccctggaacccctgagggccaaacagcagagtggagctgagctgtggacctctcgggcaactctgtgggtgtgggggccctgggtgaatgctgctgcccctgctggcagccaccttgagacctcaccgggcctgtgatatttgctctcctgaactctcactcaatcctcttcctctcctctgtggcttttcctgttattgtcccctaatgataggatattccctgctgcctacctggagattcagtaggatcttttgagtggaggtgggtagagagagcaaggagggcaggacacttagcaggcactgagcaagcaggcccccacctgcccttagtgatgtttggagtcgttttaccctcttctattgaattgccttgggatttccttctcccttntcctgcccaccctgtcccctacaatttgtgcttctgagttgaggagccttcaactctgttgctgaggaaatggtagaatgctgcctatcaacctcagcaaaaatccagcgaaaaaaggtgtgaagcaaccaccaatgtctctggaacaaatcagggttttaaataaacaccttgggcccatcaaaaaaaaaaaaaaa

SEQ ID NO: 54 - DNA, 2906 bp, Homo sapiens
  misc_feature: Incyte ID No 7510096CB1
cagcgattgatatttctaatcgtcgtgatccacctccccaggaccttggagccacgtttacaaataggaatagggtacgtgggagggatagaacgtacagccaataaaatcatgtggcgccgatgggcgtgttgaggccgctgcctggcttagggcggaaacagattctctgcataagaaggggaacgaaagatggcggcggaaacgctgctgtccagtttgttaggactgctgcttctgggactcctgttacccgcaagtctgaccggcggtgtcgggagcctgaacctggaggagctgagtgagatgcgttatgggatcgagatcctgccgttgcctgtcatgggagggcagcctttctttgcttccttgccccccgaccctcccttctttcccagagccaatcttcggacgtggtgattgtctcctctaagtacaaacagcgctatgagtgtcgcctgccagctggagctattcacttccagcgtgaaagggaggaggaaacacctgcttaccaagggcctgggatccctgagttgttgagcccaatgagagatgctccctgcttgctgaagacaaaggactggtggacatatgaattctgttatggacgccacatccagcaataccacatggaagattcagagatcaaaggtgaagtcctctatctcggctactaccaatcagccttcgactgggatgatgaaacagccaaggcctccaagcagcatcgtcttaaacgctaccacagccagacctatggcaatgggtccaagtgcgaccttaatgggaggccccgggaggccgaggttcggttcctctgtgacgagggtgcaggtatctctggggactacatcgatcgcgtggacgagcccttgtcctgctcttatgtgctgaccattcgcactcctcggctctgcccccaccctctcctccggcccccacccagtgctgcaccgcaggccatcctctgtcacccttccctacagcctgaggagtacatggcctacgttcagaggcaagccgtagactcaaagcagtatggagataaaatcatagaggagctgcaagatctaggcccccaagtgtggagtgagaccaagtctggggtggcaccccaaaagatggcaggtgcgagcccgaccaaggatgacagtaaggactcagatttctggaagatgcttaatgagccagaggaccaggccccaggaggggaggaggtgccggctgaggagcaggacccaagccctgaggcagcagattcagcttctggtgctcccaatgattttcagaacaacgtgcaggtcaaagtcattcgaagccctgcggatttgattcgattcatagaggagctgaaaggtggaacaaaaaaggggaagccaaatataggccaagagcagcctgtggatgatgctgcagaagtccctcagagggaaccagagaaggaaaggggtgatccagaacggcagagagagatggaagaagaggaggatgaggatgaggatgaggatgaagatgaggatgaacggcagttactgggagaatttgagaaggaactggaagggatcctgcttccgtcagaccgagaccggctccgttcggaggtgaaggctggcatggagcgggaactggagaacatcatccaggagacagagaaagagctggacccagatgggctgaagaaggagtcagagcgggatcgggcaatgctggctctcacatccactctcaacaaactcatcaaaagactggaggaaaaacagagtccagagctggtgaagaagcacaagaaaaagagggttgtccccaaaaagcctcccccatcaccccaacctacagaggaggatcctgagcacagagtccgggtccgggtcaccaagctccgtctcggaggccctaatcaggatctgactgtcctcgagatgaaacgggaaaacccacagctgaaacaaatcgaggggctggtgaaggagctgctggagagggagggactcacagctgcagggaaaattgagatcaaaattgtccgcccatgggctgaagggactgaagagggtgcacgttggctgactgatgaggacacgagaaacctcaaggagatcttcttcaatatcttggtgccgggagctgaagaggcccagaaggaacgccagcggcagaaagagctggagagcaattaccgccgggtgtggggctctccaggtggggagggcacaggggacctggacgaatttgacttctgagaccaacactacacttgacccttcacggaatccagactcttcctggactggcttgcctcctccccacctccccaccctggaacccctgagggccaaacagcagagtggagctgagctgtggacctctcgggcaactctgtgggtgtgggggccctgggtgaatgctgctgcccctgctggcagccaccttgagacctcaccgggcctgtgatatttgctctcctgaactctcactcaatcctcttcctctcctctgtggcttttcctgttattgtcccctaatgataggatattccctgctgcctacctggagattcagtaggatcttttgagtggaggtgggtagagagagcaaggagggcaggacacttagcaggcactgagcaagcaggcccccacctgcccttagtgatgtttggagtcgttttaccctcttctattgaattgccttgggatttccttctccctttccctgcccaccctgtcccctacaatttgtgcttctgagttgaggagccttcacctctgttgctgaggaaatggtagaatgctgcctatcacctccagcacaatcccagcgaaaaaggtgtgaagcacccaccatgttcttgaacaatcaggtttctaaataaacaccttgggcccatcaaaaaaaaaaaaaaa